(19) United States
(12) Patent Application Publication
(10) Pub. No.: US 2020/0339513 A1
(43) Pub. Date: Oct. 29, 2020

(54) HETEROCYCLIC COMPOUND AND ORGANIC LIGHT EMITTING ELEMENT COMPRISING SAME

(71) Applicant: LT MATERIALS CO., LTD., Yongin-City, Gyeonggi-do (KR)

(72) Inventors: Yu-Jin HEO, Osan-si (KR); Hyun-Ju LA, Hwaseong-si (KR); Won-Jang JEONG, Hwaseong-si (KR); Jin-Seok CHOI, Suwon-si (KR); Dae-Hyuk CHOI, Yongin-si (KR); Joo-Dong LEE, Seongnam-si (KR)

(73) Assignee: LT MATERIALS CO., LTD., Yongin-City, Gyeonggi-Do (KR)

(21) Appl. No.: 16/957,880

(22) PCT Filed: Dec. 24, 2018
(86) PCT No.: PCT/KR2018/016591

§ 371 Date: Jun. 25, 2020

(30) Foreign Application Priority Data
Dec. 26, 2017 (KR) 10-2017-0179900

(51) Int. Cl. C07D 221/18; H01L 51/00; C07D 401/10; C07D 409/10; C07D 409/14; C07D 471/04; C07D 405/14; C07D 401/14; C07D 417/14

(52) U.S. Cl. CPC C07D 221/18; H01L 51/5072; H01L 51/0072; C07D 401/10; H01L 51/0074; C07D 409/10; H01L 51/0054; C07D 409/14; C07D 471/04; C07D 405/14; H01L 51/0073; C07D 401/14; H01L 51/0069; C07D 417/14; H01L 51/0067

(57) ABSTRACT

The present specification relates to a heterocyclic compound represented by Chemical Formula 1, and an organic light emitting device comprising the same.

### TECHNICAL FIELD

This application claims priority to and the benefits of Korean Patent Application No. 10-2017-0179900, filed with the Korean Intellectual Property Office on Dec. 26, 2017, the entire contents of which are incorporated herein by reference.

The present specification relates to a heterocyclic compound and an organic light emitting device comprising the same.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

U.S. Pat. No. 4,356,429

### DISCLOSURE

### Technical Problem

The present disclosure is directed to providing a novel heterocyclic compound and an organic light emitting device comprising the same.

### Technical Solution

One embodiment of the present application provides a heterocyclic compound represented by the following Chemical Formula 1.

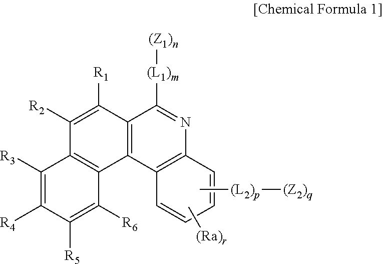

In Chemical Formula 1,

R₁ to R₆ and Ra are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other bond to each other to form a substituted or unsubstituted aliphatic or aromatic hydrocarbon ring,

L₁ is a substituted or unsubstituted arylene group; or a substituted or unsubstituted heteroarylene group,

L₂ is a direct bond; a substituted or unsubstituted arylene group; or a substituted or unsubstituted heteroarylene group,

Z₁ is selected from the group consisting of hydrogen; deuterium; a halogen group; —CN; a substituted or unsubstituted alkyl group; a substituted or unsubstituted aryl group; a substituted or unsubstituted heteroaryl group; —SiRR′R″ and —P(═O)RR′,

Z₂ is selected from the group consisting of deuterium; a halogen group; —CN; a substituted or unsubstituted alkyl group; a substituted or unsubstituted aryl group; a substituted or unsubstituted heteroaryl group; —SiRR′R″ and —P(═O)RR′,

when Z₁ is hydrogen, L₂ is a substituted or unsubstituted arylene group, and Z₂ is a substituted or unsubstituted heteroaryl group,

R, R′ and R″ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group,

p and m are an integer of 1 to 4,

q and n are an integer of 1 to 5, and

r is an integer of 0 to 3.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound according to one embodiment of the present application.

### Advantageous Effects

A compound described in the present specification can be used as an organic material layer material of an organic light emitting device. The compound is capable of performing a role of a hole injection material, a hole transfer material, a light emitting material, an electron transfer material, an electron injection material and the like in the organic light emitting device. Particularly, the compound can be used as an electron transfer layer material or a charge generation layer material of the organic light emitting device.

Specifically, when using the compound represented by Chemical Formula 1 in the organic material layer, a driving voltage is lowered and light efficiency is enhanced in the device, and device lifetime properties can be enhanced by thermal stability of the compound.

### DESCRIPTION OF DRAWINGS

FIG. 1 to FIG. 4 are diagrams each schematically illustrating a lamination structure of an organic light emitting device according to one embodiment of the present application.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present application will be described in detail.

The term “substituted” means a hydrogen atom bonding to a carbon atom of a compound is changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, the halogen may be fluorine, chlorine, bromine or iodine.

In the present specification, the alkyl group comprises linear or branched having 1 to 60 carbon atoms, and may be further substituted with other substituents. The number of carbon atoms of the alkyl group may be from 1 to 60, specifically from 1 to 40 and more specifically from 1 to 20. Specific examples thereof may comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethylbutyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group comprises linear or branched having 2 to 60 carbon atoms, and may be further substituted with other substituents. The number of carbon atoms of the alkenyl group may be from 2 to 60, specifically from 2 to 40 and more specifically from 2 to 20. Specific examples thereof may comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group comprises linear or branched having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples thereof may comprise methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the cycloalkyl group comprises monocyclic or multicyclic having 3 to 60 carbon atoms, and may be further substituted with other substituents. Herein, the multicyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group comprises O, S, Se, N or Si as a heteroatom, comprises monocyclic or multicyclic having 2 to 60 carbon atoms, and may be further substituted with other substituents. Herein, the multicyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group comprises monocyclic or multicyclic having 6 to 60 carbon atoms, and may be further substituted with other substituents. Herein, the multicyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group comprises a spiro group. The number of carbon atoms of the aryl group may be from 6 to 60, specifically from 6 to 40 and more specifically from 6 to 25. Specific examples of the aryl group may comprise a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring thereof, and the like, but are not limited thereto.

In the present specification, the silyl group is a substituent comprising Si, having the Si atom directly linked as a radical, and is represented by —SiR₁₀₄R₁₀₅R₁₀₆. R₁₀₄ to R₁₀₆ are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted,

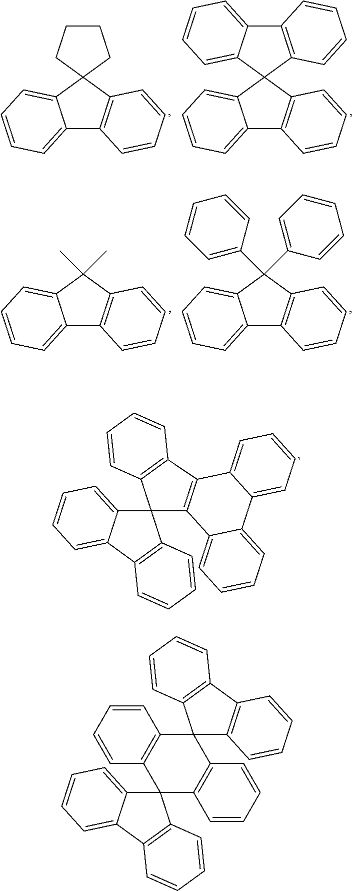

and the like may be included. However, the structure is not limited thereto.

In the present specification, the heteroaryl group comprises O, S, Se, N or Si as a heteroatom, comprises monocyclic or multicyclic having 2 to 60 carbon atoms, and may be further substituted with other substituents. Herein, the multicyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a qninozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiazinyl group, a phthalazinyl group, a naphthylidinyl group, a phenanthrolinyl group, an imidazo[1,2-a]pyridinyl group, a benzo[c][1,2,5]thiadiazolyl group, a 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. Descriptions on the aryl group provided above may be applied thereto except for each being a divalent. In addition, the heteroarylene group means the heteroaryl group having two bonding sites, that is, a divalent group. Descriptions on the heteroaryl group provided above may be applied thereto except for each being a divalent.

In the present specification, specific examples of the phosphine oxide group may comprise a diphenylphosphine oxide group, a dinaphthylphosphine oxide group and the like, but are not limited thereto.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present specification, the term “substituted” means a hydrogen atom bonding to a carbon atom of a compound is changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; P(═O)RR′; C1 to C20 alkylamine; C6 to C60 monocyclic or polycyclic arylamine; and C2 to C60 monocyclic or polycyclic heteroarylamine, or being unsubstituted, or being substituted with a substituent linking two or more substituents selected from among the substituents illustrated above, or being unsubstituted.

One embodiment of the present application provides a heterocyclic compound represented by the following Chemical Formula 1.

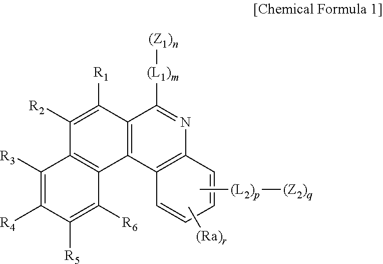

In Chemical Formula 1,

R₁ to R₆ and Ra are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other bond to each other to form a substituted or unsubstituted aliphatic or aromatic hydrocarbon ring,

L₁ is a substituted or unsubstituted arylene group; or a substituted or unsubstituted heteroarylene group,

L₂ is a direct bond; a substituted or unsubstituted arylene group; or a substituted or unsubstituted heteroarylene group,

Z₁ is selected from the group consisting of hydrogen; deuterium; a halogen group; —CN; a substituted or unsubstituted alkyl group; a substituted or unsubstituted aryl group; a substituted or unsubstituted heteroaryl group; —SiRR′R″ and —P(═O)RR′,

Z₂ is selected from the group consisting of deuterium; a halogen group; —CN; a substituted or unsubstituted alkyl group; a substituted or unsubstituted aryl group; a substituted or unsubstituted heteroaryl group; —SiRR′R″ and —P(═O)RR′,

when Z₁ is hydrogen, L₂ is a substituted or unsubstituted arylene group, and Z₂ is a substituted or unsubstituted heteroaryl group,

R, R′ and R″ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group,

p and m are an integer of 1 to 4,

q and n are an integer of 1 to 5, and

r is an integer of 0 to 3.

By having substituents of -(L₁)m-(Z₁)n and -(L₂)p-(Z₂)q in the core structure, Chemical Formula 1 has both p-type and n-type substituents in one molecule, and the p-type substituent stabilizes an unstable state of the core caused by electrons during electron injection, and by adjusting the p-type substituent and the n-type substituent, the energy level may be adjusted to effectively transfer electrons to a light emitting layer.

In one embodiment of the present application, R₁ to R₆ and Ra of Chemical Formula 1 are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other may bond to each other to form a substituted or unsubstituted aliphatic or aromatic hydrocarbon ring.

In another embodiment, R₁ to R₆ and Ra of Chemical Formula are the same as or different from each other, and each independently selected from the group consisting of hydrogen; a substituted or unsubstituted C1 to C60 aryl group; and a substituted or unsubstituted C2 to C60 heteroaryl group, or two or more groups adjacent to each other may bond to each other to form a substituted or unsubstituted aromatic hydrocarbon ring.

In another embodiment, R₁ to R₆ and Ra of Chemical Formula are the same as or different from each other, and each independently selected from the group consisting of hydrogen; a substituted or unsubstituted C1 to C30 aryl group; and a substituted or unsubstituted C2 to C30 heteroaryl group, or two or more groups adjacent to each other may bond to each other to form a substituted or unsubstituted aromatic hydrocarbon ring.

In another embodiment, R₁ to R₆ and Ra of Chemical Formula 1 are the same as or different from each other, and each independently hydrogen, or two or more groups adjacent to each other may bond to each other to form a substituted or unsubstituted C2 to C30 aromatic hydrocarbon ring.

In another embodiment, R₁ to R₆ and Ra of Chemical Formula 1 are the same as or different from each other, and may be each independently hydrogen.

In one embodiment of the present application, L₁ of Chemical Formula 1 may be a substituted or unsubstituted arylene group; or a substituted or unsubstituted heteroarylene group.

In another embodiment, L₁ of Chemical Formula 1 may be a substituted or unsubstituted C6 to C60 arylene group; or a substituted or unsubstituted C2 to C60 heteroarylene group.

In another embodiment, L₁ of Chemical Formula 1 may be a substituted or unsubstituted C6 to C40 arylene group; or a substituted or unsubstituted C2 to C40 heteroarylene group.

In another embodiment, L₁ of Chemical Formula 1 may be a substituted or unsubstituted C6 to C40 arylene group.

In another embodiment, L₁ of Chemical Formula 1 may be a C6 to C40 tricyclic or lower arylene group.

In another embodiment, L₁ of Chemical Formula 1 may be a C6 to C20 tricyclic or lower arylene group.

In another embodiment, L₁ of Chemical Formula 1 may be a phenylene group; a biphenylene group; a phenanthrene group; or a naphthalene group.

In one embodiment of the present application, L₂ of Chemical Formula 1 may be a direct bond; a substituted or unsubstituted arylene group; or a substituted or unsubstituted heteroarylene group.

In another embodiment, L₂ of Chemical Formula 1 may be a direct bond; a substituted or unsubstituted C6 to C60 arylene group; or a substituted or unsubstituted C2 to C60 heteroarylene group.

In another embodiment, L₂ of Chemical Formula 1 may be a direct bond; a substituted or unsubstituted C6 to C30 arylene group; or a substituted or unsubstituted C2 to C30 heteroarylene group.

In another embodiment, L₂ of Chemical Formula 1 may be a direct bond; or a substituted or unsubstituted C6 to C30 arylene group.

In another embodiment, L₂ of Chemical Formula 1 may be a direct bond; or a C6 to C30 monocyclic arylene group.

In another embodiment, L₂ of Chemical Formula 1 may be a direct bond; a phenylene group; or a biphenylene group.

In one embodiment of the present application, Z₁ of Chemical Formula 1 may be selected from the group consisting of hydrogen; deuterium; a halogen group; —CN; a substituted or unsubstituted alkyl group; a substituted or unsubstituted aryl group; a substituted or unsubstituted heteroaryl group; SiRR′R″ and —P(═O)RR′.

In another embodiment, Z₁ of Chemical Formula 1 may be selected from the group consisting of hydrogen; a substituted or unsubstituted aryl group; a substituted or unsubstituted heteroaryl group; and —P(═O)RR′.

In another embodiment, Z₁ of Chemical Formula 1 may be selected from the group consisting of hydrogen; a substituted or unsubstituted C6 to C60 aryl group; a substituted or unsubstituted C2 to C60 heteroaryl group; and —P(═O)RR′.

In another embodiment, Z₁ of Chemical Formula 1 may be selected from the group consisting of hydrogen; a substituted or unsubstituted C6 to C40 aryl group; a substituted or unsubstituted C2 to C40 heteroaryl group; and —P(═O)RR′.

In another embodiment, Z₁ of Chemical Formula 1 may be selected from the group consisting of hydrogen; a substituted or unsubstituted C6 to C20 aryl group; a substituted or unsubstituted C2 to C20 heteroaryl group; and —P(═O)RR′.

In another embodiment, Z₁ of Chemical Formula 1 may be selected from the group consisting of hydrogen; a substituted or unsubstituted C6 to C20 aryl group; a substituted or unsubstituted C2 to C20 heteroaryl group; and —P(═O)RR′.

In another embodiment, Z₁ of Chemical Formula 1 may be selected from the group consisting of hydrogen; a C6 to C20 aryl group unsubstituted or substituted with an alkyl group; a C2 to C20 heteroaryl group unsubstituted or substituted with one or more substituents selected from the group consisting of an alkyl group, an aryl group and a heteroaryl group; and —P(═O)RR′.

In another embodiment, Z₁ of Chemical Formula 1 may be selected from the group consisting of hydrogen; a C6 to C20 aryl group unsubstituted or substituted with a C1 to C20 alkyl group; a C2 to C20 heteroaryl group unsubstituted or substituted with one or more substituents selected from the group consisting of a C1 to C20 alkyl group, a C6 to C20 aryl group and a C2 to C20 heteroaryl group; and —P(═O)RR′.

In another embodiment, Z₁ of Chemical Formula 1 may be hydrogen; or —P(═O)RR′.

In another embodiment, Z₁ of Chemical Formula 1 may be a phenyl group unsubstituted or substituted with a methyl group; a naphthyl group; a triphenylenyl group; or a phenanthrenyl group.

In another embodiment, Z₁ of Chemical Formula 1 may be a pyridine group unsubstituted or substituted with one or more substituents selected from the group consisting of a phenyl group and a pyridine group; a pyrimidine group unsubstituted or substituted with one or more substituents selected from the group consisting of a phenyl group, a biphenyl group, a naphthyl group and a phenanthrenyl group; a triazine group unsubstituted or substituted with one or more substituents selected from the group consisting of a phenyl group, a biphenyl group, a naphthyl group and a phenanthrenyl group; a carbazole group; a dibenzothiophene group; a benzothiazole group unsubstituted or substituted with a phenyl group; a phenanthroline group unsubstituted or substituted with a phenyl group; an imidazo[1,2-a]pyridine group unsubstituted or substituted with a phenyl group; a benzimidazole group unsubstituted or substituted with an ethyl group or a phenyl group; or a quinazoline group unsubstituted or substituted with one or more substituents selected from the group consisting of a phenyl group, a biphenyl group and a naphthyl group.

In one embodiment of the present application, Z₁ of Chemical Formula 1 may be may be unsubstituted or substituted again with a C1 to C20 alkyl group.

In one embodiment of the present application, Z₂ of Chemical Formula 1 may be selected from the group consisting of deuterium; a halogen group; —CN; a substituted or unsubstituted alkyl group; a substituted or unsubstituted aryl group; a substituted or unsubstituted heteroaryl group; —SiRR′R″ and —P(═O)RR′.

In another embodiment, Z₂ of Chemical Formula 1 may be —CN; a substituted or unsubstituted C1 to C60 alkyl group; a substituted or unsubstituted C6 to C60 aryl group; or a substituted or unsubstituted C2 to C60 heteroaryl group.

In another embodiment, Z₂ of Chemical Formula 1 may be —CN; a substituted or unsubstituted C1 to C40 alkyl group; a substituted or unsubstituted C6 to C40 aryl group; or a substituted or unsubstituted C2 to C40 heteroaryl group.

In another embodiment, Z₂ of Chemical Formula 1 may be —CN; a substituted or unsubstituted C6 to C20 aryl group; or a substituted or unsubstituted C2 to C20 heteroaryl group.

In another embodiment, Z₂ of Chemical Formula 1 may be —CN; a C6 to C20 aryl group unsubstituted or substituted with a heteroaryl group; or a C2 to C20 heteroaryl group unsubstituted or substituted with one or more substituents selected from the group consisting of an alkyl group and an aryl group.

In another embodiment, Z₂ of Chemical Formula 1 may be —CN; a C6 to C20 aryl group unsubstituted or substituted with a C2 to C20 heteroaryl group; or a C2 to C20 heteroaryl group unsubstituted or substituted with one or more substituents selected from the group consisting of a C1 to C20 alkyl group and a C6 to C20 aryl group.

In another embodiment, Z₂ of Chemical Formula 1 may be —CN.

In another embodiment, Z₂ of Chemical Formula 1 may be a phenyl group unsubstituted or substituted with a carbazole group; a biphenyl group; a naphthyl group; a phenanthrenyl group; a triphenylenyl group; or a pyrene group.

In another embodiment, Z₂ of Chemical Formula 1 may be a pyridine group unsubstituted or substituted with a phenyl group; a pyrimidine group unsubstituted or substituted with one or more substituents selected from the group consisting of a phenyl group and a biphenyl group; a triazine group unsubstituted or substituted with one or more substituents selected from the group consisting of a phenyl group and a biphenyl group; a carbazole group; a dibenzothiophene group; a dibenzofuran group; or a benzimidazole group unsubstituted or substituted with an ethyl group.

In one embodiment of the present application, R, R′ and R″ are the same as or different from each other, and may be each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group.

In another embodiment, R, R′ and R″ are the same as or different from each other, and may be each independently a substituted or unsubstituted aryl group.

In another embodiment, R, R′ and R″ are the same as or different from each other, and may be each independently a substituted or unsubstituted C6 to C60 aryl group.

In another embodiment, R, R′ and R″ are the same as or different from each other, and may be each independently a substituted or unsubstituted C6 to C40 aryl group.

In another embodiment, R, R′ and R″ are the same as or different from each other, and may be each independently a C6 to C40 aryl group.

In another embodiment, R, R′ and R″ are the same as or different from each other, and may be each independently a phenyl group.

In one embodiment of the present application, when Z₁ is hydrogen, L₂ is a substituted or unsubstituted arylene group, and Z₂ may be a substituted or unsubstituted heteroaryl group.

In another embodiment, when Z₁ is hydrogen, L₂ is a substituted or unsubstituted C6 to C40 arylene group, and Z₂ may be a substituted or unsubstituted C2 to C40 heteroaryl group.

In another embodiment, when Z₁ is hydrogen, L₂ is a C6 to C40 monocyclic arylene group, and Z₂ may be an N-containing heteroaryl group unsubstituted or substituted with a C6 to C40 aryl group.

In another embodiment, when Z₁ is hydrogen, L₂ is a C6 to C20 monocyclic arylene group, and Z₂ may be a heteroaryl group containing at least two or more Ns unsubstituted or substituted with a C6 to C40 aryl group.

In another embodiment, when Z₁ is hydrogen, L₂ is a phenylene group or a biphenylene group, and Z₂ may be a pyrimidine group unsubstituted or substituted with a phenyl group; or a triazine group unsubstituted or substituted with a phenyl group.

In the compound according to one embodiment of the present application, an electron-deficient substituent and an aryl or acene-based substituent are combined, and therefore, electrons are readily supplied to the electron-deficient substituent from an electron injection layer, and by the acryl or acene-based substituent stabilizing the molecule itself and transferring the supplied electrons to a light emitting layer, excellent efficiency is obtained compared to compounds having different structures.

In the heterocyclic compound provided in one embodiment of the present application, Chemical Formula 1 is represented by any one of the following Chemical Formulae 2 to 7.

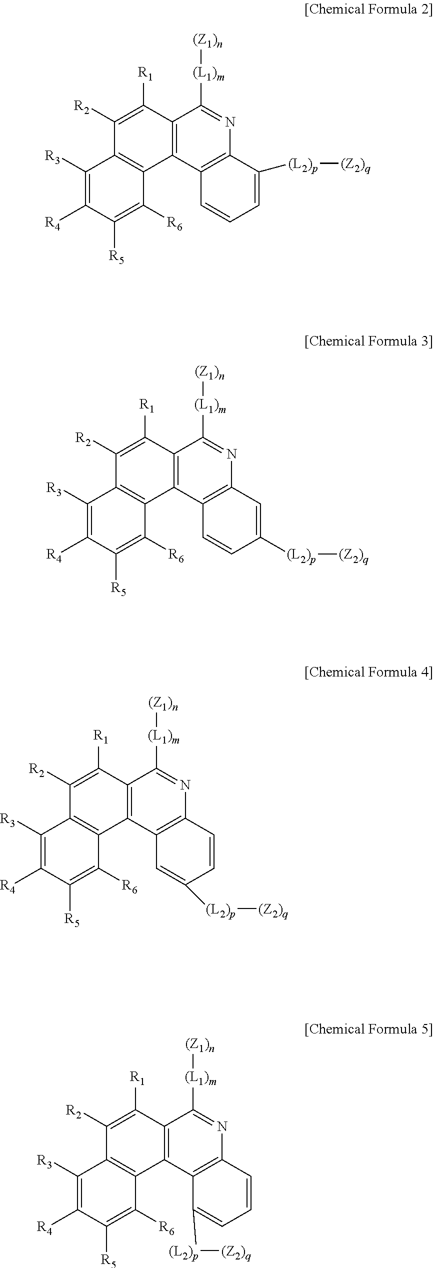

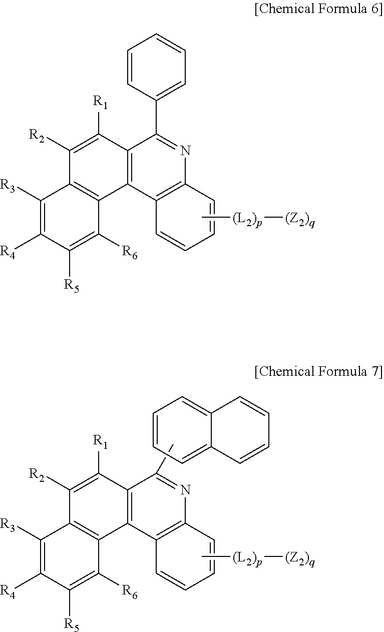

In Chemical Formulae 2 to 7,

R₁ to R₆, L₁, L₂, Z₁, Z₂, m, n, p and q have the same definitions as in Chemical Formula 1.

In the heterocyclic compound provided in one embodiment of the present application, Chemical Formula 1 is represented by any one of the following compounds.

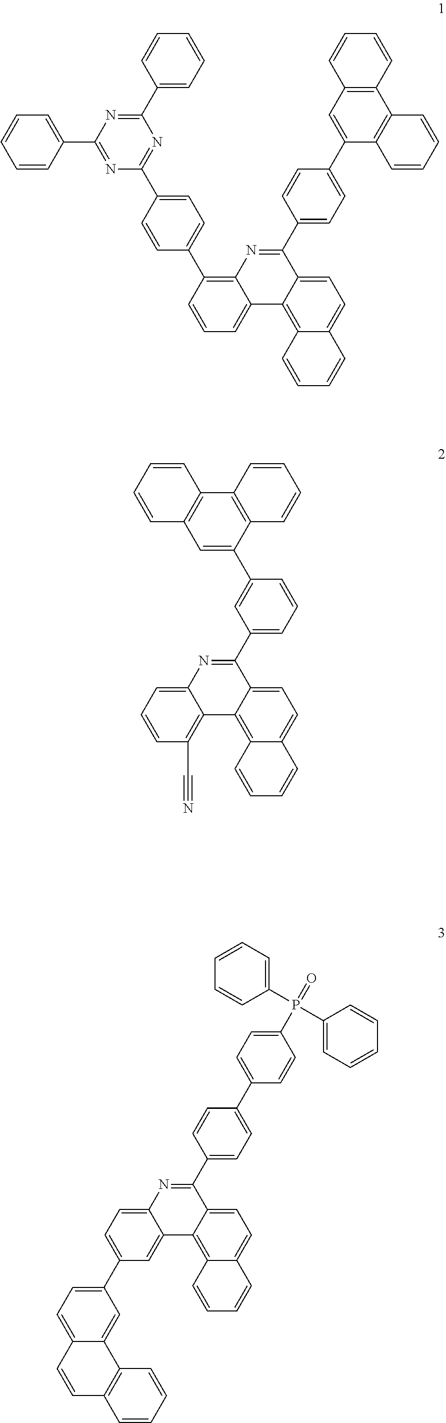 [SMILES: c1ccc(-c2nc(-c3ccccc3)nc(-c3ccc(-c4cccc5c4nc(-c4ccc(-c6cc7ccccc7c7ccccc67)cc4)c4ccc6ccccc6c45)cc3)n2)cc1 ; N#Cc1cccc2nc(-c3cccc(-c4cc5ccccc5c5ccccc45)c3)c3ccc4ccccc4c3c12 ; O=P(c1ccccc1)(c1ccccc1)c1ccc(-c2ccc(-c3nc4ccc(-c5ccc6ccc7ccccc7c6c5)cc4c4c3ccc3ccccc34)cc2)cc1]

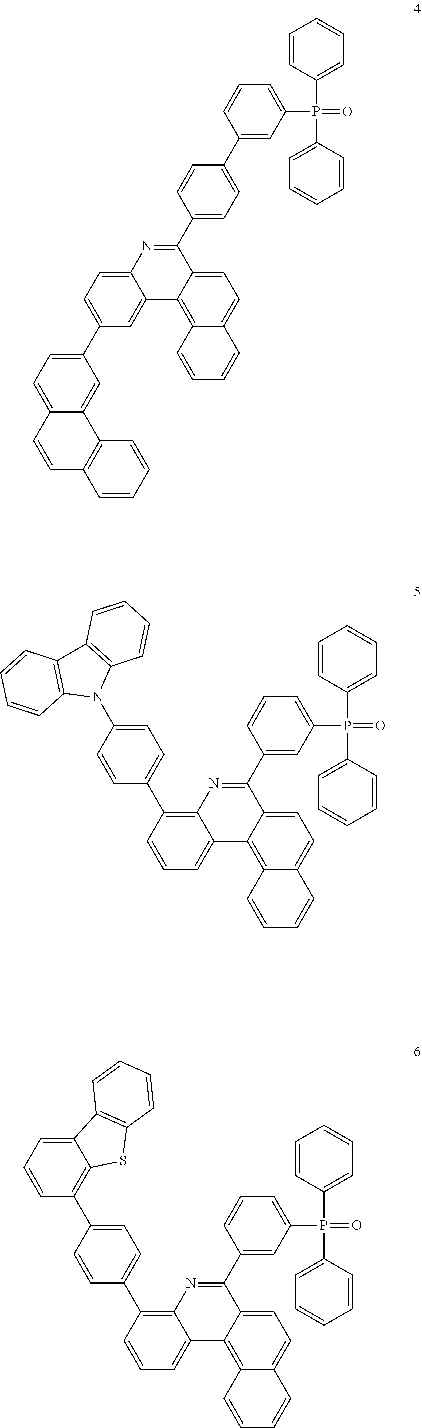 [SMILES: O=P(c1ccccc1)(c1ccccc1)c1cccc(-c2ccc(-c3nc4ccc(-c5ccc6ccc7ccccc7c6c5)cc4c4c3ccc3ccccc34)cc2)c1 ; O=P(c1ccccc1)(c1ccccc1)c1cccc(-c2nc3c(-c4ccc(-n5c6ccccc6c6ccccc65)cc4)cccc3c3c2ccc2ccccc23)c1 ; O=P(c1ccccc1)(c1ccccc1)c1cccc(-c2nc3c(-c4ccc(-c5cccc6c5sc5ccccc56)cc4)cccc3c3c2ccc2ccccc23)c1]

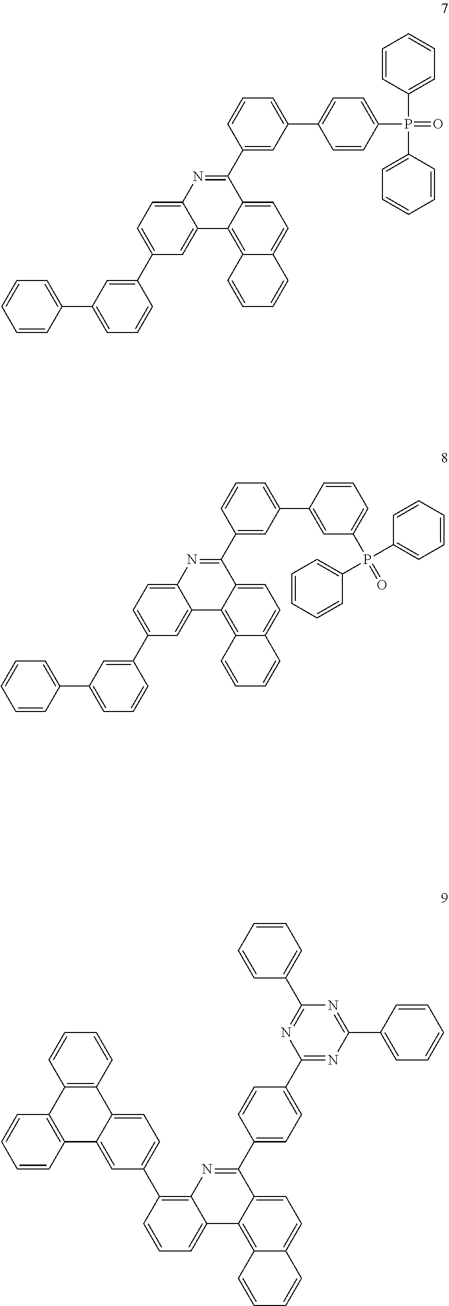 [SMILES: O=P(c1ccccc1)(c1ccccc1)c1ccc(-c2cccc(-c3nc4ccc(-c5cccc(-c6ccccc6)c5)cc4c4c3ccc3ccccc34)c2)cc1 ; O=P(c1ccccc1)(c1ccccc1)c1cccc(-c2cccc(-c3nc4ccc(-c5cccc(-c6ccccc6)c5)cc4c4c3ccc3ccccc34)c2)c1 ; c1ccc(-c2nc(-c3ccccc3)nc(-c3ccc(-c4nc5c(-c6ccc7c8ccccc8c8ccccc8c7c6)cccc5c5c4ccc4ccccc45)cc3)n2)cc1]

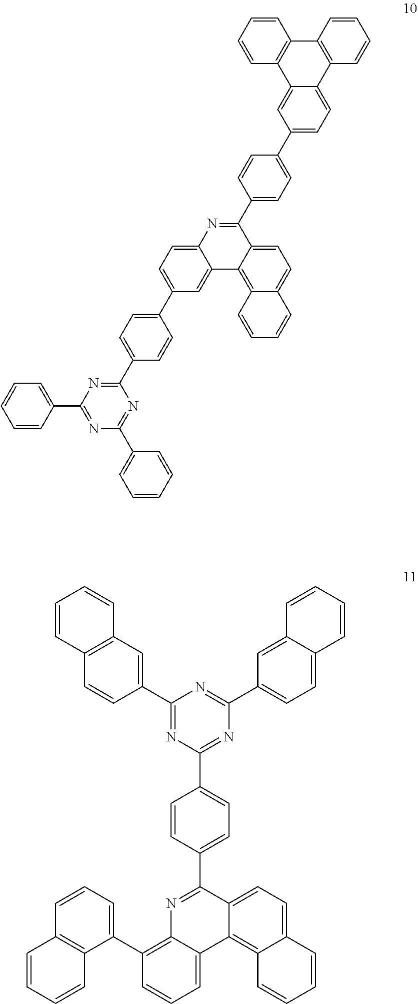 [SMILES: c1ccc(-c2nc(-c3ccccc3)nc(-c3ccc(-c4ccc5nc(-c6ccc(-c7ccc8c9ccccc9c9ccccc9c8c7)cc6)c6ccc7ccccc7c6c5c4)cc3)n2)cc1 ; c1ccc2cc(-c3nc(-c4ccc(-c5nc6c(-c7cccc8ccccc78)cccc6c6c5ccc5ccccc56)cc4)nc(-c4ccc5ccccc5c4)n3)ccc2c1]

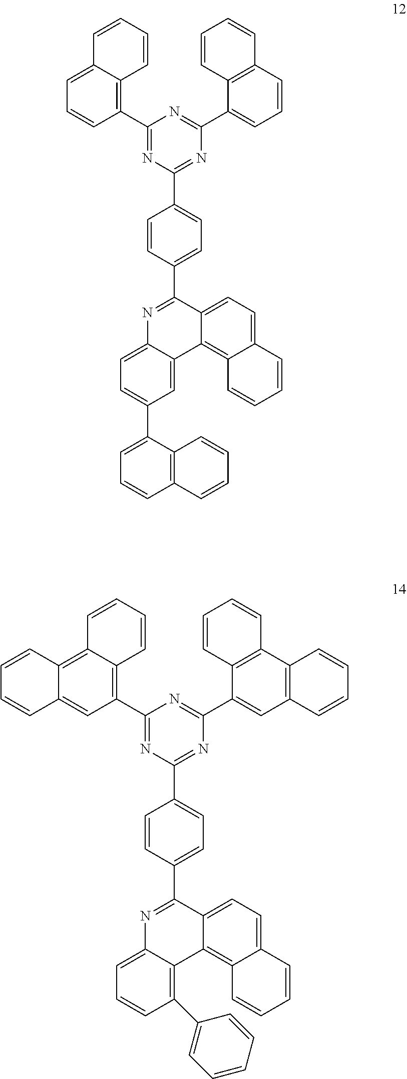 [SMILES: c1ccc2c(-c3ccc4nc(-c5ccc(-c6nc(-c7cccc8ccccc78)nc(-c7cccc8ccccc78)n6)cc5)c5ccc6ccccc6c5c4c3)cccc2c1 ; c1ccc(-c2cccc3nc(-c4ccc(-c5nc(-c6cc7ccccc7c7ccccc67)nc(-c6cc7ccccc7c7ccccc67)n5)cc4)c4ccc5ccccc5c4c23)cc1]

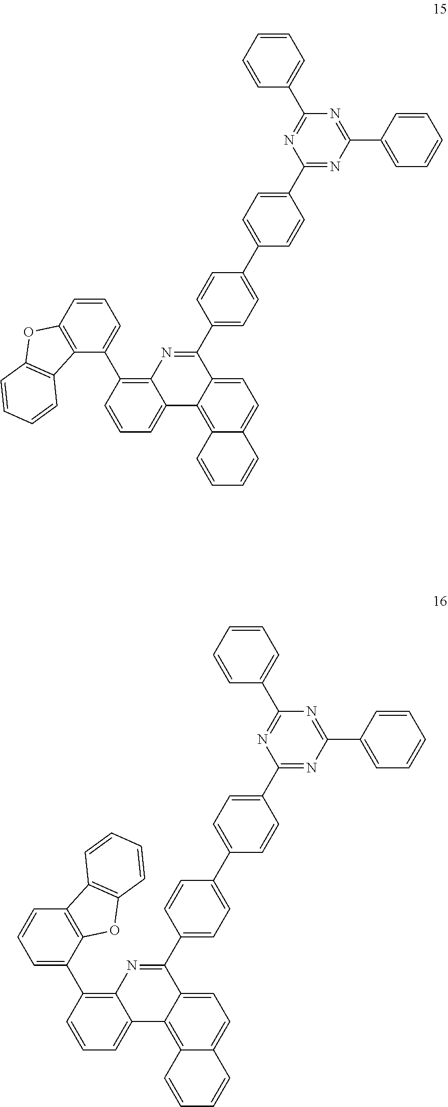 [SMILES: c1ccc(-c2nc(-c3ccccc3)nc(-c3ccc(-c4ccc(-c5nc6c(-c7cccc8oc9ccccc9c78)cccc6c6c5ccc5ccccc56)cc4)cc3)n2)cc1 ; c1ccc(-c2nc(-c3ccccc3)nc(-c3ccc(-c4ccc(-c5nc6c(-c7cccc8c7oc7ccccc78)cccc6c6c5ccc5ccccc56)cc4)cc3)n2)cc1]

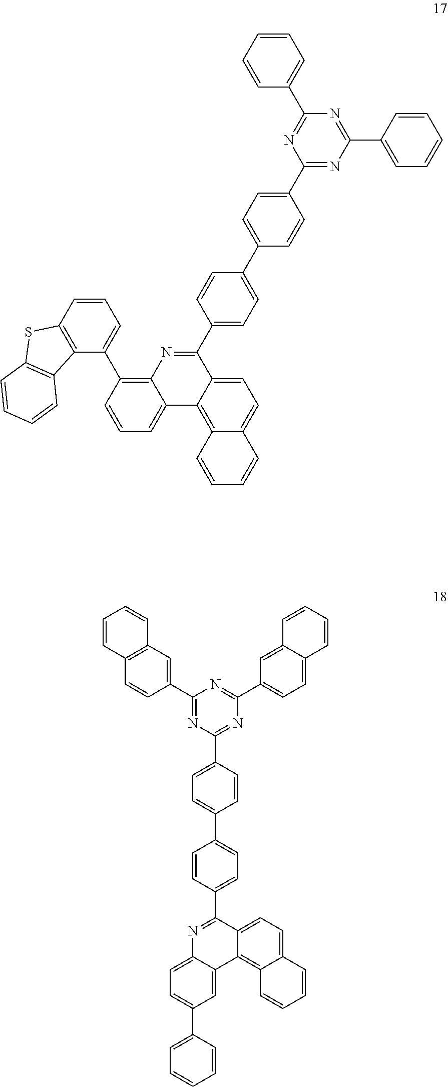 [SMILES: c1ccc(-c2nc(-c3ccccc3)nc(-c3ccc(-c4ccc(-c5nc6c(-c7cccc8sc9ccccc9c78)cccc6c6c5ccc5ccccc56)cc4)cc3)n2)cc1 ; c1ccc(-c2ccc3nc(-c4ccc(-c5ccc(-c6nc(-c7ccc8ccccc8c7)nc(-c7ccc8ccccc8c7)n6)cc5)cc4)c4ccc5ccccc5c4c3c2)cc1]

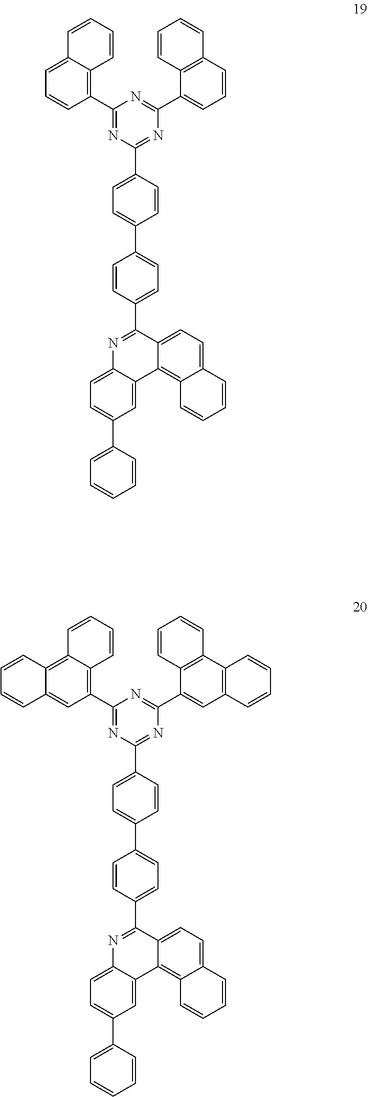 [SMILES: c1ccc(-c2ccc3nc(-c4ccc(-c5ccc(-c6nc(-c7cccc8ccccc78)nc(-c7cccc8ccccc78)n6)cc5)cc4)c4ccc5ccccc5c4c3c2)cc1 ; c1ccc(-c2ccc3nc(-c4ccc(-c5ccc(-c6nc(-c7cc8ccccc8c8ccccc78)nc(-c7cc8ccccc8c8ccccc78)n6)cc5)cc4)c4ccc5ccccc5c4c3c2)cc1]

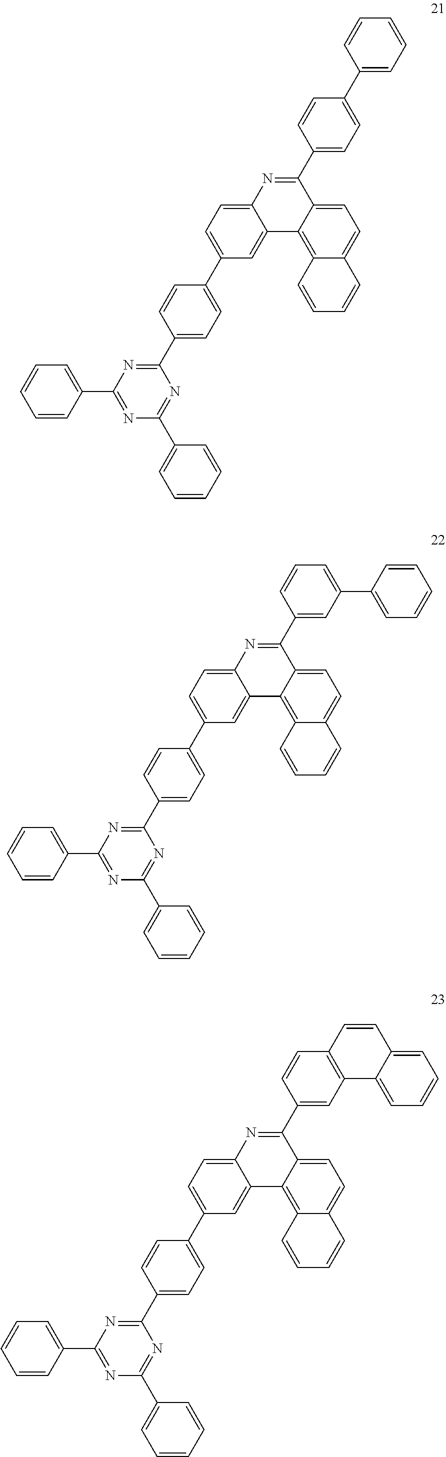 [SMILES: c1ccc(-c2ccc(-c3nc4ccc(-c5ccc(-c6nc(-c7ccccc7)nc(-c7ccccc7)n6)cc5)cc4c4c3ccc3ccccc34)cc2)cc1 ; c1ccc(-c2cccc(-c3nc4ccc(-c5ccc(-c6nc(-c7ccccc7)nc(-c7ccccc7)n6)cc5)cc4c4c3ccc3ccccc34)c2)cc1 ; c1ccc(-c2nc(-c3ccccc3)nc(-c3ccc(-c4ccc5nc(-c6ccc7ccc8ccccc8c7c6)c6ccc7ccccc7c6c5c4)cc3)n2)cc1]

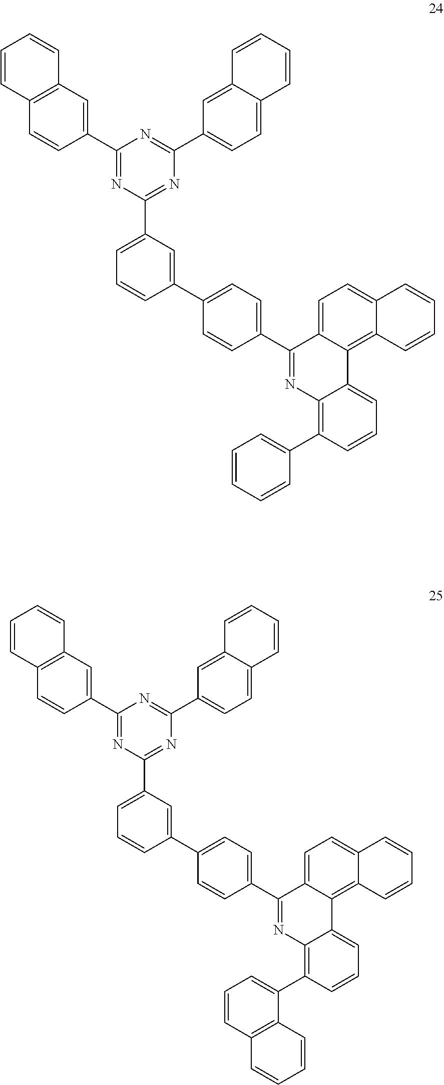 [SMILES: c1ccc(-c2cccc3c2nc(-c2ccc(-c4cccc(-c5nc(-c6ccc7ccccc7c6)nc(-c6ccc7ccccc7c6)n5)c4)cc2)c2ccc4ccccc4c23)cc1 ; c1cc(-c2ccc(-c3nc4c(-c5cccc6ccccc56)cccc4c4c3ccc3ccccc34)cc2)cc(-c2nc(-c3ccc4ccccc4c3)nc(-c3ccc4ccccc4c3)n2)c1]

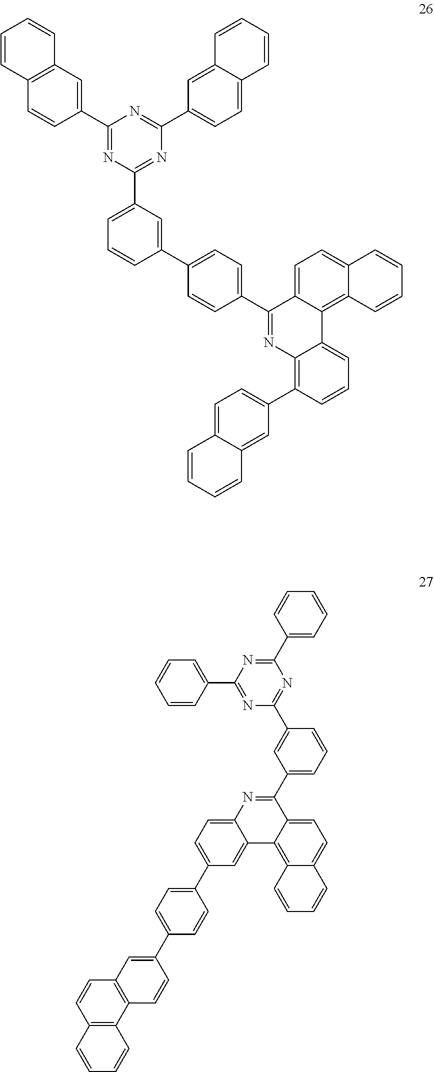 [SMILES: c1cc(-c2ccc(-c3nc4c(-c5ccc6ccccc6c5)cccc4c4c3ccc3ccccc34)cc2)cc(-c2nc(-c3ccc4ccccc4c3)nc(-c3ccc4ccccc4c3)n2)c1 ; c1ccc(-c2nc(-c3ccccc3)nc(-c3cccc(-c4nc5ccc(-c6ccc(-c7ccc8c(ccc9ccccc98)c7)cc6)cc5c5c4ccc4ccccc45)c3)n2)cc1]

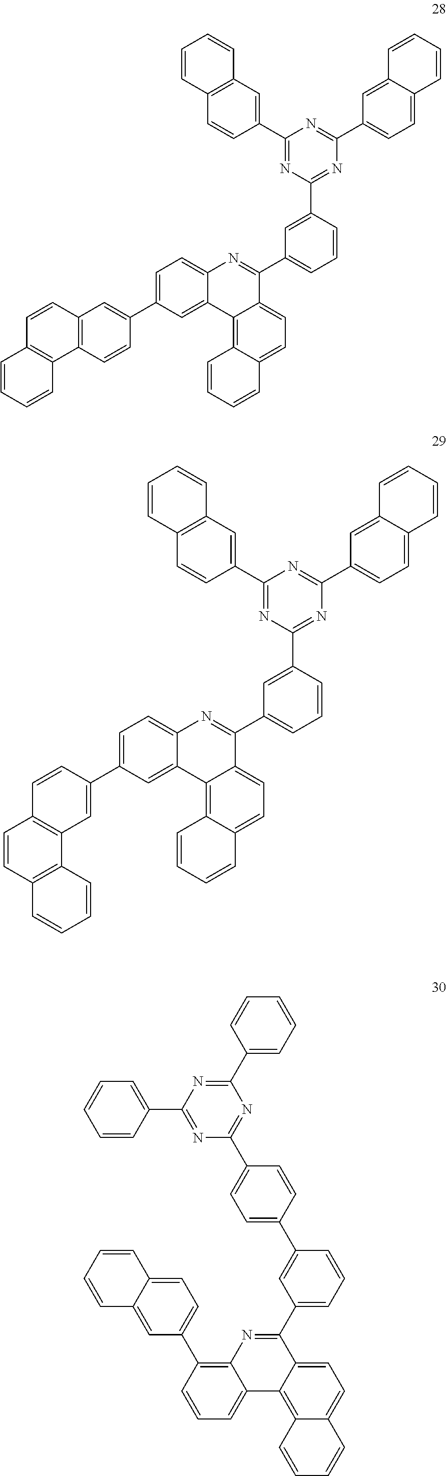 [SMILES: c1cc(-c2nc(-c3ccc4ccccc4c3)nc(-c3ccc4ccccc4c3)n2)cc(-c2nc3ccc(-c4ccc5c(ccc6ccccc65)c4)cc3c3c2ccc2ccccc23)c1 ; c1cc(-c2nc(-c3ccc4ccccc4c3)nc(-c3ccc4ccccc4c3)n2)cc(-c2nc3ccc(-c4ccc5ccc6ccccc6c5c4)cc3c3c2ccc2ccccc23)c1 ; c1ccc(-c2nc(-c3ccccc3)nc(-c3ccc(-c4cccc(-c5nc6c(-c7ccc8ccccc8c7)cccc6c6c5ccc5ccccc56)c4)cc3)n2)cc1]

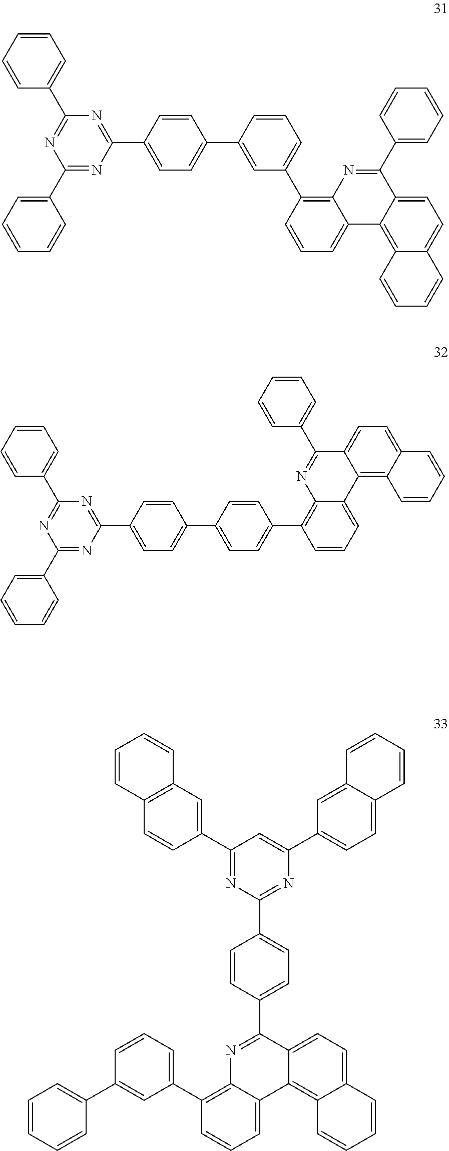 [SMILES: c1ccc(-c2nc(-c3ccccc3)nc(-c3ccc(-c4cccc(-c5cccc6c5nc(-c5ccccc5)c5ccc7ccccc7c56)c4)cc3)n2)cc1 ; c1ccc(-c2nc(-c3ccccc3)nc(-c3ccc(-c4ccc(-c5cccc6c5nc(-c5ccccc5)c5ccc7ccccc7c56)cc4)cc3)n2)cc1 ; c1ccc(-c2cccc(-c3cccc4c3nc(-c3ccc(-c5nc(-c6ccc7ccccc7c6)cc(-c6ccc7ccccc7c6)n5)cc3)c3ccc5ccccc5c34)c2)cc1]

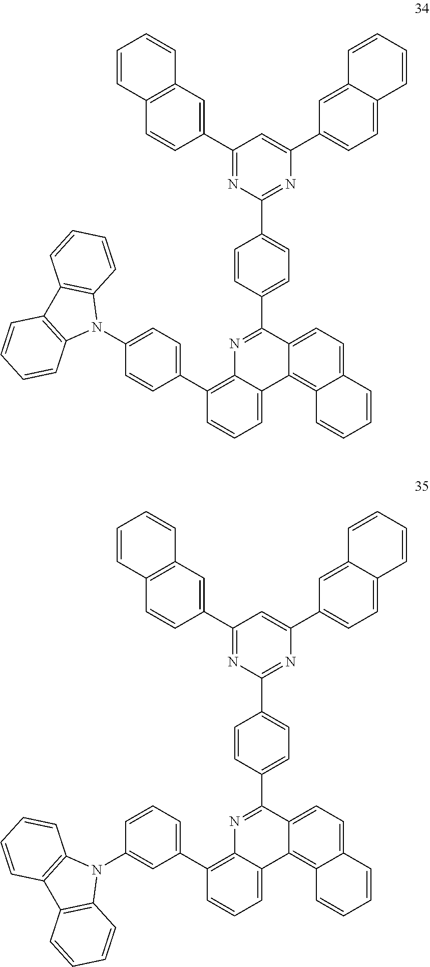 [SMILES: c1ccc2cc(-c3cc(-c4ccc5ccccc5c4)nc(-c4ccc(-c5nc6c(-c7ccc(-n8c9ccccc9c9ccccc98)cc7)cccc6c6c5ccc5ccccc56)cc4)n3)ccc2c1 ; c1cc(-c2cccc3c2nc(-c2ccc(-c4nc(-c5ccc6ccccc6c5)cc(-c5ccc6ccccc6c5)n4)cc2)c2ccc4ccccc4c23)cc(-n2c3ccccc3c3ccccc32)c1]

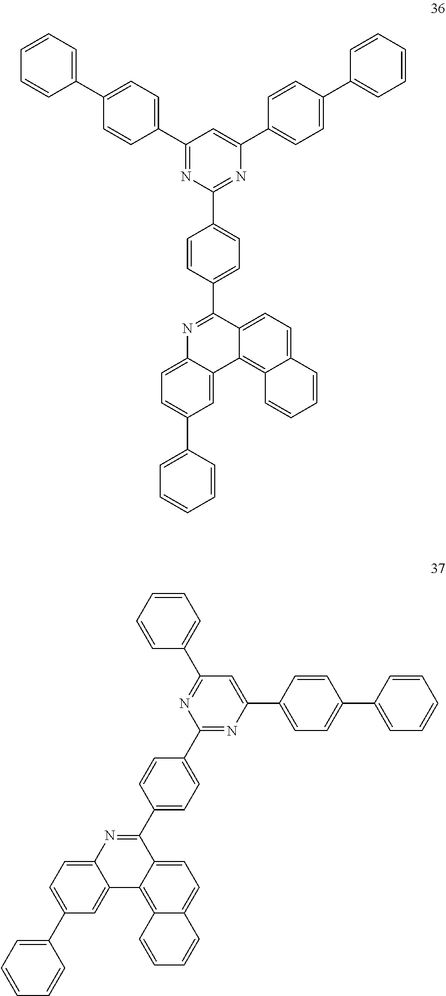 [SMILES: c1ccc(-c2ccc(-c3cc(-c4ccc(-c5ccccc5)cc4)nc(-c4ccc(-c5nc6ccc(-c7ccccc7)cc6c6c5ccc5ccccc56)cc4)n3)cc2)cc1 ; c1ccc(-c2ccc(-c3cc(-c4ccccc4)nc(-c4ccc(-c5nc6ccc(-c7ccccc7)cc6c6c5ccc5ccccc56)cc4)n3)cc2)cc1]

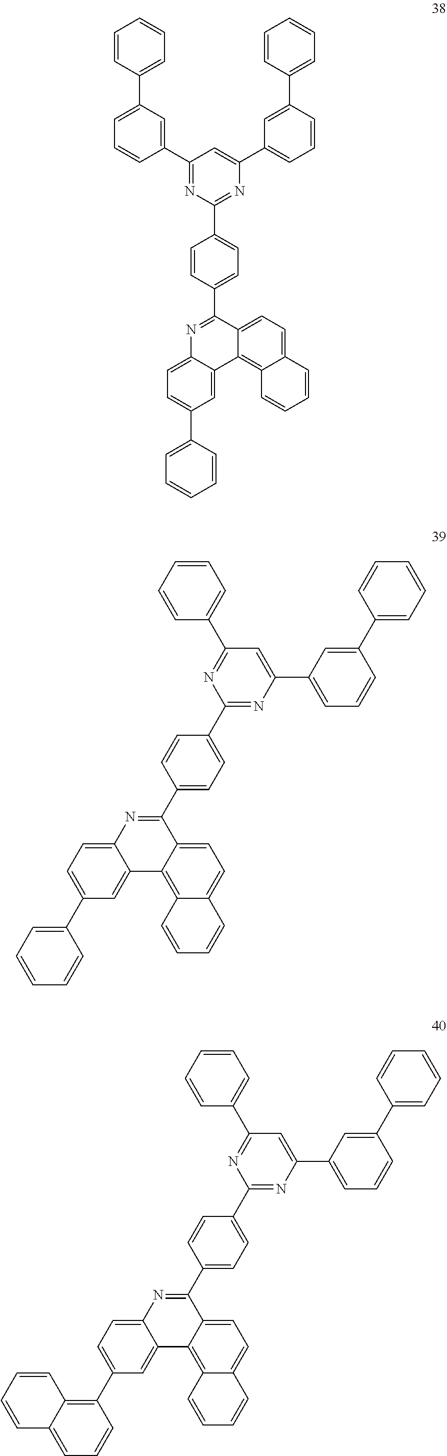 [SMILES: c1ccc(-c2cccc(-c3cc(-c4cccc(-c5ccccc5)c4)nc(-c4ccc(-c5nc6ccc(-c7ccccc7)cc6c6c5ccc5ccccc56)cc4)n3)c2)cc1 ; c1ccc(-c2cccc(-c3cc(-c4ccccc4)nc(-c4ccc(-c5nc6ccc(-c7ccccc7)cc6c6c5ccc5ccccc56)cc4)n3)c2)cc1 ; c1ccc(-c2cccc(-c3cc(-c4ccccc4)nc(-c4ccc(-c5nc6ccc(-c7cccc8ccccc78)cc6c6c5ccc5ccccc56)cc4)n3)c2)cc1]

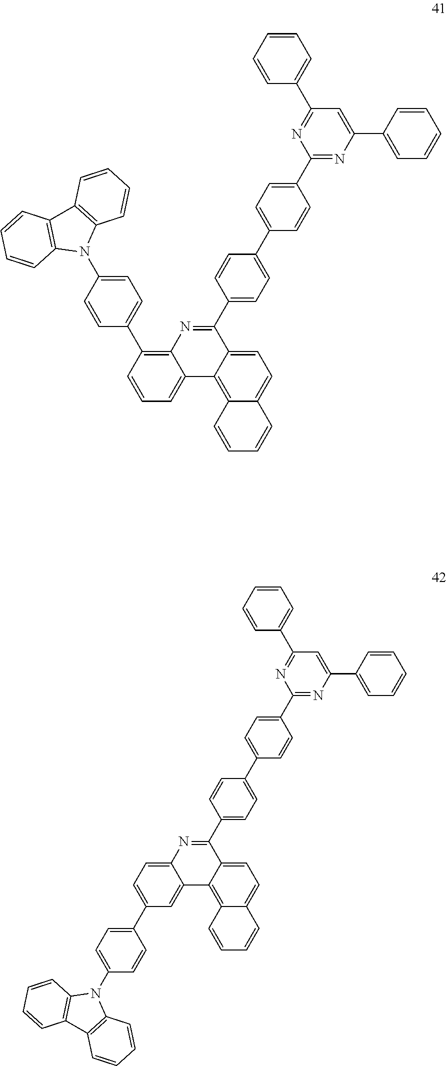 [SMILES: c1ccc(-c2cc(-c3ccccc3)nc(-c3ccc(-c4ccc(-c5nc6c(-c7ccc(-n8c9ccccc9c9ccccc98)cc7)cccc6c6c5ccc5ccccc56)cc4)cc3)n2)cc1 ; c1ccc(-c2cc(-c3ccccc3)nc(-c3ccc(-c4ccc(-c5nc6ccc(-c7ccc(-n8c9ccccc9c9ccccc98)cc7)cc6c6c5ccc5ccccc56)cc4)cc3)n2)cc1]

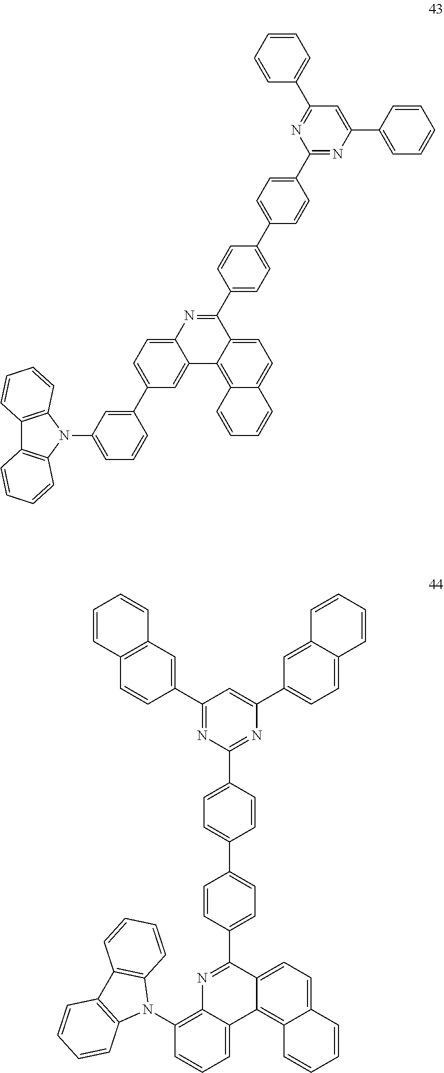 [SMILES: c1ccc(-c2cc(-c3ccccc3)nc(-c3ccc(-c4ccc(-c5nc6ccc(-c7cccc(-n8c9ccccc9c9ccccc98)c7)cc6c6c5ccc5ccccc56)cc4)cc3)n2)cc1 ; c1ccc2cc(-c3cc(-c4ccc5ccccc5c4)nc(-c4ccc(-c5ccc(-c6nc7c(-n8c9ccccc9c9ccccc98)cccc7c7c6ccc6ccccc67)cc5)cc4)n3)ccc2c1]

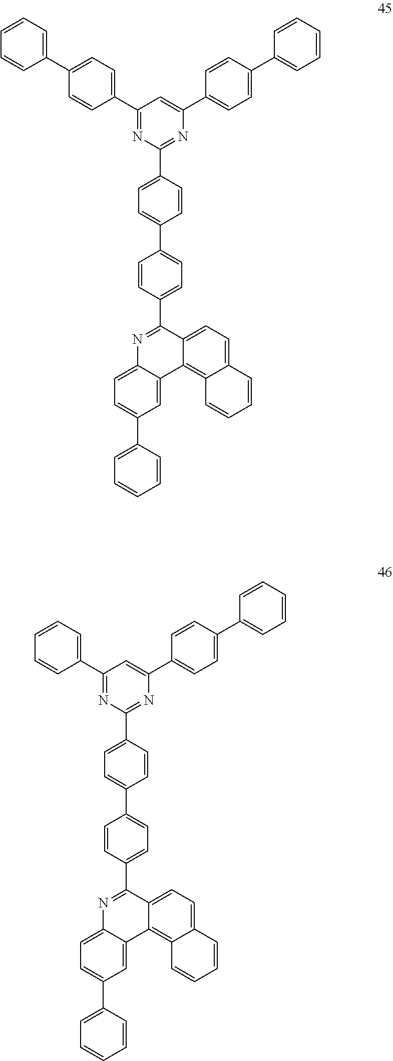 [SMILES: c1ccc(-c2ccc(-c3cc(-c4ccc(-c5ccccc5)cc4)nc(-c4ccc(-c5ccc(-c6nc7ccc(-c8ccccc8)cc7c7c6ccc6ccccc67)cc5)cc4)n3)cc2)cc1 ; c1ccc(-c2ccc(-c3cc(-c4ccccc4)nc(-c4ccc(-c5ccc(-c6nc7ccc(-c8ccccc8)cc7c7c6ccc6ccccc67)cc5)cc4)n3)cc2)cc1]

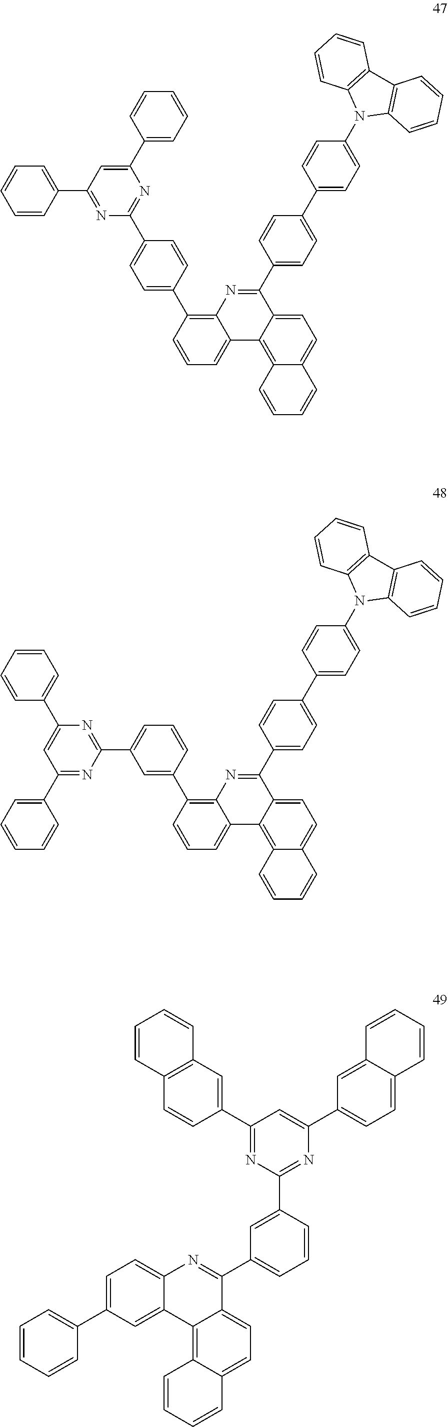 [SMILES: c1ccc(-c2cc(-c3ccccc3)nc(-c3ccc(-c4cccc5c4nc(-c4ccc(-c6ccc(-n7c8ccccc8c8ccccc87)cc6)cc4)c4ccc6ccccc6c45)cc3)n2)cc1 ; c1ccc(-c2cc(-c3ccccc3)nc(-c3cccc(-c4cccc5c4nc(-c4ccc(-c6ccc(-n7c8ccccc8c8ccccc87)cc6)cc4)c4ccc6ccccc6c45)c3)n2)cc1 ; c1ccc(-c2ccc3nc(-c4cccc(-c5nc(-c6ccc7ccccc7c6)cc(-c6ccc7ccccc7c6)n5)c4)c4ccc5ccccc5c4c3c2)cc1]

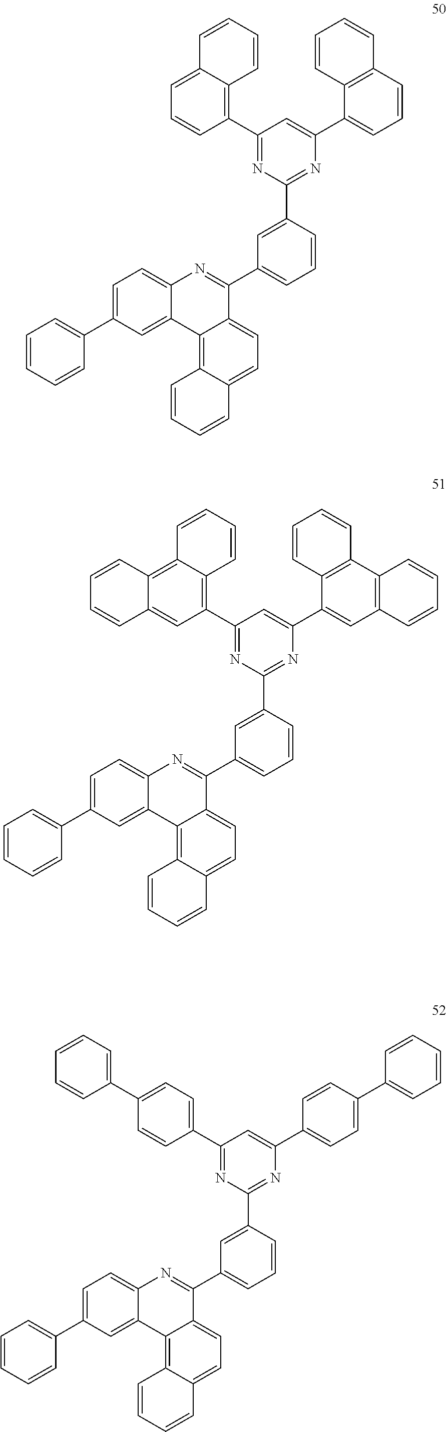 [SMILES: c1ccc(-c2ccc3nc(-c4cccc(-c5nc(-c6cccc7ccccc67)cc(-c6cccc7ccccc67)n5)c4)c4ccc5ccccc5c4c3c2)cc1 ; c1ccc(-c2ccc3nc(-c4cccc(-c5nc(-c6cc7ccccc7c7ccccc67)cc(-c6cc7ccccc7c7ccccc67)n5)c4)c4ccc5ccccc5c4c3c2)cc1 ; c1ccc(-c2ccc(-c3cc(-c4ccc(-c5ccccc5)cc4)nc(-c4cccc(-c5nc6ccc(-c7ccccc7)cc6c6c5ccc5ccccc56)c4)n3)cc2)cc1]

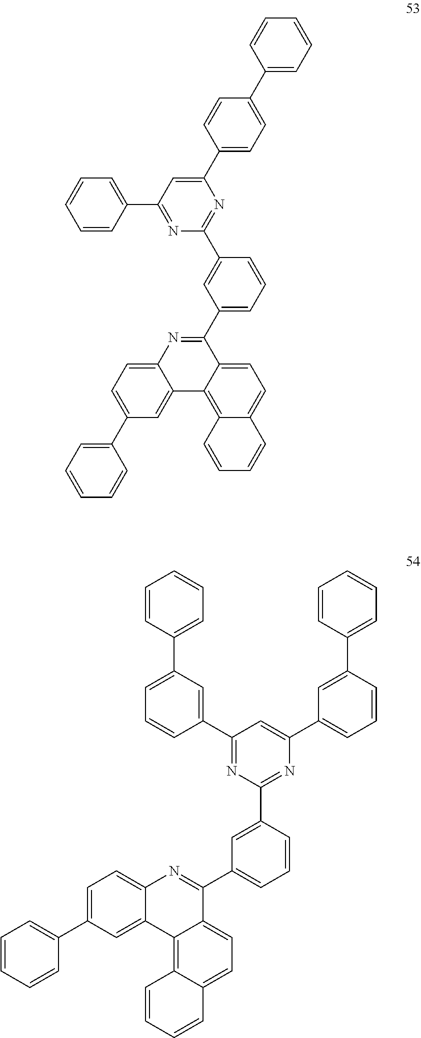 [SMILES: c1ccc(-c2ccc(-c3cc(-c4ccccc4)nc(-c4cccc(-c5nc6ccc(-c7ccccc7)cc6c6c5ccc5ccccc56)c4)n3)cc2)cc1 ; c1ccc(-c2cccc(-c3cc(-c4cccc(-c5ccccc5)c4)nc(-c4cccc(-c5nc6ccc(-c7ccccc7)cc6c6c5ccc5ccccc56)c4)n3)c2)cc1]

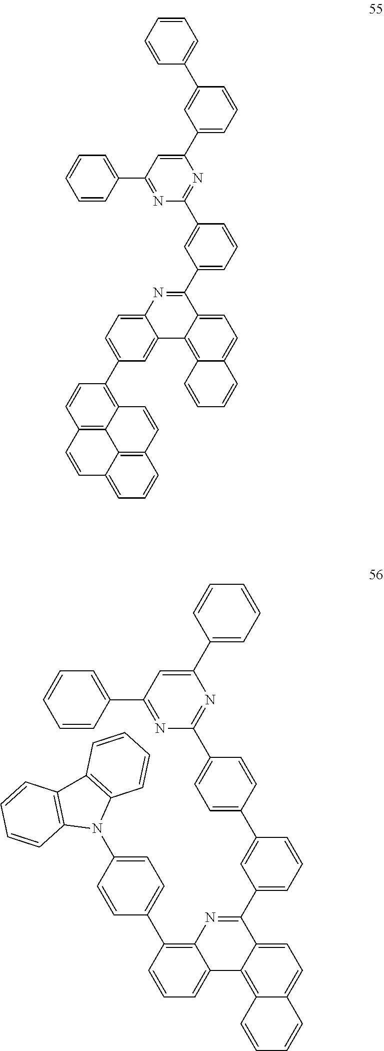 [SMILES: c1ccc(-c2cccc(-c3cc(-c4ccccc4)nc(-c4cccc(-c5nc6ccc(-c7ccc8ccc9cccc%10ccc7c8c9%10)cc6c6c5ccc5ccccc56)c4)n3)c2)cc1 ; c1ccc(-c2cc(-c3ccccc3)nc(-c3ccc(-c4cccc(-c5nc6c(-c7ccc(-n8c9ccccc9c9ccccc98)cc7)cccc6c6c5ccc5ccccc56)c4)cc3)n2)cc1]

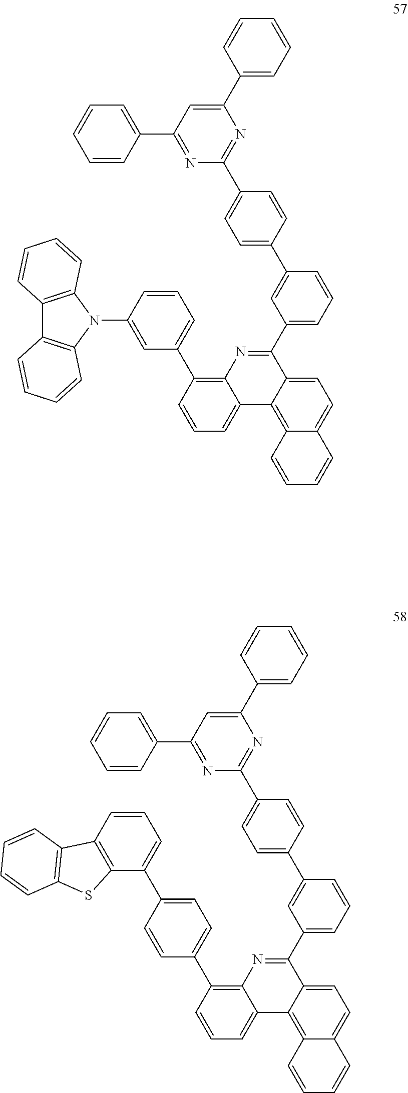 [SMILES: c1ccc(-c2cc(-c3ccccc3)nc(-c3ccc(-c4cccc(-c5nc6c(-c7cccc(-n8c9ccccc9c9ccccc98)c7)cccc6c6c5ccc5ccccc56)c4)cc3)n2)cc1 ; c1ccc(-c2cc(-c3ccccc3)nc(-c3ccc(-c4cccc(-c5nc6c(-c7ccc(-c8cccc9c8sc8ccccc89)cc7)cccc6c6c5ccc5ccccc56)c4)cc3)n2)cc1]

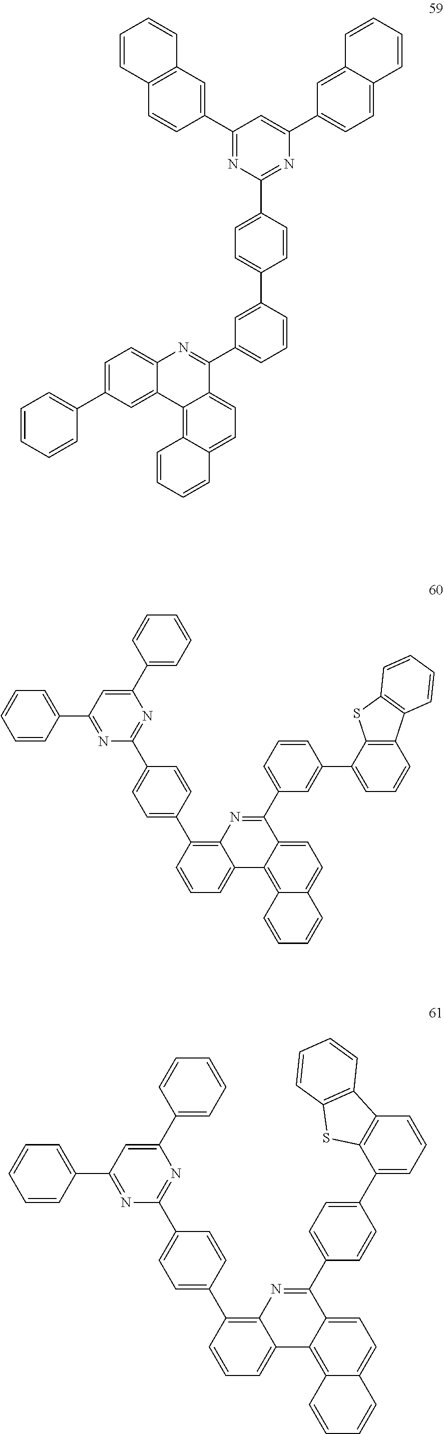 [SMILES: c1ccc(-c2ccc3nc(-c4cccc(-c5ccc(-c6nc(-c7ccc8ccccc8c7)cc(-c7ccc8ccccc8c7)n6)cc5)c4)c4ccc5ccccc5c4c3c2)cc1 ; c1ccc(-c2cc(-c3ccccc3)nc(-c3ccc(-c4cccc5c4nc(-c4cccc(-c6cccc7c6sc6ccccc67)c4)c4ccc6ccccc6c45)cc3)n2)cc1 ; c1ccc(-c2cc(-c3ccccc3)nc(-c3ccc(-c4cccc5c4nc(-c4ccc(-c6cccc7c6sc6ccccc67)cc4)c4ccc6ccccc6c45)cc3)n2)cc1]

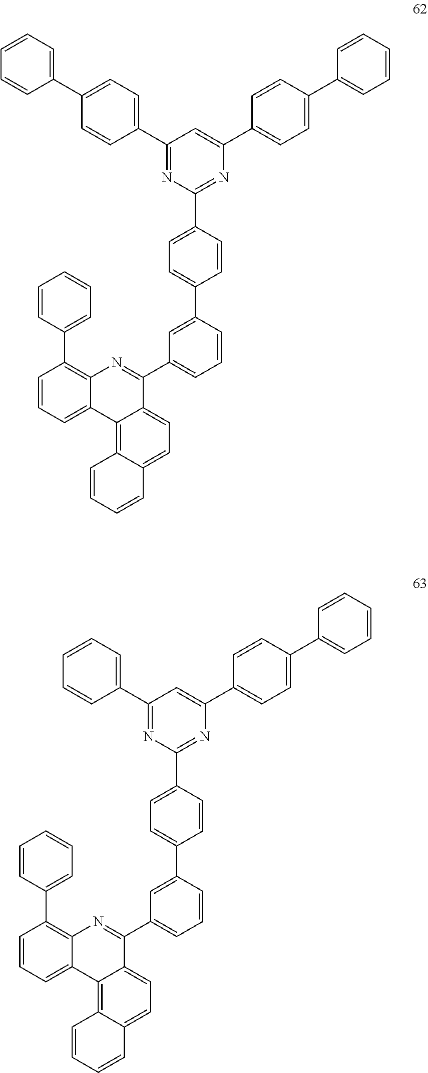 [SMILES: c1ccc(-c2ccc(-c3cc(-c4ccc(-c5ccccc5)cc4)nc(-c4ccc(-c5cccc(-c6nc7c(-c8ccccc8)cccc7c7c6ccc6ccccc67)c5)cc4)n3)cc2)cc1 ; c1ccc(-c2ccc(-c3cc(-c4ccccc4)nc(-c4ccc(-c5cccc(-c6nc7c(-c8ccccc8)cccc7c7c6ccc6ccccc67)c5)cc4)n3)cc2)cc1]

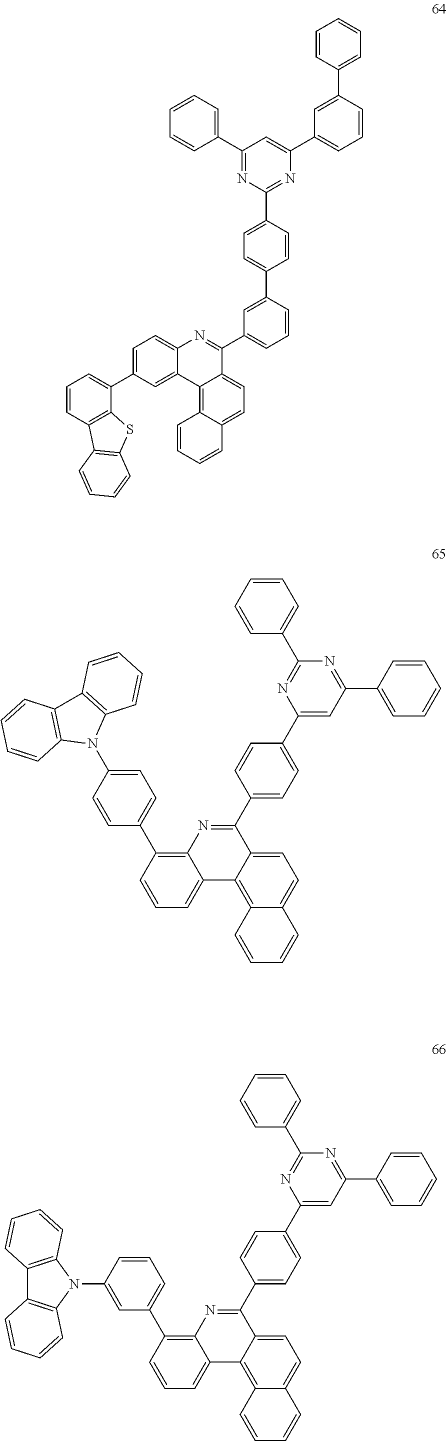 [SMILES: c1ccc(-c2cccc(-c3cc(-c4ccccc4)nc(-c4ccc(-c5cccc(-c6nc7ccc(-c8cccc9c8sc8ccccc89)cc7c7c6ccc6ccccc67)c5)cc4)n3)c2)cc1 ; c1ccc(-c2cc(-c3ccc(-c4nc5c(-c6ccc(-n7c8ccccc8c8ccccc87)cc6)cccc5c5c4ccc4ccccc45)cc3)nc(-c3ccccc3)n2)cc1 ; c1ccc(-c2cc(-c3ccc(-c4nc5c(-c6cccc(-n7c8ccccc8c8ccccc87)c6)cccc5c5c4ccc4ccccc45)cc3)nc(-c3ccccc3)n2)cc1]

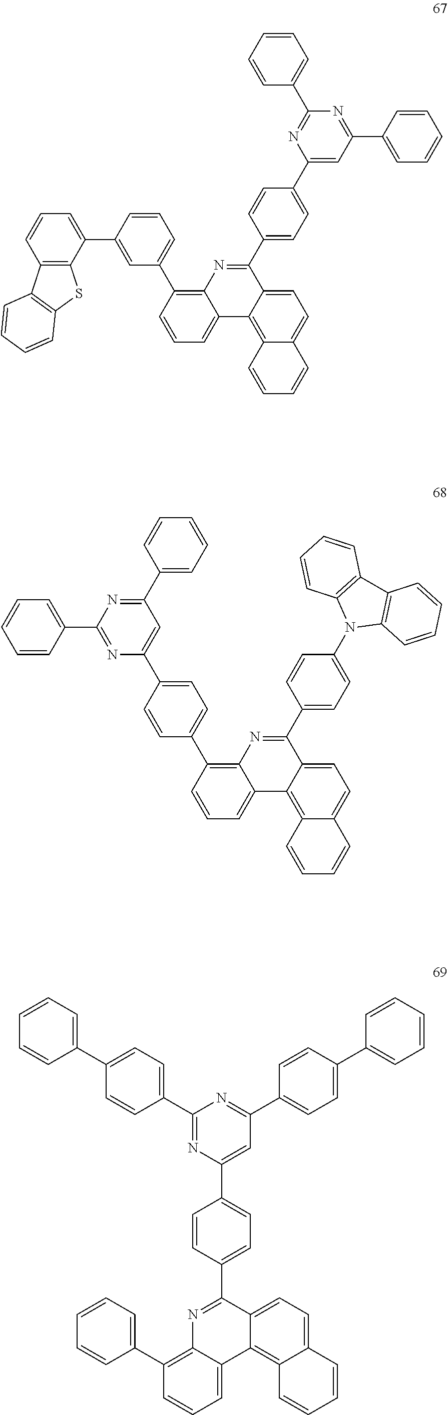 [SMILES: c1ccc(-c2cc(-c3ccc(-c4nc5c(-c6cccc(-c7cccc8c7sc7ccccc78)c6)cccc5c5c4ccc4ccccc45)cc3)nc(-c3ccccc3)n2)cc1 ; c1ccc(-c2cc(-c3ccc(-c4cccc5c4nc(-c4ccc(-n6c7ccccc7c7ccccc76)cc4)c4ccc6ccccc6c45)cc3)nc(-c3ccccc3)n2)cc1 ; c1ccc(-c2ccc(-c3cc(-c4ccc(-c5nc6c(-c7ccccc7)cccc6c6c5ccc5ccccc56)cc4)nc(-c4ccc(-c5ccccc5)cc4)n3)cc2)cc1]

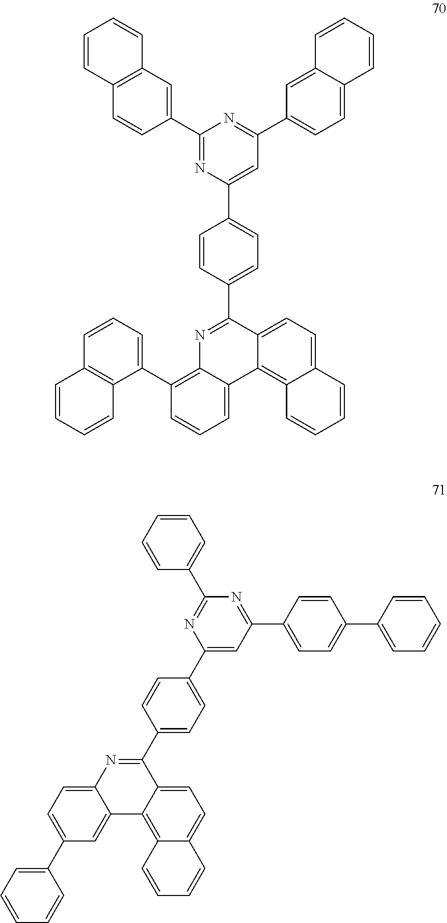 [SMILES: c1ccc2cc(-c3cc(-c4ccc(-c5nc6c(-c7cccc8ccccc78)cccc6c6c5ccc5ccccc56)cc4)nc(-c4ccc5ccccc5c4)n3)ccc2c1 ; c1ccc(-c2ccc(-c3cc(-c4ccc(-c5nc6ccc(-c7ccccc7)cc6c6c5ccc5ccccc56)cc4)nc(-c4ccccc4)n3)cc2)cc1]

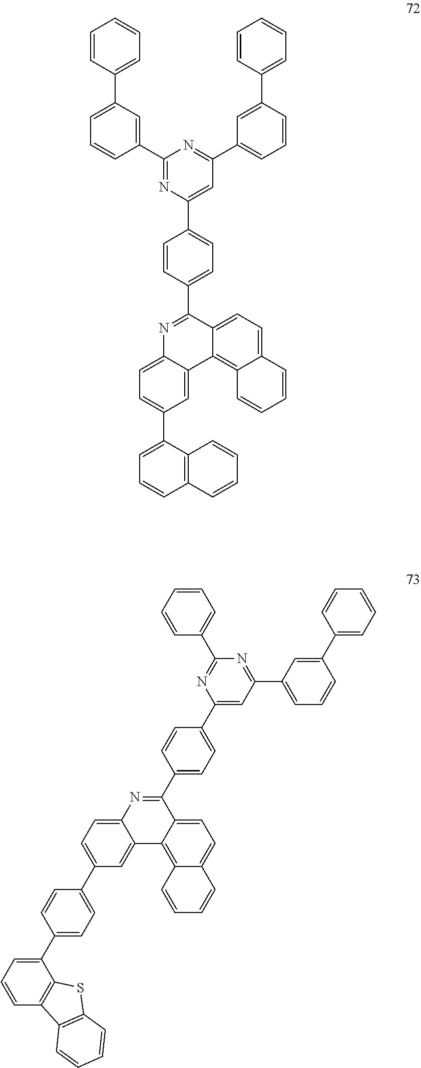 [SMILES: c1ccc(-c2cccc(-c3cc(-c4ccc(-c5nc6ccc(-c7cccc8ccccc78)cc6c6c5ccc5ccccc56)cc4)nc(-c4cccc(-c5ccccc5)c4)n3)c2)cc1 ; c1ccc(-c2cccc(-c3cc(-c4ccc(-c5nc6ccc(-c7ccc(-c8cccc9c8sc8ccccc89)cc7)cc6c6c5ccc5ccccc56)cc4)nc(-c4ccccc4)n3)c2)cc1]

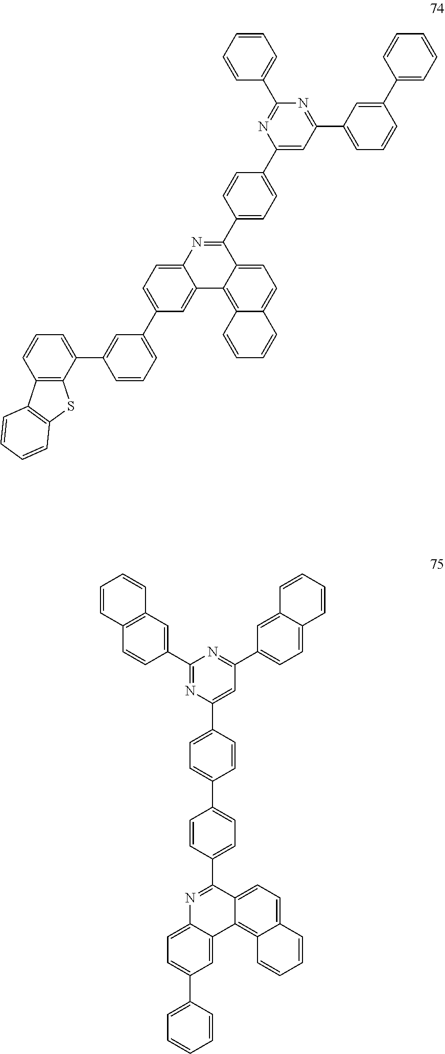 [SMILES: c1ccc(-c2cccc(-c3cc(-c4ccc(-c5nc6ccc(-c7cccc(-c8cccc9c8sc8ccccc89)c7)cc6c6c5ccc5ccccc56)cc4)nc(-c4ccccc4)n3)c2)cc1 ; c1ccc(-c2ccc3nc(-c4ccc(-c5ccc(-c6cc(-c7ccc8ccccc8c7)nc(-c7ccc8ccccc8c7)n6)cc5)cc4)c4ccc5ccccc5c4c3c2)cc1]

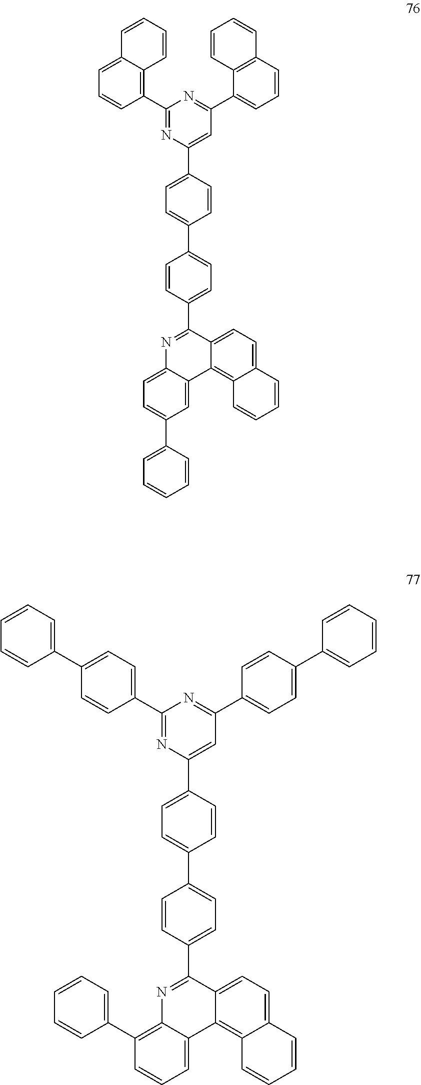 [SMILES: c1ccc(-c2ccc3nc(-c4ccc(-c5ccc(-c6cc(-c7cccc8ccccc78)nc(-c7cccc8ccccc78)n6)cc5)cc4)c4ccc5ccccc5c4c3c2)cc1 ; c1ccc(-c2ccc(-c3cc(-c4ccc(-c5ccc(-c6nc7c(-c8ccccc8)cccc7c7c6ccc6ccccc67)cc5)cc4)nc(-c4ccc(-c5ccccc5)cc4)n3)cc2)cc1]

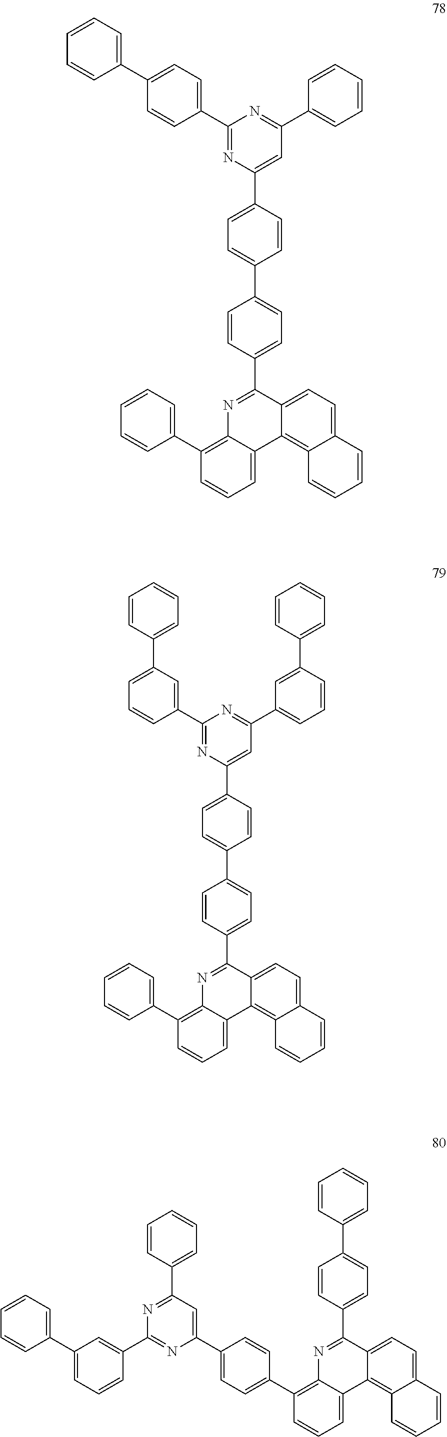 [SMILES: c1ccc(-c2ccc(-c3nc(-c4ccccc4)cc(-c4ccc(-c5ccc(-c6nc7c(-c8ccccc8)cccc7c7c6ccc6ccccc67)cc5)cc4)n3)cc2)cc1 ; c1ccc(-c2cccc(-c3cc(-c4ccc(-c5ccc(-c6nc7c(-c8ccccc8)cccc7c7c6ccc6ccccc67)cc5)cc4)nc(-c4cccc(-c5ccccc5)c4)n3)c2)cc1 ; c1ccc(-c2ccc(-c3nc4c(-c5ccc(-c6cc(-c7ccccc7)nc(-c7cccc(-c8ccccc8)c7)n6)cc5)cccc4c4c3ccc3ccccc34)cc2)cc1]

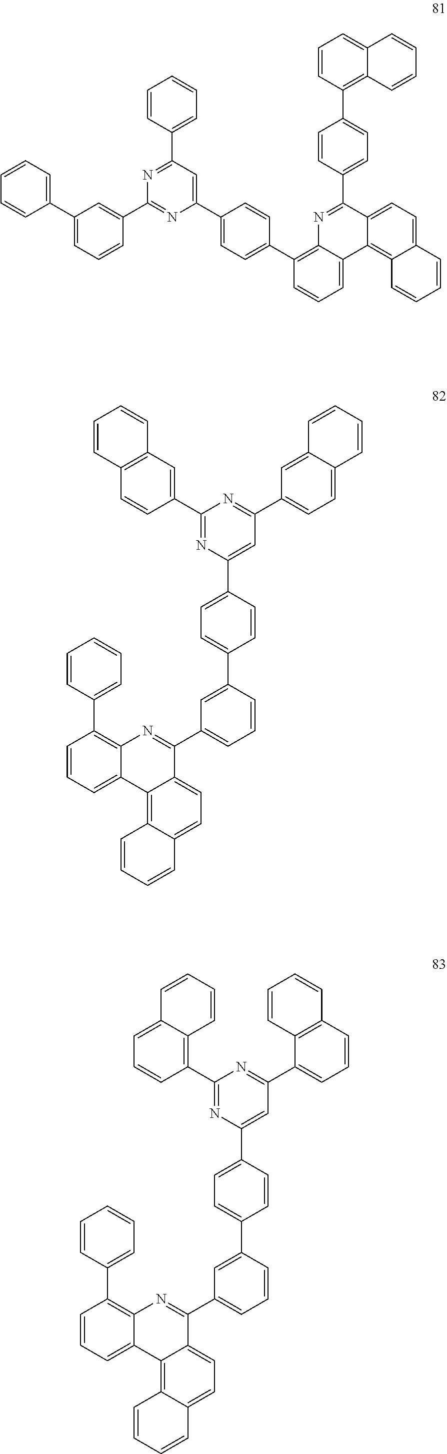 [SMILES: c1ccc(-c2cccc(-c3nc(-c4ccccc4)cc(-c4ccc(-c5cccc6c5nc(-c5ccc(-c7cccc8ccccc78)cc5)c5ccc7ccccc7c56)cc4)n3)c2)cc1 ; c1ccc(-c2cccc3c2nc(-c2cccc(-c4ccc(-c5cc(-c6ccc7ccccc7c6)nc(-c6ccc7ccccc7c6)n5)cc4)c2)c2ccc4ccccc4c23)cc1 ; c1ccc(-c2cccc3c2nc(-c2cccc(-c4ccc(-c5cc(-c6cccc7ccccc67)nc(-c6cccc7ccccc67)n5)cc4)c2)c2ccc4ccccc4c23)cc1]

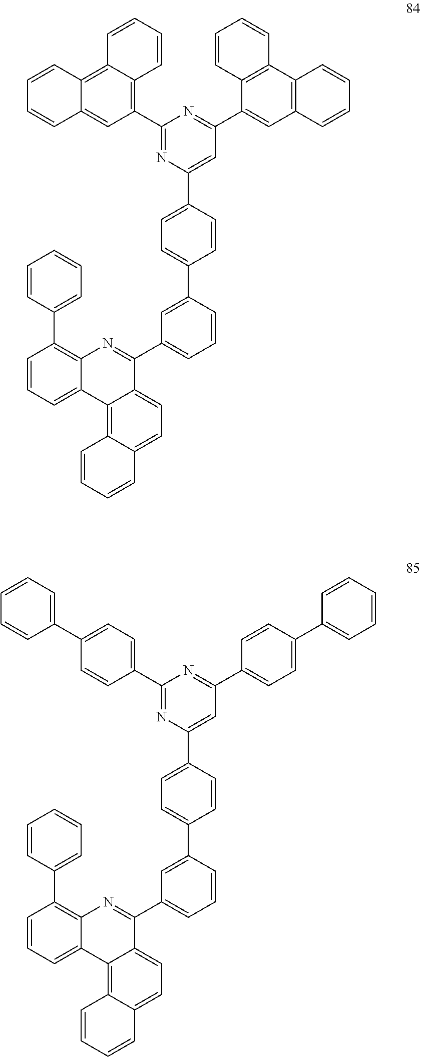 [SMILES: c1ccc(-c2cccc3c2nc(-c2cccc(-c4ccc(-c5cc(-c6cc7ccccc7c7ccccc67)nc(-c6cc7ccccc7c7ccccc67)n5)cc4)c2)c2ccc4ccccc4c23)cc1 ; c1ccc(-c2ccc(-c3cc(-c4ccc(-c5cccc(-c6nc7c(-c8ccccc8)cccc7c7c6ccc6ccccc67)c5)cc4)nc(-c4ccc(-c5ccccc5)cc4)n3)cc2)cc1]

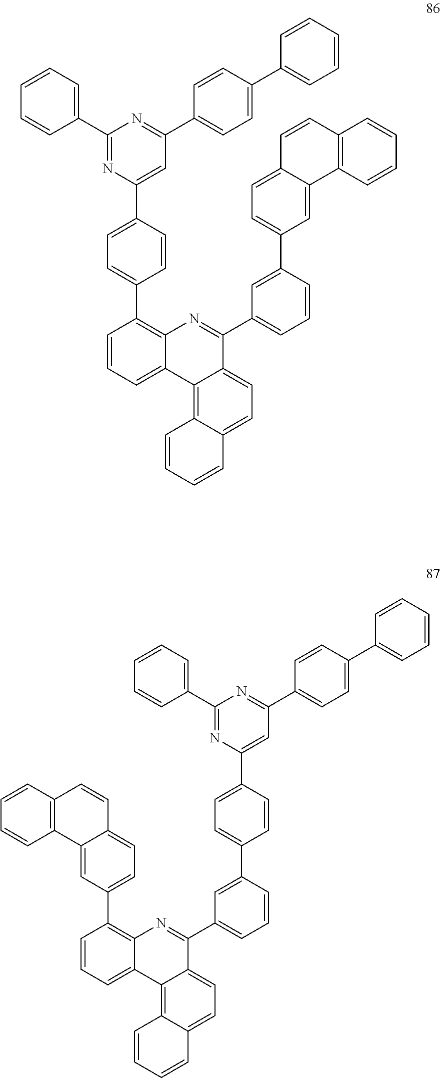 [SMILES: c1ccc(-c2ccc(-c3cc(-c4ccc(-c5cccc6c5nc(-c5cccc(-c7ccc8ccc9ccccc9c8c7)c5)c5ccc7ccccc7c56)cc4)nc(-c4ccccc4)n3)cc2)cc1 ; c1ccc(-c2ccc(-c3cc(-c4ccc(-c5cccc(-c6nc7c(-c8ccc9ccc%10ccccc%10c9c8)cccc7c7c6ccc6ccccc67)c5)cc4)nc(-c4ccccc4)n3)cc2)cc1]

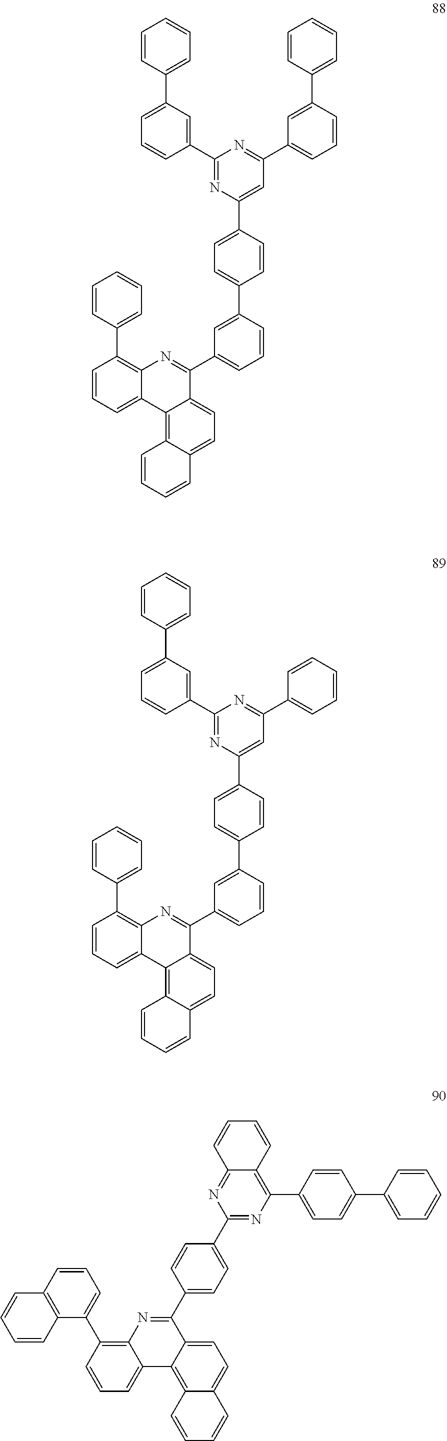 [SMILES: c1ccc(-c2cccc(-c3cc(-c4ccc(-c5cccc(-c6nc7c(-c8ccccc8)cccc7c7c6ccc6ccccc67)c5)cc4)nc(-c4cccc(-c5ccccc5)c4)n3)c2)cc1 ; c1ccc(-c2cccc(-c3nc(-c4ccccc4)cc(-c4ccc(-c5cccc(-c6nc7c(-c8ccccc8)cccc7c7c6ccc6ccccc67)c5)cc4)n3)c2)cc1 ; c1ccc(-c2ccc(-c3nc(-c4ccc(-c5nc6c(-c7cccc8ccccc78)cccc6c6c5ccc5ccccc56)cc4)nc4ccccc34)cc2)cc1]

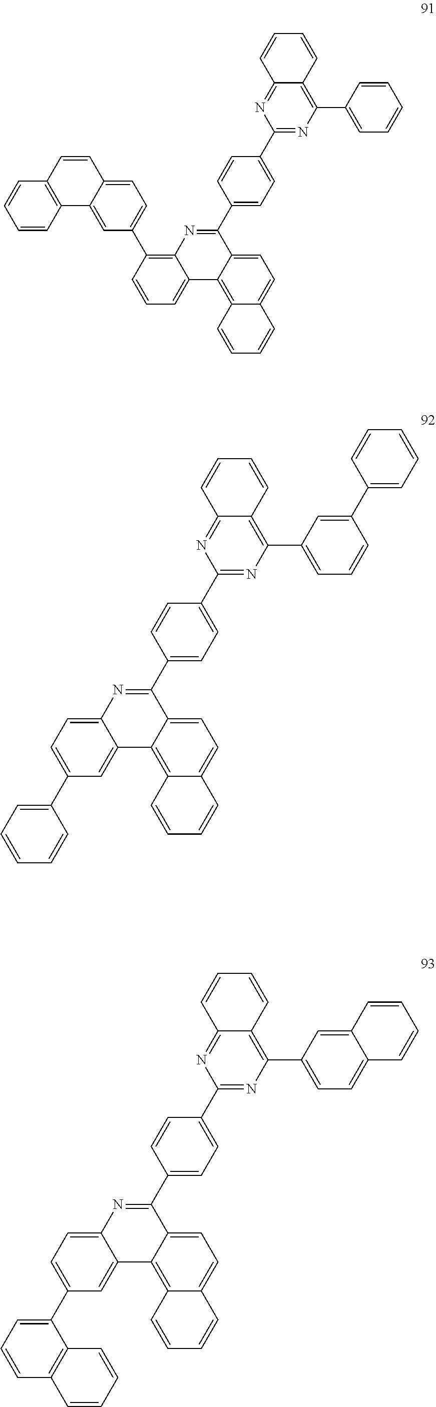 [SMILES: c1ccc(-c2nc(-c3ccc(-c4nc5c(-c6ccc7ccc8ccccc8c7c6)cccc5c5c4ccc4ccccc45)cc3)nc3ccccc23)cc1 ; c1ccc(-c2cccc(-c3nc(-c4ccc(-c5nc6ccc(-c7ccccc7)cc6c6c5ccc5ccccc56)cc4)nc4ccccc34)c2)cc1 ; c1ccc2cc(-c3nc(-c4ccc(-c5nc6ccc(-c7cccc8ccccc78)cc6c6c5ccc5ccccc56)cc4)nc4ccccc34)ccc2c1]

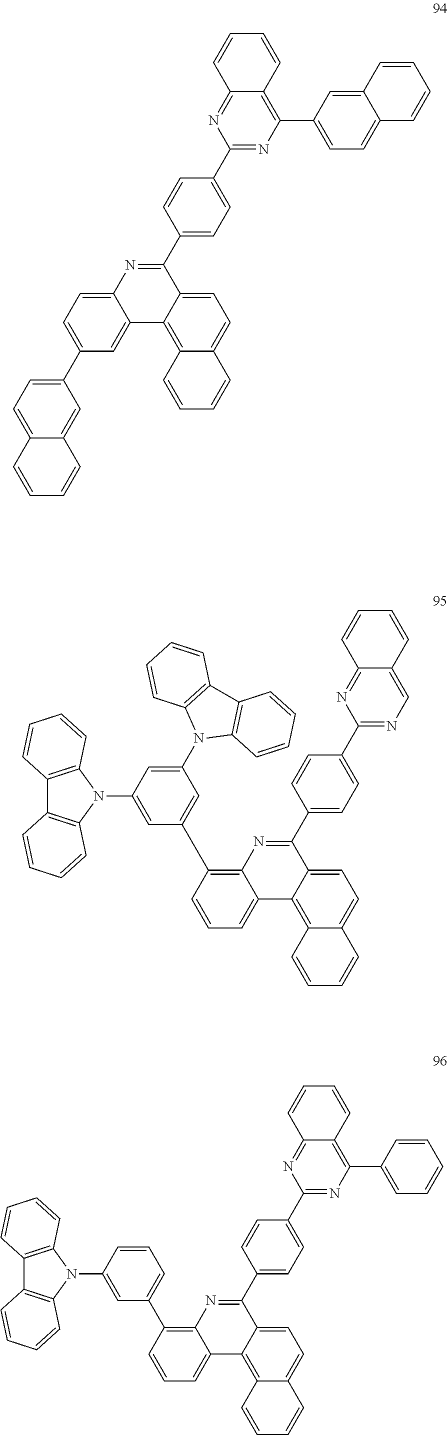 [SMILES: c1ccc2cc(-c3ccc4nc(-c5ccc(-c6nc(-c7ccc8ccccc8c7)c7ccccc7n6)cc5)c5ccc6ccccc6c5c4c3)ccc2c1 ; c1ccc2nc(-c3ccc(-c4nc5c(-c6cc(-n7c8ccccc8c8ccccc87)cc(-n7c8ccccc8c8ccccc87)c6)cccc5c5c4ccc4ccccc45)cc3)ncc2c1 ; c1ccc(-c2nc(-c3ccc(-c4nc5c(-c6cccc(-n7c8ccccc8c8ccccc87)c6)cccc5c5c4ccc4ccccc45)cc3)nc3ccccc23)cc1]

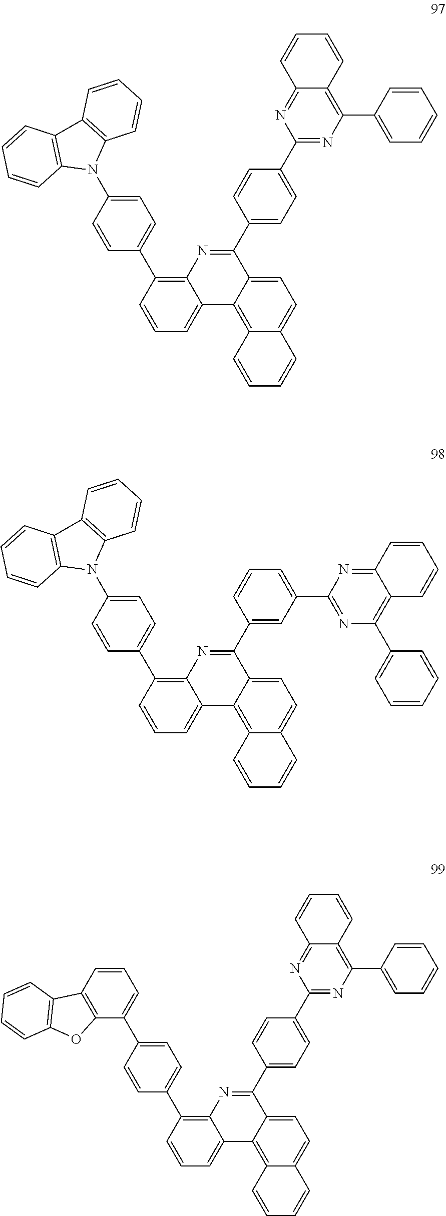 [SMILES: c1ccc(-c2nc(-c3ccc(-c4nc5c(-c6ccc(-n7c8ccccc8c8ccccc87)cc6)cccc5c5c4ccc4ccccc45)cc3)nc3ccccc23)cc1 ; c1ccc(-c2nc(-c3cccc(-c4nc5c(-c6ccc(-n7c8ccccc8c8ccccc87)cc6)cccc5c5c4ccc4ccccc45)c3)nc3ccccc23)cc1 ; c1ccc(-c2nc(-c3ccc(-c4nc5c(-c6ccc(-c7cccc8c7oc7ccccc78)cc6)cccc5c5c4ccc4ccccc45)cc3)nc3ccccc23)cc1]

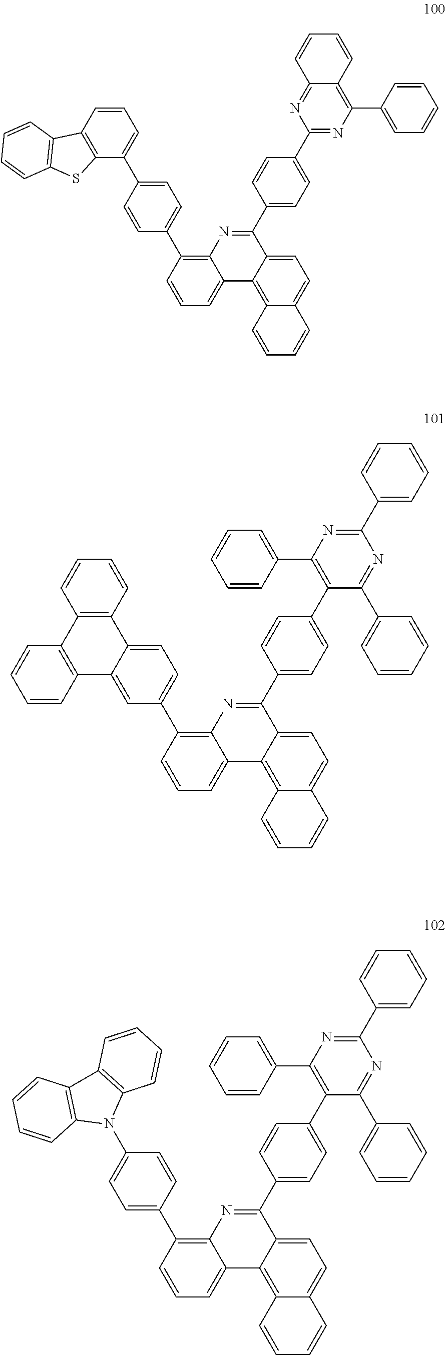 [SMILES: c1ccc(-c2nc(-c3ccc(-c4nc5c(-c6ccc(-c7cccc8c7sc7ccccc78)cc6)cccc5c5c4ccc4ccccc45)cc3)nc3ccccc23)cc1 ; c1ccc(-c2nc(-c3ccccc3)c(-c3ccc(-c4nc5c(-c6ccc7c8ccccc8c8ccccc8c7c6)cccc5c5c4ccc4ccccc45)cc3)c(-c3ccccc3)n2)cc1 ; c1ccc(-c2nc(-c3ccccc3)c(-c3ccc(-c4nc5c(-c6ccc(-n7c8ccccc8c8ccccc87)cc6)cccc5c5c4ccc4ccccc45)cc3)c(-c3ccccc3)n2)cc1]

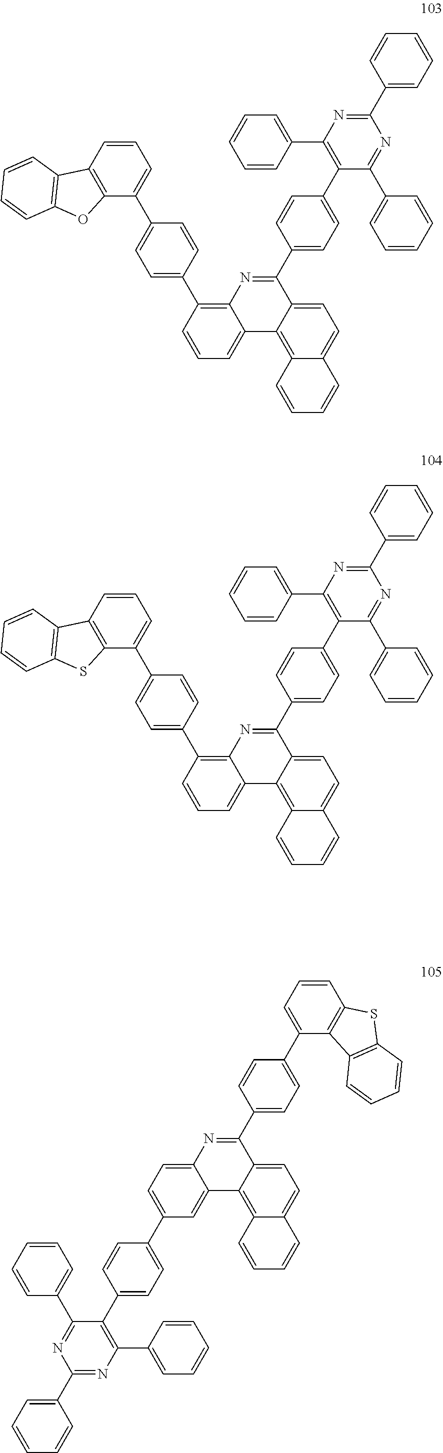 [SMILES: c1ccc(-c2nc(-c3ccccc3)c(-c3ccc(-c4nc5c(-c6ccc(-c7cccc8c7oc7ccccc78)cc6)cccc5c5c4ccc4ccccc45)cc3)c(-c3ccccc3)n2)cc1 ; c1ccc(-c2nc(-c3ccccc3)c(-c3ccc(-c4nc5c(-c6ccc(-c7cccc8c7sc7ccccc78)cc6)cccc5c5c4ccc4ccccc45)cc3)c(-c3ccccc3)n2)cc1 ; c1ccc(-c2nc(-c3ccccc3)c(-c3ccc(-c4ccc5nc(-c6ccc(-c7cccc8sc9ccccc9c78)cc6)c6ccc7ccccc7c6c5c4)cc3)c(-c3ccccc3)n2)cc1]

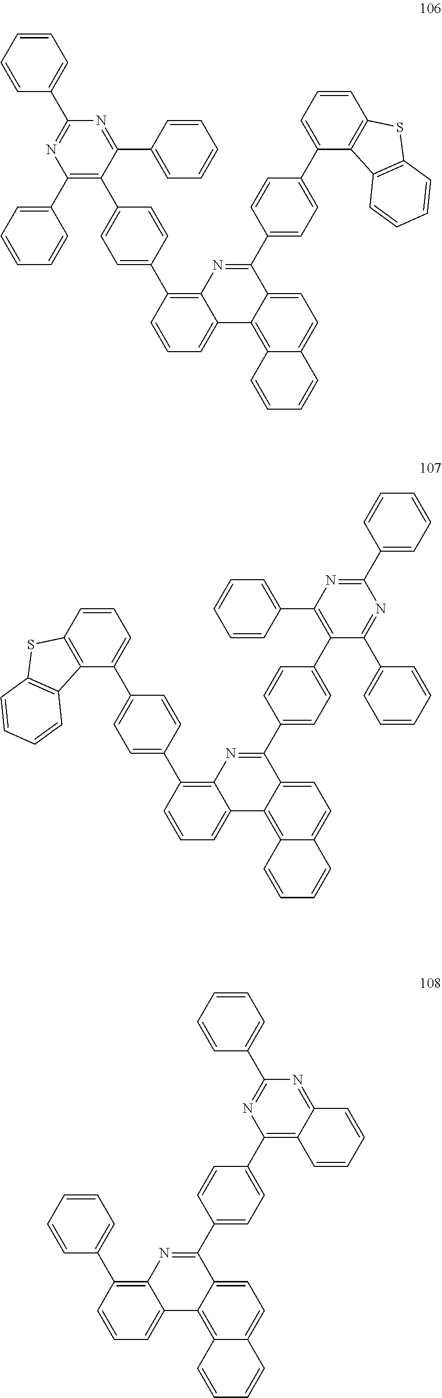 [SMILES: c1ccc(-c2nc(-c3ccccc3)c(-c3ccc(-c4cccc5c4nc(-c4ccc(-c6cccc7sc8ccccc8c67)cc4)c4ccc6ccccc6c45)cc3)c(-c3ccccc3)n2)cc1 ; c1ccc(-c2nc(-c3ccccc3)c(-c3ccc(-c4nc5c(-c6ccc(-c7cccc8sc9ccccc9c78)cc6)cccc5c5c4ccc4ccccc45)cc3)c(-c3ccccc3)n2)cc1 ; c1ccc(-c2nc(-c3ccc(-c4nc5c(-c6ccccc6)cccc5c5c4ccc4ccccc45)cc3)c3ccccc3n2)cc1]

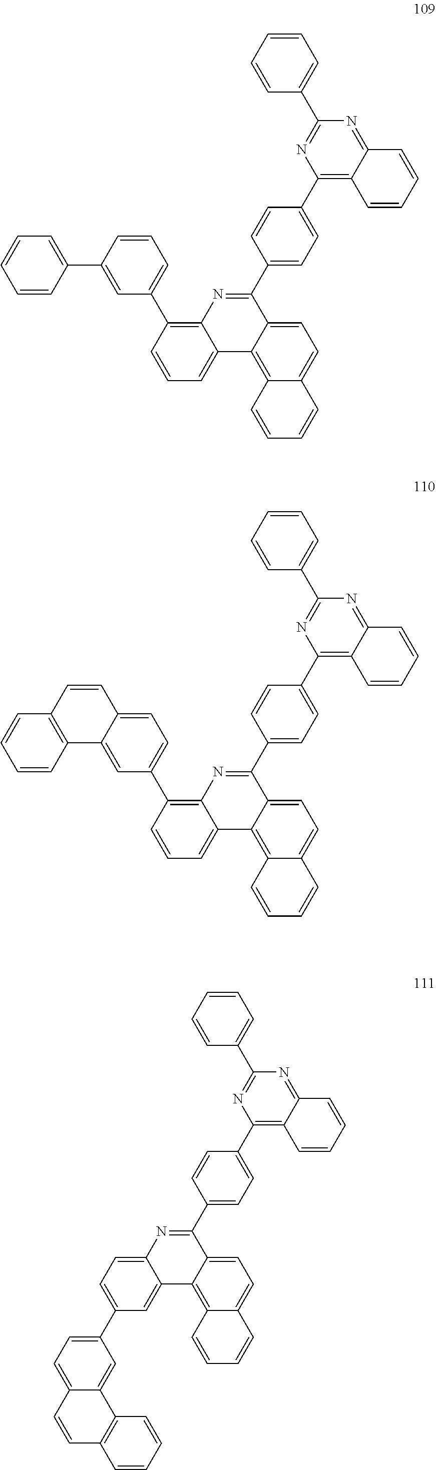 [SMILES: c1ccc(-c2cccc(-c3cccc4c3nc(-c3ccc(-c5nc(-c6ccccc6)nc6ccccc56)cc3)c3ccc5ccccc5c34)c2)cc1 ; c1ccc(-c2nc(-c3ccc(-c4nc5c(-c6ccc7ccc8ccccc8c7c6)cccc5c5c4ccc4ccccc45)cc3)c3ccccc3n2)cc1 ; c1ccc(-c2nc(-c3ccc(-c4nc5ccc(-c6ccc7ccc8ccccc8c7c6)cc5c5c4ccc4ccccc45)cc3)c3ccccc3n2)cc1]

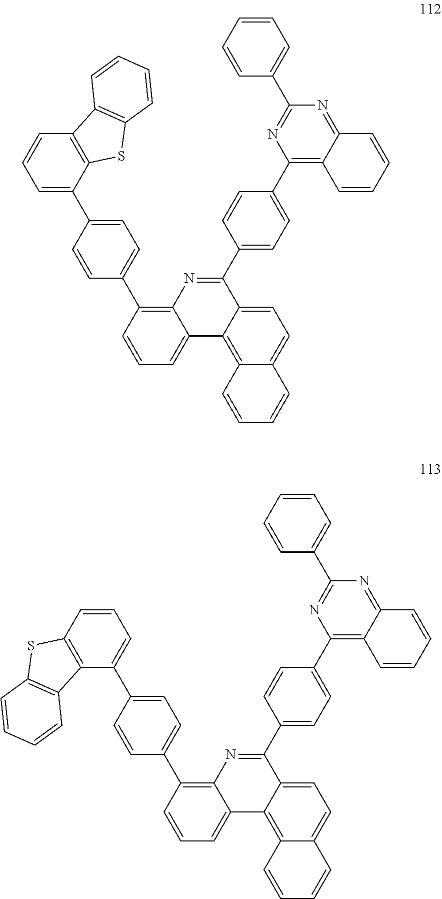 [SMILES: c1ccc(-c2nc(-c3ccc(-c4nc5c(-c6ccc(-c7cccc8c7sc7ccccc78)cc6)cccc5c5c4ccc4ccccc45)cc3)c3ccccc3n2)cc1 ; c1ccc(-c2nc(-c3ccc(-c4nc5c(-c6ccc(-c7cccc8sc9ccccc9c78)cc6)cccc5c5c4ccc4ccccc45)cc3)c3ccccc3n2)cc1]

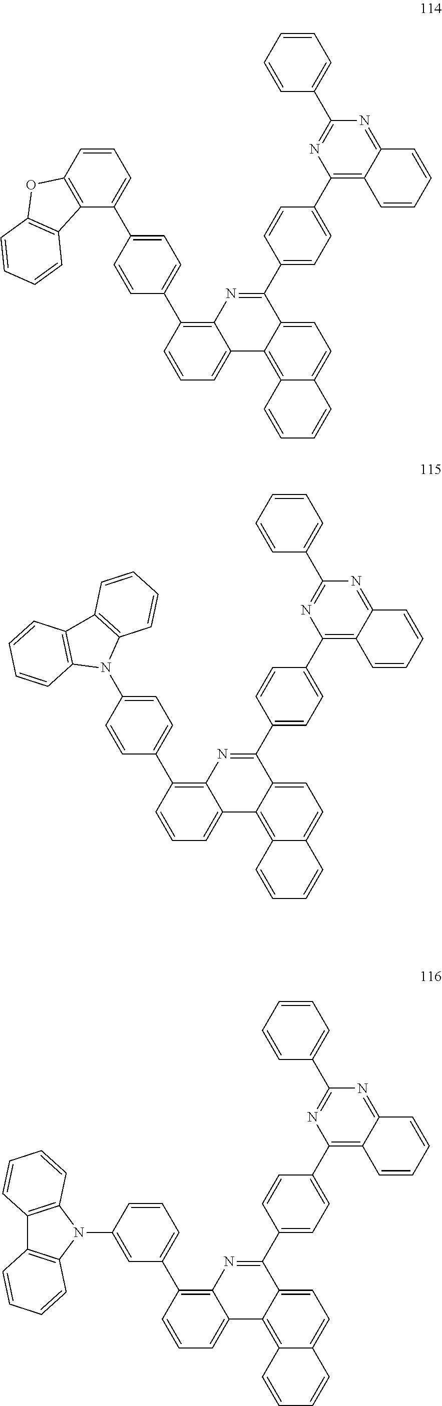 [SMILES: c1ccc(-c2nc(-c3ccc(-c4nc5c(-c6ccc(-c7cccc8oc9ccccc9c78)cc6)cccc5c5c4ccc4ccccc45)cc3)c3ccccc3n2)cc1 ; c1ccc(-c2nc(-c3ccc(-c4nc5c(-c6ccc(-n7c8ccccc8c8ccccc87)cc6)cccc5c5c4ccc4ccccc45)cc3)c3ccccc3n2)cc1 ; c1ccc(-c2nc(-c3ccc(-c4nc5c(-c6cccc(-n7c8ccccc8c8ccccc87)c6)cccc5c5c4ccc4ccccc45)cc3)c3ccccc3n2)cc1]

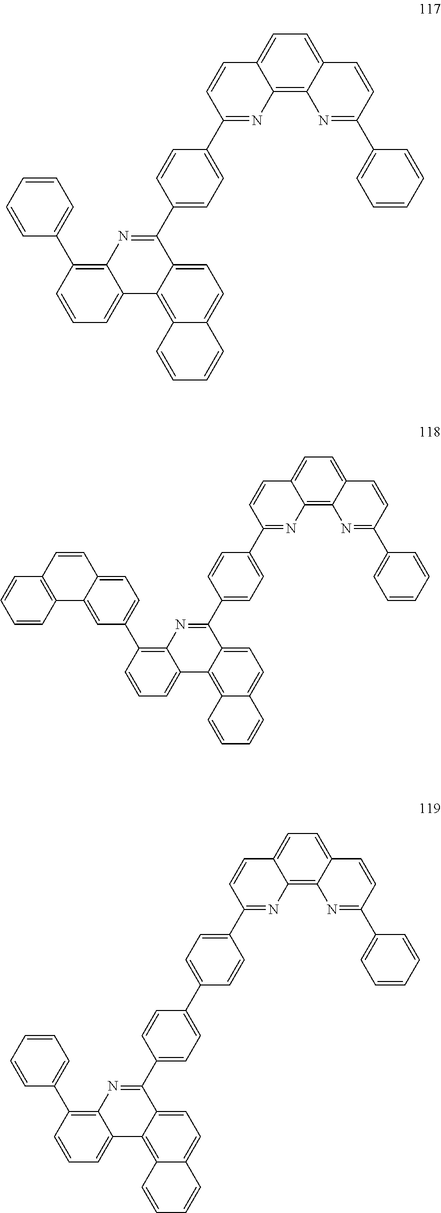 [SMILES: c1ccc(-c2ccc3ccc4ccc(-c5ccc(-c6nc7c(-c8ccccc8)cccc7c7c6ccc6ccccc67)cc5)nc4c3n2)cc1 ; c1ccc(-c2ccc3ccc4ccc(-c5ccc(-c6nc7c(-c8ccc9ccc%10ccccc%10c9c8)cccc7c7c6ccc6ccccc67)cc5)nc4c3n2)cc1 ; c1ccc(-c2ccc3ccc4ccc(-c5ccc(-c6ccc(-c7nc8c(-c9ccccc9)cccc8c8c7ccc7ccccc78)cc6)cc5)nc4c3n2)cc1]

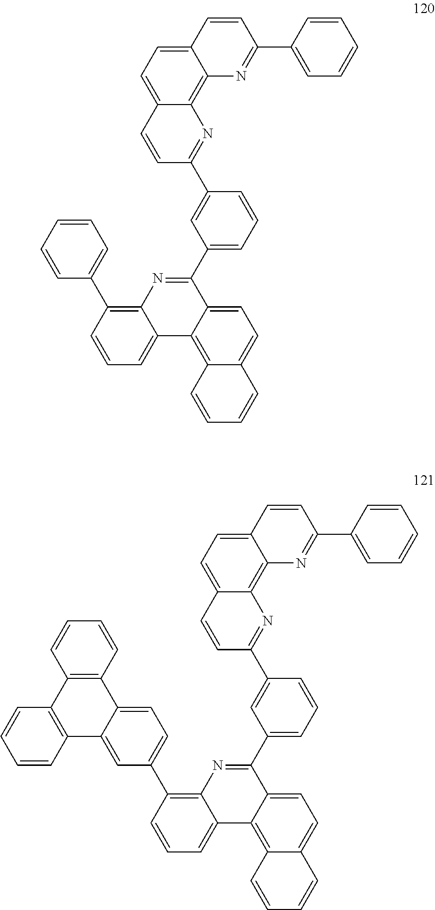 [SMILES: c1ccc(-c2ccc3ccc4ccc(-c5cccc(-c6nc7c(-c8ccccc8)cccc7c7c6ccc6ccccc67)c5)nc4c3n2)cc1 ; c1ccc(-c2ccc3ccc4ccc(-c5cccc(-c6nc7c(-c8ccc9c%10ccccc%10c%10ccccc%10c9c8)cccc7c7c6ccc6ccccc67)c5)nc4c3n2)cc1]

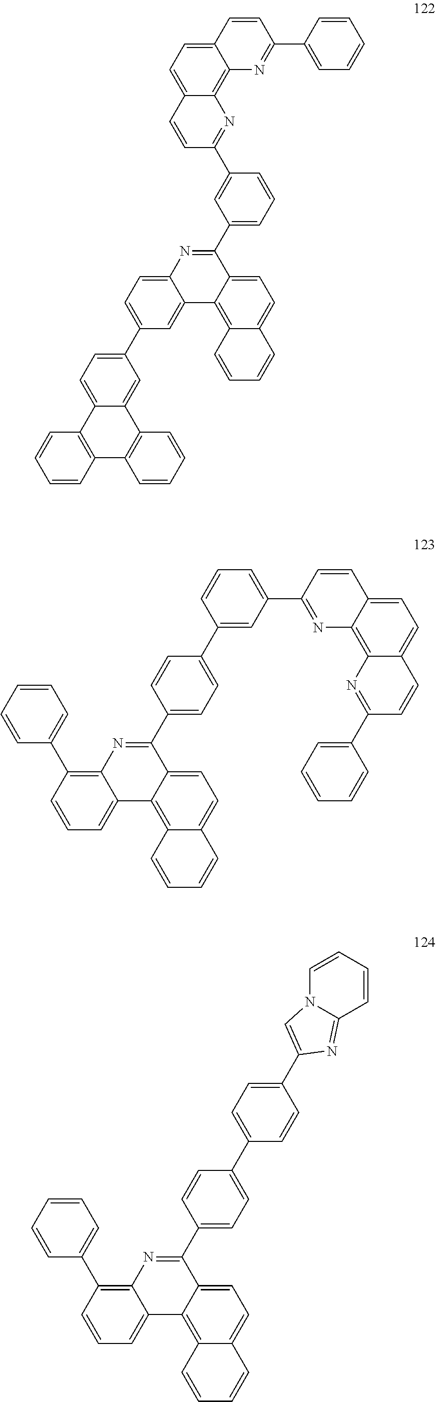 [SMILES: c1ccc(-c2ccc3ccc4ccc(-c5cccc(-c6nc7ccc(-c8ccc9c%10ccccc%10c%10ccccc%10c9c8)cc7c7c6ccc6ccccc67)c5)nc4c3n2)cc1 ; c1ccc(-c2ccc3ccc4ccc(-c5cccc(-c6ccc(-c7nc8c(-c9ccccc9)cccc8c8c7ccc7ccccc78)cc6)c5)nc4c3n2)cc1 ; c1ccc(-c2cccc3c2nc(-c2ccc(-c4ccc(-c5cn6ccccc6n5)cc4)cc2)c2ccc4ccccc4c23)cc1]

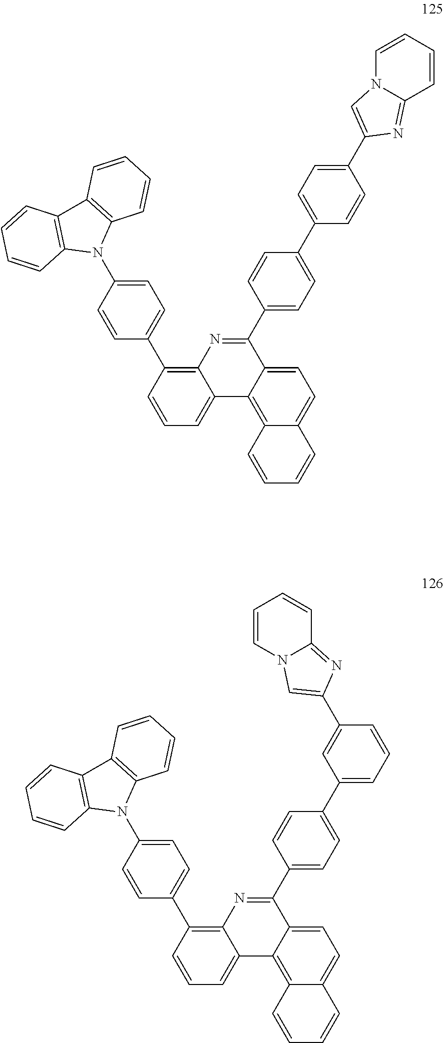 [SMILES: c1ccc2c(c1)ccc1c(-c3ccc(-c4ccc(-c5cn6ccccc6n5)cc4)cc3)nc3c(-c4ccc(-n5c6ccccc6c6ccccc65)cc4)cccc3c12 ; c1cc(-c2ccc(-c3nc4c(-c5ccc(-n6c7ccccc7c7ccccc76)cc5)cccc4c4c3ccc3ccccc34)cc2)cc(-c2cn3ccccc3n2)c1]

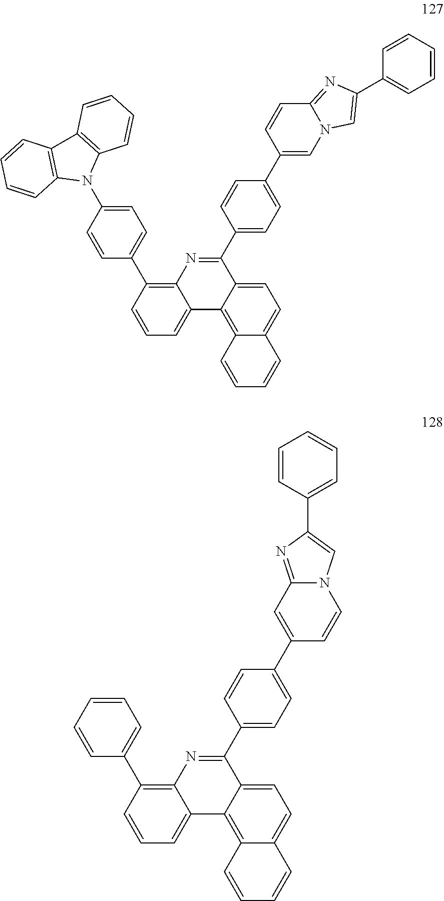 [SMILES: c1ccc(-c2cn3cc(-c4ccc(-c5nc6c(-c7ccc(-n8c9ccccc9c9ccccc98)cc7)cccc6c6c5ccc5ccccc56)cc4)ccc3n2)cc1 ; c1ccc(-c2cn3ccc(-c4ccc(-c5nc6c(-c7ccccc7)cccc6c6c5ccc5ccccc56)cc4)cc3n2)cc1]

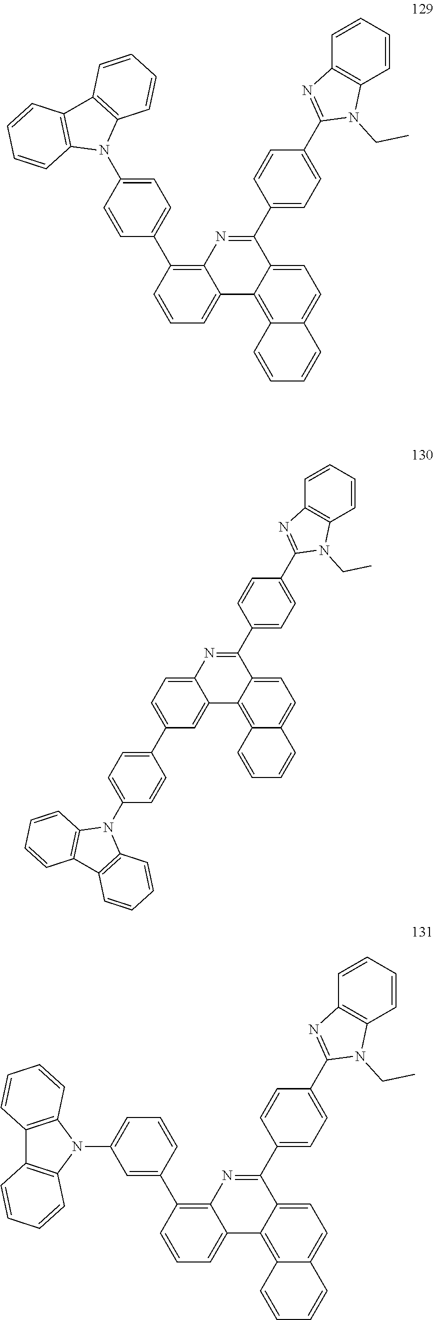 [SMILES: CCn1c(-c2ccc(-c3nc4c(-c5ccc(-n6c7ccccc7c7ccccc76)cc5)cccc4c4c3ccc3ccccc34)cc2)nc2ccccc21 ; CCn1c(-c2ccc(-c3nc4ccc(-c5ccc(-n6c7ccccc7c7ccccc76)cc5)cc4c4c3ccc3ccccc34)cc2)nc2ccccc21 ; CCn1c(-c2ccc(-c3nc4c(-c5cccc(-n6c7ccccc7c7ccccc76)c5)cccc4c4c3ccc3ccccc34)cc2)nc2ccccc21]

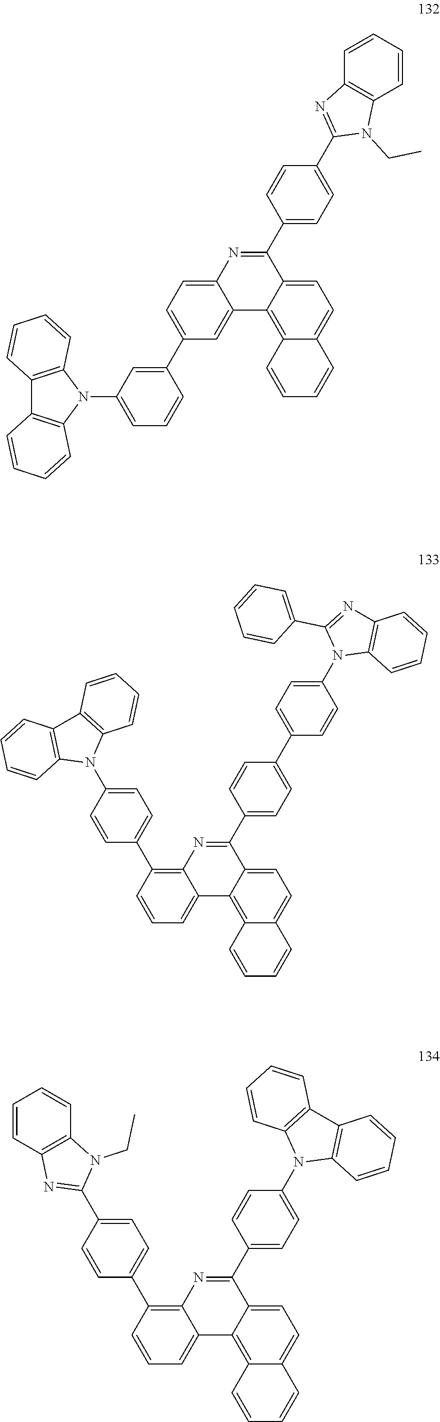 [SMILES: CCn1c(-c2ccc(-c3nc4ccc(-c5cccc(-n6c7ccccc7c7ccccc76)c5)cc4c4c3ccc3ccccc34)cc2)nc2ccccc21 ; c1ccc(-c2nc3ccccc3n2-c2ccc(-c3ccc(-c4nc5c(-c6ccc(-n7c8ccccc8c8ccccc87)cc6)cccc5c5c4ccc4ccccc45)cc3)cc2)cc1 ; CCn1c(-c2ccc(-c3cccc4c3nc(-c3ccc(-n5c6ccccc6c6ccccc65)cc3)c3ccc5ccccc5c34)cc2)nc2ccccc21]

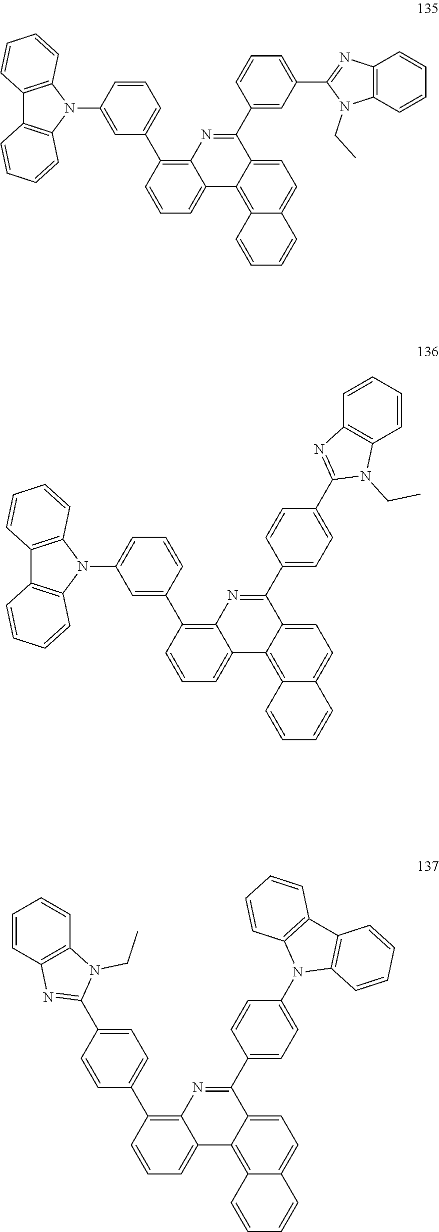 [SMILES: CCn1c(-c2cccc(-c3nc4c(-c5cccc(-n6c7ccccc7c7ccccc76)c5)cccc4c4c3ccc3ccccc34)c2)nc2ccccc21 ; CCn1c(-c2ccc(-c3nc4c(-c5cccc(-n6c7ccccc7c7ccccc76)c5)cccc4c4c3ccc3ccccc34)cc2)nc2ccccc21 ; CCn1c(-c2ccc(-c3cccc4c3nc(-c3ccc(-n5c6ccccc6c6ccccc65)cc3)c3ccc5ccccc5c34)cc2)nc2ccccc21]

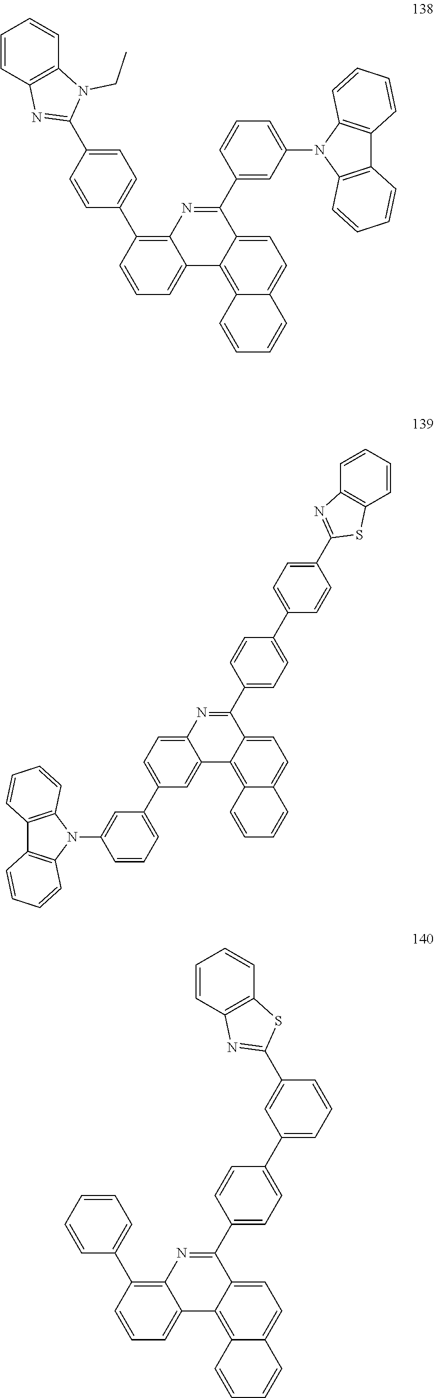 [SMILES: CCn1c(-c2ccc(-c3cccc4c3nc(-c3cccc(-n5c6ccccc6c6ccccc65)c3)c3ccc5ccccc5c34)cc2)nc2ccccc21 ; c1cc(-c2ccc3nc(-c4ccc(-c5ccc(-c6nc7ccccc7s6)cc5)cc4)c4ccc5ccccc5c4c3c2)cc(-n2c3ccccc3c3ccccc32)c1 ; c1ccc(-c2cccc3c2nc(-c2ccc(-c4cccc(-c5nc6ccccc6s5)c4)cc2)c2ccc4ccccc4c23)cc1]

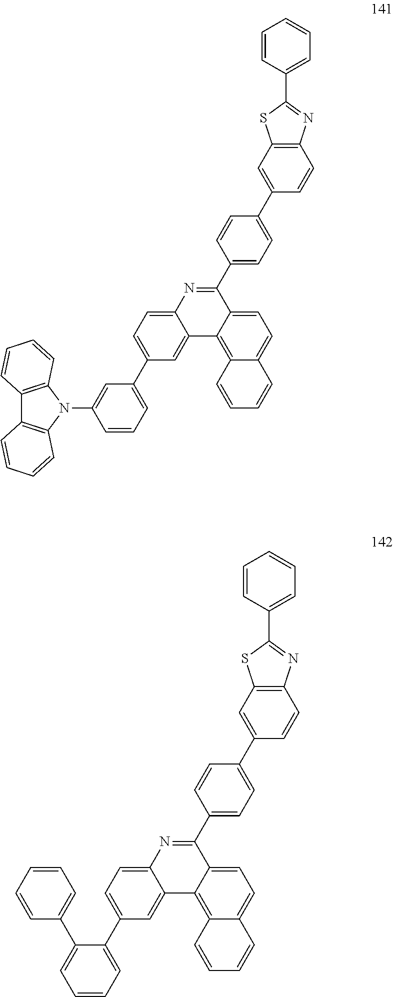 [SMILES: c1ccc(-c2nc3ccc(-c4ccc(-c5nc6ccc(-c7cccc(-n8c9ccccc9c9ccccc98)c7)cc6c6c5ccc5ccccc56)cc4)cc3s2)cc1 ; c1ccc(-c2nc3ccc(-c4ccc(-c5nc6ccc(-c7ccccc7-c7ccccc7)cc6c6c5ccc5ccccc56)cc4)cc3s2)cc1]

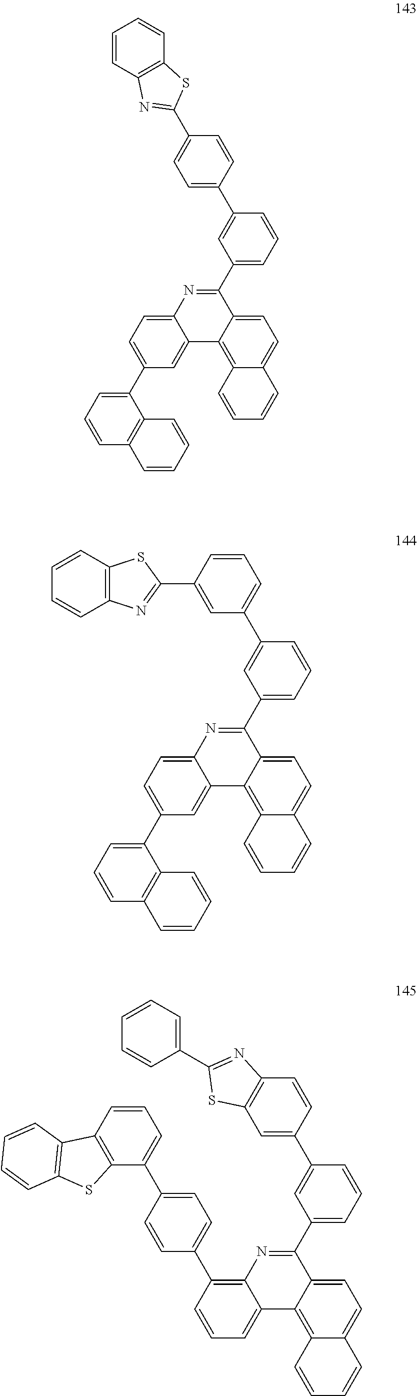 [SMILES: c1cc(-c2ccc(-c3nc4ccccc4s3)cc2)cc(-c2nc3ccc(-c4cccc5ccccc45)cc3c3c2ccc2ccccc23)c1 ; c1cc(-c2cccc(-c3nc4ccc(-c5cccc6ccccc56)cc4c4c3ccc3ccccc34)c2)cc(-c2nc3ccccc3s2)c1 ; c1ccc(-c2nc3ccc(-c4cccc(-c5nc6c(-c7ccc(-c8cccc9c8sc8ccccc89)cc7)cccc6c6c5ccc5ccccc56)c4)cc3s2)cc1]

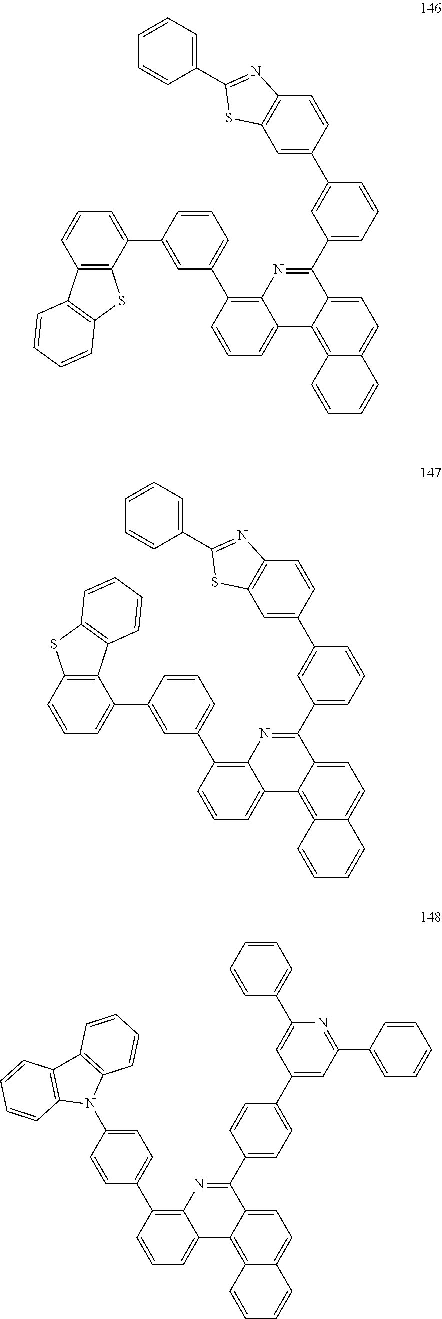 [SMILES: c1ccc(-c2nc3ccc(-c4cccc(-c5nc6c(-c7cccc(-c8cccc9c8sc8ccccc89)c7)cccc6c6c5ccc5ccccc56)c4)cc3s2)cc1 ; c1ccc(-c2nc3ccc(-c4cccc(-c5nc6c(-c7cccc(-c8cccc9sc%10ccccc%10c89)c7)cccc6c6c5ccc5ccccc56)c4)cc3s2)cc1 ; c1ccc(-c2cc(-c3ccc(-c4nc5c(-c6ccc(-n7c8ccccc8c8ccccc87)cc6)cccc5c5c4ccc4ccccc45)cc3)cc(-c3ccccc3)n2)cc1]

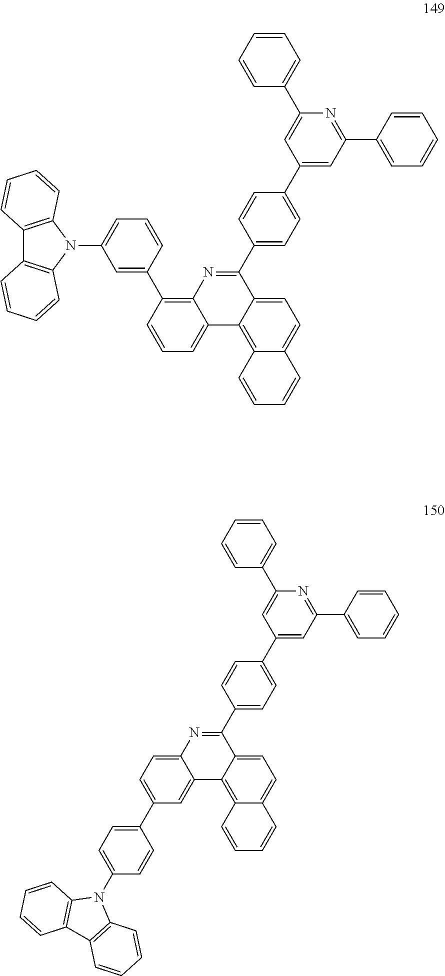 [SMILES: c1ccc(-c2cc(-c3ccc(-c4nc5c(-c6cccc(-n7c8ccccc8c8ccccc87)c6)cccc5c5c4ccc4ccccc45)cc3)cc(-c3ccccc3)n2)cc1 ; c1ccc(-c2cc(-c3ccc(-c4nc5ccc(-c6ccc(-n7c8ccccc8c8ccccc87)cc6)cc5c5c4ccc4ccccc45)cc3)cc(-c3ccccc3)n2)cc1]

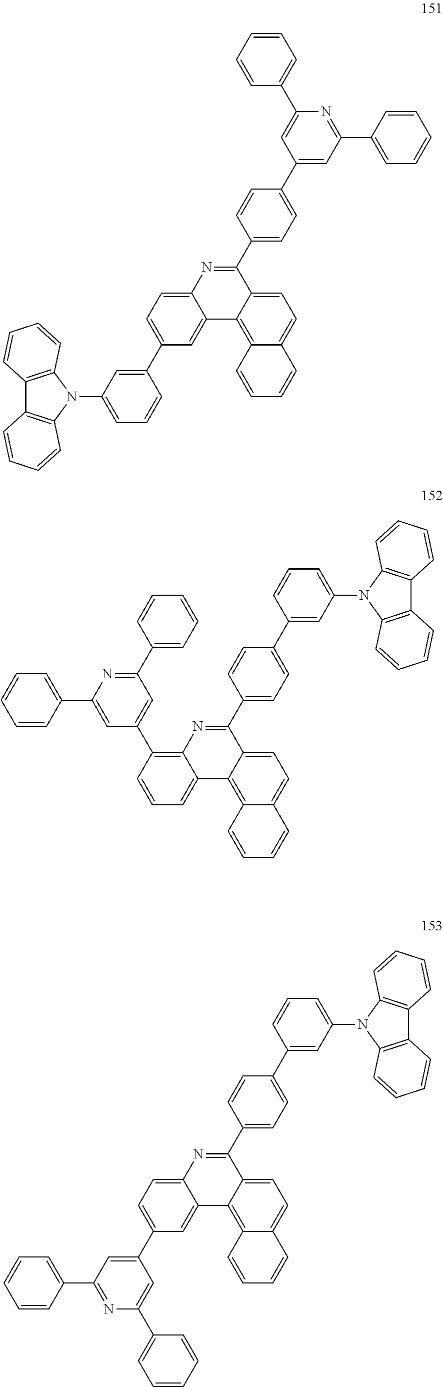 [SMILES: c1ccc(-c2cc(-c3ccc(-c4nc5ccc(-c6cccc(-n7c8ccccc8c8ccccc87)c6)cc5c5c4ccc4ccccc45)cc3)cc(-c3ccccc3)n2)cc1 ; c1ccc(-c2cc(-c3cccc4c3nc(-c3ccc(-c5cccc(-n6c7ccccc7c7ccccc76)c5)cc3)c3ccc5ccccc5c34)cc(-c3ccccc3)n2)cc1 ; c1ccc(-c2cc(-c3ccc4nc(-c5ccc(-c6cccc(-n7c8ccccc8c8ccccc87)c6)cc5)c5ccc6ccccc6c5c4c3)cc(-c3ccccc3)n2)cc1]

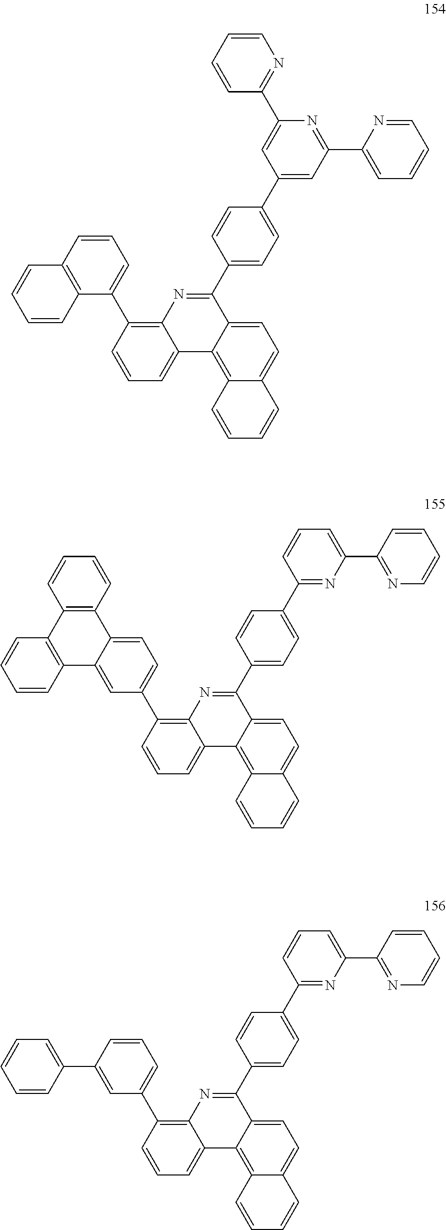 [SMILES: c1ccc(-c2cc(-c3ccc(-c4nc5c(-c6cccc7ccccc67)cccc5c5c4ccc4ccccc45)cc3)cc(-c3ccccn3)n2)nc1 ; c1ccc(-c2cccc(-c3ccc(-c4nc5c(-c6ccc7c8ccccc8c8ccccc8c7c6)cccc5c5c4ccc4ccccc45)cc3)n2)nc1 ; c1ccc(-c2cccc(-c3cccc4c3nc(-c3ccc(-c5cccc(-c6ccccn6)n5)cc3)c3ccc5ccccc5c34)c2)cc1]

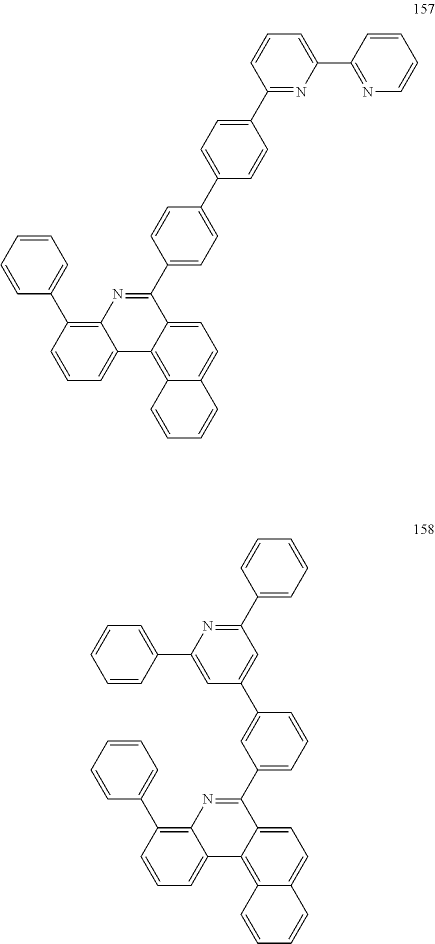 [SMILES: c1ccc(-c2cccc3c2nc(-c2ccc(-c4ccc(-c5cccc(-c6ccccn6)n5)cc4)cc2)c2ccc4ccccc4c23)cc1 ; c1ccc(-c2cc(-c3cccc(-c4nc5c(-c6ccccc6)cccc5c5c4ccc4ccccc45)c3)cc(-c3ccccc3)n2)cc1]

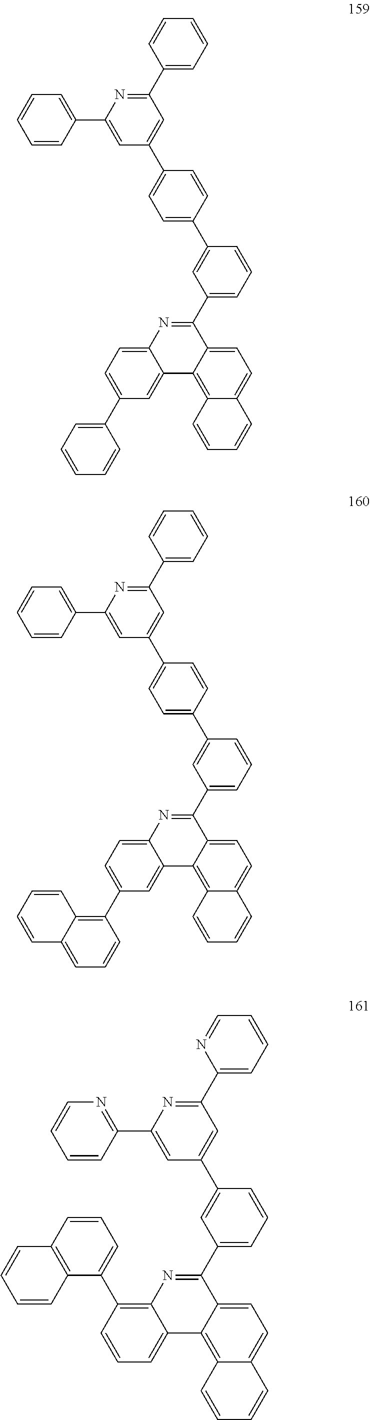 [SMILES: c1ccc(-c2ccc3nc(-c4cccc(-c5ccc(-c6cc(-c7ccccc7)nc(-c7ccccc7)c6)cc5)c4)c4ccc5ccccc5c4c3c2)cc1 ; c1ccc(-c2cc(-c3ccc(-c4cccc(-c5nc6ccc(-c7cccc8ccccc78)cc6c6c5ccc5ccccc56)c4)cc3)cc(-c3ccccc3)n2)cc1 ; c1ccc(-c2cc(-c3cccc(-c4nc5c(-c6cccc7ccccc67)cccc5c5c4ccc4ccccc45)c3)cc(-c3ccccn3)n2)nc1]

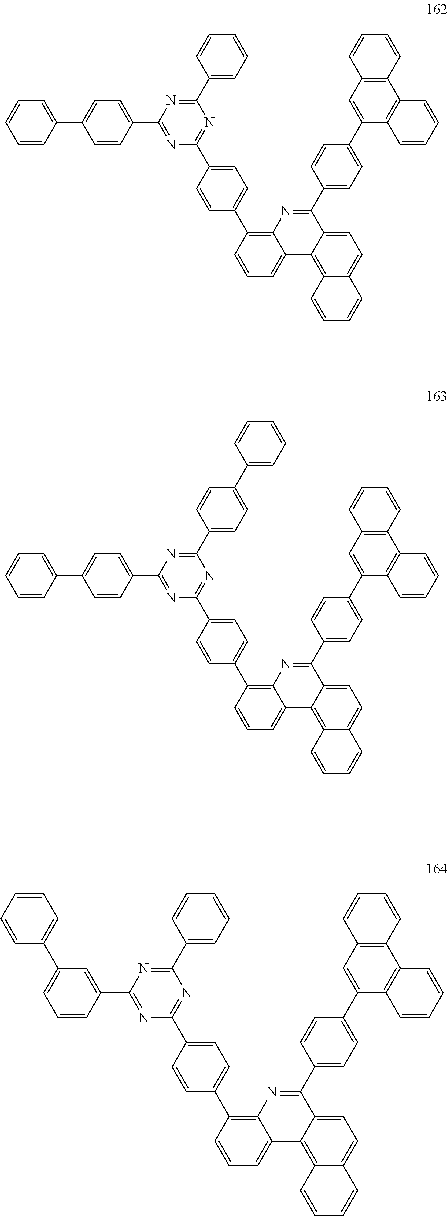 [SMILES: c1ccc(-c2ccc(-c3nc(-c4ccccc4)nc(-c4ccc(-c5cccc6c5nc(-c5ccc(-c7cc8ccccc8c8ccccc78)cc5)c5ccc7ccccc7c56)cc4)n3)cc2)cc1 ; c1ccc(-c2ccc(-c3nc(-c4ccc(-c5ccccc5)cc4)nc(-c4ccc(-c5cccc6c5nc(-c5ccc(-c7cc8ccccc8c8ccccc78)cc5)c5ccc7ccccc7c56)cc4)n3)cc2)cc1 ; c1ccc(-c2cccc(-c3nc(-c4ccccc4)nc(-c4ccc(-c5cccc6c5nc(-c5ccc(-c7cc8ccccc8c8ccccc78)cc5)c5ccc7ccccc7c56)cc4)n3)c2)cc1]

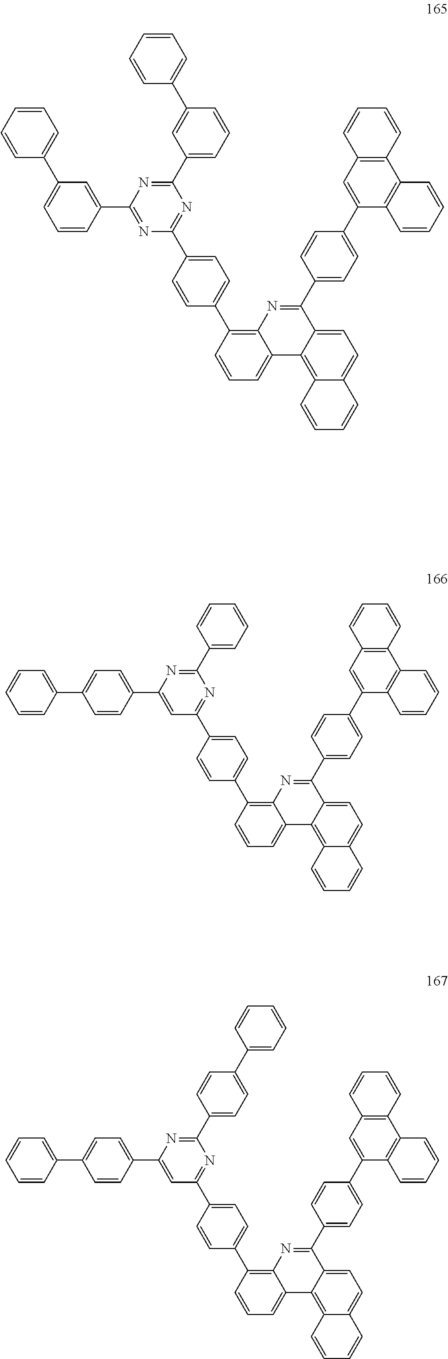 [SMILES: c1ccc(-c2cccc(-c3nc(-c4ccc(-c5cccc6c5nc(-c5ccc(-c7cc8ccccc8c8ccccc78)cc5)c5ccc7ccccc7c56)cc4)nc(-c4cccc(-c5ccccc5)c4)n3)c2)cc1 ; c1ccc(-c2ccc(-c3cc(-c4ccc(-c5cccc6c5nc(-c5ccc(-c7cc8ccccc8c8ccccc78)cc5)c5ccc7ccccc7c56)cc4)nc(-c4ccccc4)n3)cc2)cc1 ; c1ccc(-c2ccc(-c3cc(-c4ccc(-c5cccc6c5nc(-c5ccc(-c7cc8ccccc8c8ccccc78)cc5)c5ccc7ccccc7c56)cc4)nc(-c4ccc(-c5ccccc5)cc4)n3)cc2)cc1]

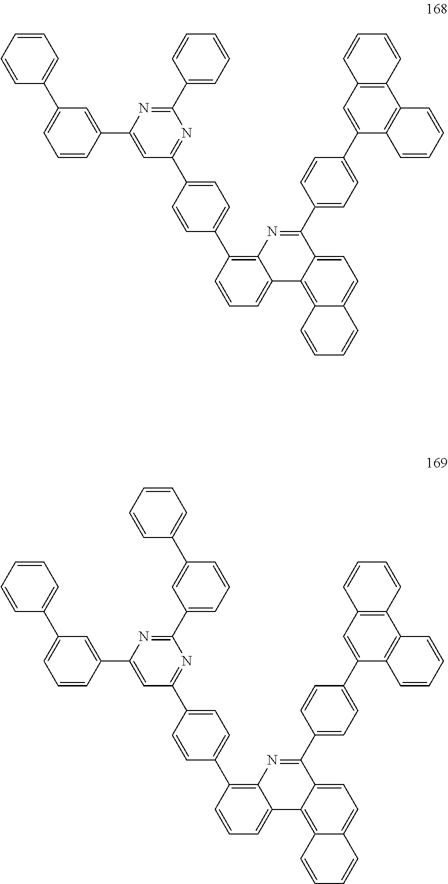 [SMILES: c1ccc(-c2cccc(-c3cc(-c4ccc(-c5cccc6c5nc(-c5ccc(-c7cc8ccccc8c8ccccc78)cc5)c5ccc7ccccc7c56)cc4)nc(-c4ccccc4)n3)c2)cc1 ; c1ccc(-c2cccc(-c3cc(-c4ccc(-c5cccc6c5nc(-c5ccc(-c7cc8ccccc8c8ccccc78)cc5)c5ccc7ccccc7c56)cc4)nc(-c4cccc(-c5ccccc5)c4)n3)c2)cc1]

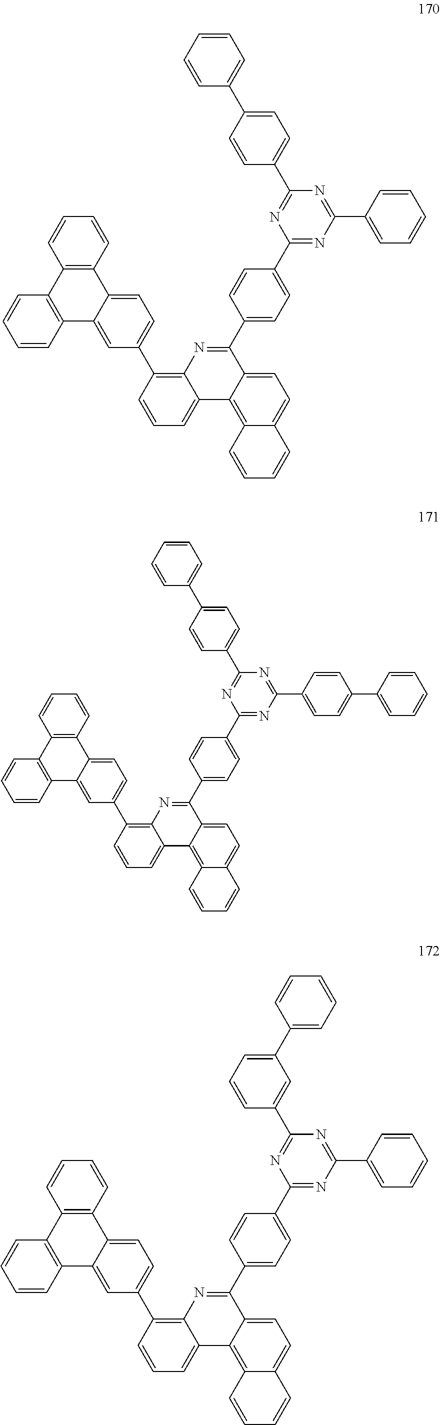 [SMILES: c1ccc(-c2ccc(-c3nc(-c4ccccc4)nc(-c4ccc(-c5nc6c(-c7ccc8c9ccccc9c9ccccc9c8c7)cccc6c6c5ccc5ccccc56)cc4)n3)cc2)cc1 ; c1ccc(-c2ccc(-c3nc(-c4ccc(-c5ccccc5)cc4)nc(-c4ccc(-c5nc6c(-c7ccc8c9ccccc9c9ccccc9c8c7)cccc6c6c5ccc5ccccc56)cc4)n3)cc2)cc1 ; c1ccc(-c2cccc(-c3nc(-c4ccccc4)nc(-c4ccc(-c5nc6c(-c7ccc8c9ccccc9c9ccccc9c8c7)cccc6c6c5ccc5ccccc56)cc4)n3)c2)cc1]

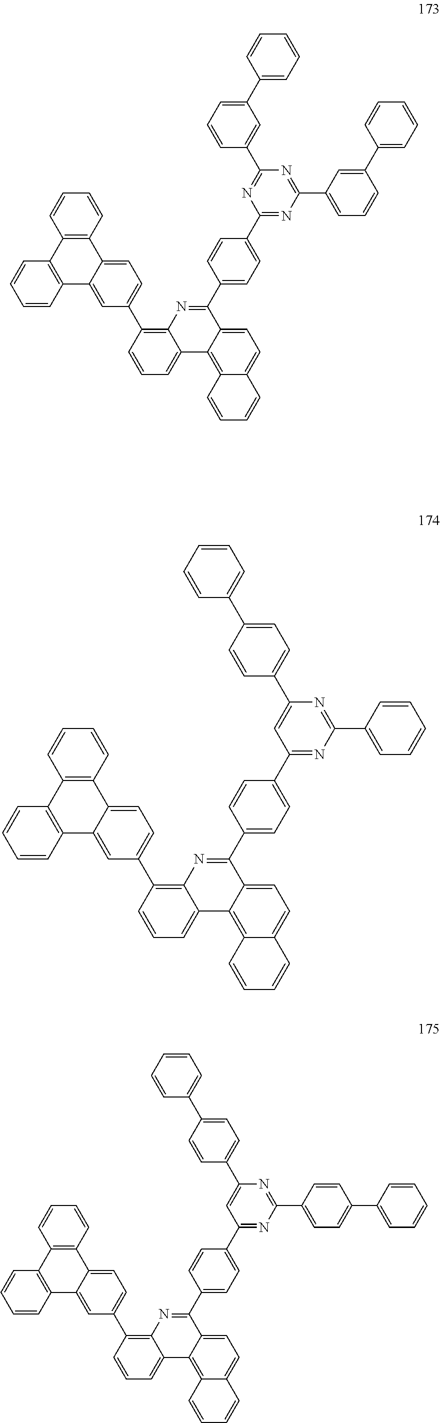 [SMILES: c1ccc(-c2cccc(-c3nc(-c4ccc(-c5nc6c(-c7ccc8c9ccccc9c9ccccc9c8c7)cccc6c6c5ccc5ccccc56)cc4)nc(-c4cccc(-c5ccccc5)c4)n3)c2)cc1 ; c1ccc(-c2ccc(-c3cc(-c4ccc(-c5nc6c(-c7ccc8c9ccccc9c9ccccc9c8c7)cccc6c6c5ccc5ccccc56)cc4)nc(-c4ccccc4)n3)cc2)cc1 ; c1ccc(-c2ccc(-c3cc(-c4ccc(-c5nc6c(-c7ccc8c9ccccc9c9ccccc9c8c7)cccc6c6c5ccc5ccccc56)cc4)nc(-c4ccc(-c5ccccc5)cc4)n3)cc2)cc1]

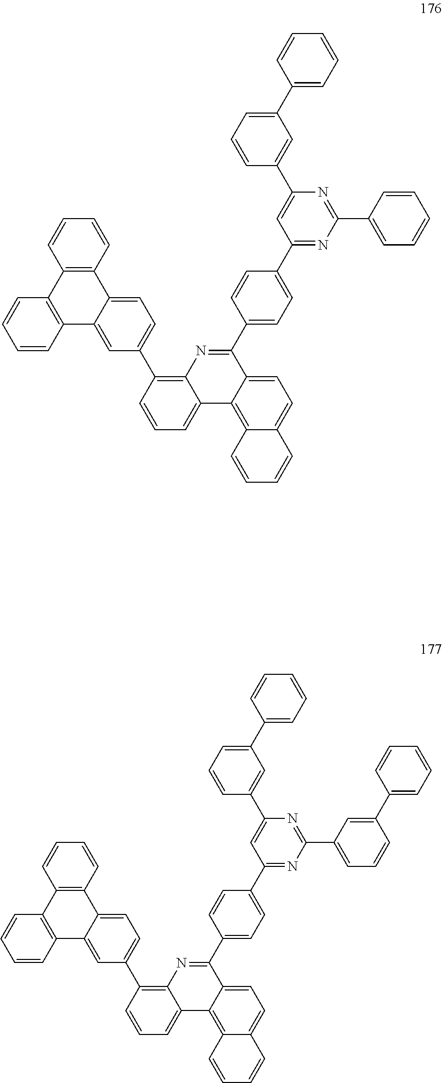 [SMILES: c1ccc(-c2cccc(-c3cc(-c4ccc(-c5nc6c(-c7ccc8c9ccccc9c9ccccc9c8c7)cccc6c6c5ccc5ccccc56)cc4)nc(-c4ccccc4)n3)c2)cc1 ; c1ccc(-c2cccc(-c3cc(-c4ccc(-c5nc6c(-c7ccc8c9ccccc9c9ccccc9c8c7)cccc6c6c5ccc5ccccc56)cc4)nc(-c4cccc(-c5ccccc5)c4)n3)c2)cc1]

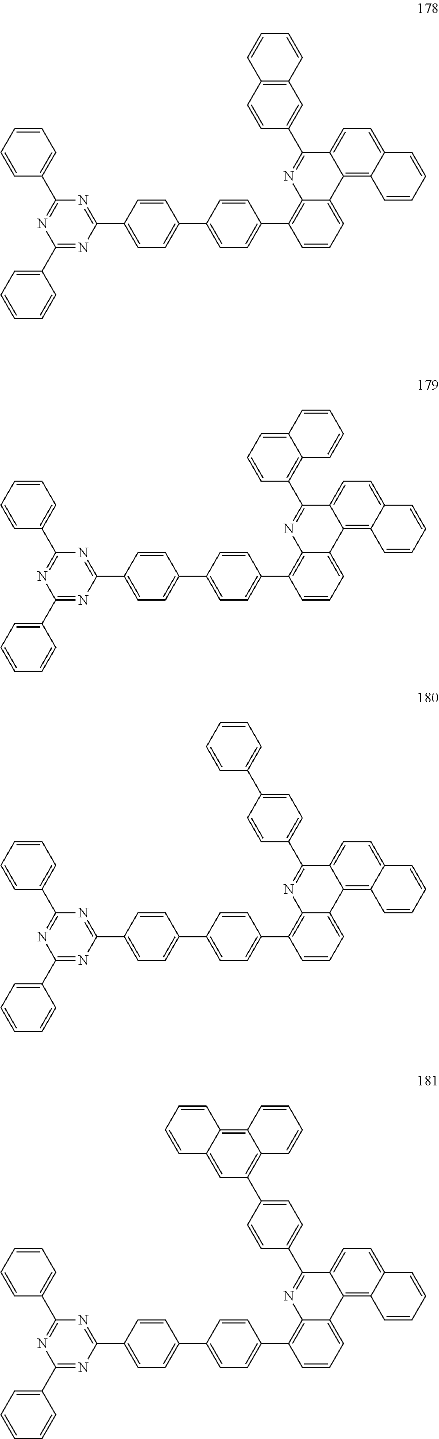 [SMILES: c1ccc(-c2nc(-c3ccccc3)nc(-c3ccc(-c4ccc(-c5cccc6c5nc(-c5ccc7ccccc7c5)c5ccc7ccccc7c56)cc4)cc3)n2)cc1 ; c1ccc(-c2nc(-c3ccccc3)nc(-c3ccc(-c4ccc(-c5cccc6c5nc(-c5cccc7ccccc57)c5ccc7ccccc7c56)cc4)cc3)n2)cc1 ; c1ccc(-c2ccc(-c3nc4c(-c5ccc(-c6ccc(-c7nc(-c8ccccc8)nc(-c8ccccc8)n7)cc6)cc5)cccc4c4c3ccc3ccccc34)cc2)cc1 ; c1ccc(-c2nc(-c3ccccc3)nc(-c3ccc(-c4ccc(-c5cccc6c5nc(-c5ccc(-c7cc8ccccc8c8ccccc78)cc5)c5ccc7ccccc7c56)cc4)cc3)n2)cc1]

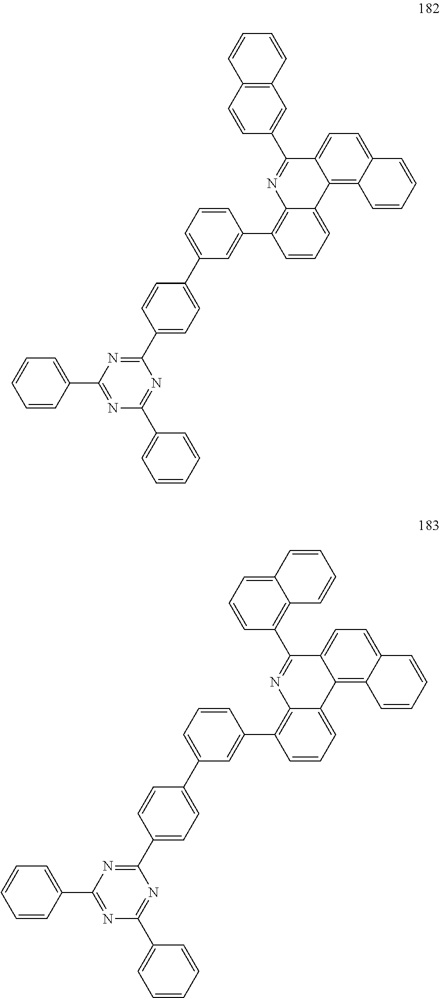 [SMILES: c1ccc(-c2nc(-c3ccccc3)nc(-c3ccc(-c4cccc(-c5cccc6c5nc(-c5ccc7ccccc7c5)c5ccc7ccccc7c56)c4)cc3)n2)cc1 ; c1ccc(-c2nc(-c3ccccc3)nc(-c3ccc(-c4cccc(-c5cccc6c5nc(-c5cccc7ccccc57)c5ccc7ccccc7c56)c4)cc3)n2)cc1]

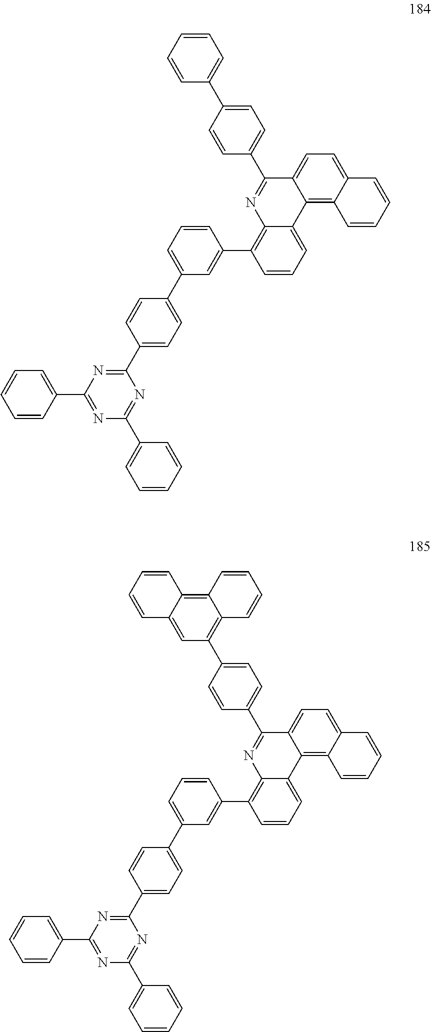 [SMILES: c1ccc(-c2ccc(-c3nc4c(-c5cccc(-c6ccc(-c7nc(-c8ccccc8)nc(-c8ccccc8)n7)cc6)c5)cccc4c4c3ccc3ccccc34)cc2)cc1 ; c1ccc(-c2nc(-c3ccccc3)nc(-c3ccc(-c4cccc(-c5cccc6c5nc(-c5ccc(-c7cc8ccccc8c8ccccc78)cc5)c5ccc7ccccc7c56)c4)cc3)n2)cc1]

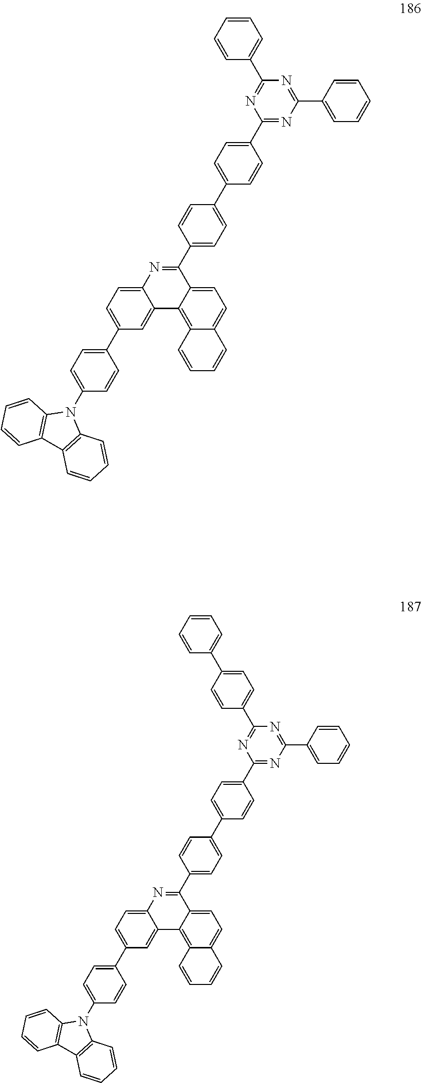 [SMILES: c1ccc(-c2nc(-c3ccccc3)nc(-c3ccc(-c4ccc(-c5nc6ccc(-c7ccc(-n8c9ccccc9c9ccccc98)cc7)cc6c6c5ccc5ccccc56)cc4)cc3)n2)cc1 ; c1ccc(-c2ccc(-c3nc(-c4ccccc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)cc2)cc1]

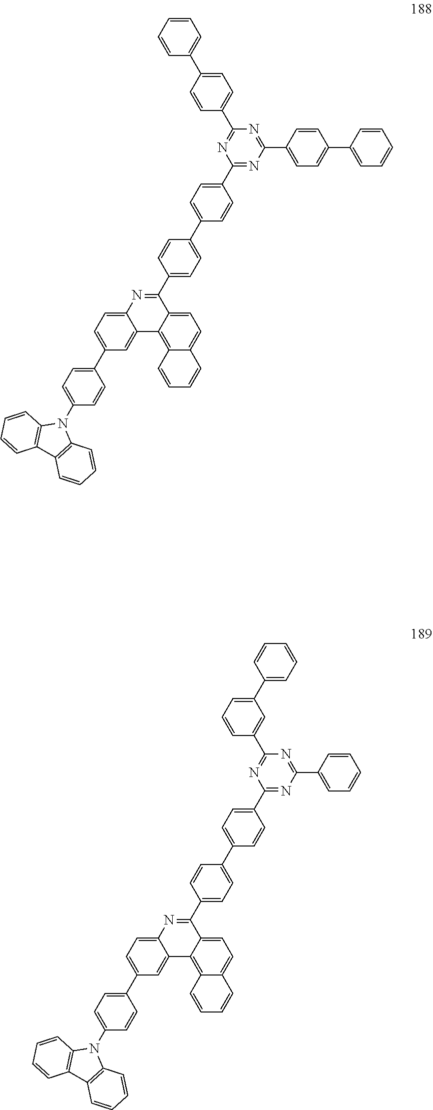 [SMILES: c1ccc(-c2ccc(-c3nc(-c4ccc(-c5ccccc5)cc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)cc2)cc1 ; c1ccc(-c2cccc(-c3nc(-c4ccccc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)c2)cc1]

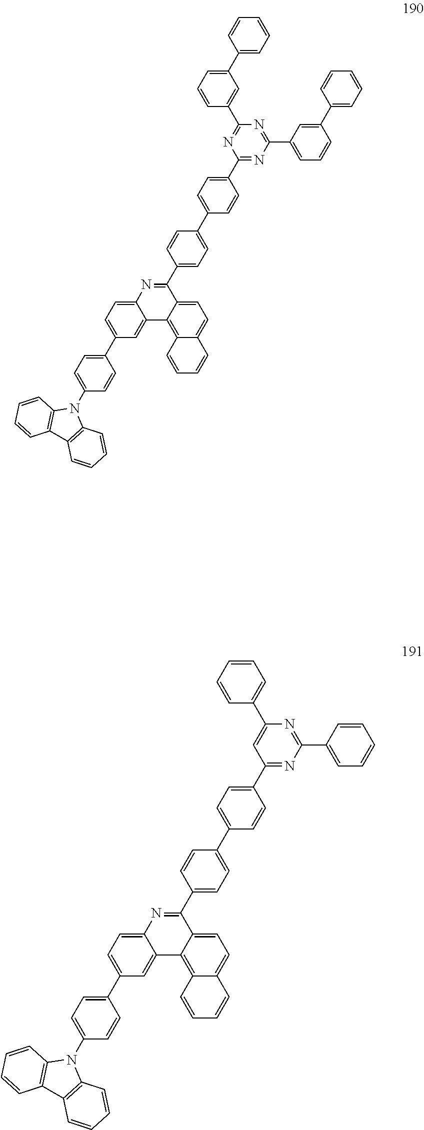 [SMILES: c1ccc(-c2cccc(-c3nc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4cccc(-c5ccccc5)c4)n3)c2)cc1 ; c1ccc(-c2cc(-c3ccc(-c4ccc(-c5nc6ccc(-c7ccc(-n8c9ccccc9c9ccccc98)cc7)cc6c6c5ccc5ccccc56)cc4)cc3)nc(-c3ccccc3)n2)cc1]

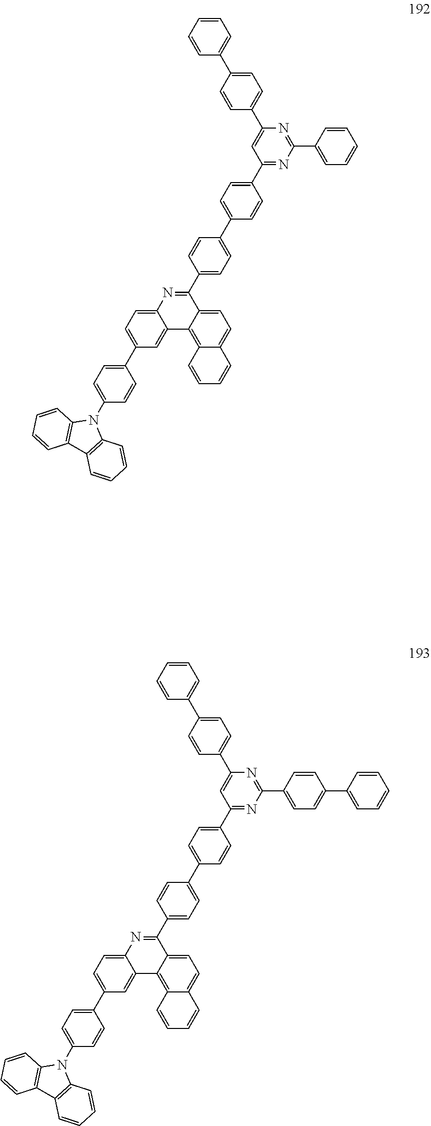 [SMILES: c1ccc(-c2ccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccccc4)n3)cc2)cc1 ; c1ccc(-c2ccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccc(-c5ccccc5)cc4)n3)cc2)cc1]

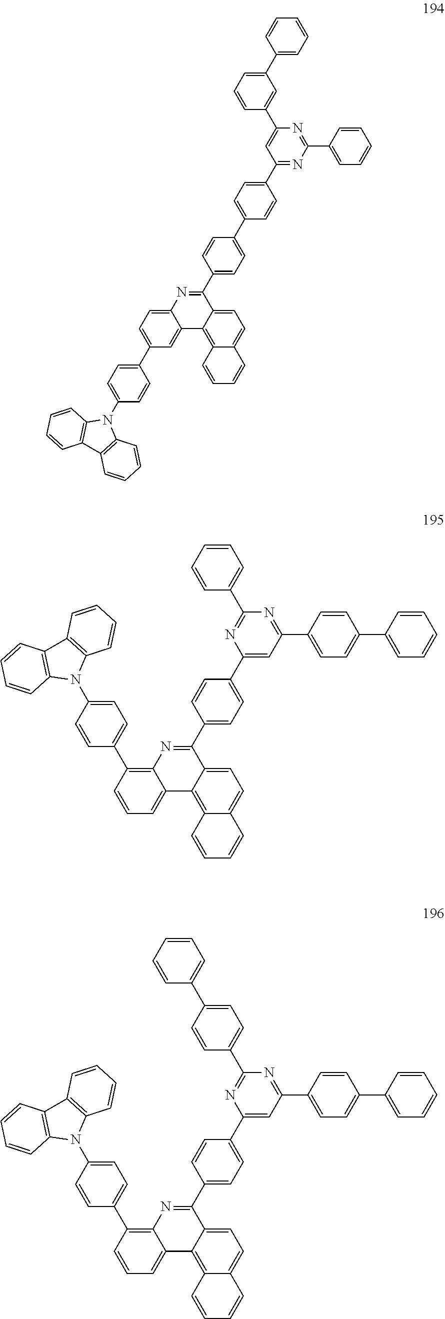 [SMILES: c1ccc(-c2cccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccccc4)n3)c2)cc1 ; c1ccc(-c2ccc(-c3cc(-c4ccc(-c5nc6c(-c7ccc(-n8c9ccccc9c9ccccc98)cc7)cccc6c6c5ccc5ccccc56)cc4)nc(-c4ccccc4)n3)cc2)cc1 ; c1ccc(-c2ccc(-c3cc(-c4ccc(-c5nc6c(-c7ccc(-n8c9ccccc9c9ccccc98)cc7)cccc6c6c5ccc5ccccc56)cc4)nc(-c4ccc(-c5ccccc5)cc4)n3)cc2)cc1]

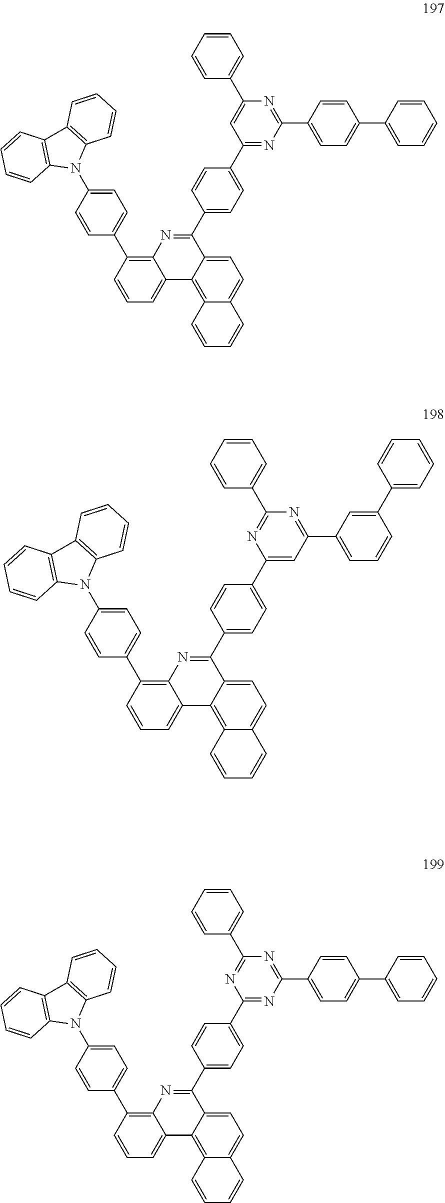 [SMILES: c1ccc(-c2ccc(-c3nc(-c4ccccc4)cc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2cccc(-c3cc(-c4ccc(-c5nc6c(-c7ccc(-n8c9ccccc9c9ccccc98)cc7)cccc6c6c5ccc5ccccc56)cc4)nc(-c4ccccc4)n3)c2)cc1 ; c1ccc(-c2ccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)cc2)cc1]

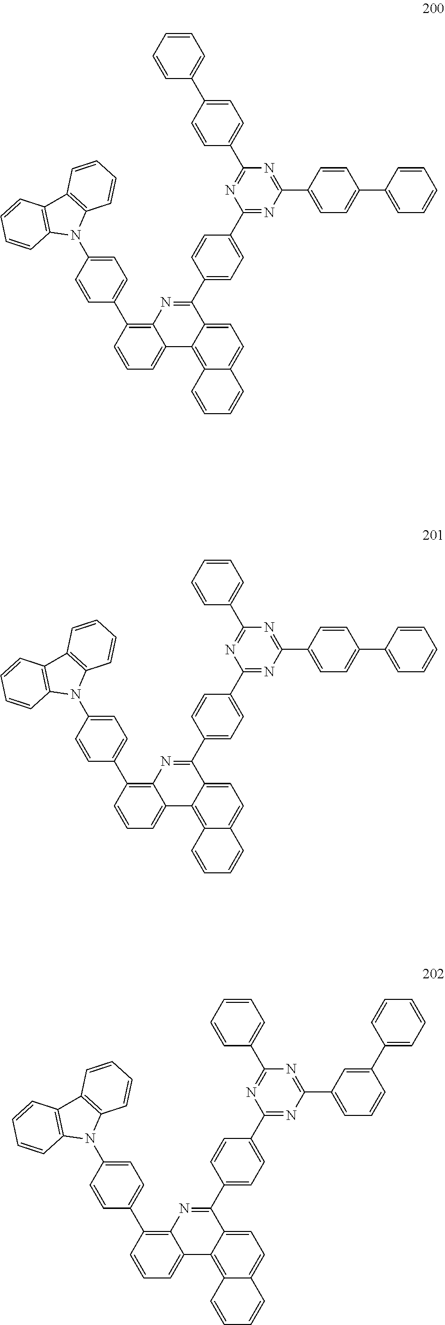 [SMILES: c1ccc(-c2ccc(-c3nc(-c4ccc(-c5ccccc5)cc4)nc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2ccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2cccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)c2)cc1]

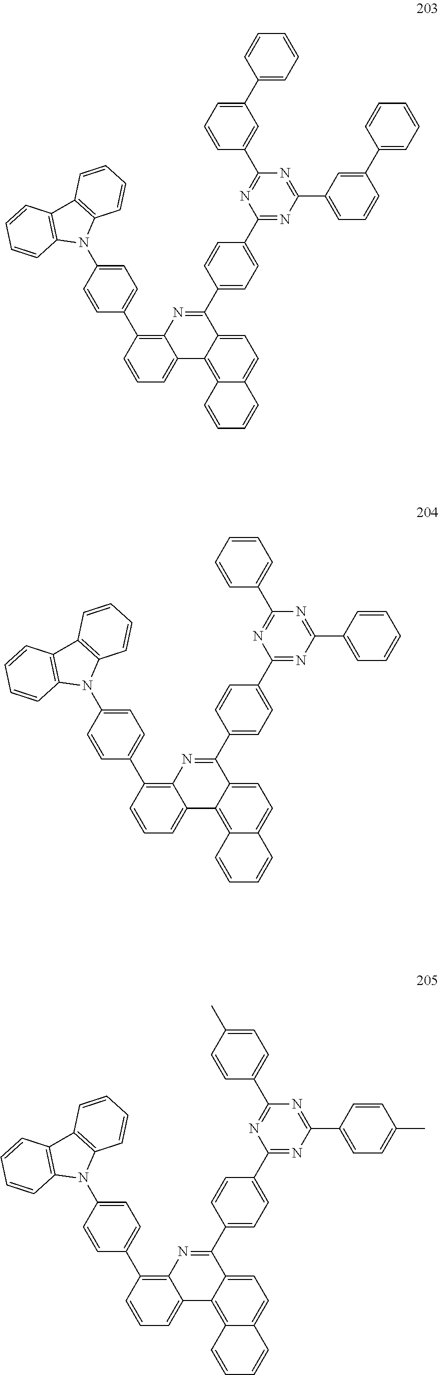 [SMILES: c1ccc(-c2cccc(-c3nc(-c4ccc(-c5nc6c(-c7ccc(-n8c9ccccc9c9ccccc98)cc7)cccc6c6c5ccc5ccccc56)cc4)nc(-c4cccc(-c5ccccc5)c4)n3)c2)cc1 ; c1ccc(-c2nc(-c3ccccc3)nc(-c3ccc(-c4nc5c(-c6ccc(-n7c8ccccc8c8ccccc87)cc6)cccc5c5c4ccc4ccccc45)cc3)n2)cc1 ; Cc1ccc(-c2nc(-c3ccc(C)cc3)nc(-c3ccc(-c4nc5c(-c6ccc(-n7c8ccccc8c8ccccc87)cc6)cccc5c5c4ccc4ccccc45)cc3)n2)cc1]

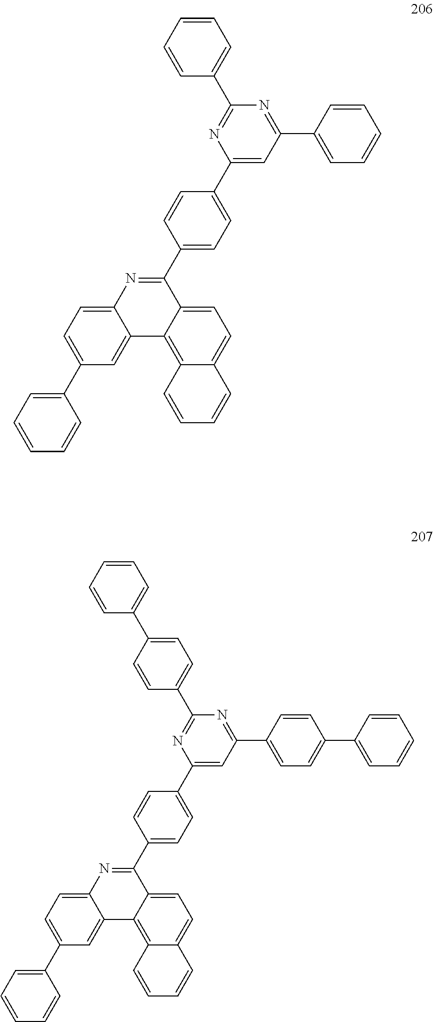 [SMILES: c1ccc(-c2ccc3nc(-c4ccc(-c5cc(-c6ccccc6)nc(-c6ccccc6)n5)cc4)c4ccc5ccccc5c4c3c2)cc1 ; c1ccc(-c2ccc(-c3cc(-c4ccc(-c5nc6ccc(-c7ccccc7)cc6c6c5ccc5ccccc56)cc4)nc(-c4ccc(-c5ccccc5)cc4)n3)cc2)cc1]

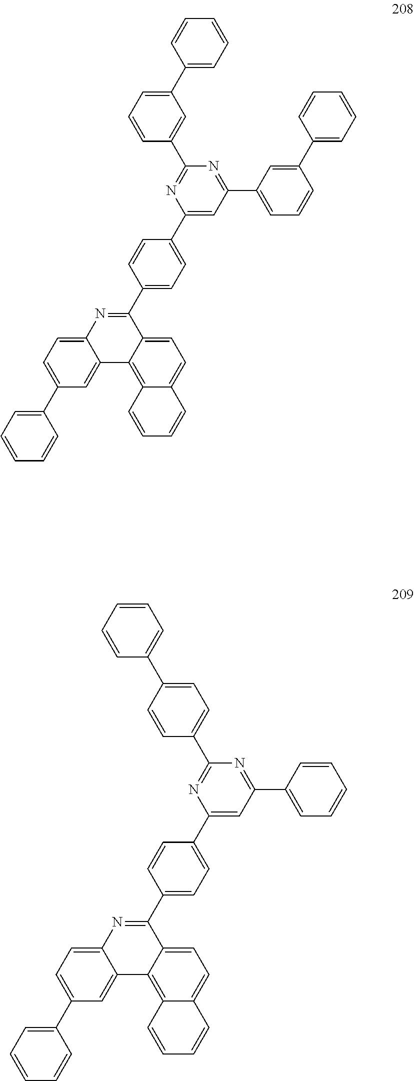 [SMILES: c1ccc(-c2cccc(-c3cc(-c4ccc(-c5nc6ccc(-c7ccccc7)cc6c6c5ccc5ccccc56)cc4)nc(-c4cccc(-c5ccccc5)c4)n3)c2)cc1 ; c1ccc(-c2ccc(-c3nc(-c4ccccc4)cc(-c4ccc(-c5nc6ccc(-c7ccccc7)cc6c6c5ccc5ccccc56)cc4)n3)cc2)cc1]

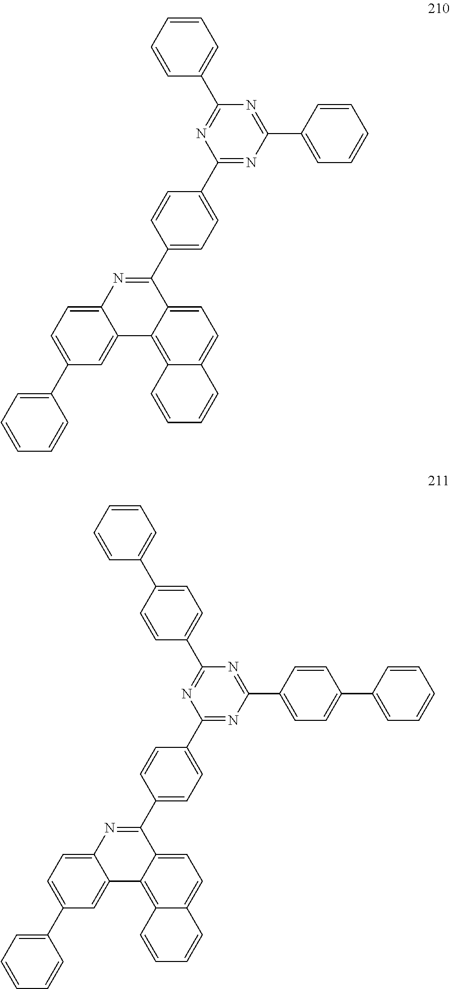 [SMILES: c1ccc(-c2ccc3nc(-c4ccc(-c5nc(-c6ccccc6)nc(-c6ccccc6)n5)cc4)c4ccc5ccccc5c4c3c2)cc1 ; c1ccc(-c2ccc(-c3nc(-c4ccc(-c5ccccc5)cc4)nc(-c4ccc(-c5nc6ccc(-c7ccccc7)cc6c6c5ccc5ccccc56)cc4)n3)cc2)cc1]

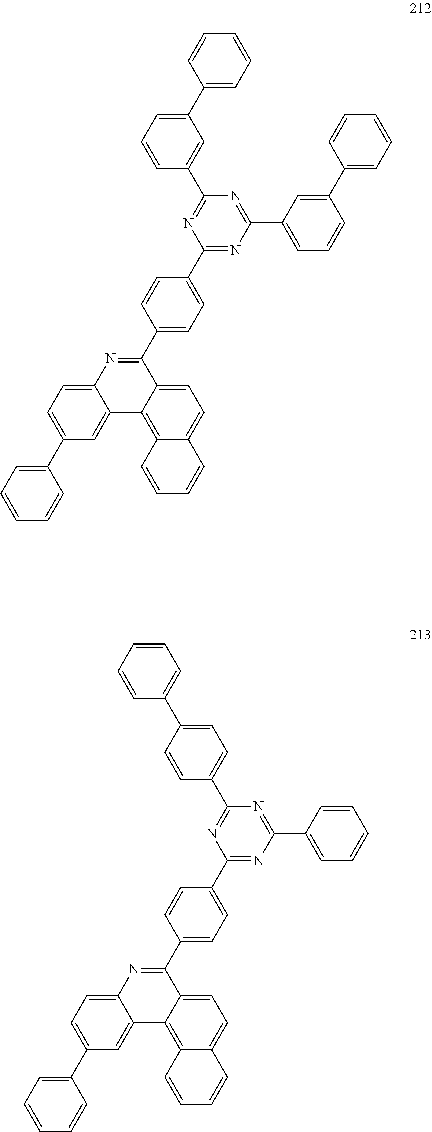 [SMILES: c1ccc(-c2cccc(-c3nc(-c4ccc(-c5nc6ccc(-c7ccccc7)cc6c6c5ccc5ccccc56)cc4)nc(-c4cccc(-c5ccccc5)c4)n3)c2)cc1 ; c1ccc(-c2ccc(-c3nc(-c4ccccc4)nc(-c4ccc(-c5nc6ccc(-c7ccccc7)cc6c6c5ccc5ccccc56)cc4)n3)cc2)cc1]

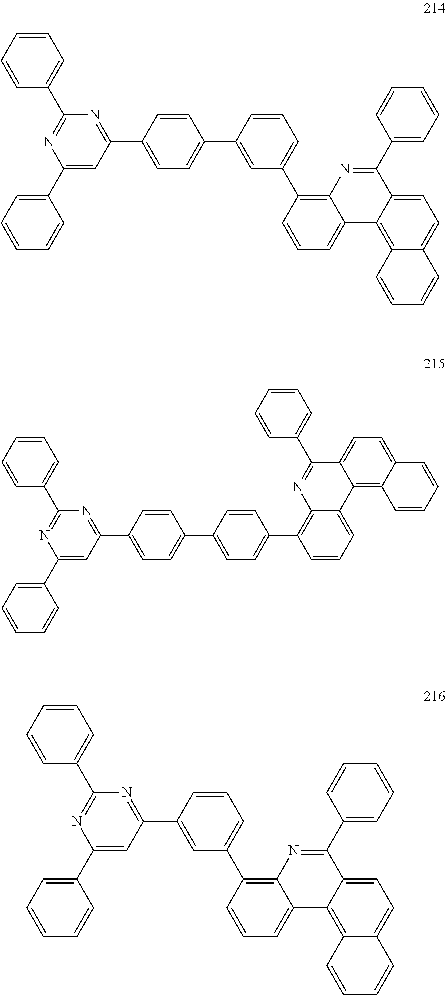 [SMILES: c1ccc(-c2cc(-c3ccc(-c4cccc(-c5cccc6c5nc(-c5ccccc5)c5ccc7ccccc7c56)c4)cc3)nc(-c3ccccc3)n2)cc1 ; c1ccc(-c2cc(-c3ccc(-c4ccc(-c5cccc6c5nc(-c5ccccc5)c5ccc7ccccc7c56)cc4)cc3)nc(-c3ccccc3)n2)cc1 ; c1ccc(-c2cc(-c3cccc(-c4cccc5c4nc(-c4ccccc4)c4ccc6ccccc6c45)c3)nc(-c3ccccc3)n2)cc1]

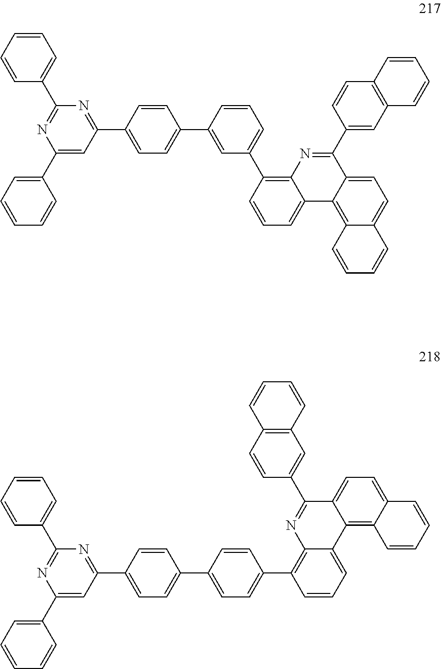 [SMILES: c1ccc(-c2cc(-c3ccc(-c4cccc(-c5cccc6c5nc(-c5ccc7ccccc7c5)c5ccc7ccccc7c56)c4)cc3)nc(-c3ccccc3)n2)cc1 ; c1ccc(-c2cc(-c3ccc(-c4ccc(-c5cccc6c5nc(-c5ccc7ccccc7c5)c5ccc7ccccc7c56)cc4)cc3)nc(-c3ccccc3)n2)cc1]

The compound according to one embodiment of the present application may be prepared according to the following General Formula 1.

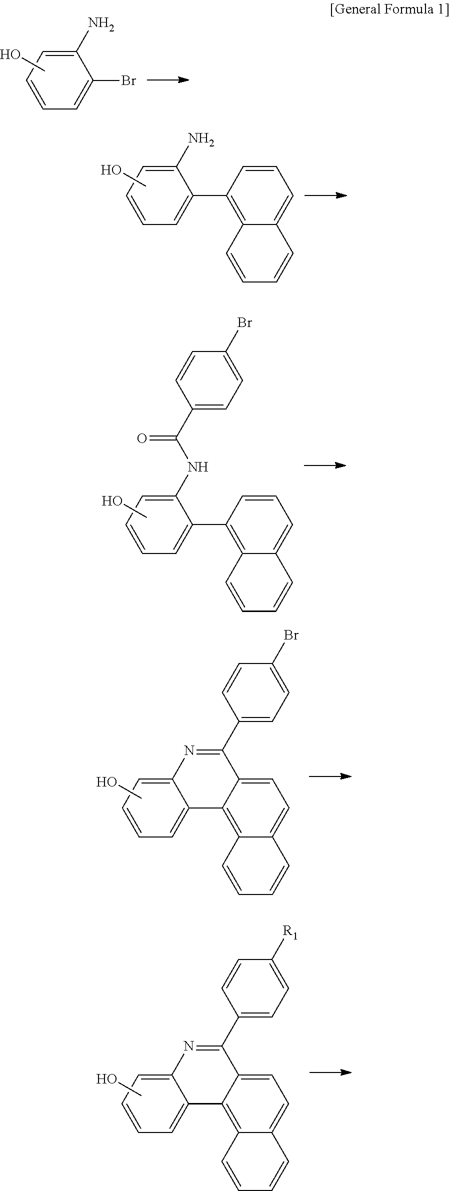

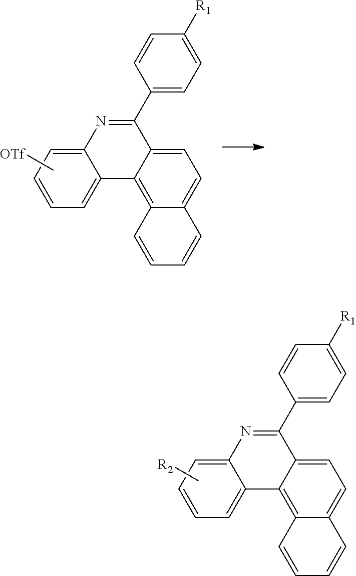

R1 in General Formula 1 has the same definition as -(L₁)m-(Z₁)n in Chemical Formula 1, and R2 in General Formula 1 has the same definition as -(L₂)p-(Z₂)q in Chemical Formula 1.

In addition, by introducing various substituents to the structure of Chemical Formulae 1 to 7, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formulae 1 to 7, the energy band gap may be finely controlled, and meanwhile, properties at interfaces between organic materials are enhanced, and material applications may become diverse.

Meanwhile, the compound has a high glass transition temperature (Tg), and has excellent thermal stability. Such an increase in the thermal stability becomes an important factor providing driving stability to a device.

The heterocyclic compound according to one embodiment of the present application may be prepared through a multistep chemical reaction. Some intermediate compounds are prepared first, and the compound of Chemical Formula 1 may be prepared from the intermediate compounds. More specifically, the heterocyclic compound according to one embodiment of the present application may be prepared based on preparation examples to describe later.

Another embodiment of the present application provides an organic light emitting device comprising a first electrode; a second electrode provided opposite to the first electrode; and one or more organic material layers provided between the first electrode and the second electrode, wherein one or more layers of the organic material layers comprise the heterocyclic compound according to Chemical Formula 1.

In one embodiment of the present application, the first electrode may be an anode, and the second electrode may be a cathode.

In another embodiment, the first electrode may be a cathode, and the second electrode may be an anode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound according to Chemical Formula 1 may be used as a material of the green organic light emitting device.

In one embodiment of the present application, the organic light emitting device may be a red organic light emitting device, and the heterocyclic compound according to Chemical Formula 1 may be used as a material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into an organic material layer through a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, or may also be formed in a multilayer structure in which two or more organic material layers are laminated. For example, the organic light emitting device according to one embodiment of the present disclosure may have a structure comprising a hole injection layer, a hole transfer layer, a light emitting layer, an electron transfer layer, an electron injection layer and the like as the organic material layer. However, the structure of the organic light emitting device is not limited thereto, and may comprise less numbers of organic material layers.

In the organic light emitting device of the present disclosure, the organic material layer comprises an electron injection layer or an electron transfer layer, and the electron injection layer or the electron transfer layer may comprise the heterocyclic compound.

In the organic light emitting device of the present disclosure, the organic material layer comprises an electron transfer layer, and the electron transfer layer may comprise the heterocyclic compound.

In another organic light emitting device, the organic material layer comprises an electron blocking layer or a hole blocking layer, and the electron blocking layer or the hole blocking layer may comprise the heterocyclic compound.

In another organic light emitting device, the organic material layer comprises a hole blocking layer, and the hole blocking layer may comprise the heterocyclic compound.

In another organic light emitting device, the organic material layer comprises an electron transfer layer, a light emitting layer or a hole blocking layer, and the electron transfer layer, the light emitting layer or the hole blocking layer may comprise the heterocyclic compound.

The organic light emitting device of the present disclosure may further comprise one, two or more layers selected from the group consisting of a light emitting layer, a hole injection layer, a hole transfer layer, an electron injection layer, an electron transfer layer, an electron blocking layer and a hole blocking layer.

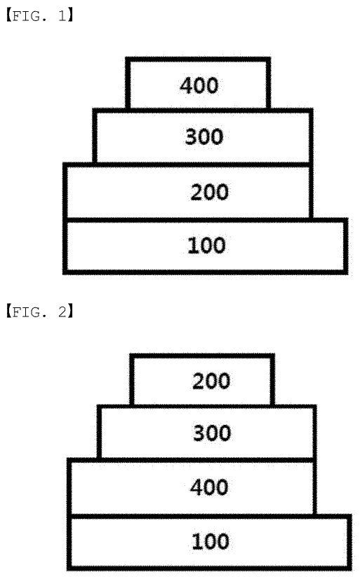

FIGS. 1 to 3 illustrate a lamination order of electrodes and organic material layers of an organic light emitting device according to one embodiment of the present application. However, the scope of the present application is not limited to these diagrams, and structures of organic light emitting devices known in the art may also be used in the present application.

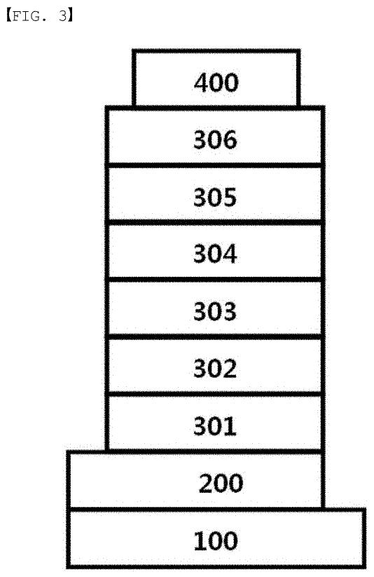

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

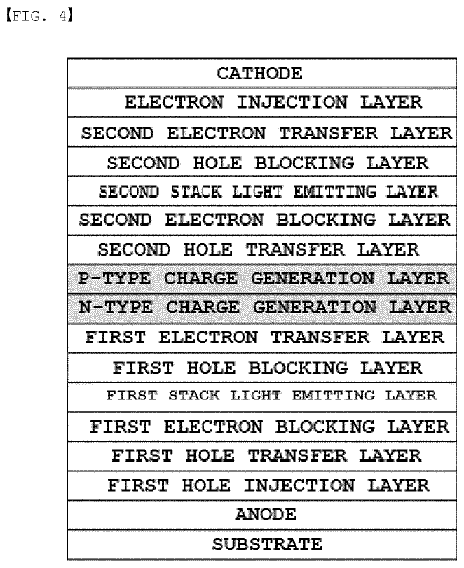

FIG. 3 illustrates a case of the organic material layer being a multilayer. The organic light emitting device according to FIG. 3 comprises a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, other layers except the light emitting layer may not be included, and other necessary functional layers may be further included.

The organic material layer comprising Chemical Formulae 1 to 7 may further comprise other materials as necessary.

In addition, the organic light emitting device according to one embodiment of the present application comprises an anode, a cathode, and two or more stacks provided between the anode and the cathode, wherein the two or more stacks each independently comprise a light emitting layer, a charge generation layer is included between the two or more stacks, and the charge generation layer comprises the heterocyclic compound represented by Chemical Formula 1.

In addition, the organic light emitting device according to one embodiment of the present application may comprise an anode, a first stack provided on the anode and comprising a first light emitting layer, a charge generation layer provided on the first stack, a second stack provided on the charge generation layer and comprising a second light emitting layer, and a cathode provided on the second stack. Herein, the charge generation layer may comprise the heterocyclic compound represented by Chemical Formula 1. In addition, the first stack and the second stack may each independently further comprise one or more types of the hole injection layer, the hole transfer layer, the hole blocking layer, the electron transfer layer, the electron injection layer described above and the like.

The charge generation layer may be an N-type charge generation layer, and the charge generation layer may further comprise a dopant known in the art in addition to the heterocyclic compound represented by Chemical Formula 1.

As the organic light emitting device according to one embodiment of the present application, an organic light emitting device having a 2-stack tandem structure is schematically illustrated in FIG. 4.

Herein, the first electron blocking layer, the first hole blocking layer and the second hole blocking layer and the like described in FIG. 4 may not be included in some cases.

In the organic light emitting device according to one embodiment of the present application, materials other than the compounds of Chemical Formulae 1 to 7 are illustrated below, however, these are for illustrative purposes only and not for limiting the scope of the present application, and may be replaced by materials known in the art.

As the anode material, materials having relatively large work function may be used, and transparent conductive oxides, metals, conductive polymers or the like may be used. Specific examples of the anode material comprise metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the cathode material, materials having relatively small work function may be used, and metals, metal oxides, conductive polymers or the like may be used. Specific examples of the cathode material comprise metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrene-sulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials.

As examples of the electron injection material, LiF is typically used in the art, however, the present application is not limited thereto.

As the light emitting material, red, green or blue light emitting materials may be used, and as necessary, two or more light emitting materials may be mixed and used. Herein, two or more light emitting materials may be used by being deposited as individual sources of supply or by being premixed and deposited as one source of supply. In addition, fluorescent materials may also be used as the light emitting material, however, phosphorescent materials may also be used. As the light emitting material, materials emitting light by bonding electrons and holes injected from an anode and a cathode, respectively, may be used alone, however, materials having a host material and a dopant material involved in light emission together may also be used.

When mixing light emitting material hosts, same series hosts may be mixed, or different series hosts may be mixed. For example, any two or more types of materials among n-type host materials or p-type host materials may be selected, and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device comprising an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### PREPARATION EXAMPLE

### <Preparation Example 1> Preparation of Compound 1

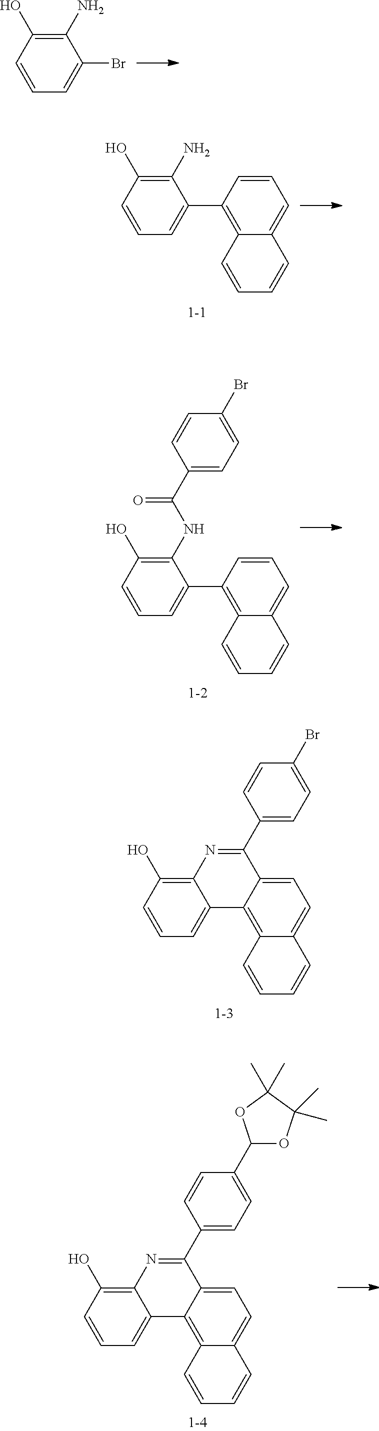 [SMILES: Nc1c(O)cccc1Br ; Nc1c(O)cccc1-c1cccc2ccccc12 ; O=C(Nc1c(O)cccc1-c1cccc2ccccc12)c1ccc(Br)cc1 ; Oc1cccc2c1nc(-c1ccc(Br)cc1)c1ccc3ccccc3c12 ; CC1(C)OC(c2ccc(-c3nc4c(O)cccc4c4c3ccc3ccccc34)cc2)OC1(C)C]

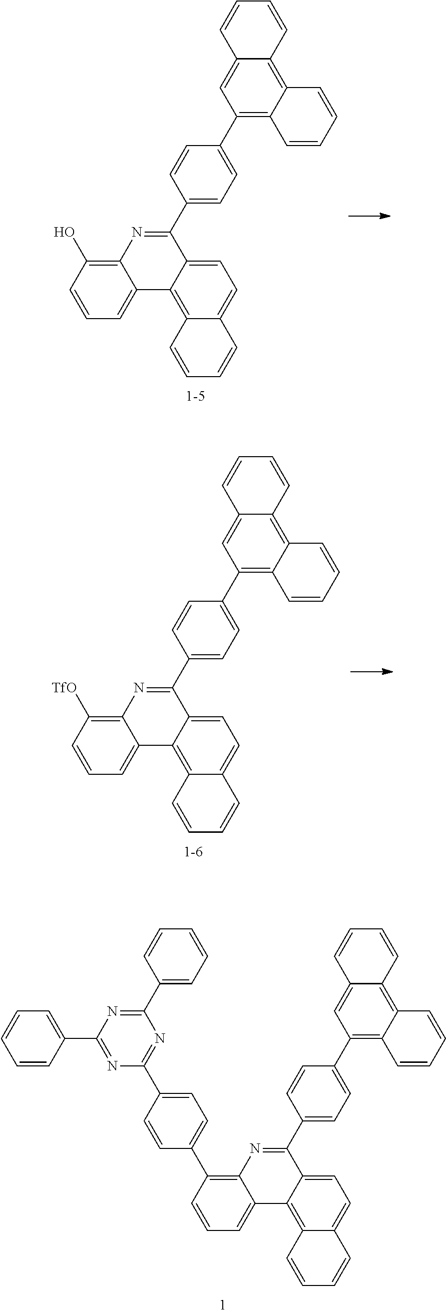 [SMILES: Oc1cccc2c1nc(-c1ccc(-c3cc4ccccc4c4ccccc34)cc1)c1ccc3ccccc3c12 ; O=S(=O)(Oc1cccc2c1nc(-c1ccc(-c3cc4ccccc4c4ccccc34)cc1)c1ccc3ccccc3c12)C(F)(F)F ; c1ccc(-c2nc(-c3ccccc3)nc(-c3ccc(-c4cccc5c4nc(-c4ccc(-c6cc7ccccc7c7ccccc67)cc4)c4ccc6ccccc6c45)cc3)n2)cc1]

Preparation of Compound 1-1

After adding a compound 2-amino-3-bromophenol (20 g, 106 mmol), 1-naphthylboronic acid (20 g, 117 mmol), Pd(PPh₃)₄ (6.1 g, 5.30 mmol), a 2 M aqueous K₂CO₃ solution (100 ml), toluene (400 ml) and ethanol (100 ml), the result was refluxed for 12 hours. After the reaction was completed, the result was cooled to room temperature, and then extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 1-1 (15.7 g, 63%).

Preparation of Compound 1-2

After dissolving Compound 1-1 (15.7 g, 66.7 mmol) in THF, 4-bromobenzoyl chloride (21.9 g, 100 mmol) and TEA (20.2 g, 200 eq.) were added thereto at 0° C., and the result was stirred for 2 hours at room temperature. After the reaction was completed, EA and distilled water were added to the reaction container for solidification, and produced solids were collected to obtain target Compound 1-2 (27 g, 99%).

Preparation of Compound 1-3

After dissolving Compound 1-2 (27 g, 66.0 mmol) in nitrobenzene, POCl₃ (7.5 ml, 1.0 eq.) was added thereto, and the result was stirred for 18 hours at 150° C. After the reaction was completed, the result was vacuum distilled to remove the nitrobenzene, cooled to room temperature and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 1-3 (19 g, 72%).

Preparation of Compound 1-4

After dissolving Compound 1-3 (10 g, 25.0 mmol) in 1,4-dioxane, bis(pinacolato)diboron, Pd(dppf)Cl₂ and potassium acetate were added thereto, and the result was stirred for 2 hours at 110° C. After the reaction was completed, the result was extracted with distilled water and EA. After drying the organic layer with anhydrous MgSO₄, the solvent was removed using a rotary evaporator, and the result was passed through silica gel to obtain target Compound 1-4 (10.4 g, 93%).

Preparation of Compound 1-5

After adding 9-bromophenanthrene (6.6 g, 25.5 mmol), Pd(PPh₃)₄ (1.3 g, 1.16 mmol), K₂CO₃ (9.6 g, 69.6 mmol) and toluene/EtOH/H₂O to Compound 1-4 (10.4 g, 23.2 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 1-5 (9.6 g, 83%).

Preparation of Compound 1-6

After dissolving Compound 1-5 (9.6 g, 19.2 mmol) in dichloromethane and adding pyridine (2.2 g, 28.7 mmol) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 1-6 (12.1 g, 96%).

Preparation of Compound 1

After adding 2-(4-bromophenyl)-4,6-diphenyl-1,3,5-triazine (7.2 g, 20.2 mmol), Pd(PPh₃)₄ (1.0 g, 0.92 mmol), K₂CO₃ (7.6 g, 55.2 mmol) and toluene/EtOH/H₂O to Compound 1-6 (12.1 g, 18.4 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 1 (11.8 g, 81%).

### <Preparation Example 2> Preparation of Compound 3

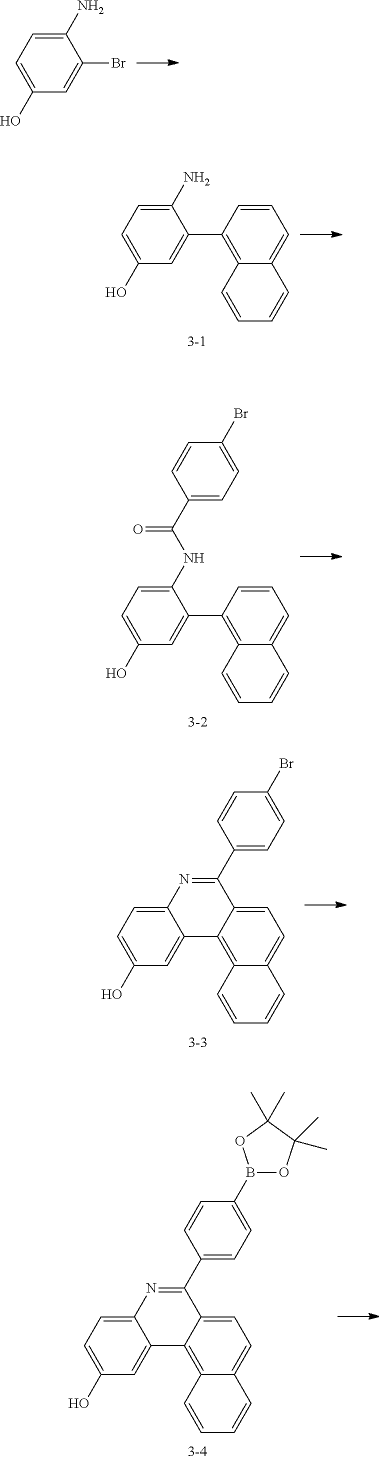 [SMILES: Nc1ccc(O)cc1Br ; Nc1ccc(O)cc1-c1cccc2ccccc12 ; O=C(Nc1ccc(O)cc1-c1cccc2ccccc12)c1ccc(Br)cc1 ; Oc1ccc2nc(-c3ccc(Br)cc3)c3ccc4ccccc4c3c2c1 ; CC1(C)OB(c2ccc(-c3nc4ccc(O)cc4c4c3ccc3ccccc34)cc2)OC1(C)C]

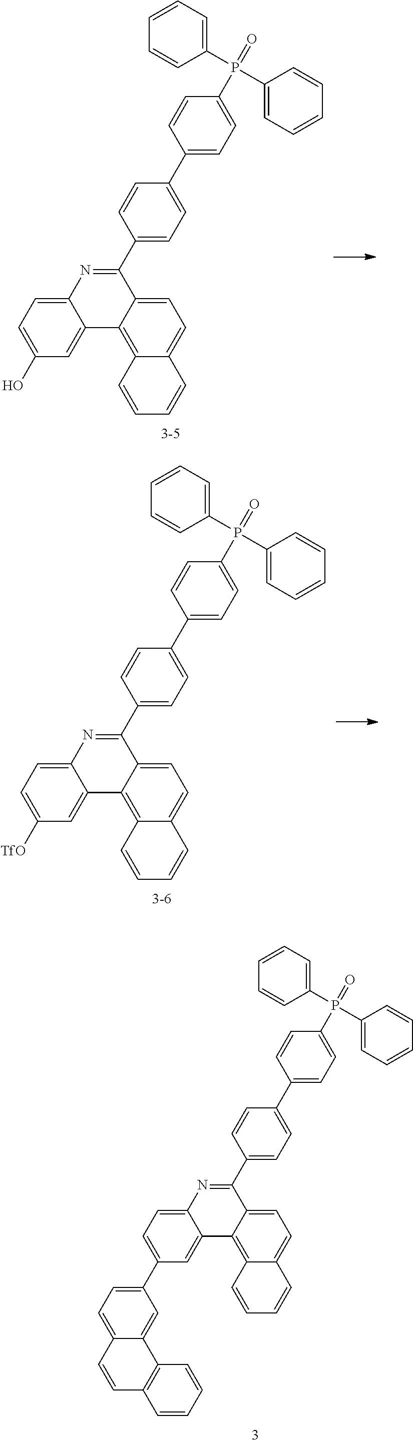 [SMILES: O=P(c1ccccc1)(c1ccccc1)c1ccc(-c2ccc(-c3nc4ccc(O)cc4c4c3ccc3ccccc34)cc2)cc1 ; O=P(c1ccccc1)(c1ccccc1)c1ccc(-c2ccc(-c3nc4ccc(OS(=O)(=O)C(F)(F)F)cc4c4c3ccc3ccccc34)cc2)cc1 ; O=P(c1ccccc1)(c1ccccc1)c1ccc(-c2ccc(-c3nc4ccc(-c5ccc6ccc7ccccc7c6c5)cc4c4c3ccc3ccccc34)cc2)cc1]

Preparation of Compound 3-1

After adding a compound 4-amino-3-bromophenol (20 g, 106 mmol), 1-naphthylboronic acid (20 g, 117 mmol), Pd(PPh₃)₄ (6.1 g, 5.30 mmol), a 2 M aqueous K₂CO₃ solution (100 ml), toluene (400 ml) and ethanol (100 ml), the result was refluxed for 12 hours. After the reaction was completed, the result was cooled to room temperature, and then extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 3-1 (20.2 g, 81%).

Preparation of Compound 3-2

After dissolving Compound 3-1 (20.2 g, 85.8 mmol) in THF, 4-bromobenzoyl chloride (28.2 g, 128 mmol) and TEA (26 g, 257 eq.) were added thereto at 0° C., and the result was stirred for 2 hours at room temperature. After the reaction was completed, EA and distilled water were added to the reaction container for solidification, and produced solids were collected to obtain target Compound 3-2 (35 g, 99%).

Preparation of Compound 3-3

After dissolving Compound 3-2 (35 g, 84.9 mmol) in nitrobenzene, POCl₃ (7.9 ml, 1.0 eq.) was added thereto, and the result was stirred for 18 hours at 150° C. After the reaction was completed, the result was vacuum distilled to remove the nitrobenzene, cooled to room temperature and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 3-3 (26 g, 78%).

Preparation of Compound 3-4

After dissolving Compound 3-3 (10 g, 25.0 mmol) in 1,4-dioxane, bis(pinacolato)diboron, Pd(dppf)Cl₂ and potassium acetate were added thereto, and the result was stirred for 2 hours at 110° C. After the reaction was completed, the result was extracted with distilled water and EA. After drying the organic layer with anhydrous MgSO₄, the solvent was removed using a rotary evaporator, and the result was passed through silica gel to obtain target Compound 3-4 (10.4 g, 93%).

Preparation of Compound 3-5

After adding (4-bromophenyl)diphenylphosphine oxide (9.1 g, 25.5 mmol), Pd(PPh₃)₄ (1.3 g, 1.16 mmol), K₂CO₃ (9.6 g, 69.6 mmol) and toluene/EtOH/H₂O to Compound 3-4 (10.4 g, 23.2 mmol), the result was stirred for 4 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 3-5 (11.2 g, 81%).

Preparation of Compound 3-6

After dissolving Compound 3-5 (11 g, 18.8 mmol) in dichloromethane and adding pyridine (2.2 g, 28.2 mmol) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 3-6 (12.5 g, 91%).

Preparation of Compound 3

After adding 3-bromophenanthrene (5.2 g, 20.2 mmol), Pd(PPh₃)₄ (1.0 g, 0.92 mmol), K₂CO₃ (7.6 g, 55.2 mmol) and toluene/EtOH/H₂O to Compound 3-6 (12.5 g, 18.4 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 3 (11.7 g, 84%).

### <Preparation Example 3> Preparation of Compound 4

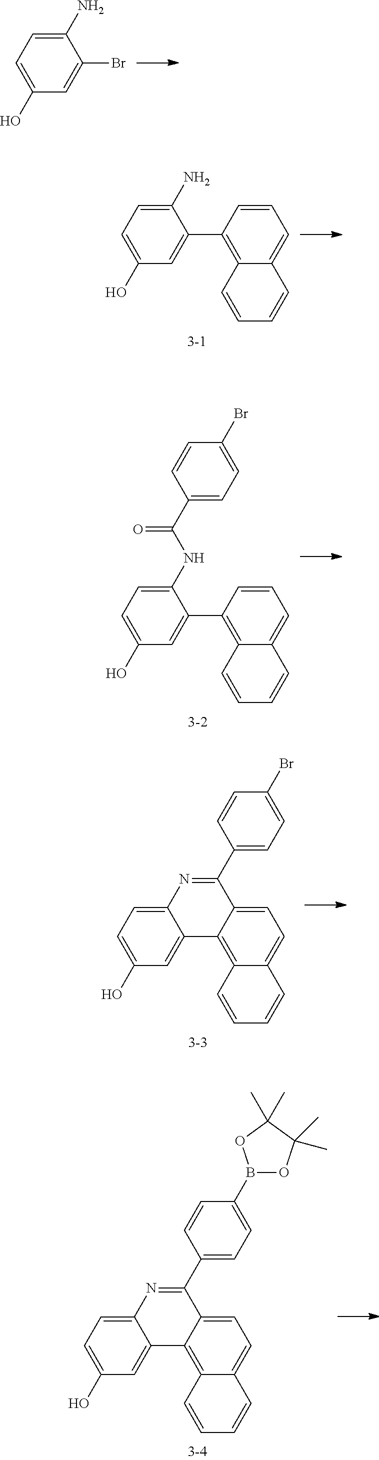 [SMILES: Nc1ccc(O)cc1Br ; Nc1ccc(O)cc1-c1cccc2ccccc12 ; O=C(Nc1ccc(O)cc1-c1cccc2ccccc12)c1ccc(Br)cc1 ; Oc1ccc2nc(-c3ccc(Br)cc3)c3ccc4ccccc4c3c2c1 ; CC1(C)OB(c2ccc(-c3nc4ccc(O)cc4c4c3ccc3ccccc34)cc2)OC1(C)C]

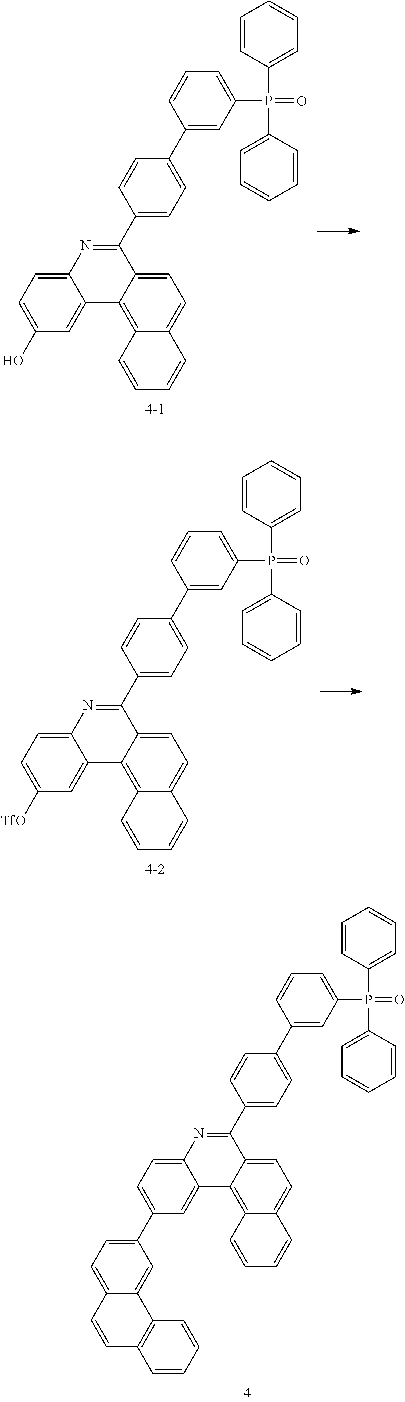 [SMILES: O=P(c1ccccc1)(c1ccccc1)c1cccc(-c2ccc(-c3nc4ccc(O)cc4c4c3ccc3ccccc34)cc2)c1 ; O=P(c1ccccc1)(c1ccccc1)c1cccc(-c2ccc(-c3nc4ccc(OS(=O)(=O)C(F)(F)F)cc4c4c3ccc3ccccc34)cc2)c1 ; O=P(c1ccccc1)(c1ccccc1)c1cccc(-c2ccc(-c3nc4ccc(-c5ccc6ccc7ccccc7c6c5)cc4c4c3ccc3ccccc34)cc2)c1]

Preparation of Compound 4-1

After adding (3-bromophenyl)diphenylphosphine oxide (9.1 g, 25.5 mmol), Pd(PPh₃)₄ (1.3 g, 1.16 mmol), K₂CO₃ (9.6 g, 69.6 mmol) and toluene/EtOH/H₂O to Compound 3-4 (10.4 g, 23.2 mmol), the result was stirred for 4 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 4-1 (10.8 g, 78%).

Preparation of Compound 4-2

After dissolving Compound 4-1 (10.8 g, 18.1 mmol) in dichloromethane and adding pyridine (2.1 g, 27.1 mmol) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 4-2 (12.3 g, 93%).

Preparation of Compound 4

After adding 3-bromophenanthrene (4.8 g, 18.5 mmol), Pd(PPh₃)₄, K₂CO₃ and toluene/EtOH/H₂O to Compound 4-2 (12.3 g, 16.8 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 4 (10.9 g, 86%).

### <Preparation Example 4> Preparation of Compound 15

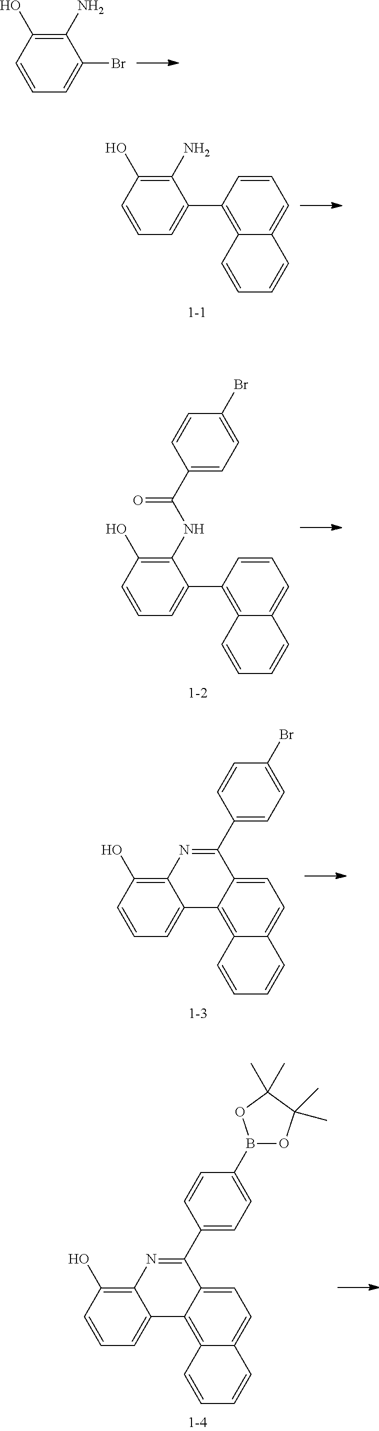 [SMILES: Nc1c(O)cccc1Br ; Nc1c(O)cccc1-c1cccc2ccccc12 ; O=C(Nc1c(O)cccc1-c1cccc2ccccc12)c1ccc(Br)cc1 ; Oc1cccc2c1nc(-c1ccc(Br)cc1)c1ccc3ccccc3c12 ; CC1(C)OB(c2ccc(-c3nc4c(O)cccc4c4c3ccc3ccccc34)cc2)OC1(C)C]

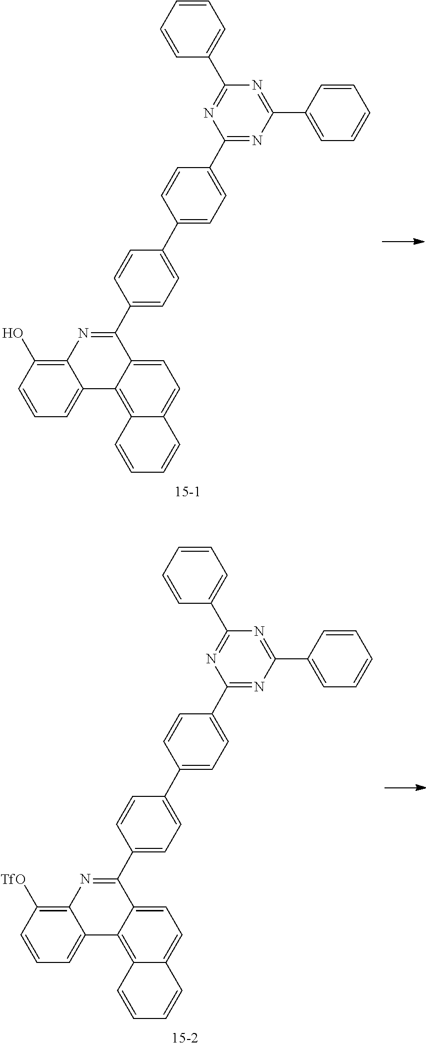 [SMILES: Oc1cccc2c1nc(-c1ccc(-c3ccc(-c4nc(-c5ccccc5)nc(-c5ccccc5)n4)cc3)cc1)c1ccc3ccccc3c12 ; O=S(=O)(Oc1cccc2c1nc(-c1ccc(-c3ccc(-c4nc(-c5ccccc5)nc(-c5ccccc5)n4)cc3)cc1)c1ccc3ccccc3c12)C(F)(F)F]

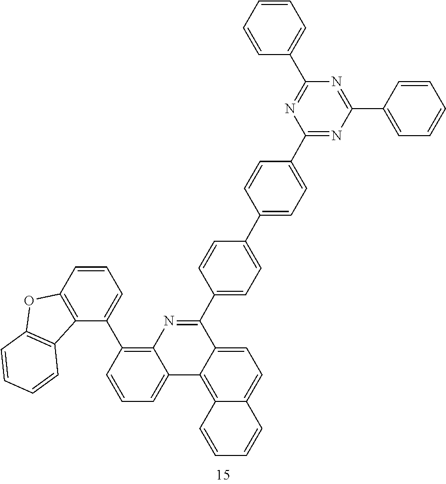 [SMILES: c1ccc(-c2nc(-c3ccccc3)nc(-c3ccc(-c4ccc(-c5nc6c(-c7cccc8oc9ccccc9c78)cccc6c6c5ccc5ccccc56)cc4)cc3)n2)cc1]

Preparation of Compound 15-1

After adding 2-(4-bromophenyl)-4,6-diphenyl-1,3,5-triazine (9.5 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 15-1 (12.3 g, 88%).

Preparation of Compound 15-2

After dissolving Compound 15-1 (12.3 g, 19.6 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 15-2 (14.0 g, 94%).

Preparation of Compound 15

After adding dibenzo[b,d]furan-1-ylboronic acid (4.3 g, 20.2 mmol), Pd(PPh₃)₄ (1.0 g, 0.92 mmol), K₂CO₃ (7.6 g, 55.2 mmol) and toluene/EtOH/H₂O to Compound 15-2 (14.0 g, 18.4 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 15 (11.2 g, 78%).

### <Preparation Example 5> Preparation of Compound 16

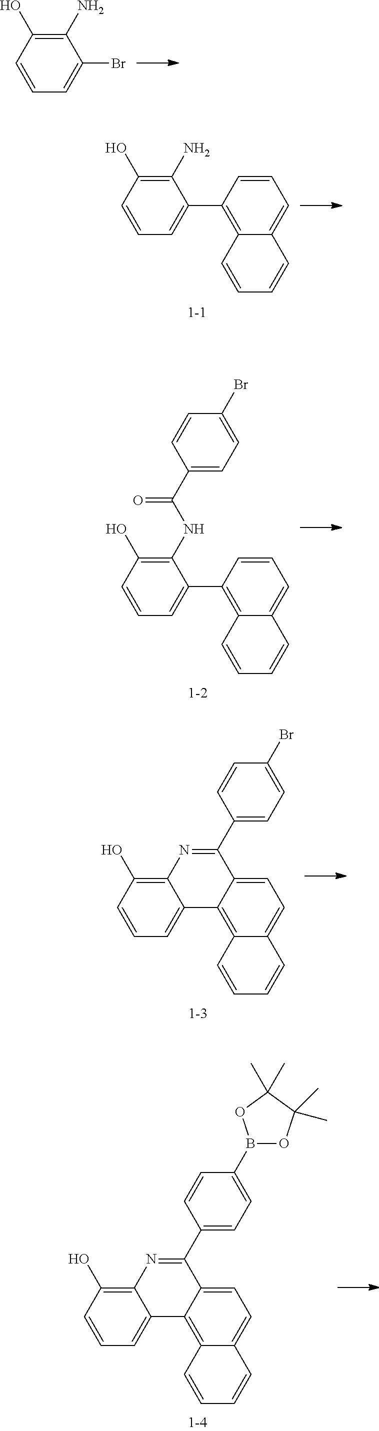 [SMILES: Nc1c(O)cccc1Br ; Nc1c(O)cccc1-c1cccc2ccccc12 ; O=C(Nc1c(O)cccc1-c1cccc2ccccc12)c1ccc(Br)cc1 ; Oc1cccc2c1nc(-c1ccc(Br)cc1)c1ccc3ccccc3c12 ; CC1(C)OB(c2ccc(-c3nc4c(O)cccc4c4c3ccc3ccccc34)cc2)OC1(C)C]

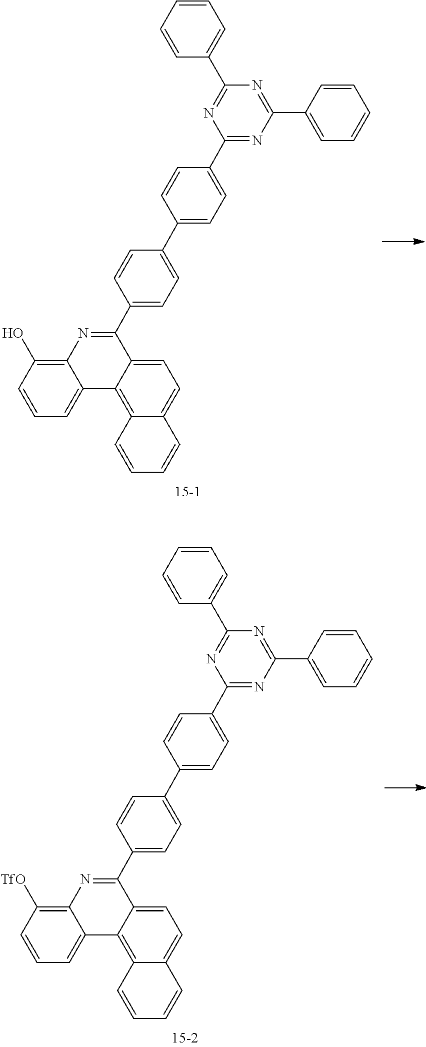 [SMILES: Oc1cccc2c1nc(-c1ccc(-c3ccc(-c4nc(-c5ccccc5)nc(-c5ccccc5)n4)cc3)cc1)c1ccc3ccccc3c12 ; O=S(=O)(Oc1cccc2c1nc(-c1ccc(-c3ccc(-c4nc(-c5ccccc5)nc(-c5ccccc5)n4)cc3)cc1)c1ccc3ccccc3c12)C(F)(F)F]

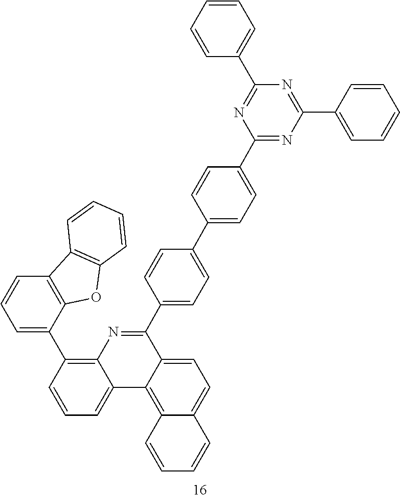 [SMILES: c1ccc(-c2nc(-c3ccccc3)nc(-c3ccc(-c4ccc(-c5nc6c(-c7cccc8c7oc7ccccc78)cccc6c6c5ccc5ccccc56)cc4)cc3)n2)cc1]

Preparation of Compound 16

Target Compound 16 was obtained in the same manner as in the preparation of Compound 15 of Preparation Example 4 except that dibenzo[b,d]furan-4-ylboronic acid was used instead of dibenzo[b,d]furan-1-ylboronic acid.

### <Preparation Example 6> Preparation of Compound 17

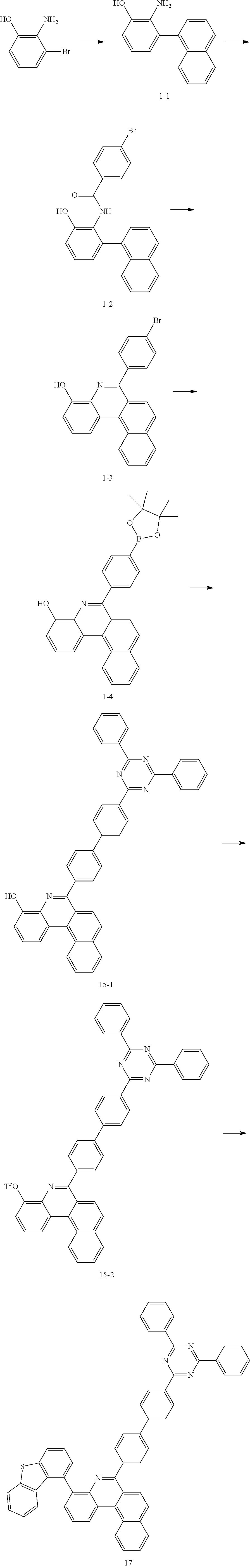

Preparation of Compound 17

Target Compound 17 was obtained in the same manner as in the preparation of Compound 15 of Preparation Example 4 except that dibenzo[b,d]thiophen-1-ylboronic acid was used instead of dibenzo[b,d]furan-1-ylboronic acid.

### <Preparation Example 7> Preparation of Compound 21

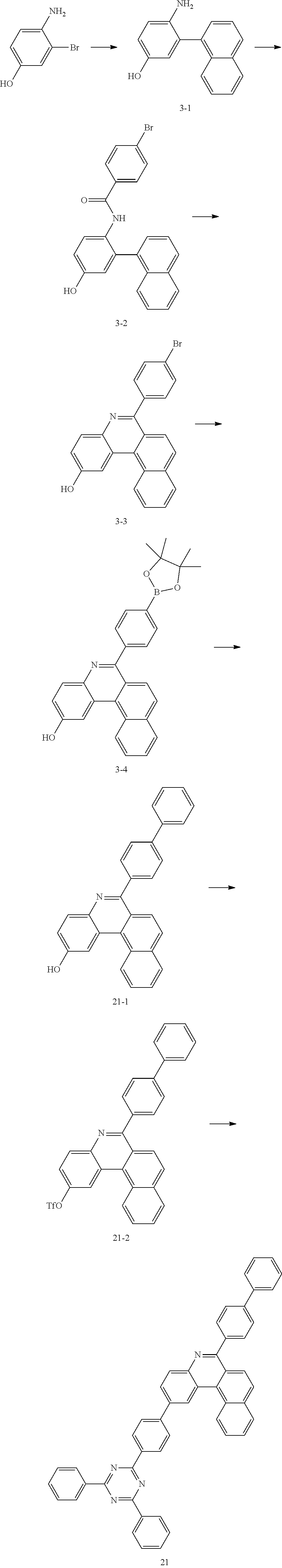

Preparation of Compound 21-1

After adding phenyl bromide (3.5 g, 24.6 mmol), Pd(PPh₃)₄ (1.3 g, 1.16 mmol), K₂CO₃ (9.6 g, 69.6 mmol) and toluene/EtOH/H₂O to Compound 3-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 21-1 (8.4 g, 95%).

Preparation of Compound 21-2

After dissolving Compound 21-1 (8.4 g, 21.1 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 21-2 (10.5 g, 94%).

Preparation of Compound 21

After adding 2-(4-bromophenyl)-4,6-diphenyl-1,3,5-triazine (7.8 g, 20.2 mmol), Pd(PPh₃)₄ (1.0 g, 0.92 mmol), K₂CO₃ (7.6 g, 55.2 mmol) and toluene/EtOH/H₂O to Compound 21-2 (10.5 g, 19.8 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 21 (12.7 g, 93%).

### <Preparation Example 8> Preparation of Compound 34

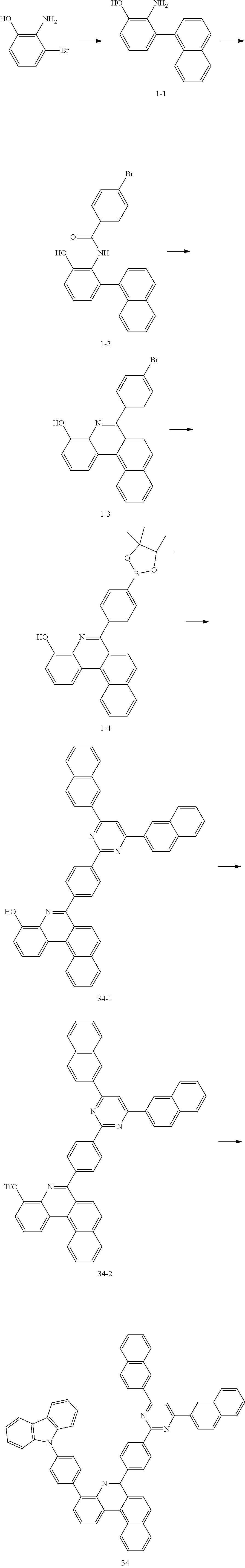

Preparation of Compound 34-1

After adding 2-chloro-4,6-di(naphthalen-2-yl)pyrimidine (9.0 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 34-1 (13.7 g, 94%).

Preparation of Compound 34-2

After dissolving Compound 34-1 (13.7 g, 20.9 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 34-2 (15.2 g, 93%).

Preparation of Compound 34

After adding 9-(4-(4,4,5,5-tetramethyl-1,3,2-dioxaborolan-2-yl)phenyl)-9H-carbazole (7.5 g, 20.2 mmol), Pd(PPh₃)₄ (1.0 g, 0.92 mmol), K₂CO₃ (7.6 g, 55.2 mmol) and toluene/EtOH/H₂O to Compound 34-2 (15.2 g, 19.4 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 34 (13.8 g, 81%).

### <Preparation Example 9> Preparation of Compound 35

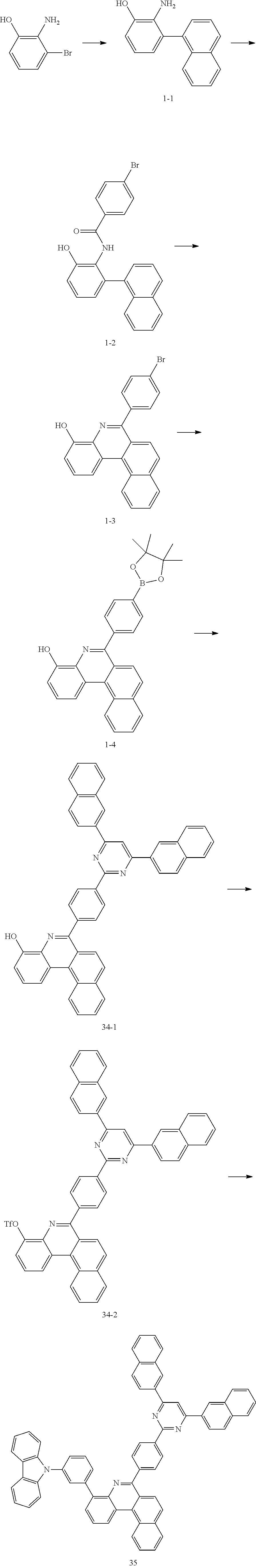

Preparation of Compound 35

Target Compound 35 was obtained in the same manner as in the preparation of Compound 34 of Preparation Example 8 except that 9-(3-(4,4,5,5-tetramethyl-1,3,2-dioxaborolan-2-yl)phenyl)-9H-carbazole was used instead of 9-(4-(4,4,5,5-tetramethyl-1,3,2-dioxaborolan-2-yl)phenyl)-9H-carbazole.

### <Preparation Example 10> Preparation of Compound 69

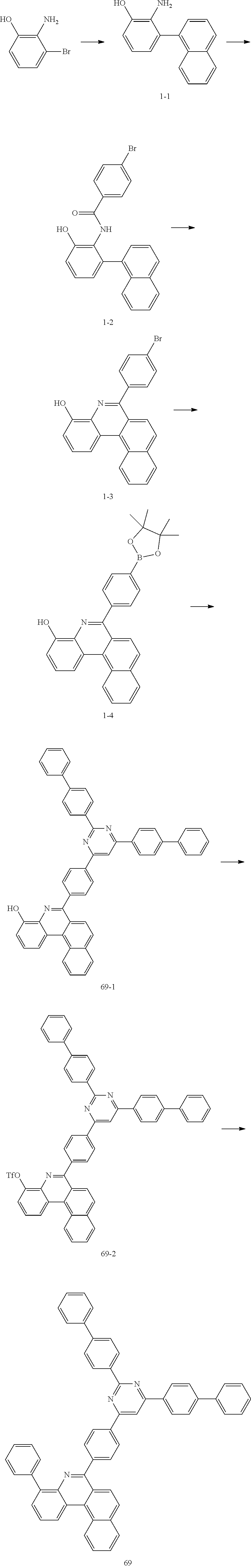

Preparation of Compound 69-1

After adding 2,4-di([1,1′-biphenyl]-4-yl)-6-chloropyrimidine (10.3 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 69-1 (13.9 g, 89%).

Preparation of Compound 69-2

After dissolving Compound 69-1 (13.9 g, 19.8 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 69-2 (15.0 g, 91%).

Preparation of Compound 69

After adding phenylboronic acid (2.5 g, 20.2 mmol), Pd(PPh₃)₄ (1.0 g, 0.92 mmol), K₂CO₃ (7.6 g, 55.2 mmol) and toluene/EtOH/H₂O to Compound 69-2 (15.0 g, 18.0 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 69 (12.1 g, 88%).

### <Preparation Example 11> Preparation of Compound 77

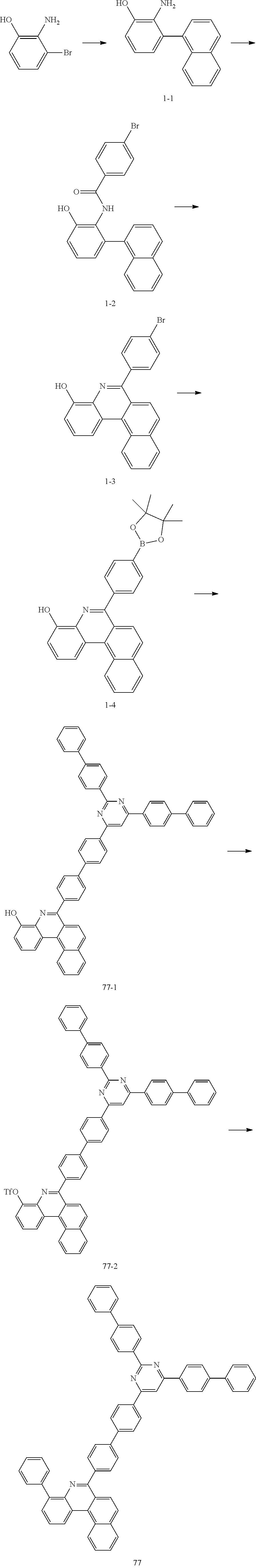

Preparation of Compound 77-1

After adding 2,4-di([1,1′-biphenyl]-4-yl)-6-(4-bromophenyl)pyrimidine (13.2 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 77-1 (15.8 g, 91%).

Preparation of Compound 77-2

After dissolving Compound 77-1 (15.8 g, 20.3 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 77-2 (16.8 g, 91%).

Preparation of Compound 77

After adding phenylboronic acid (2.5 g, 20.2 mmol), Pd(PPh₃)₄ (1.0 g, 0.92 mmol), K₂CO₃ (7.6 g, 55.2 mmol) and toluene/EtOH/H₂O to Compound 77-2 (16.8 g, 18.5 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 77 (13.5 g, 87%).

### <Preparation Example 12> Preparation of Compound 80

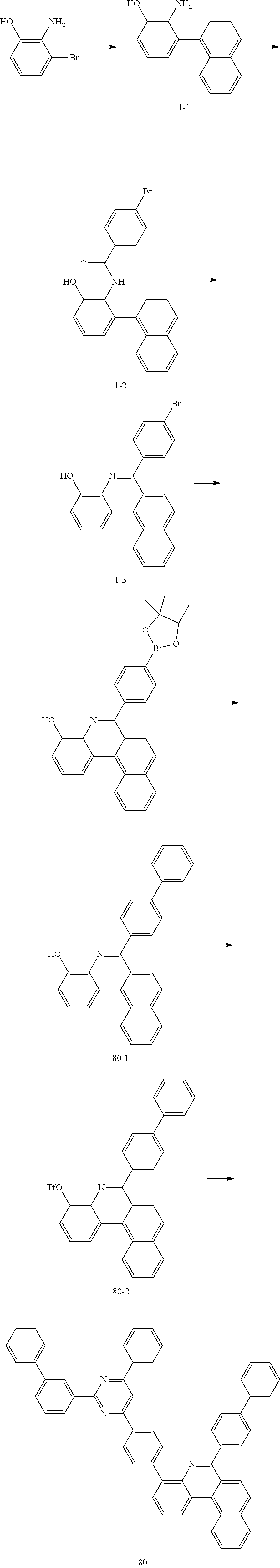

Preparation of Compound 80-1

After adding phenylboronic acid (24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 80-1 (7.5 g, 85%).

Preparation of Compound 80-2

After dissolving Compound 80-1 (7.5 g, 18.9 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 80-2 (9.4 g, 94%).

Preparation of Compound 80

After adding 2-([1,1′-biphenyl]-3-yl)-4-phenyl-6-(4-(4,4,5,5-tetramethyl-1,3,2-dioxaborolan-2-yl)phenyl)pyrimidine (10.3 g, 20.2 mmol), Pd(PPh₃)₄ (1.0 g, 0.92 mmol), K₂CO₃ (7.6 g, 55.2 mmol) and toluene/EtOH/H₂O to Compound 77-2 (9.4 g, 17.7 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 80 (12.4 g, 92%).

### <Preparation Example 13> Preparation of Compound 85

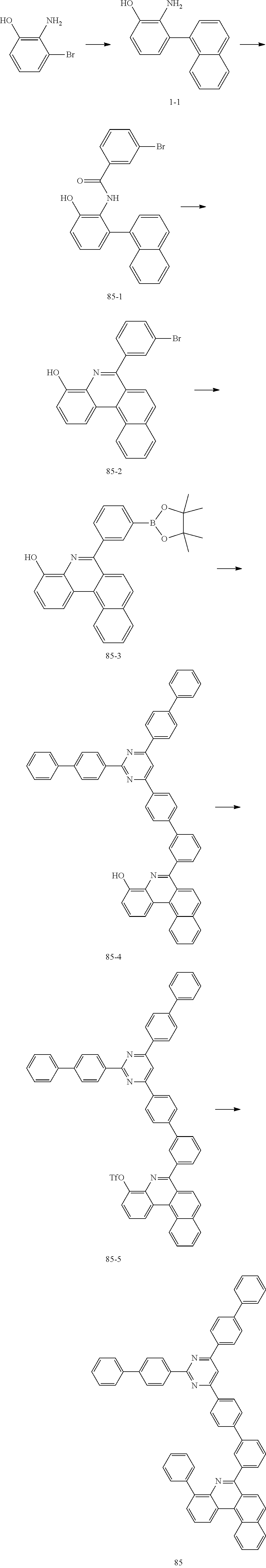

Preparation of Compound 85-1

After dissolving Compound 1-1 (20.0 g, 85.0 mmol) in THF, 3-bromobenzoyl chloride (27.9 g, 127 mmol) and TEA (38 g, 381 mmol) were added thereto at 0° C., and the result was stirred for 2 hours at room temperature. After the reaction was completed, EA and distilled water were added to the reaction container for solidification, and produced solids were collected to obtain target Compound 85-1 (35 g, 99%).

Preparation of Compound 85-2

After dissolving Compound 85-1 (35 g, 84.1 mmol) in nitrobenzene, POCl₃ (1.0 eq.) was added thereto, and the result was stirred for 18 hours at 150° C. After the reaction was completed, the result was vacuum distilled to remove the nitrobenzene, cooled to room temperature and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 85-2 (26 g, 78%).

Preparation of Compound 85-3

After dissolving Compound 85-2 (26 g, 65.6 mmol) in 1,4-dioxane, bis(pinacolato)diboron, Pd(dppf)Cl₂ and potassium acetate were added thereto, and the result was stirred for 2 hours at 110° C. After the reaction was completed, the result was extracted with distilled water and EA. After drying the organic layer with anhydrous MgSO₄, the solvent was removed using a rotary evaporator, and the result was passed through silica gel to obtain target Compound 85-3 (28.5 g, 97%).

Preparation of Compound 85-4

After adding 2,4-di([1,1′-biphenyl]-4-yl)-6-(4-bromophenyl)pyrimidine (13.2 g, 24.6 mmol), Pd(PPh₃)₄ (1.3 g, 1.16 mmol), K₂CO₃ (9.6 g, 69.6 mmol) and toluene/EtOH/H₂O to Compound 85-3 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 85-4 (14.8 g, 85%).

Preparation of Compound 85-5

After dissolving Compound 85-4 (14.8 g, 18.9 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 85-5 (16 g, 93%).

Preparation of Compound 85

After adding phenylboronic acid (1.5 eq.), Pd(PPh₃)₄ (0.05 eq.), K₂CO₃ (3.0 eq.) and toluene/EtOH/H₂O to Compound 85-5 (16 g, 17.6 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 85 (12.4 g, 84%).

### <Preparation Example 14> Preparation of Compound 89

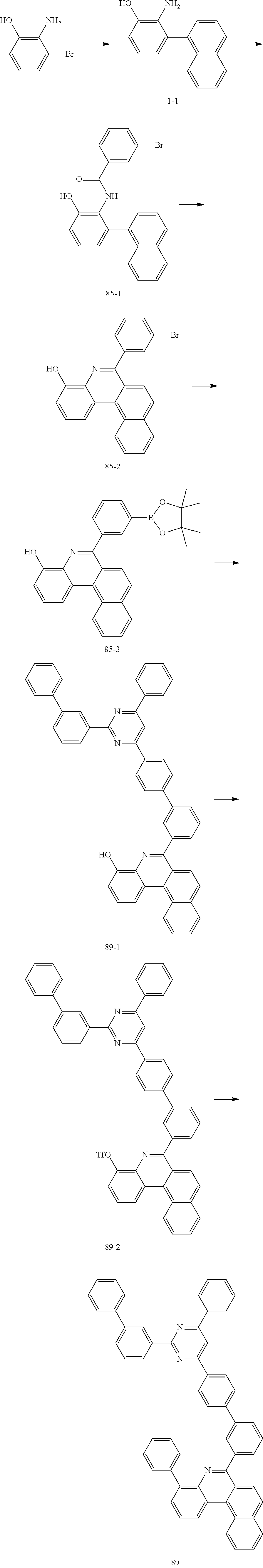

Preparation of Compound 89-1

After adding 2-([1,1′-biphenyl]-3-yl)-4-(4-bromophenyl)-6-phenylpyrimidine (11.4 g, 24.6 mmol), Pd(PPh₃)₄ (1.3 g, 1.16 mmol), K₂CO₃ (9.6 g, 69.6 mmol) and toluene/EtOH/H₂O to Compound 85-3 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 89-1 (13.8 g, 88%).

Preparation of Compound 89-2

After dissolving Compound 89-1 (13.8 g, 19.6 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 89-2 (15.4 g, 94%).

Preparation of Compound 89

After adding phenylboronic acid (1.5 eq.), Pd(PPh₃)₄ (0.05 eq.), K₂CO₃ (3.0 eq.) and toluene/EtOH/H₂O to Compound 89-2 (15.4 g, 18.4 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 89 (12.2 g, 87%).

### <Preparation Example 15> Preparation of Compound 96

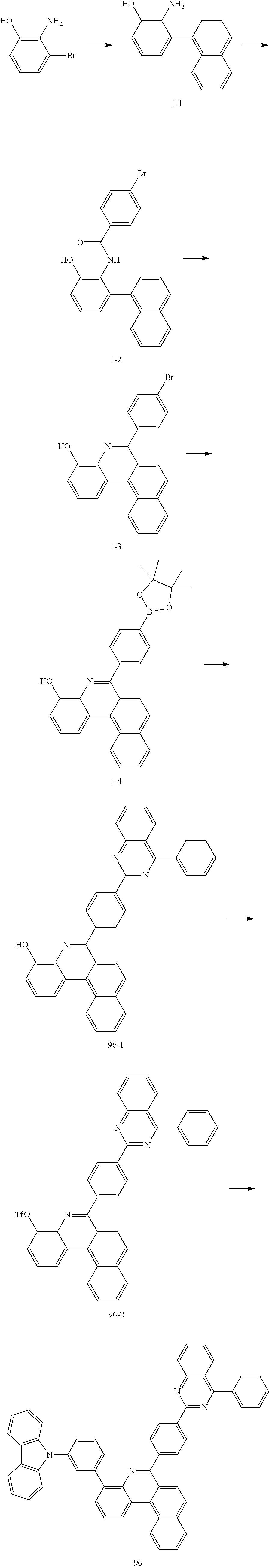

Preparation of Compound 96-1

After adding 2-chloro-4-phenylquinazoline (5.92 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 96-1 (11.4 g, 88%).

Preparation of Compound 96-2

After dissolving Compound 96-1 (11.4 g, 21.6 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 96-2 (13.0 g, 92%).

Preparation of Compound 96

After adding 9-(3-(4,4,5,5-tetramethyl-1,3,2-dioxaborolan-2-yl)phenyl)-9H-carbazole (1.5 eq.), Pd(PPh₃)₄ (0.05 eq.), K₂CO₃ (3.0 eq.) and toluene/EtOH/H₂O to Compound 96-2 (13.0 g, 19.8 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 96 (13.5 g, 91%).

### <Preparation Example 16> Preparation of Compound 97

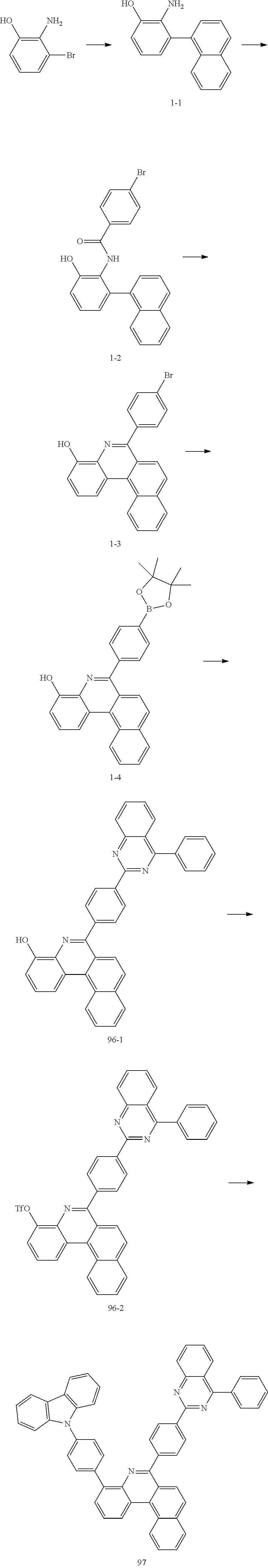

Target Compound 97 was obtained in the same manner as in the preparation of Compound 96 of Preparation Example 15 except that 9-(4-(4,4,5,5-tetramethyl-1,3,2-dioxaborolan-2-yl)phenyl)-9H-carbazole was used instead of 9-(3-(4,4,5,5-tetramethyl-1,3,2-dioxaborolan-2-yl)phenyl)-9H-carbazole.

### <Preparation Example 17> Preparation of Compound 99

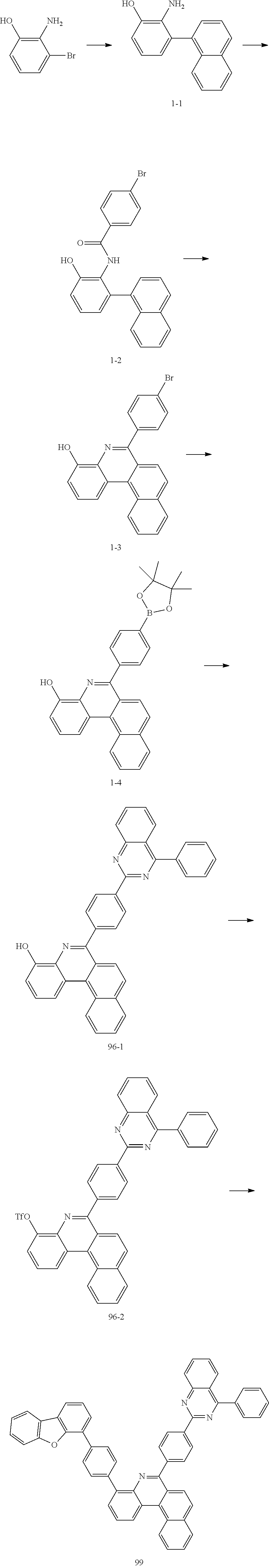

Target Compound 99 was obtained in the same manner as in the preparation of Compound 96 of Preparation Example 15 except that 2-(4-(dibenzo[b,d]furan-4-yl)phenyl)-4,4,5,5-tetramethyl-1,3,2-dioxaborolane was used instead of 9-(3-(4,4,5,5-tetramethyl-1,3,2-dioxaborolan-2-yl)phenyl)-9H-carbazole.

### <Preparation Example 18> Preparation of Compound 100

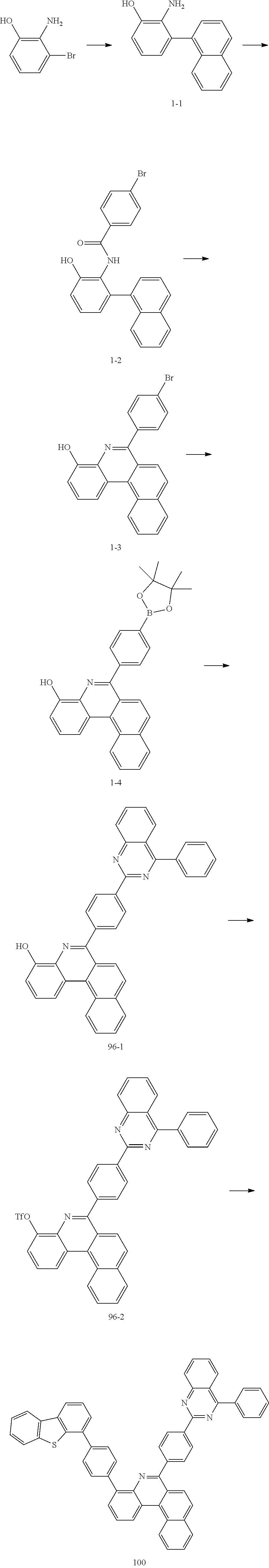

Target Compound 100 was obtained in the same manner as in the preparation of Compound 96 of Preparation Example 15 except that 2-(4-(dibenzo[b,d]thiophen-4-yl)phenyl)-4,4,5,5-tetramethyl-1,3,2-dioxaborolane was used instead of 9-(3-(4,4,5,5-tetramethyl-1,3,2-dioxaborolan-2-yl)phenyl)-9H-carbazole.

### <Preparation Example 19> Preparation of Compound 112

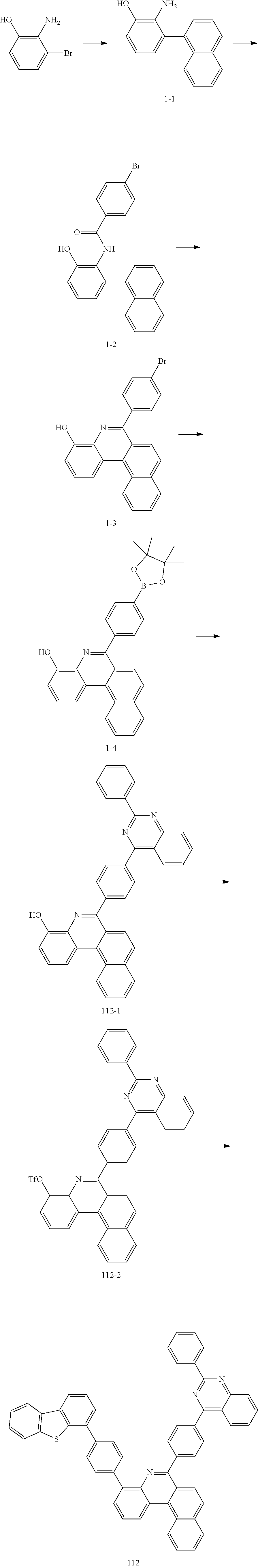

Preparation of Compound 112-1

After adding 4-chloro-2-phenylquinazoline (5.92 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 112-1 (11.6 g, 90%).

Preparation of Compound 112-2

After dissolving Compound 112-1 (11.6 g, 22.1 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 112-2 (13.3 g, 92%).

Preparation of Compound 112

After adding 2-(4-(dibenzo[b,d]thiophen-4-yl)phenyl)-4,4,5,5-tetramethyl-1,3,2-dioxaborolane (1.5 eq.), Pd(PPh₃)₄ (0.05 eq.), K₂CO₃ (3.0 eq.) and toluene/EtOH/H₂O to Compound 96-2 (13.3 g, 20.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 112 (14.5 g, 93%).

### <Preparation Example 20> Preparation of Compound 113

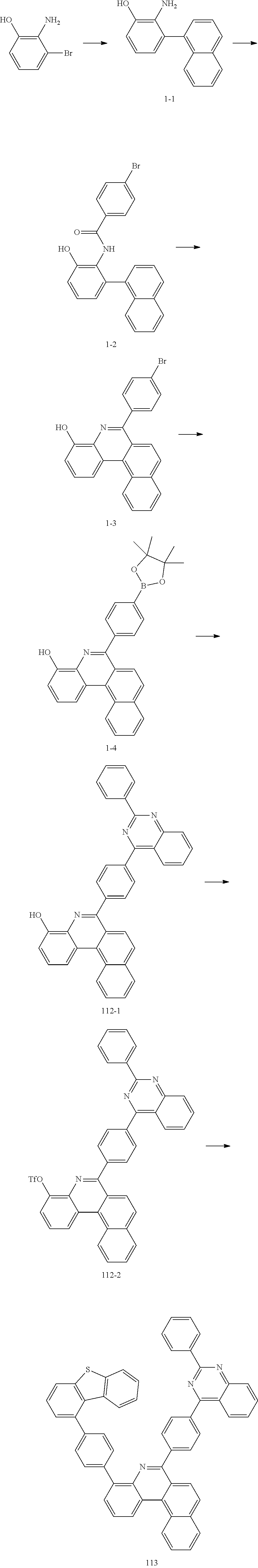

Target Compound 113 was obtained in the same manner as in the preparation of Compound 112 of Preparation Example 19 except that 2-(4-(dibenzo[b,d]thiophen-1-yl)phenyl)-4,4,5,5-tetramethyl-1,3,2-dioxaborolane was used instead of 2-(4-(dibenzo[b,d]thiophen-4-yl)phenyl)-4,4,5,5-tetramethyl-1,3,2-dioxaborolane.

### <Preparation Example 21> Preparation of Compound 114

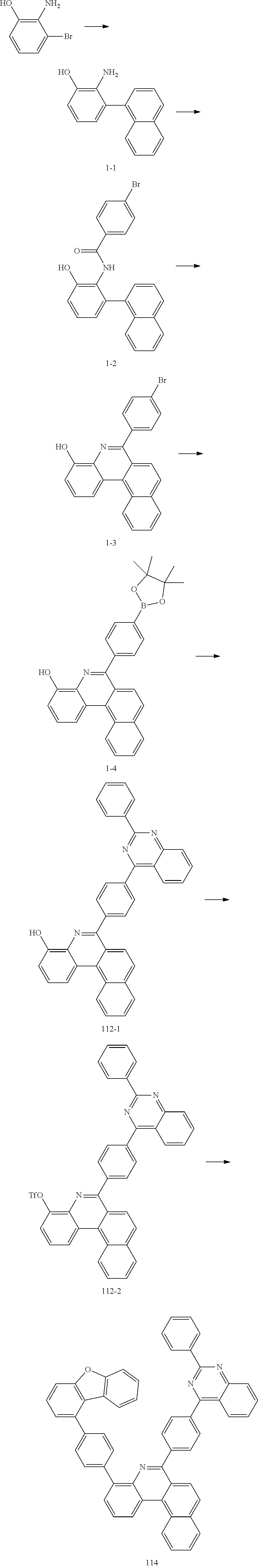

Target Compound 114 was obtained in the same manner as in the preparation of Compound 112 of Preparation Example 19 except that 2-(4-(dibenzo[b,d]furan-1-yl)phenyl)-4,4,5,5-tetramethyl-1,3,2-dioxaborolane was used instead of 2-(4-(dibenzo[b,d]thiophen-4-yl)phenyl)-4,4,5,5-tetramethyl-1,3,2-dioxaborolane.

### <Preparation Example 22> Preparation of Compound 115

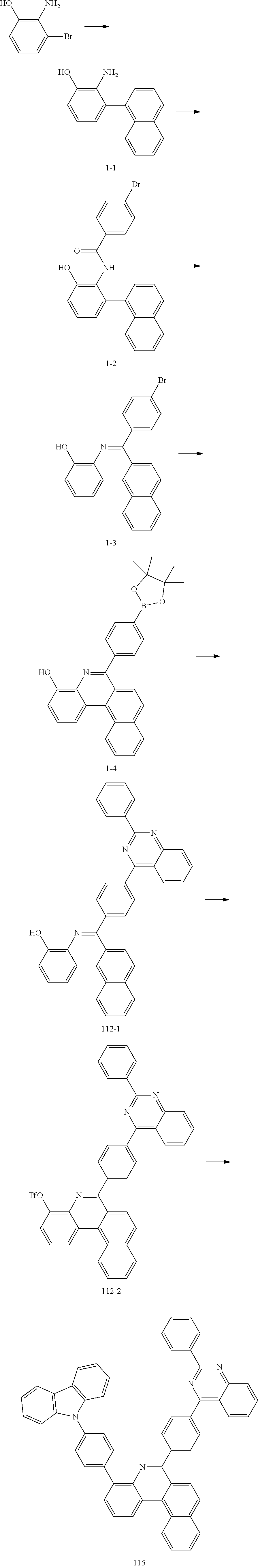

Target Compound 115 was obtained in the same manner as in the preparation of Compound 112 of Preparation Example 19 except that 9-(4-(4,4,5,5-tetramethyl-1,3,2-dioxaborolan-2-yl)phenyl)-9H-carbazole was used instead of 2-(4-(dibenzo[b,d]thiophen-4-yl)phenyl)-4,4,5,5-tetramethyl-1,3,2-dioxaborolane.

### <Preparation Example 23> Preparation of Compound 118

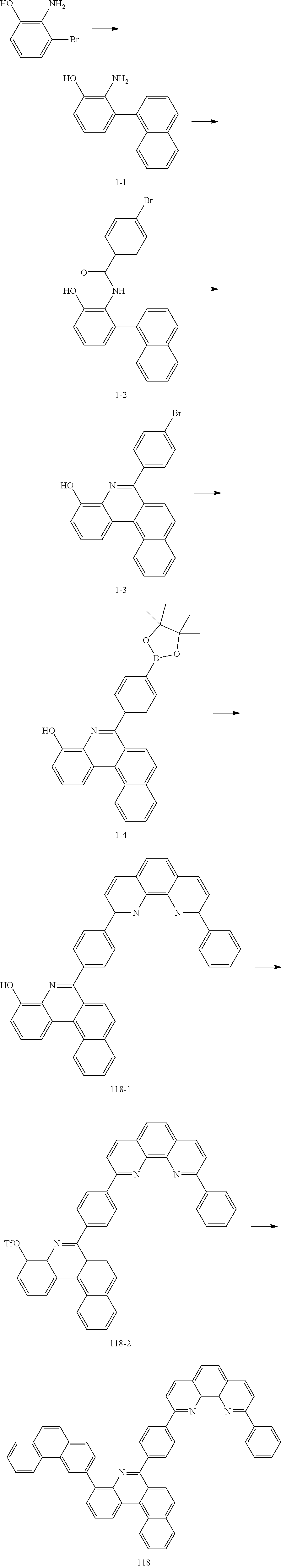

Preparation of Compound 118-1

After adding 2-bromo-9-phenyl-1,10-phenanthroline (8.2 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 118-1 (11.8 g, 92%).

Preparation of Compound 118-2

After dissolving Compound 118-1 (11.8 g, 20.5 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 118-2 (13.6 g, 94%).

Preparation of Compound 118

After adding 4,4,5,5-tetramethyl-2-(phenanthren-3-yl)-1,3,2-dioxaborolane (1.5 eq.), Pd(PPh₃)₄ (0.05 eq.), K₂CO₃ (3.0 eq.) and toluene/EtOH/H₂O to Compound 118-2 (13.6 g, 19.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 118 (13.2 g, 93%).

### <Preparation Example 24> Preparation of Compound 123

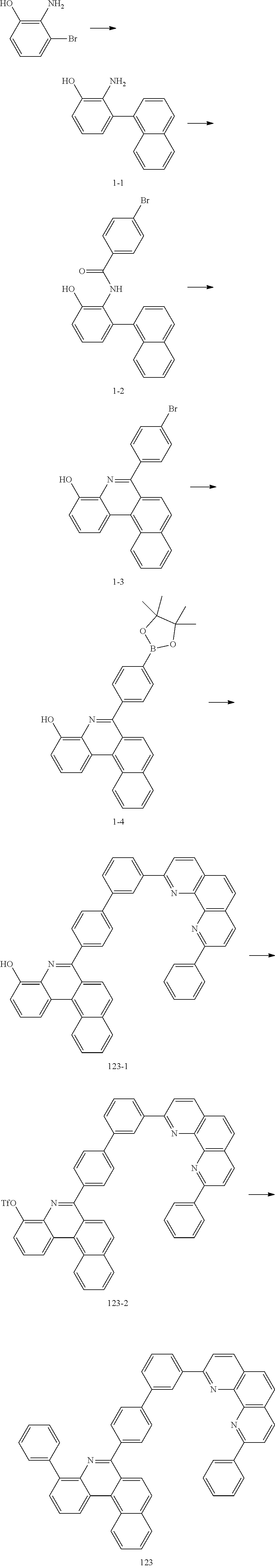

Preparation of Compound 123-1

After adding 2-(3-bromophenyl)-9-phenyl-1,10-phenanthroline (10.1 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 123-1 (12.8 g, 88%).

Preparation of Compound 123-2

After dissolving Compound 123-1 (12.8 g, 19.6 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 123-2 (13.5 g, 88%).

Preparation of Compound 123

After adding phenylboronic acid (1.1 eq.), Pd(PPh₃)₄ (0.05 eq.), K₂CO₃ (3.0 eq.) and toluene/EtOH/H₂O to Compound 123-2 (13.5 g, 17.2 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 123 (10.9 g, 89%).

### <Preparation Example 25> Preparation of Compound 124

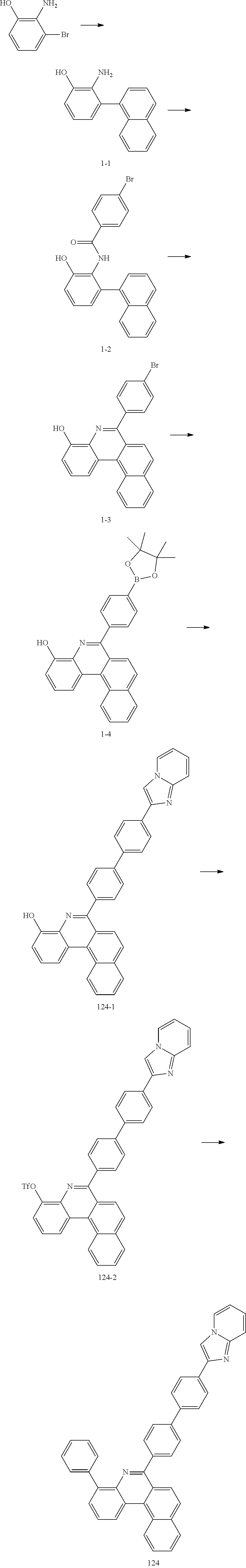

Preparation of Compound 124-1

After adding 2-bromoimidazo[1,2-a]pyridine (4.8 g, 24.6 mmol), Pd(PPh₃)₄ (1.3 g, 1.16 mmol), K₂CO₃ (9.6 g, 69.6 mmol) and toluene/EtOH/H₂O to Compound 1-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 124-1 (10.5 g, 92%).

Preparation of Compound 124-2

After dissolving Compound 124-1 (10.5 g, 20.5 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 124-2 (11.8 g, 89%).

Preparation of Compound 124

After adding phenylboronic acid (1.1 eq.), Pd(PPh₃)₄ (0.05 eq.), K₂CO₃ (3.0 eq.) and toluene/EtOH/H₂O to Compound 124-2 (11.8 g, 18.2 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 124 (9.2 g, 90%).

### <Preparation Example 26> Preparation of Compound 130

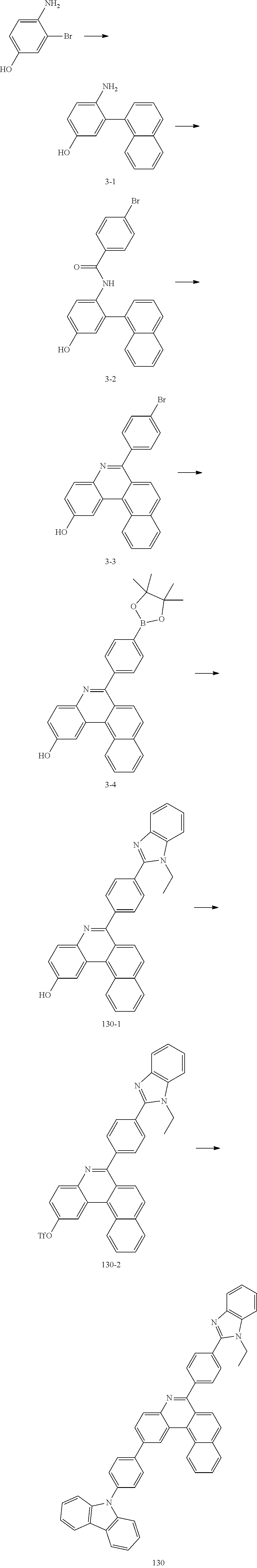

Preparation of Compound 130-1

After adding 2-bromo-1-ethyl-1H-benzo[d]imidazole (5.5 g, 24.6 mmol), Pd(PPh₃)₄ (1.3 g, 1.16 mmol), K₂CO₃ (9.6 g, 69.6 mmol) and toluene/EtOH/H₂O to Compound 3-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 130-1 (9.6 g, 93%).

Preparation of Compound 130-2

After dissolving Compound 130-1 (9.6 g, 20.7 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 130-2 (11.4 g, 92%).

Preparation of Compound 130

After adding 9-(4-(4,4,5,5-tetramethyl-1,3,2-dioxaborolan-2-yl)phenyl)-9H-carbazole (7.7 g, 20.9 mmol), Pd(PPh₃)₄ (0.05 eq.), K₂CO₃ (3.0 eq.) and toluene/EtOH/H₂O to Compound 130-2 (11.4 g, 19.0 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 130 (12.0 g, 92%).

### <Preparation Example 27> Preparation of Compound 139

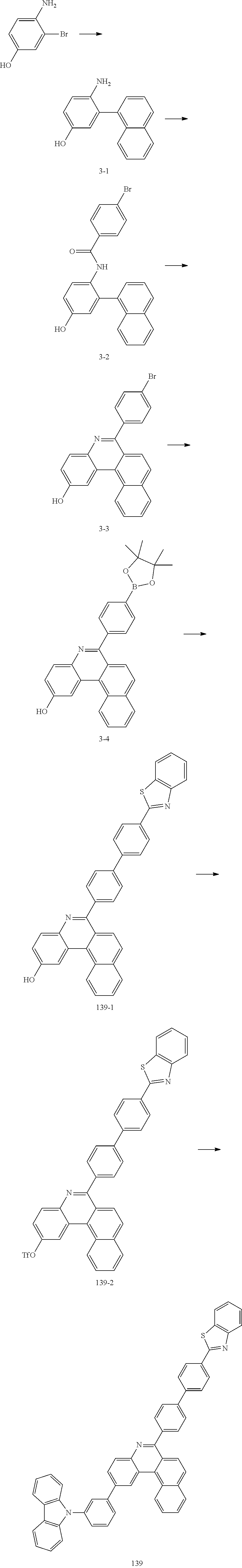

Preparation of Compound 139-1

After adding 2-(4-bromophenyl)benzo[d]thiazole (7.1 g, 24.6 mmol), Pd(PPh₃)₄ (1.3 g, 1.16 mmol), K₂CO₃ (9.6 g, 69.6 mmol) and toluene/EtOH/H₂O to Compound 3-4 (10 g, 22.3 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 139-1 (10.5 g, 89%).

Preparation of Compound 139-2

After dissolving Compound 139-1 (10.5 g, 19.8 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 139-2 (11.8 g, 90%).

Preparation of Compound 139

After adding 9-(3-(4,4,5,5-tetramethyl-1,3,2-dioxaborolan-2-yl)phenyl)-9H-carbazole (7.7 g, 20.9 mmol), Pd(PPh₃)₄ (0.05 eq.), K₂CO₃ (3.0 eq.) and toluene/EtOH/H₂O to Compound 139-2 (11.8 g, 17.8 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 139 (12.2 g, 91%).

### <Preparation Example 28> Preparation of Compound 31

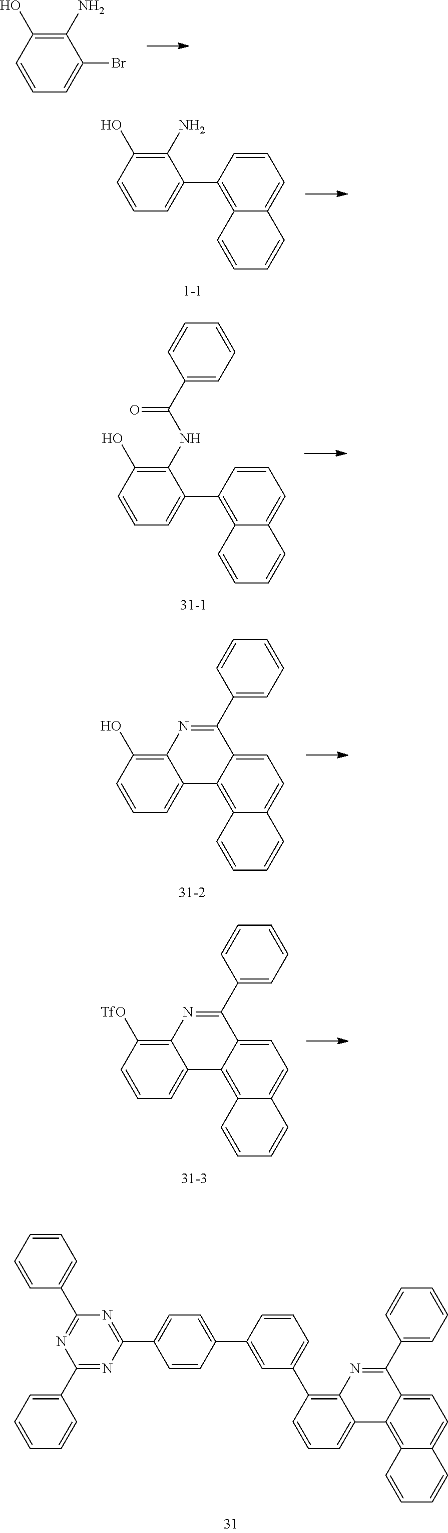 [SMILES: Nc1c(O)cccc1Br ; Nc1c(O)cccc1-c1cccc2ccccc12 ; O=C(Nc1c(O)cccc1-c1cccc2ccccc12)c1ccccc1 ; Oc1cccc2c1nc(-c1ccccc1)c1ccc3ccccc3c12 ; O=S(=O)(Oc1cccc2c1nc(-c1ccccc1)c1ccc3ccccc3c12)C(F)(F)F ; c1ccc(-c2nc(-c3ccccc3)nc(-c3ccc(-c4cccc(-c5cccc6c5nc(-c5ccccc5)c5ccc7ccccc7c56)c4)cc3)n2)cc1]

Preparation of Compound 31-1

After dissolving Compound 1-1 (20.0 g, 85.0 mmol) in THF, benzoyl chloride (13.1 g, 93.5 mmol) and TEA (25.8 g, 255 mmol) were added thereto at 0° C., and the result was stirred for 2 hours at room temperature. After the reaction was completed, EA and distilled water were added to the reaction container for solidification, and produced solids were collected to obtain target Compound 31-1 (28.5 g, 99%).

Preparation of Compound 31-2

After dissolving Compound 31-1 (28.5 g, 84.1 mmol) in nitrobenzene, POCl₃ (1.0 eq.) was added thereto, and the result was stirred for 18 hours at 150° C. After the reaction was completed, the result was vacuum distilled to remove the nitrobenzene, cooled to room temperature and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 31-2 (21 g, 78%).

Preparation of Compound 31-3

After dissolving Compound 31-2 (21 g, 65.5 mmol) in dichloromethane and adding pyridine (1.5 eq.) thereto, triflic anhydride was added dropwise thereto at 0° C. After that, the result was stirred for 5 hours at room temperature. After the reaction was completed, the reaction solution was passed through silica, the solvent of the filtrate was removed using a rotary evaporator, and with dichloromethane and methanol as a developing solvent, the result was purified using column chromatography to obtain target Compound 31-3 (26.4 g, 89%).

Preparation of Compound 31

After adding 2,4-diphenyl-6-(3′-(4,4,5,5-tetramethyl-1,3,2-dioxaborolan-2-yl)-[1,1′-biphenyl]-4-yl)-1,3,5-triazine (1.1 eq.), Pd(PPh₃)₄ (0.05 eq.), K₂CO₃ (3.0 eq.) and toluene/EtOH/H₂O to Compound 31-3 (10.0 g, 22.0 mmol), the result was stirred for 6 hours at 110° C. After the reaction was completed, the result was cooled to room temperature, and extracted with distilled water and EA. After drying the organic layer with anhydrous MgSO₄, the solvent was removed using a rotary evaporator, and with dichloromethane and hexane as a developing solvent, the result was purified using column chromatography to obtain target Compound 31 (13.9 g, 92%).

### <Preparation Example 29> Preparation of Compound 32

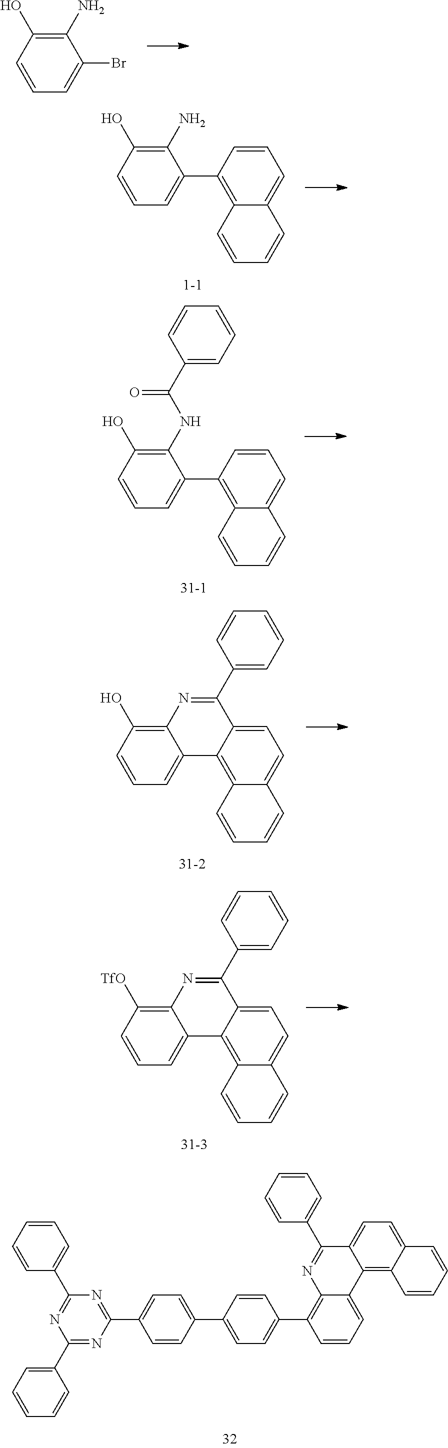

Target Compound 32 was obtained in the same manner as in the preparation of Compound 31 of Preparation Example 28 except that 2,4-diphenyl-6-(4′-(4,4,5,5-tetramethyl-1,3,2-dioxaborolan-2-yl)-[1,1′-biphenyl]-4-yl)-1,3,5-triazine was used instead of 2,4-diphenyl-6-(3′-(4,4,5,5-tetramethyl-1,3,2-dioxaborolan-2-yl)-[1,1′-biphenyl]-4-yl)-1,3,5-triazine.

### <Preparation Example 30> Preparation of Compound 214

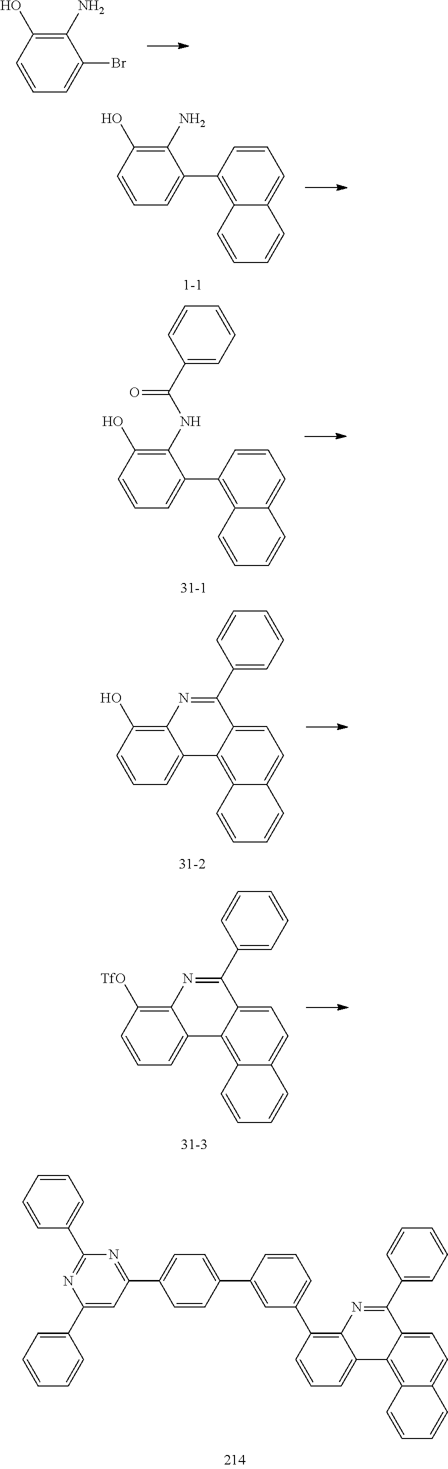

Target Compound 214 was obtained in the same manner as in the preparation of Compound 31 of Preparation Example 28 except that 2,4-diphenyl-6-(3′-(4,4,5,5-tetramethyl-1,3,2-dioxaborolan-2-yl)-[1,1′-biphenyl]-4-yl)pyrimidine was used instead of 2,4-diphenyl-6-(3′-(4,4,5,5-tetramethyl-1,3,2-dioxaborolan-2-yl)-[1,1′-biphenyl]-4-yl)-1,3,5-triazine.

Compounds other than the compounds described in the preparation examples were also prepared in the same manner as in the preparation examples described above.

The following Table 1 and Table 2 present 41 NMR data and FD-MS data of the synthesized compounds, and through the following data, syntheses of target compounds may be identified.

TABLE 1

| NO | ¹H NMR (CDCl₃, 300 Mz) |
|---|---|
| 1 | 7.25-7.28 (m, 4H), 7.41-7.55 (m, 7H), 7.66-7.67 (m, 3H), |
| | 7.82-7.93 (m, 8H), 8.01-8.03 (d, 1H), 8.12-8.28 (m, 8H), |
| | 8.53-8.55 (m, 1H), 8.80-8.82 (d, 2H), 8.92-8.94 (d, 2H) |
| 3 | 7.44-7.46 (m, 6H), 7.54-7.56 (d, 1H), 7.67-7.92 (m, |
| | 18H), 8.03-8.05 (d, 2H), 8.12-8.21 (m, 4H), 8.53-8.55 |
| | (m, 1H), 8.80-8.82 (d, 2H), 8.92-8.94 (d, 1H), 9.14-9.16 |
| | (d, 1H) |
| 4 | 7.45-7.55 (m, 9H), 7.66-7.92 (m, 16H), 8.03-8.05 (d, |
| | 2H), 8.17-8.22 (m, 3H), 8.53-8.55 (m, 1H), 8.80-8.82 (d, |
| | 2H), 8.92-8.94 (d, 1H), 9.14-9.16 (d, 1H) |
| 5 | 7.25-7.33 (m, 3H), 7.45-7.55 (m, 9H), 7.66-7.79 (m, |
| | 13H), 7.92-7.94 (m, 2H), 8.01-8.03 (d, 1H), 8.12-8.21 |
| | (m, 4H), 8.29-8.31 (d, 1H), 8.53-8.56 (m, 2H) |
| 6 | 7.25 (s, 4H), 7.45-7.67 (m, 14H), 7.77-7.79 (m, 5H), |
| | 7.92-8.02 (m, 3H), 8.20-8.21 (m, 2H), 8.29-8.31 (m, 1H), |
| | 8.41-8.45(m, 2H), 8.53-8.55 (m, 1H) |
| 9 | 7.41-7.55 (m, 7H), 7.66-7.67 (m, 3H), 7.82-7.92 (m, 7H), |
| | 8.02-8.28 (m, 11H), 8.53-8.55 (m, 1H), 8.80-8.82 (d, |
| | 2H), 8.92-8.93 (d, 2H), 9.15 (s, 1H), |
| 15 | 7.25-7.75 (m, 18H), 7.85-7.92 (m, 6H), 8.02 (m, 1H), |
| | 8.16-8.28 (m, 6H), 8.54 (m, 1H), 8.80-8.82 (d, 2H) |
| 16 | 7.25-7.55 (m, 12H), 7.66-7.68 (m, 4H), 7.66-7.92 (m, |
| | 8H), 8.01-8.02 (d, 1H), 8.16-8.28 (m, 6H), 8.54-8.55 (m, |
| | 1H), 8.80-8.82 (d, 2H) |
| 17 | 7.24-7.26 (d, 2H), 7.41-7.56 (m, 10H), 7.65-7.68 (m, |
| | 3H), 7.82-8.02 (m, 9H), 8.16-8.28 (m, 6H), 8.44-8.46 (m, |
| | 1H), 8.53-8.55 (m, 1H), 8.80-8.82 (d, 2H) |
| 21 | 7.24-7.26 (d, 2H), 7.41-7.55 (m, 12H), 7.66-7.68 (m, |
| | 1H), 7.85-7.92 (m, 6H), 8.03-8.05 (d, 1H), 8.16-8.28 (m, |
| | 6H), 8.53-8.55 (m, 1H), 8.80-8.82 (d, 2H) |
| 27 | 7.24-7.26 (d, 4H), 7.41-7.71 (m, 12H), 7.82-7.92 (m, |
| | 4H), 8.04-8.34 (m, 13H), 8.53-8.55 (m, 1H), 8.93-8.99 |
| | (m, 2H) |
| 30 | 7.24-7.26 (d, 2H), 7.41-7.67 (m, 12H), 7.78-7.98 (m, |
| | 5H), 8.05-8.07 (d, 1H), 8.16-8.28 (m, 7H), 8.53-8.55 (m, |
| | 1H) |
| 31 | 7.25-7.27 (d, 2H), 7.41-7.70 (m, 17H), 7.85-7.92 (m, |
| | 3H), 8.01-8.03 (d, 1H), 8.16-8.30 (m, 8H), 8.53-8.56 (m, |
| | 2H), 8.80-8.82 (d, 2H) |
| 34 | 7.25-7.33 (m, 3H), 7.50-7.68 (m, 12H), 7.79-8.02 (m, |
| | 15H), 8.12-8.23 (m, 4H), 8.34 (s, 2H), 8.53-8.56 (m, |
| | 2H), 8.80-8.82 (d, 2H) |
| 35 | 7.25-7.33 (m, 3H), 7.46-7.67 (m, 13H), 7.87-8.23 (m, |
| | 18H), 8.34 (s, 2H), 8.54-8.55 (m, 2H), 8.80-8.82 (m, 2H) |
| 37 | 7.41-7.55 (m, 14H), 7.66-7.68 (m, 2H), 7.79-7.92 (m, |
| | 8H), 8.03-8.05 (d, 1H), 8.16-8.23 (m, 3H), 8.29-8.30 (d, |
| | 2H), 8.53-8.54 (m, 1H), 8.80-8.82 (d, 2H) |
| 41 | 7.25-7.55 (m, 13H), 7.63-7.68 (m, 6H), 7.79-7.94 (m, |
| | 12H), 8.01-8.03 (d, 1H), 8.12-8.23 (m, 4H), 8.54-8.55 |
| | (m, 2H), 8.80-8.82 (d, 2H) |
| 46 | 7.24-7.25 (d, 2H), 7.41-7.55 (m, 14H), 7.66-7.68 (m, |
| | 2H), 7.79-7.92 (m, 10H), 8.03-8.04 (d, 1H), 8.16-8.23 |
| | (m, 3H), 8.29-8.30 (m, 9H), 8.53-8.54 (m, 1H) |
| 52 | 7.41 (m, 3H), 7.51-7.55 (m, 13H), 7.60 (t, 1H), 7.67 (m, |
| | 2H), 7.85 (d, 4H), 7.90 (s, 1H), 8.04 (d, 1H), 8.16 (d, |
| | 1H), 8.21 (m, 2H), 8.23-8.26 (m, 2H), 8.30 (m, 5H), 8.54 |
| | (m, 1H) |
| 57 | 7.25-7.67 (m, 20H), 7.79-7.94 (m, 8H), 8.01-8.03 (d, |
| | 1H), 8.09-8.26 (m, 7H), 8.54-8.55 (m, 2H) |
| 60 | 7.24-7.25 (d, 2H), 7.41-7.67 (m, 15H), 7.79-8.02 (m, |
| | 9H), 8.16-8.26 (m, 6H), 8.41-8.45 (m, 2H), 8.53-8.55 (m, |
| | 1H) |
| 63 | 7.18-7.20 (d, 2H), 7.24-7.26 (d, 2H), 7.41-7.67 (m, |
| | 17H), 7.85-7.89 (m, 7H), 8.01-8.03 (d, 1H), 8.16-8.30 |
| | (m, 7H), 8.53-8.55 (m, 1H) |
| 68 | 7.25-7.55 (m, 13H), 7.63-7.79 (m, 8H), 7.92-7.94 (m, |
| | 2H), 8.01-8.03 (d, 1H), 8.12-8.30 (m, 10H), 8.53-8.56 |
| | (m, 2H) |
| 69 | 7.19-7.21 (d, 2H), 7.24-7.26 (d, 2H), 7.41-7.55 (m, |
| | 14H), 7.66-7.68 (m, 3H), 7.84-7.86 (d, 4H), 7.91-7.93 |
| | (d, 1H), 8.02-8.03 (d, 1H), 8.16-8.23 (m, 3H), 8.30-8.34 |
| | (m, 4H), 8.54-8.55 (m, 1H), 8.80-8.82 (d, 2H) |
| 72 | 7.41-7.75 (m, 23H) 7.90-7.92 (d, 2H), 8.04-8.08 (m, 3H), |
| | 8.16-8.24 (m, 4H) 8.32-8.34 (d, 2H), 8.41-8.43 (d, 1H), |
| | 8.54-8.56 (m, 2H), 8.80-8.82 (d, 2H) |
| 77 | 7.19-7.25 (m, 4H), 7.41-7.55 (m, 14H), 7.66-7.67 (m, |
| | 3H), 7.85-7.92 (m, 9H), 8.01-8.03 (d, 1H), 8.16-8.23 (m, |
| | 3H), 8.29-8.31 (m, 4H), 8.53-8.55(d, 1H), 8.80-8.82 (d, |
| | 2H) |
| 80 | 7.24-7.26 (d, 2H), 7.41-7.70 (m, 20H), 7.78-7.80 (d, |
| | 2H), 7.88-7.92 (m, 3H), 8.01-8.03 (d, 1H), 8.16-8.30 (m, |
| | 6H), 8.53-8.55 (m, 1H), 8.80-8.82 (d, 2H) |
| 85 | 7.19-7.26 (m, 4H), 7.41-7.67 (m, 19H), 7.85-7.92 (m, |
| | 7H), 8.01-8.03 (d, 1H), 8.16-8.30 (m, 9H), 8.53-8.55 (m, |
| | 1H) |
| 87 | 7.41-7.71 (m, 16H), 7.82-7.92 (m, 7H), 8.02-8.04 (m, |
| | 2H), 8.12-8.30 (m, 13H), 8.53-8.55 (m, 1H), 8.93-8.95 |
| | (d, 1H), 9.15 (s, 1H0 |
| 89 | 7.18-7.20 (d, 2H), 7.41-7.70 (m, 20H), 7.79-7.92 (m, |
| | 5H), 8.02-8.03 (d, 1H), 8.16-8.30 (m, 8H), 8.53-8.55 (m, |
| | 1H) |
| 90 | 7.41-7.67 (m, 13H), 7.83-7.92 (m, 7H) 8.02-8.21 (m, 6H), |
| | 8.30-8.32 (d, 2H), 8.41-8.43 (m, 1H), 8.53-8.56 (m, 2H), |
| | 8.81-8.83 (d, 2H) |
| 92 | 7.41-7.58 (m, 14H), 7.67-7.92 (m, 10H), 8.03-8.05 (d, |
| | 1H), 8.16-8.21 (m, 3H), 8.54-8.55 (m, 1H), 8.80-8.82 (d, |
| | 2H) |
| 95 | 7.25-7.40 (m, 7H), 7.50-7.67 (m, 8H), 7.83-8.21 (m, |
| | 15H), 8.54-8.56 (m, 3H), 8.80-8.82 (d, 2H), 9.71 (s, 1H) |
| 96 | 7.25-7.67 (m, 16H), 7.79-7.94 (m, 8H), 8.02-8.21 (m, |
| | 6H), 8.54-8.55 (m, 2H), 8.80-8.82 (d, 2H) |
| 97 | 7.25-7.68 (m, 15H), 7.79-7.94 (m, 10H), 8.01-8.03 (d, |
| | 1H), 8.12-8.21 (m, 4H), 8.53-8.56 (m, 2H), 8.80-8.82 (d, |
| | 2H) |
| 99 | 7.25-7.67 (m, 16H), 7.81-7.92 (m, 10H), 8.01-8.03 (d, |
| | 1H), 8.16-8.21 (m, 2H), 8.53-8.55 (m, 1H), 8.80-8.82 (d, |
| | 2H) |
| 100 | 7.25 (s, 4H), 7.41-7.67 (m, 1H), 7.79-8.02 (m, 9H), |
| | 8.16-8.21 (m, 4H), 8.41-8.45 (m, 2H), 8.53-8.55 (m, 1H), |
| | 8.80-8.82 (d, 2H) |
| 102 | 7.25-7.79 (m, 16H), 7.63-7.68 (m, 6H), 7.78-7.80 (d, |
| | 6H), 7.92-7.94 (m, 2H), 8.01-8.03 (d, 1H), 8.12-8.28 (m, |
| | 5H), 8.54-8.56 (m, 2H), 8.80-8.81 (d, 2H) |
| 105 | 7.25-7.28 (m, 6H), 7.41-7.56 (m, 13H), 7.66-7.68 (m, |
| | 2H), 7.78-7.82 (m, 5H), 7.90-8.04 (m, 5H), 8.16-8.28 (m, |
| | 4H), 8.44-8.46 (m, 1H), 8.54-8.56 (m, 1H), 8.80-8.81 (d, |
| | 1H) |
| 111 | 7.41-7.71 (m, 9H), 7.82-7.92 (m, 6H), 8.04-8.33 (m, |
| | 11H), 8.54-8.56 (m, 1H), 8.80-8.82 (d, 2H), 8.92-8.94 |
| | (d, 1H), 9.15 (s, 1H) |
| 112 | 7.25 (s, 4H), 7.41-7.67 (m, 11H), 7.83-8.02 (m, 5H), |
| | 8.16-8.54 (m, 10H), 8.80-8.82 (d, 2H) |
| 113 | 7.25 (s, 4H), 7.41-7.67 (m, 11H), 7.82-8.02 (m, 7H), |
| | 8.16-8.33 (m, 7H), 8.45-8.56 (m, 2H), 8.80-8.82 (d, 2H) |
| 114 | 7.25 (s, 4H), 7.32-7.92 (m, 18H), 8.01-8.03 (d, 1H), |
| | 8.16-8.33 (m, 7H), 8.53-8.55 (m, 1H), 8.80-8.82 (d, 2H) |
| 115 | 7.25-7.68 (m, 15H), 7.79-7.94 (m, 6H), 8.01-8.03 (d, |
| | 1H), 8.12-8.33 (m, 8H), 8.53-8.56 (m, 2H), 8.80-8.82 (d, |
| | 2H |
| 118 | 7.34-7.36 (d, 2H), 7.47-7.55 (m, 4H), 7.66-7.71 (m, 5H), |
| | 7.81-7.92 (m, 4H), 8.02-8.21 (m, 9H), 8.29-8.31 (d, 2H), |
| | 8.54-8.55 (m, 1H), 8.84 (s, 4H), 8.92-8.94 (d, 1H), 9.15 |
| | (s, 1H) |
| 123 | 7.19-7.21 (d, 2H), 7.35-7.67 (m, 14H), 7.81-7.92 (m, |
| | 4H), 8.02-8.30 (m, 10H), 8.53-8.55 (m, 1H), 8.80-8.82 |
| | (d, 2H |
| 124 | 6.85-6.87 (t, 1H), 7.19-7.22 (m, 3H), 7.41-7.55 (m, 5H), |
| | 7.65-7.68 (m, 3H), 7.85-7.92 (m, 5H), 8.01-8.03 (d, 1H), |
| | 8.16-8.21 (m, 2H), 8.29-8.31 (d, 2H), 8.37 (s, 1H), |
| | 8.48-8.54 (m, 2H), 8.80-8.82 (d, 2H) |
| 126 | 6.85-6.87 (t, 1H), 7.21-7.33 (m, 4H), 7.48-7.79 (m, |
| | 15H),, 7.88-7.94 (m, 4H), 8.01-8.03 (d, 1H), 8.12-8.21 |
| | (m, 3H), 8.37 (s, 1H), 8.48-8.55 (m, 3H), 8.80-8.82 (d, |
| | 2H) |
| 130 | 1.28-1.31 (t, 3H), 4.10-4.14 (m, 2H), 7.21-7.30 (m, 5H), |
| | 7.50-7.68 (m, 9H), 7.78-7.80 (d, 2H), 7.88-7.94 (m, 5H), |
| | 8.03-8.05 (d, 1H), 8.12-8.21 (m, 3H), 8.53-8.55 (m, 2H), |
| | 8.80-8.82 (d, 2H) |
| 134 | 1.28-1.31 (t, 3H), 4.10-4.14 (m, 2H), 7.21-7.33 (m, 5H), |
| | 7.50-7.71 (m, 10H), 7.85-7.94 (m, 4H), 8.01-8.03 (d, |
| | 1H), 8.12-8.21 (m, 3H), 8.29-8.31 (d, 2H), 8.53-8.55 (m, |
| | 2H) |
| 135 | 1.28-1.31 (t, 3H), 4.10-4.14 (m, 2H), 7.21-7.33 (m, 5H), |
| | 7.46-7.67 (m, 12H), 7.92-8.30 (m, 10H), 8.53-8.55 (m, |
| | 2H) |
| 139 | 7.25-7.33 (m, 5H), 7.46-7.67 (m, 10H), 7.85-8.21 (m, |
| | 14H), 8.53-8.55 (m, 2H), 8.80-8.82 (d, 2H) |
| 142 | 7.41-7.55 (m, 9H), 7.66-7.68 (m, 2H), 7.77-7.92 (m, 9H), |
| | 8.02-8.04 (m, 3H), 8.16-8.21 (m, 2H), 8.35 (s, 1H), |
| | 8.53-8.55 (m, 1H), 8.80-8.82 (d, 2H) |
| 147 | 7.41-7.82 (m, 19H), 7.92-8.03 (m, 6H), 8.16-8.26 (m, |
| | 4H), 8.34 (s, 1H), 8.45-8.54 (m, 2H) |
| 148 | 7.25-7.33 (m, 3H), 7.47-7.68 (m, 14H), 7.78-7.80 (d, |
| | 2H), 7.88-7.94 (m, 4H), 8.01-8.03 (d, 1H), 8.12-8.30 (m, |
| | 9H), 8.53-8.55 (d, 2H), 8.80-8.82 (d, 2H) |
| 151 | 7.25-7.33 (m, 3H), 7.46-7.67 (m, 14H), 7.88-7.94 (m, |
| | 5H), 8.04-8.21 (m, 7H), 8.29-8.31 (d, 4H), 8.53-8.55 (m, |
| | 2H), 8.80-8.82 (d, 2H) |
| 152 | 7.25-7.33 (m, 3H), 7.46-7.67 (m, 15H), 7.88-7.94 (m, |
| | 4H), 8.01-8.03 (d, 1H), 8.09-8.21 (m, 6H) 8.29-8.31 (d, |
| | 4H), 8.53-8.55 (m, 2H), 8.80-8.82 (d, 2H) |
| 155 | 6.99-7.01 (d, 1H), 7.13-.7.15 (t, 1H), 7.55-7.70 (m, |
| | 6H), 7.82-7.92 (m, 5H), 8.02-8.21 (m, 7H), 8.53-8.55 (m, |
| | 2H), 8.84-8.93 (m, 7H), 9.14-9.15 (s, 1H), 9.29-9.31 (d, |
| | 1H) |
| 161 | 7.13-7.15 (t, 2H), 7.54-7.70 (m, 11H), 7.91-7.93 (d, |
| | 1H), 8.02-8.26 (m, 7H), 8.41-8.43 (d, 1H), 8.53-8.55 (m, |
| | 4H), 9.14-9.15 (s, 2H), 9.29-9.31 (d, 2H) |
| 162 | 7.25-7.28 (m, 6H), 7.41 (t, 2H), 7.51-7.52 (m, 6H), 7.55 |
| | (d, 1H), 7.66-7.67 (m, 3H), 7.82-7.92 (m, 10H), 8.02 (d, |
| | 1H), 8.12 (d, 2H), 8.16 (d, 1H), 8.21 (d, 1H), 8.28 (d, |
| | 2H), 8.54 (d, 1H), 8.81 (d, 2H), 8.93 (d, 2H) |
| 164 | 7.25-7.28 (m, 4H), 7.41-7.48 (m,3H), 7.51-7.57 (m, 8H), |
| | 7.66=7.67 (m, 3H), 7.70 (s, 1H), 7.82-7.92 (m, 8H), 8.02 |
| | (d, 1H), 8.12-8.28 (m, 7H), 8.54 (m, 1H), 8.81 (d, 2H), |
| | 8.93 (d, 2H) |
| 166 | 7.25-7.28 (m, 4H), 7.41 (m, 2H), 7.51-7.55 (m, 7H), |
| | 7.66-7.67 (m, 3H), 7.82-7.93 (m, 8H), 8.02 (d, 1H), 8.12 |
| | (d, 2H), 8.16 (d, 1H), 8.21-8.23 (m, 2H), 8.28-8.30 (m, |
| | 6H), 8.54 (m, 1H), 8.81 (d, 2H), 8.93 (d, 2H) |
| 172 | 7.41-7.48 (m, 3H), 7.51-7.57 (m, 8H), 7.67-7.70 (m, 4H), |
| | 7.82 (t, 2H), 7.88 (m, 4H), 7.92 (d, 1H), 8.04 (d, 2H), |
| | 8.12-8.28 (m, 8H), 8.54 (d, 1H), 8.81 (d, 2H), 8.93 (d, |
| | 2H), 9.15 (s, 1H) |
| 174 | 7.41-7.48 (m, 3H), 7.51-7.57 (m, 7H), 7.67-7.70 (m, 3H), |
| | 7.82-7.88 (m, 6H), 7.92 (d, 1H), 8.12 (d, 2H), 8.16-8.23 |
| | (m, 5H), 8.28-8.30 (m, 6H), 8.54 (m, 1H), 8.81 (d, 2H), |
| | 8.93 (d, 2H), 9.15 (s, 1H) |
| 176 | 7.41 (m, 2H), 7.48-7.52 (m, 7H), 7.55-7.57 (m, 2H), |
| | 7.66-7.70 (m, 4H), 7.75 (d, 1H), 7.82-7.88 (m, 4H), |
| | 8.02-8.04 (m, 2H), 8.12-8.18 (m, 4H), 8.21-8.23 (m, 2H), |
| | 8.28-8.33 (m, 4H), 8.54 (m, 1H), 8.81 (d, 2H), 8.93 (d, |
| | 2H), 9.15 (s, 1H) |
| 178 | 7.24-7.25 (m, 6H), 7.41 (m, 2H), 7.51-7.59 (m, 7H), |
| | 7.66-7.67 (m, 3H), 7.85 (d, 2H), 7.92-7.95 (m, 2H), |
| | 8.00-8.02 (m, 3H), 8.16 (d, 1H), 8.21 (d, 1H), 8.24 (d, |
| | 4H), 8.38 (d, 1H), 8.54 (d, 1H), 8.85 (s, 1H) |
| 186 | 7.25-7.41 (m, 6H), 7.50-7.55 (m, 6H), 7.67-7.68 (m, 5H), |
| | 7.79 (d, 2H), 7.85 (d, 2H), 7.88 (d, 2H), 7.90-7.94 (m, |
| | 3H), 8.04 (d, 1H), 8.12 (d, 1H), 8.16 (d, 1H), 8.21 (d, |
| | 1H), 8.28 (d, 4H), 8.54-8.85 (m, 2H), 8.81 (d, 2H) |
| 191 | 7.25-7.33 (m, 3H), 7.41 (t, 2H), 7.50-7.55 (m, 6H), |
| | 7.67-7.68 (m, 5H), 7.79 (m, 4H), 7.85 (d, 2H), 7.88 (d, |
| | 2H), 7.90-7.94 (m, 3H), 8.04 (d, 1H), 8.12 (d, 1H), 8.16 |
| | (d, 1H), 8.21-8.23 (m, 2H), 8.28 (d, 2H), 8.30 (d, 2H), |
| | 8.54-8.85 (m, 2H), 8.81 (d, 2H) |
| 195 | 7.16-7.20 (m, 2H), 7.35-7.58 (m, 13H), 7.75 (d, 2H), |
| | 7.85-7.99 (m, 10H), 8.14-8.35 (m, 9H), 8.54-8.55 (m, |
| | 2H), 8.69 (d, 2H) |
| 198 | 7.16-7.20 (m, 2H), 7.35-7.75 (m, 17H), 7.91-7.99 (m, |
| | 10H), 8.14-8.23 (m, 3H), 8.30-8.35 (m, 4H), 8.54-8.55 |
| | (m, 2H), 8.69 (d, 2H) |
| 202 | 7.16-7.20 (m, 2H), 7.35-7.75 (m, 17H), 7.91-7.99 (m, |
| | 11H), 8.14-8.19 (m, 2H), 8.36-8.38 (m, 3H), 8.54-8.55 |
| | (m, 2H), 8.69 (d, 2H) |
| 204 | 7.16-7.20 (m, 2H), 7.35 (t, 1H), 7.50-7.65 (m, 12H), |
| | 7.91-7.99 (m, 10H), 8.14-8.19 (m, 2H), 8.36 (m, 4H), |
| | 8.54-8.55 (m, 2H), 8.69 (d, 2H) |
| 205 | 2.34 (s, 6H), 7.16-7.20 (m, 2H), 7.35 (t, 1H), 7.45 (d, |
| | 4H), 7.50-7.65 (m, 6H), 7.91-7.99 (m, 10H), 8.14-8.19 |
| | (d, 2H), 8.54-8.60 (m, 6H), 8.69 (d, 2H) |
| 206 | 7.41-7.62 (m, 12H), 7.75 (d, 2H), 7.94-7.99 (m, 5H), |
| | 8.09 (d, 1H), 8.23 (s, 1H), 8.30-8.35 (m, 5H), 8.54 (d, |
| | 1H), 8.69 (d, 2H) |
| 209 | 7.25 (d, 2H), 7.41-7.62 (m, 12H), 7.75 (d, 4H), 7.94- |
| | 7.99 (m, 7H), 8.09 (d, 1H), 8.23 (s, 1H), 8.30-8.31 (m, |
| | 3H), 8.54 (d, 1H), 8.69 (d, 2H) |
| 210 | 7.41-7.62 (m, 12H), 7.75 (d, 2H), 7.94-7.99 (m, 3H), |
| | 8.09 (d, 2H), 8.31-8.36 (m, 5H), 8.54 (d, 1H), 8.69 (d, |
| | 2H) |
| 212 | 7.41-7.61 (m, 14H), 7.73-7.75 (m, 7H), 7.94-7.99 (m, |
| | 7H), 8.09 (d, 1H), 8.31-8.38 (m, 3H), 8.54 (d, 1H), 8.69 |
| | (d, 2H) |
| 214 | 7.41-7.48 (m, 5H), 7.51-7.57 (m, 8H), 7.66-7.70 (m, 4H), |
| | 7.79 (d, 2H), 7.85 (d, 2H), 7.92 (d, 1H), 8.02 (d, 1H), |
| | 8.16 (d, 1H), 8.21-8.23 (m, 2H), 8.28-8.30 (m, 6H), 8.54 |
| | (m, 1H) |
| 216 | 7.41-7.48 (m, 4H), 7.51-7.57 (m, 8H), 7.66-7.70 (m, 4H), |
| | 7.79 (d, 2H), 7.92 (d, 1H), 8.02 (d, 1H), 8.16 (d, 1H), |
| | 8.21 (d, 1H), 8.28-8.30 (m, 4H), 8.54 (m, 1H) |

TABLE 2

| Compound | FD-MS | Compound | FD-MS |
|---|---|---|---|
| 1 | m/z =: 788.93 | 2 | m/z = 506.59 |
| | (C58H36N4 = 788.29) | | (C38H22N2 = 506.18) |
| 3 | m/z = 757.85 | 4 | m/z = 757.85 |
| | (C55H36NOP = 757.25) | | (C55H36NOP = 757.25) |
| 5 | m/z = 746.83 | 6 | m/z = 763.88 |
| | (C53H35N2OP = 746.25) | | (C53H34NOPS = 763.21) |
| 7 | m/z = 733.83 | 8 | m/z = 733.83 |
| | (C53H36NOP = 733.25) | | (C53H36NOP = 733.25) |
| 9 | m/z = 762.90 | 10 | m/z = 838.99 |
| | (C56H34N4 = 762.28) | | (C62H38N4 = 838.31) |
| 11 | m/z = 762.90 | 12 | m/z =: 762.90 |
| | (C56H34N4 = 762.28) | | (C56H34N4 = 762.28) |
| 13 | m/z = 812.95 | 14 | m/z = 778.90 |
| | (C60H36N4 = 812.29) | | (C56H34N4O = 778.27) |
| 15 | m/z = 778.90 | 16 | m/z = 794.96 |
| | (C56H34N4O = 778.27) | | (C56H34N4S = 794.25) |
| 17 | m/z = 794.96 | 18 | m/z = 788.93 |
| | (C56H34N4S = 794.25) | | (C58H36N4 = 788.29) |
| 19 | m/z = 788.93 | 20 | m/z = 889.05 |
| | (C58H36N4 = 788.29) | | (C66H40N4 = 888.33) |
| 21 | m/z = 688.82 | 22 | m/z = 688.82 |
| | (C50H32N4 = 688.26) | | (C50H32N4 = 688.26) |
| 23 | m/z = 712.84 | 24 | m/z = 788.93 |
| | (C52H32N4 = 712.26) | | (C58H36N4 = 788.29) |
| 25 | m/z = 838.99 | 26 | m/z = 838.99 |
| | (C62H38N4 = 838.31) | | (C62H38N4 = 838.31) |
| 27 | m/z = 788.93 | 28 | m/z = 812.95 |
| | (C58H36N4 = 788.29) | | (C60H36N4 = 812.29) |
| 29 | m/z = 812.95 | 30 | m/z = 612.72 |
| | (C60H36N4 = 812.29) | | (C44H28N4 = 612.23) |
| 31 | m/z = 688.82 | 32 | m/z = 688.82 |
| | (C50H32N4 = 688.26) | | (C50H32N4 = 688.26) |
| 33 | m/z = 787.96 | 34 | m/z = 877.04 |
| | (C59H37N3 = 787.30) | | (C65H40N4 = 876.33) |
| 35 | m/z = 877.04 | 36 | m/z = 763.92 |
| | (C65H40N4 = 876.33) | | (C57H37N3 = 763.30) |
| 37 | m/z = 687.83 | 38 | m/z =: 763.92 |
| | (C51H33N3 = 687.27) | | (C57H37N3 = 763.30) |
| 39 | m/z = 687.83 | 40 | m/z =: 737.89 |
| | (C51H33N3 = 687.27) | | (C55H35N3 = 737.28) |
| 41 | m/z = 853.02 | 42 | m/z = 853.02 |
| | (C63H40N4 = 852.33) | | (C63H40N4 = 852.33) |
| 43 | m/z = 853.02 | 44 | m/z = 877.04 |
| | (C63H40N4 = 852.33) | | (C65H40N4 = 876.33) |
| 45 | m/z = 840.02 | 46 | m/z = 763.92 |
| | (C63H41N3 = 839.33) | | (C57H37N3 = 763.30) |
| 47 | m/z = 853.02 | 48 | m/z = 853.02 |
| | (C63H40N4 = 852.33) | | (C63H40N4 = 852.33) |
| 49 | m/z = 711.85 | 50 | m/z = 711.85 |
| | (C53H33N3 = 711.27) | | (C53H33N3 = 711.27) |
| 51 | m/z = 811.97 | 52 | m/z = 763.92 |
| | (C61H37N3 = 811.30) | | (C57H37N3 = 763.30) |
| 53 | m/z = 687.83 | 54 | m/z = 763.92 |
| | (C51H33N3 = 687.27) | | (C57H37N3 = 763.30) |
| 55 | m/z = 811.97 | 56 | m/z = 853.02 |
| | (C61H37N3 = 811.30) | | (C63H40N4 = 852.33) |
| 57 | m/z = 853.02 | 58 | m/z = 870.07 |
| | (C63H40N4 = 852.33) | | (C63H39N3S = 869.29) |
| 59 | m/z = 787.95 | 60 | m/z = 793.97 |
| | (C59H37N3 = 787.30) | | (C57H35N3S = 793.26) |
| 61 | m/z = 793.97 | 62 | m/z = 840.02 |
| | (C57H35N3S = 793.26) | | (C63H41N3 = 839.33) |
| 63 | m/z = 763.92 | 64 | m/z = 870.07 |
| | (C57H37N3 = 763.30) | | (C63H39N3S = 869.29) |
| 65 | m/z = 776.92 | 66 | m/z = 776.92 |
| | (C57H36N4 = 776.29) | | (C57H36N4 = 776.29) |
| 67 | m/z = 793.97 | 68 | m/z = 776.92 |
| | (C57H35N3S = 793.26) | | (C57H36N4 = 776.29) |
| 69 | m/z = 763.92 | 70 | m/z = 761.91 |
| | (C57H37N3 = 763.30) | | (C57H35N3 = 761.28) |
| 71 | m/z = 687.83 | 72 | m/z = 813.98 |
| | (C51H33N3 = 687.27) | | (C61H39N3 = 813.31) |
| 73 | m/z = 870.07 | 74 | m/z = 870.07 |
| | (C63H39N3S = 869.29) | | (C63H39N3S = 869.29) |
| 75 | m/z = 787.95 | 76 | m/z = 787.95 |
| | (C59H37N3 = 787.30) | | (C59H37N3 = 787.30) |
| 77 | m/z = 840.02 | 78 | m/z = 763.92 |
| | (C63H41N3 = 839.33) | | (C57H37N3 = 763.30) |
| 79 | m/z = 840.02 | 80 | m/z = 763.92 |
| | (C63H41N3 = 839.33) | | (C57H37N3 = 763.30) |
| 81 | m/z = 813.98 | 82 | m/z = 787.95 |
| | (C61H39N3 = 813.31) | | (C59H37N3 = 787.30) |
| 83 | m/z = 787.95 | 84 | m/z = 888.06 |
| | (C59H37N3 = 787.30) | | (C67H41N3 = 887.33) |
| 85 | m/z = 840.02 | 86 | m/z = 864.04 |
| | (C63H41N3 = 839.33) | | (C59H37N3 = 863.33) |
| 87 | m/z = 864.04 | 88 | m/z = 840.02 |
| | (C65H41N3 = 863.33) | | (C63H41N3 = 839.33) |
| 89 | m/z = 763.92 | 90 | m/z = 711.85 |
| | (C57H37N3 = 763.30) | | (C53H33N3 = 711.27) |
| 91 | m/z = 685.81 | 92 | m/z = 661.79 |
| | (C51H31N3 = 685.25) | | (C49H31N3 = 661.25) |
| 93 | m/z = 685.81 | 94 | m/z = 685.81 |
| | (C51H31N3 = 685.25) | | (C51H31N3 = 685.25) |
| 95 | m/z = 839.98 | 96 | m/z = 750.89 |
| | (C61H37N5 = 839.30) | | (C55H34N4 = 750.28) |
| 97 | m/z = 750.89 | 98 | m/z = 750.89 |
| | (C55H34N4 = 750.28) | | (C55H34N4 = 750.28) |
| 99 | m/z = 751.87 | 100 | m/z = 767.94 |
| | (C55H33N3O = 751.26) | | (C55H33N3S = 767.24) |
| 101 | m/z = 838.00 | 102 | m/z = 853.02 |
| | (C63H39N3 = 837.31) | | (C63H40N4 = 852.33) |
| 103 | m/z = 854.00 | 104 | m/z = 870.07 |
| | (C63H39N3O = 853.31) | | (C63H39N3S = 869.29) |
| 105 | m/z = 870.07 | 106 | m/z = 870.07 |
| | (C63H39N3S = 869.29) | | (C63H39N3S = 869.29) |
| 107 | m/z = 870.07 | 108 | m/z = 585.69 |
| | (C63H39N3S = 869.29) | | (C43H27N3 = 585.22) |
| 109 | m/z = 661.7 9 | 110 | m/z = 685.81 |
| | (C49H31N3 = 661.25) | | (C51H31N3 = 685.25) |
| 111 | m/z = 685.81 | 112 | m/z = 767.94 |
| | (C51H31N3 = 685.25) | | (C55H33N3S = 767.24) |
| 113 | m/z = 767.94 | 114 | m/z = 751.87 |
| | (C55H33N3S = 767.24) | | (C55H33N3O = 751.26) |
| 115 | m/z = 750.89 | 116 | m/z = 750.89 |
| | (C55H34N4 = 750.28) | | (C55H34N4 = 750.28) |
| 117 | m/z = 635.75 | 118 | m/z = 735.87 |
| | (C47H29N3 = 635.24) | | (C55H33N3 = 735.27) |
| 119 | m/z = 711.85 | 120 | m/z = 635.75 |
| | (C53H33N3 = 711.27) | | (C47H29N3 = 635.24) |
| 121 | m/z = 785.93 | 122 | m/z = 785.93 |
| | (C59H35N3 = 785.28) | | (C59H35N3 = 785.28) |
| 123 | m/z = 711.85 | 124 | m/z = 573.68 |
| | (C53H33N3 = 711.27) | | (C42H27N3 = 573.22) |
| 125 | m/z = 738.87 | 126 | m/z = 738.87 |
| | (C54H34N4 = 738.28) | | (C54H34N4 = 738.28) |
| 127 | m/z = 738.87 | 128 | m/z = 573.68 |
| | (C54H34N4 = 738.28) | | (C42H27N3 = 573.22) |
| 129 | m/z = 690.83 | 130 | m/z = 690.83 |
| | (C50H34N4 = 690.28) | | (C50H34N4 = 690.28) |
| 131 | m/z = 690.83 | 132 | m/z = 690.83 |
| | (C50H34N4 = 690.28) | | (C50H34N4 = 690.28) |
| 133 | m/z = 814.97 | 134 | m/z = 690.83 |
| | (C60H38N4 = 814.31) | | (C50H34N4 = 690.28) |
| 135 | m/z = 690.83 | 136 | m/z = 690.83 |
| | (C50H34N4 = 690.28) | | (C50H34N4 = 690.28) |
| 137 | m/z = 690.83 | 138 | m/z = 690.83 |
| | (C50H34N4 = 690.28) | | (C50H34N4 = 690.28) |
| 139 | m/z = 755.92 | 140 | m/z = 590.73 |
| | (C54H33N3S = 755.24) | | (C42H26N2S = 590/.18) |
| 141 | m/z = 755.92 | 142 | m/z = 666.83 |
| | (C54H33N3S = 755.24) | | (C48H30N2S = 666.21) |
| 143 | m/z = 640.79 | 144 | m/z = 640.79 |
| | (C46H28N2S = 640.20) | | (C46H28N2S = 640.20) |
| 145 | m/z = 772.98 | 146 | m/z = 772.98 |
| | (C54H32N2S2 = 772.20) | | (C54H32N2S2 = 772.20) |
| 147 | m/z = 772.98 | 148 | m/z = 775.93 |
| | (C54H32N2S2 = 772.20) | | (C58H37N3 = 775.30) |
| 149 | m/z = 775.93 | 150 | m/z = 775.93 |
| | (C58H37N3 = 775.30) | | (C58H37N3 = 775.30) |
| 151 | m/z = 775.93 | 152 | m/z =: 775.93 |
| | (C58H37N3 = 775.30) | | (C58H37N3 = 775.30) |
| 153 | m/z = 775.93 | 154 | m/z = 662.78 |
| | (C58H37N3 = 775.30) | | (C48H30N4 = 662.25) |
| 155 | m/z = 685.81 | 156 | m/z = 611.73 |
| | (C51H31N3 = 685.25) | | (C45H29N3 = 611.24) |
| 157 | m/z = 611.73 | 158 | m/z = 610.74 |
| | (C45H29N3 = 611.24) | | (C46H40N2 = 610.24) |
| 159 | m/z = 686.84 | 160 | m/z = 736.90 |
| | (C52H34N2 = 686.27) | | (C56H36N2 = 736.29) |
| 161 | m/z = 662.78 | 162 | m/z = 865.03 |
| | (C48H30N4 = 662.25) | | (C64H40N4 = 864.33) |
| 163 | m/z = 941.13 | 164 | m/z = 865.03 |
| | (C70H44N4 = 940.36) | | (C64H40N4 = 864.33) |
| 165 | m/z = 941.13 | 166 | m/z = 864.04 |
| | (C70H44N4 = 940.36) | | (C65H41N3 = 863.33) |
| 167 | m/z = 940.14 | 168 | m/z = 864.04 |
| | (C71H45N3 = 939.36) | | (C65H41N3 = 863.33) |
| 169 | m/z = 940.14 | 170 | m/z = 838.99 |
| | (C71H45N3 = 939.36) | | (C62H38N4 = 838.31) |
| 171 | m/z = 915.09 | 172 | m/z = 838.99 |
| | (C68H42N4 = 914.34) | | (C62H38N4 = 838.31) |
| 173 | m/z = 915.09 | 174 | m/z = 838.00 |
| | (C68H42N4 = 914.34) | | (C63H39N3 = 837.31) |
| 175 | m/z = 914.10 | 176 | m/z = 838.00 |
| | (C69H43N3 = 913.35) | | (C63H39N3 = 837.31) |
| 177 | m/z = 914.10 | 178 | m/z = 738.87 |
| | (C69H43N3 = 913.35) | | (C54H34N4 = 738.28) |
| 179 | m/z = 738.87 | 180 | m/z = 764.91 |
| | (C54H34N4 = 738.28) | | (C56H36N4 = 764.29) |
| 181 | m/z = 865.03 | 182 | m/z = 738.87 |
| | (C64H40N4 = 864.33) | | (C54H34N4 = 738.28) |
| 183 | m/z = 738.87 | 184 | m/z = 764.91 |
| | (C54H34N4 = 738.28) | | (C56H36N4 = 764.29) |
| 185 | m/z = 865.03 | 186 | m/z = 854.01 |
| | (C64H40N4 = 864.33) | | (C62H39N5 = 853.32) |
| 187 | m/z = 930.10 | 188 | m/z = 1006.20 |
| | (C68H43N5 = 929.35) | | (C74H47N5 = 1005.38) |
| 189 | m/z = 930.10 | 190 | m/z = 1006.20 |
| | (C68H43N5 = 929.35) | | (C74H47N5 = 1005.38) |
| 191 | m/z = 853.02 | 192 | m/z = 929.11 |
| | (C63H40N4 = 852.33) | | (C69H44N4 = 928.36) |
| 193 | m/z = 1005.21 | 194 | m/z = 929.11 |
| | (C75H48N4 = 1004.39) | | (C69H44N4 = 928.36) |
| 195 | m/z = 853.02 | 196 | m/z = 929.11 |
| | (C63H40N4 = 852.33) | | (C69H44N4 = 928.36) |
| 197 | m/z = 853.02 | 198 | m/z = 853.02 |
| | (C63H40N4 = 852.33) | | (C63H40N4 = 852.33) |
| 199 | m/z = 854.01 | 200 | m/z = 930.10 |
| | (C62H39N5 = 853.32) | | (C68H43N5 = 929.35) |
| 201 | m/z = 854.01 | 202 | m/z = 854.01 |
| | (C62H39N5 = 853.32) | | (C62H39N5 = 853.32) |
| 203 | m/z = 930.10 | 204 | m/z = 777.91 |
| | (C68H43N5 = 929.35) | | (C56H35N5 = 777.29) |
| 205 | m/z = 805.96 | 206 | m/z = 611.73 |
| | (C58H39N5 = 805.32) | | (C45H29N3 = 611.24) |
| 207 | m/z = 763.92 | 208 | m/z = 763.92 |
| | (C57H37N3 = 763.30) | | (C57H37N3 = 763.30) |
| 209 | m/z = 687.83 | 210 | m/z = 612.72 |
| | (C51H33N3 = 687.27) | | (C44H28N4 = 612.23) |
| 211 | m/z = 764.91 | 212 | m/z = 764.91 |
| | (C56H36N4 = 764.29) | | (C56H36N4 = 764.29) |
| 213 | m/z = 688.82 | 214 | m/z = 687.83 |
| | (C50H32N4 = 688.26) | | (C51H33N3 = 687.27) |
| 215 | m/z = 687.83 | 216 | m/z = 611.73 |
| | (C51H33N3 = 687.27) | | (C45H29N3 = 611.24) |
| 217 | m/z = 737.89 | 218 | m/z = 737.89 |
| | (C55H35N3 = 737.28) | | (C55H35N3 = 737.28) |

### EXPERIMENTAL EXAMPLE

### <Experimental Example 1> Manufacture of Organic Light Emitting Device

1) Manufacture of Organic Light Emitting Device

A glass substrate on which indium tin oxide (ITO) was coated as a thin film to a thickness of 1500 Å was cleaned with distilled water ultrasonic waves. After the cleaning with distilled water was finished, the substrate was ultrasonic cleaned with solvents such as acetone, methanol and isopropyl alcohol, then dried, and UVO treatment was performed for 5 minutes using UV in a UV cleaner. After that, the substrate was transferred to a plasma cleaner (PT), and plasma treatment was performed under vacuum for ITO work function and residual film removal, and the substrate was transferred to a thermal deposition apparatus for organic deposition.

On the transparent ITO electrode (anode), an organic material was formed in a 2 stack white organic light emitting device (WOLED) structure. As for the first stack, TAPC was thermal vacuum deposited to a thickness of 300 Å first to form a hole transfer layer. After forming the hole transfer layer, a light emitting layer was thermal vacuum deposited thereon as follows. The light emitting layer was deposited to 300 Å by doping FIrpic to TCz1, a host, by 8% as a blue phosphorescent dopant. After forming an electron transfer layer to 400 Å using TmPyPB, a charge generation layer was formed to 100 Å by doping Cs₂CO₃ to the compound listed in the following Table 3 by 20%.

As for the second stack, MoO₃ was thermal vacuum deposited to a thickness of 50 Å first to form a hole injection layer. A hole transfer layer, a common layer, was formed by doping MoO₃ to TAPC by 20% to 100 Å and then depositing TAPC to 300 Å. A light emitting layer was deposited thereon to 300 Å by doping Ir(ppy)₃, a green phosphorescent dopant, to TCz1, a host, by 8%, and an electron transfer layer was formed to 600 Å using TmPyPB. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å to manufacture an organic light emitting device.

Meanwhile, all the organic compounds required to manufacture the OLED were vacuum sublimation purified under 10⁻⁶ torr to 10⁻⁸ torr by each material to be used in the OLED manufacture.

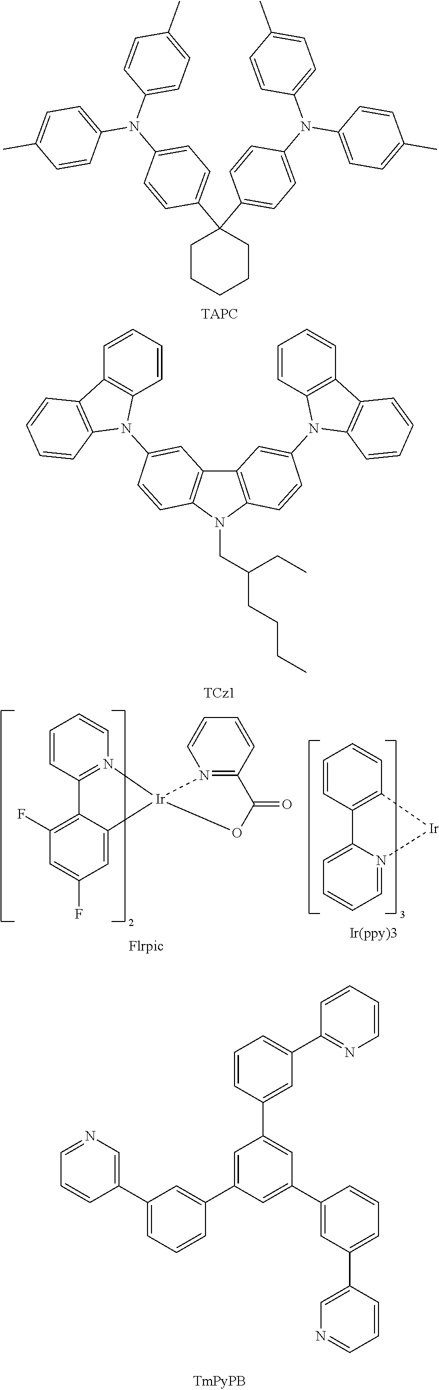

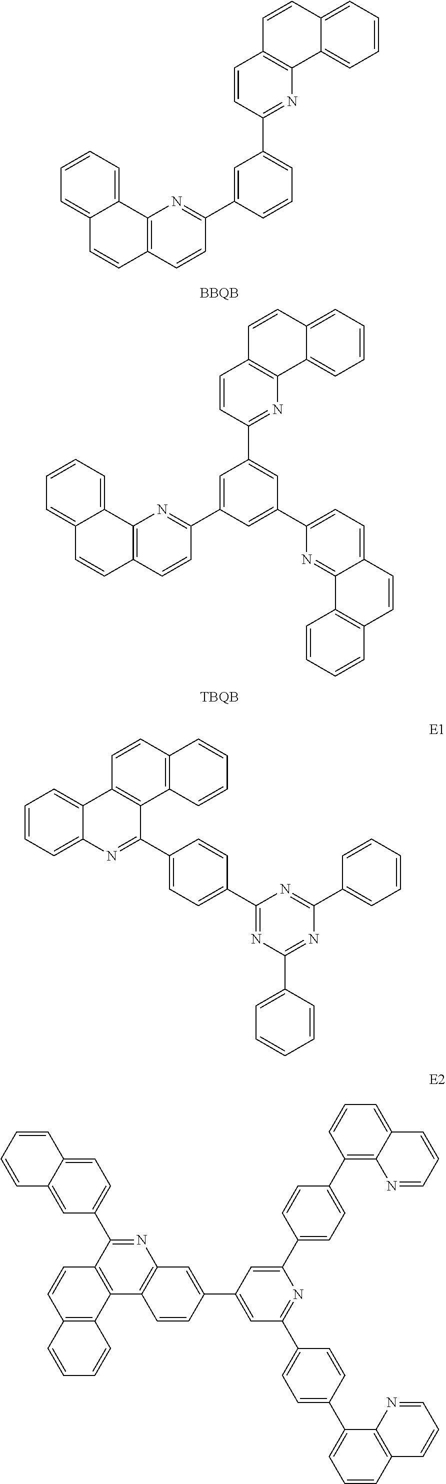 [SMILES: c1cc(-c2ccc3ccc4ccccc4c3n2)cc(-c2ccc3ccc4ccccc4c3n2)c1 ; c1ccc2c(c1)ccc1ccc(-c3cc(-c4ccc5ccc6ccccc6c5n4)cc(-c4ccc5ccc6ccccc6c5n4)c3)nc12 ; c1ccc(-c2nc(-c3ccccc3)nc(-c3ccc(-c4nc5ccccc5c5ccc6ccccc6c45)cc3)n2)cc1 ; c1ccc2cc(-c3nc4cc(-c5cc(-c6ccc(-c7cccc8cccnc78)cc6)nc(-c6ccc(-c7cccc8cccnc78)cc6)c5)ccc4c4c3ccc3ccccc34)ccc2c1]

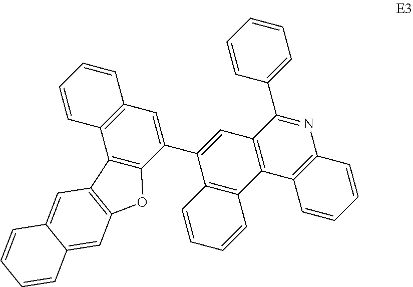 [SMILES: c1ccc(-c2nc3ccccc3c3c2cc(-c2cc4ccccc4c4c2oc2cc5ccccc5cc24)c2ccccc23)cc1]

2) Driving Voltage and Light Emission Efficiency of Organic Light Emitting Device

For the organic light emitting devices manufactured as above, electroluminescent light emission (EL) properties were measured using M7000 manufactured by McScience Inc., and with the measurement results, T₉₅ when standard luminance was 3,500 cd/m² was measured using a lifetime measurement system (M6000) manufactured by McScience Inc. Results of measuring a driving voltage, light emission efficiency, external quantum efficiency and a color coordinate (CIE) of the white organic light emitting device manufactured according to the present disclosure are as shown in Table 3.

TABLE 3

| | | | Light | | |
|---|---|---|---|---|---|
| | | Driving | Emission | | |
| | | Voltage | Efficiency | CIE | Lifetime |
| | Compound | (V) | (cd/A) | (x, y) | (T₉₅) |
| Example 1 | 2 | 7.78 | 59.95 | (0.218, 0.427) | 27 |
| Example 2 | 3 | 7.95 | 59.45 | (0.220, 0.431) | 28 |
| Example 3 | 4 | 7.92 | 59.88 | (0.200, 0.421) | 29 |
| Example 4 | 5 | 7.02 | 68.99 | (0.228, 0.436) | 41 |
| Example 5 | 10 | 7.08 | 69.74 | (0.243, 0.442) | 43 |
| Example 6 | 11 | 7.37 | 59.99 | (0.221, 0.433) | 47 |
| Example 7 | 15 | 7.41 | 67.13 | (0.233, 0.433) | 31 |
| Example 8 | 16 | 7.44 | 58.89 | (0.238, 0.438) | 33 |
| Example 9 | 17 | 7.07 | 70.01 | (0.225, 0.429) | 42 |
| Example 10 | 25 | 7.12 | 67.56 | (0.209, 0.415) | 44 |
| Example 11 | 26 | 7.00 | 69.89 | (0.231, 0.440) | 42 |
| Example 12 | 27 | 7.49 | 57.94 | (0.211, 0.419) | 37 |
| Example 13 | 28 | 7.66 | 58.26 | (0.209, 0.419) | 31 |
| Example 14 | 30 | 7.58 | 59.11 | (0.207, 0.409) | 38 |
| Example 15 | 34 | 7.50 | 56.66 | (0,208, 0.415) | 35 |
| Example 16 | 35 | 7.56 | 59.06 | (0.214, 0.420) | 33 |
| Example 17 | 43 | 7.21 | 69.56 | (0.224, 0.429) | 40 |
| Example 18 | 52 | 7.13 | 69.21 | (0.221, 0.434) | 42 |
| Example 19 | 55 | 7.45 | 56.45 | (0.212, 0.422) | 38 |
| Example 20 | 67 | 7.49 | 55.10 | (0.228, 0.418) | 38 |
| Example 21 | 70 | 7.54 | 56.89 | (0.231, 0.420) | 36 |
| Example 22 | 89 | 7.89 | 58.98 | (0.219, 0.411) | 35 |
| Example 23 | 90 | 7.80 | 59.11 | (0.210, 0.412) | 35 |
| Example 24 | 91 | 7.87 | 65.84 | (0.218, 0.421) | 30 |
| Example 25 | 94 | 8.00 | 59.21 | (0.209, 0.432) | 26 |
| Example 26 | 101 | 7.88 | 57.04 | (0.231, 0.418) | 37 |
| Example 27 | 104 | 7.39 | 63.38 | (0.243, 0.442) | 40 |
| Example 28 | 124 | 7.17 | 66.23 | (0.205, 0.411) | 49 |
| Example 29 | 126 | 7.22 | 66.22 | (0.243, 0.442) | 32 |
| Example 30 | 131 | 7.80 | 55.88 | (0.209, 0.419) | 37 |
| Example 31 | 133 | 7.79 | 56.18 | (0.210, 0.420) | 35 |
| Example 32 | 136 | 7.69 | 54.20 | (0.231, 0.419) | 33 |
| Example 33 | 138 | 7.76 | 53.88 | (0.229. 0.424) | 35 |
| Example 34 | 141 | 7.51 | 54.99 | (0.229, 0.430) | 30 |
| Example 35 | 147 | 7.01 | 69.82 | (0.220, 0.440) | 43 |
| Example 36 | 151 | 7.56 | 56.88 | (0.231, 0.419) | 35 |
| Example 37 | 152 | 7.60 | 56.80 | (0.229, 0.423) | 36 |
| Example 38 | 153 | 7.77 | 56.67 | (0.230, 0.421) | 38 |
| Example 39 | 162 | 7.78 | 60.30 | (0.221, 0.429) | 38 |
| Example 40 | 164 | 7.75 | 60.80 | (0.223, 0.431) | 37 |
| Example 41 | 166 | 7.68 | 60.54 | (0.220, 0.428) | 33 |
| Example 42 | 172 | 7.63 | 61.69 | (0.222, 0.430) | 39 |
| Example 43 | 174 | 7.58 | 61.47 | (0.219, 0.431) | 35 |
| Example 44 | 176 | 7.55 | 61.53 | (0.223, 0.430) | 34 |
| Example 45 | 178 | 7.89 | 60.12 | (0.220, 0.433) | 38 |
| Example 46 | 186 | 7.58 | 62.11 | (0.218, 0.430) | 37 |
| Example 47 | 191 | 7.49 | 62.07 | (0.222, 0.428) | 34 |
| Example 48 | 195 | 7.75 | 61.77 | (0.224, 0.430) | 40 |
| Example 49 | 198 | 7.71 | 62.09 | (0.221, 0.429) | 40 |
| Example 50 | 202 | 7.80 | 62.54 | (0.222, 0.430) | 43 |
| Example 51 | 204 | 7.82 | 62.01 | (0.220, 0.431) | 42 |
| Example 52 | 205 | 7.81 | 62.14 | (0.219, 0.429) | 41 |
| Example 53 | 206 | 7.74 | 61.50 | (0.223, 0.431) | 34 |
| Example 54 | 209 | 7.71 | 61.39 | (0.224, 0.430) | 35 |
| Example 55 | 210 | 7.79 | 61.33 | (0.222, 0.428) | 37 |
| Example 56 | 212 | 7.80 | 61.35 | (0.225, 0.429) | 38 |
| Example 57 | 31 | 7.49 | 62.01 | (0.223, 0.431) | 46 |
| Example 58 | 32 | 7.52 | 58.30 | (0.220, 0.428) | 47 |
| Example 59 | 214 | 7.48 | 62.89 | (0.222, 0.430) | 44 |
| Comparative | TmPyPB | 8.57 | 57.61 | (0.212, 0.433) | 24 |
| Example 1-1 | | | | | |
| Comparative | BBQB | 8.43 | 58.11 | (0.220, 0.429) | 27 |
| Example 1-2 | | | | | |
| Comparative | TBQB | 8.47 | 58.90 | (0.222, 0.430) | 28 |
| Example 1-3 | | | | | |
| Comparative | E1 | 8.01 | 59.20 | (0.223, 0.430) | 28 |
| Example 1-4 | | | | | |
| Comparative | E2 | 8.38 | 56.08 | (0.220, 0.428) | 23 |
| Example 1-5 | | | | | |
| Comparative | E3 | 8.35 | 54.21 | (0.218, 0.430) | 24 |
| Example 1-6 | | | | | |

As seen from the results of Table 3, the organic light emitting device using the charge generation layer material of the 2-stack white organic light emitting device of the present disclosure had a lower driving voltage and improved light emission efficiency compared to Comparative Example 1. Particularly, it was identified that Compounds 5, 10, 11, 17, 25, 26, 31, 32, 43, 52, 124, 147 and 214 were significantly superior in all aspects of driving, efficiency and lifetime.

Such a result is considered to be due to the fact that the compound of the present disclosure used as the N-type charge generation layer formed with the disclosed skeleton having proper length, strength and flat properties and a proper hetero-compound capable of binding with a metal forms a gap state in the N-type charge generation layer by doping an alkali metal or an alkaline earth metal, and electrons produced from the P-type charge generation layer are readily injected to the electron transfer layer through the gap state produced in the N-type charge generation layer.

Accordingly, it is considered that the P-type charge generation layer favorably injects and transfers electrons to the N-type charge generation layer, and as a result, a driving voltage was lowered, and efficiency and lifetime were improved in the organic light emitting device.

In addition, when compared with the compound of Comparative Example 1-5, the compound of the present application combined an electron-deficient substituent and an aryl or acene-based substituent so that the electron-deficient substituent readily receives electrons from the electron injection layer, exhibited excellent efficiency by the aryl or acene-based substituent stabilizing the molecule itself or transferring the supplied electrons to a light emitting layer, and particularly exhibited excellent results as a bipolar material by introducing carbazoles having strong hole properties.

### <Experimental Example 2> Manufacture of Organic Light Emitting Device

1) Manufacture of Organic Light Emitting Device

A transparent ITO electrode thin film obtained from glass for an OLED (manufactured by Samsung-Corning Co., Ltd.) was ultrasonic cleaned using trichloroethylene, acetone, ethanol and distilled water consecutively for 5 minutes each, stored in isopropanol, and used.

Next, an ITO substrate was installed in a substrate folder of a vacuum deposition apparatus, and the following 4,4′,4″-tris(N,N-(2-naphthyl)-phenylamino)triphenylamine (2-TNATA) was introduced to a cell in the vacuum deposition apparatus.

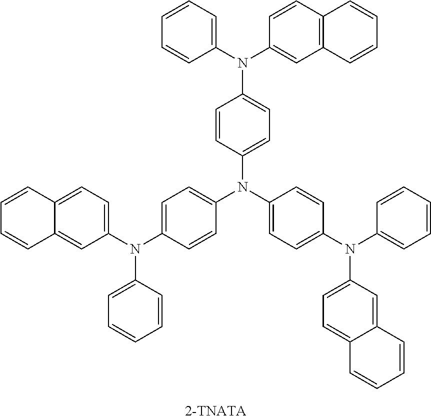 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then 2-TNATA was evaporated by applying a current to the cell to deposit a hole injection layer having a thickness of 600 Å on the ITO substrate.

To another cell of the vacuum deposition apparatus, the following N,N′-bis(α-naphthyl)-N,N′-diphenyl-4,4′-diamine (NPB) was introduced, and evaporated by applying a current to the cell to deposit a hole transfer layer having a thickness of 300 Å on the hole injection layer.

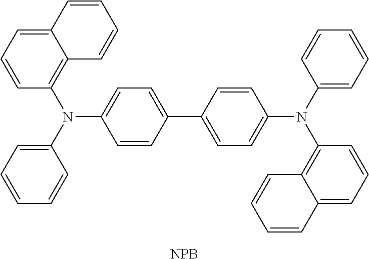 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transfer layer as above, a blue light emitting material having a structure as below was deposited thereon as a light emitting layer. Specifically, in one side cell in the vacuum deposition apparatus, H1, a blue light emitting host material, was vacuum deposited to a thickness of 200 Å, and D1, a blue light emitting dopant material, was vacuum deposited thereon by 5% with respect to the host material.

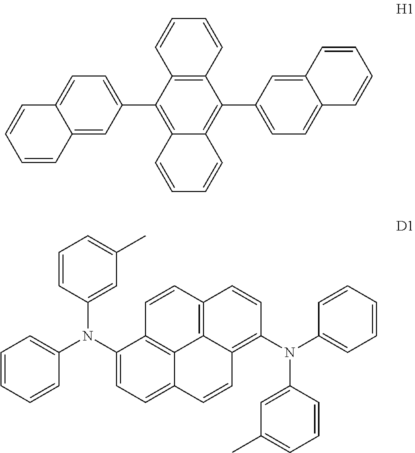

Subsequently, a compound of the following Table 4 was deposited to a thickness of 300 Å as an electron transfer layer.

As an electron injection layer, lithium fluoride (LiF) was deposited to a thickness of 10 Å, and an Al cathode was employed to a thickness of 1,000 Å to manufacture an OLED.

Meanwhile, all the organic compounds required to manufacture the OLED were vacuum sublimation purified under 10⁻⁶ torr to 10⁻⁸ torr by each material to be used in the OLED manufacture.

2) Driving Voltage and Light Emission Efficiency of Organic Light Emitting Device

For the organic light emitting devices manufactured as above, electroluminescent light emission (EL) properties were measured using M7000 manufactured by McScience Inc., and with the measurement results, T₉₅ when standard luminance was 700 cd/m² was measured using a lifetime measurement system (M6000) manufactured by McScience Inc. Results of measuring a driving voltage, light emission efficiency, external quantum efficiency and a color coordinate (CIE) of the blue organic light emitting device manufactured according to the present disclosure are as shown in Table 4.

TABLE 4

| | | | Light | | |
|---|---|---|---|---|---|
| | | Driving | Emission | | |
| | | Voltage | Efficiency | CIE | Lifetime |
| | Compound | (V) | (cd/A) | (x, y) | (T₉₅) |
| Example 39 | 2 | 5.48 | 6.22 | (0.134, 0.101) | 38 |
| Example 40 | 3 | 5.44 | 6.25 | (0.134, 0.102) | 40 |
| Example 41 | 4 | 5.50 | 6.32 | (0.134, 0.101) | 33 |
| Example 42 | 5 | 4.72 | 6.53 | (0.134, 0.102) | 66 |
| Example 43 | 10 | 4.51 | 6.93 | (0.134, 0.100) | 40 |
| Example 44 | 11 | 4.56 | 6.88 | (0.134, 0.100) | 41 |
| Example 45 | 15 | 5.15 | 6.10 | (0.134, 0.103) | 39 |
| Example 46 | 16 | 5.20 | 6.15 | (0.134, 0.101) | 40 |
| Example 47 | 17 | 4.45 | 6.98 | (0.134, 0.100) | 40 |
| Example 48 | 25 | 4.50 | 6.99 | (0.134, 0.101) | 41 |
| Example 49 | 26 | 4.48 | 6.85 | (0.134, 0.099) | 40 |
| Example 50 | 27 | 5.07 | 6.24 | (0.134, 0.100) | 33 |
| Example 51 | 28 | 5.05 | 6.31 | (0.134, 0.100) | 31 |
| Example 52 | 30 | 5.09 | 6.22 | (0.134, 0.101) | 32 |
| Example 53 | 34 | 5.11 | 6.22 | (0.134, 0.103) | 36 |
| Example 54 | 35 | 5.11 | 6.20 | (0.134, 0.100) | 40 |
| Example 55 | 43 | 4.45 | 7.03 | (0.134, 0.100) | 33 |
| Example 56 | 52 | 4.41 | 6.95 | (0.134, 0.100) | 39 |
| Example 57 | 55 | 4.98 | 6.22 | (0.134, 0.100) | 40 |
| Example 58 | 67 | 5.02 | 6.34 | (0.134, 0.101) | 39 |
| Example 59 | 70 | 5.05 | 6.14 | (0.134, 0.101) | 40 |
| Example 60 | 89 | 5.44 | 6.22 | (0.134, 0.102) | 30 |
| Example 61 | 90 | 5.32 | 5.95 | (0.134, 0.101) | 29 |
| Example 62 | 91 | 5.40 | 6.13 | (0.134, 0.101) | 31 |
| Example 63 | 94 | 5.44 | 5.89 | (0.134, 0.100) | 32 |
| Example 64 | 101 | 5.39 | 6.01 | (0.134, 0.101) | 29 |
| Example 65 | 104 | 5.11 | 6.11 | (0.134, 0.100) | 40 |
| Example 66 | 124 | 4.44 | 7.14 | (0.134, 0.102) | 38 |
| Example 67 | 126 | 5.19 | 6.28 | (0.134, 0.102) | 40 |
| Example 68 | 131 | 5.24 | 6.10 | (0.134, 0.102) | 38 |
| Example 69 | 133 | 5.41 | 6.21 | (0.134, 0.100) | 39 |
| Example 70 | 136 | 5.33 | 6.19 | (0.134, 0.101) | 30 |
| Example 71 | 138 | 5.37 | 6.05 | (0.134, 0.100) | 28 |
| Example 72 | 141 | 5.10 | 6.21 | (0.134, 0.101) | 40 |
| Example 73 | 147 | 4.45 | 7.03 | (0.134, 0.100) | 40 |
| Example 74 | 151 | 5.05 | 6.23 | (0.134, 0.101) | 41 |
| Example 75 | 152 | 4.98 | 6.03 | (0.134, 0.101) | 35 |
| Example 76 | 153 | 5.40 | 6.21 | (0.134, 0.103) | 39 |
| Example 77 | 162 | 5.20 | 6.40 | (0.134, 0.103) | 38 |
| Example 78 | 164 | 5.21 | 6.43 | (0.134, 0.101) | 39 |
| Example 79 | 166 | 5.15 | 6.35 | (0.134, 0.101) | 35 |
| Example 80 | 172 | 5.16 | 6.33 | (0.134, 0.103) | 35 |
| Example 81 | 174 | 4.97 | 6.49 | (0.134, 0.102) | 37 |
| Example 82 | 176 | 4.95 | 6.51 | (0.134, 0.102) | 38 |
| Example 83 | 178 | 5.11 | 6.57 | (0.134, 0.101) | 39 |
| Example 84 | 186 | 5.30 | 6.40 | (0.134, 0.101) | 42 |
| Example 85 | 191 | 5.20 | 6.31 | (0.134, 0.103) | 40 |
| Example 86 | 195 | 5.07 | 6.55 | (0.134, 0.103) | 40 |
| Example 87 | 198 | 5.09 | 6.57 | (0.134, 0.102) | 41 |
| Example 88 | 202 | 5.15 | 6.67 | (0.134, 0.101) | 43 |
| Example 89 | 204 | 5.14 | 6.70 | (0.134, 0.101) | 43 |
| Example 90 | 205 | 5.18 | 6.71 | (0.134, 0.100) | 42 |
| Example 91 | 206 | 5.25 | 6.55 | (0.134, 0.100) | 39 |
| Example 92 | 209 | 5.27 | 6.57 | (0.134, 0.103) | 40 |
| Example 93 | 210 | 5.31 | 6.60 | (0.134, 0.103) | 42 |
| Example 94 | 212 | 5.33 | 6.63 | (0.134, 0.101) | 41 |
| Example 95 | 31 | 4.46 | 6.20 | (0.134, 0.101) | 45 |
| Example 96 | 32 | 4.49 | 5.83 | (0.134, 0.103) | 47 |
| Example 97 | 214 | 4.44 | 6.89 | (0.134, 0.103) | 42 |
| Comparative | E1 | 5.56 | 5.91 | (0.134, 0.100) | 28 |
| Example 2-1 | | | | | |
| Comparative | E2 | 5.62 | 6.03 | (0.223, 0,430) | 27 |
| Example 2-2 | | | | | |
| Comparative | E3 | 5.47 | 6.12 | (0.134, 0.100) | 25 |
| Example2-3 | | | | | |
| Comparative | E4 | 5.45 | 6.14 | (0.134, 0.100) | 26 |
| Example 2-4 | | | | | |
| Comparative | BBQB | 5.50 | 6.10 | (0.134, 0.101) | 30 |
| Example 2-5 | | | | | |
| Comparative | TBQB | 5.51 | 6.15 | (0.134, 0.102) | 29 |
| Example 2-6 | | | | | |

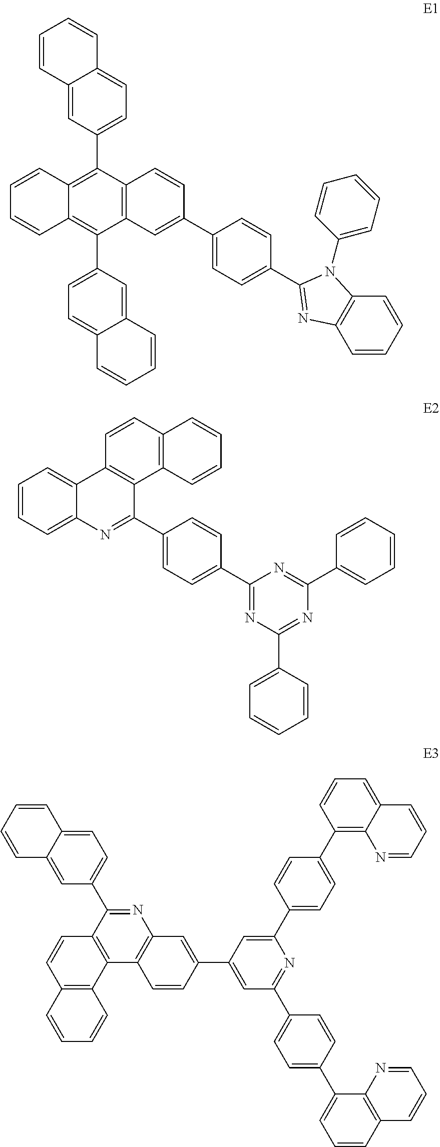

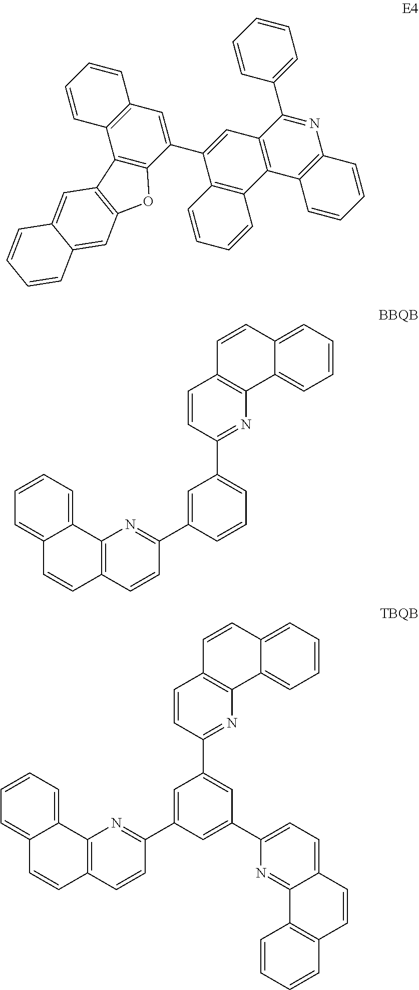 [SMILES: c1ccc(-c2nc3ccccc3c3c2cc(-c2cc4ccccc4c4c2oc2cc5ccccc5cc24)c2ccccc23)cc1 ; c1cc(-c2ccc3ccc4ccccc4c3n2)cc(-c2ccc3ccc4ccccc4c3n2)c1 ; c1ccc2c(c1)ccc1ccc(-c3cc(-c4ccc5ccc6ccccc6c5n4)cc(-c4ccc5ccc6ccccc6c5n4)c3)nc12]

As seen from the results of Table 4, the organic light emitting device using the electron transfer layer material of the blue organic light emitting device of the present disclosure had a lower driving voltage and significantly improved light emission efficiency and lifetime compared to Comparative Example 3. Particularly, it was identified that Compounds 5, 10, 11, 17, 25, 26, 31, 32, 43, 52, 124, 147 and 214 were superior in all aspects of driving, efficiency and lifetime.

Such a result is considered to be due to the fact that, when using the disclosed compound having proper length, strength and flat properties as an electron transfer layer, a compound in an excited state is made by receiving electrons under a specific condition, and particularly when a hetero-skeleton site of the compound is formed in an excited state, excited energy moves to a stable state before the excited hetero-skeleton site goes through other reactions, and a relatively stabilized compound is capable of efficiently transferring electrons without the compound being decomposed or destroyed. For reference, those that are stable when excited are considered to be aryl or acene-based compounds or polycyclic hetero-compounds. Accordingly, it is considered that excellent results in all aspects of driving, efficiency and lifetime were obtained by the compound of the present disclosure enhancing enhanced electron-transfer properties or improved stability.

1. A heterocyclic compound represented by the following Chemical Formula 1: 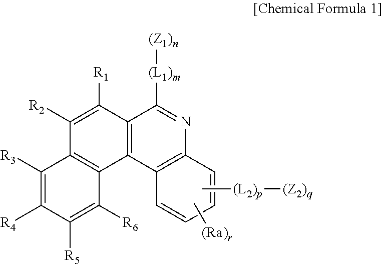
wherein, in Chemical Formula 1,
R₁ to R₆ and Ra are the same as or different from each other, and each independently selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group unsubstituted or substituted with a substituted or unsubstituted alkyl group, a substituted or unsubstituted aryl group, or a substituted or unsubstituted heteroaryl group, or two or more groups adjacent to each other bond to each other to form a substituted or unsubstituted aliphatic or aromatic hydrocarbon ring;
L₁ is a substituted or unsubstituted arylene group; or a substituted or unsubstituted heteroarylene group;
L₂ is a direct bond; a substituted or unsubstituted arylene group; or a substituted or unsubstituted heteroarylene group;
Z₁ is selected from the group consisting of hydrogen; deuterium; a halogen group; —CN; a substituted or unsubstituted alkyl group; a substituted or unsubstituted aryl group; a substituted or unsubstituted heteroaryl group; —SiRR′R″ and —P(═O)RR′;
Z₂ is selected from the group consisting of deuterium; a halogen group; —CN; a substituted or unsubstituted alkyl group; a substituted or unsubstituted aryl group; a substituted or unsubstituted heteroaryl group; —SiRR′R″ and —P(═O)RR′;
when Z₁ is hydrogen, L₂ is a substituted or unsubstituted arylene group, and Z₂ is a substituted or unsubstituted heteroaryl group;
R, R′ and R″ are the same as or different from each other, and each independently hydrogen; deuterium; —CN; a substituted or unsubstituted alkyl group; a substituted or unsubstituted cycloalkyl group; a substituted or unsubstituted aryl group; or a substituted or unsubstituted heteroaryl group;
p and m are an integer of 1 to 4;
q and n are an integer of 1 to 5; and
r is an integer of 0 to 3.

2. The heterocyclic compound of claim 1, wherein the “substituted or unsubstituted” means being substituted with one or more substituents selected from the group consisting of C1 to C60 linear or branched alkyl; C2 to C60 linear or branched alkenyl; C2 to C60 linear or branched alkynyl; C3 to C60 monocyclic or polycyclic cycloalkyl; C2 to C60 monocyclic or polycyclic heterocycloalkyl; C6 to C60 monocyclic or polycyclic aryl; C2 to C60 monocyclic or polycyclic heteroaryl; —SiRR′R″; —P(═O)RR′; C1 to C20 alkylamine; C6 to C60 monocyclic polycyclic arylamine; and C2 to C60 monocyclic or polycyclic heteroarylamine, or being unsubstituted, or being substituted with a substituent linking; two or more substituents selected from among the substituents illustrated above, or being unsubstituted; and
R′ and R″ have the same definitions as in Chemical Formula 1.

3. The heterocyclic compound of claim 1, wherein R₁ to R₆ and Ra of Chemical Formula 1 are the same as or different from each other, and each independently hydrogen.

4. The heterocyclic compound of claim 1, wherein L₁ of Chemical Formula 1 is a C6 to C40 tricyclic or lower arylene group; and
L₂ of Chemical Formula 1 is a direct bond; or a C6 to C30 monocyclic arylene group.

5. The heterocyclic compound of claim 1, wherein Z₁ of Chemical Formula 1 is selected from the group consisting of hydrogen; a C6 to C20 aryl group unsubstituted or substituted with an alkyl group; a C2 to C20 heteroaryl group unsubstituted or substituted with one or more substituents selected from the group consisting of an alkyl group, an aryl group and a heteroaryl group; and —P(═O)RR′;
Z₂ of Chemical Formula 1 is —CN; a C6 to C20 aryl group unsubstituted or substituted with a heteroaryl group; or a C2 to C20 heteroaryl group unsubstituted or substituted with one or more substituents selected from the group consisting of an alkyl group and an aryl group; and
R and R′ have the same definitions as in Chemical Formula 1.

6. The heterocyclic compound of claim 1, wherein Chemical Formula 1 is represented by any one of the following Chemical Formulae 2 to 7: 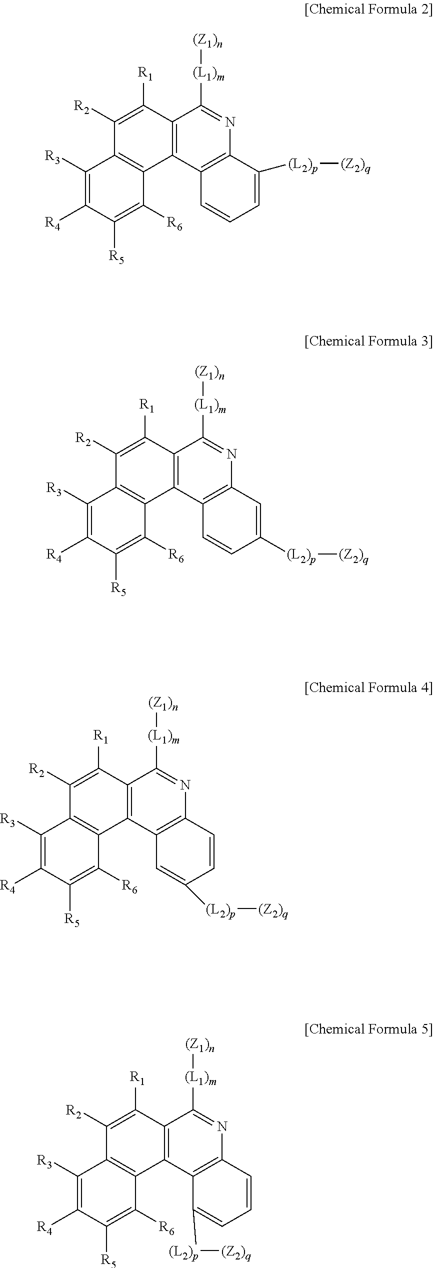 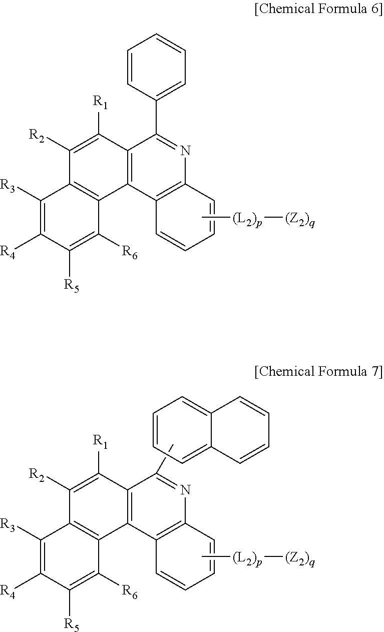
in Chemical Formulae 2 to 7,
R₁ to R₆, L₁, L₂, Z₁, Z₂, m, n, p and q have the same definitions as in Chemical Formula 1.

7. The heterocyclic compound of claim 1, wherein Chemical Formula 1 is represented by any one of the following compounds:

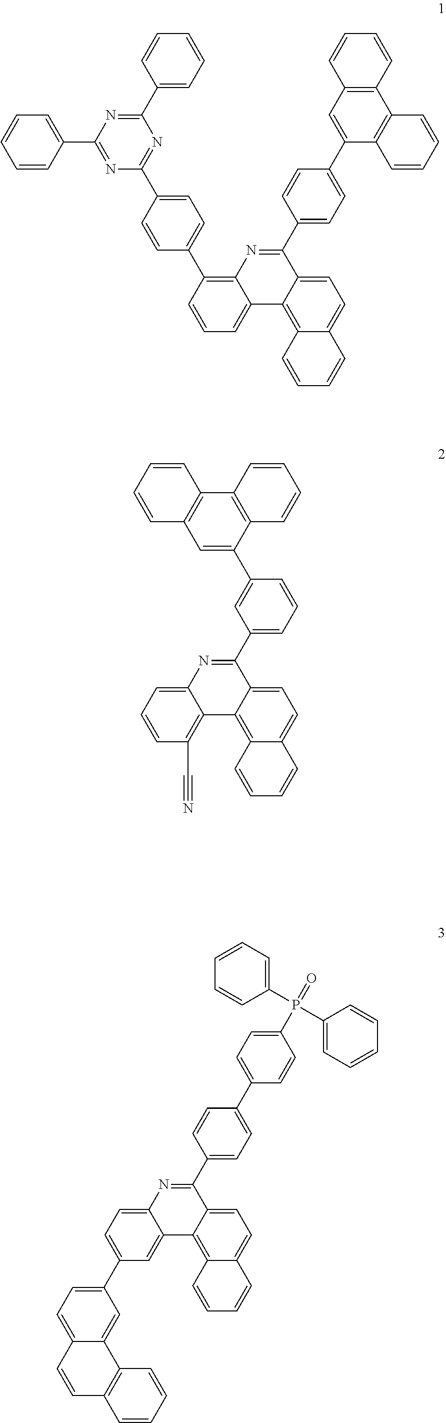 [SMILES: c1ccc(-c2nc(-c3ccccc3)nc(-c3ccc(-c4cccc5c4nc(-c4ccc(-c6cc7ccccc7c7ccccc67)cc4)c4ccc6ccccc6c45)cc3)n2)cc1 ; N#Cc1cccc2nc(-c3cccc(-c4cc5ccccc5c5ccccc45)c3)c3ccc4ccccc4c3c12 ; O=P(c1ccccc1)(c1ccccc1)c1ccc(-c2ccc(-c3nc4ccc(-c5ccc6ccc7ccccc7c6c5)cc4c4c3ccc3ccccc34)cc2)cc1]

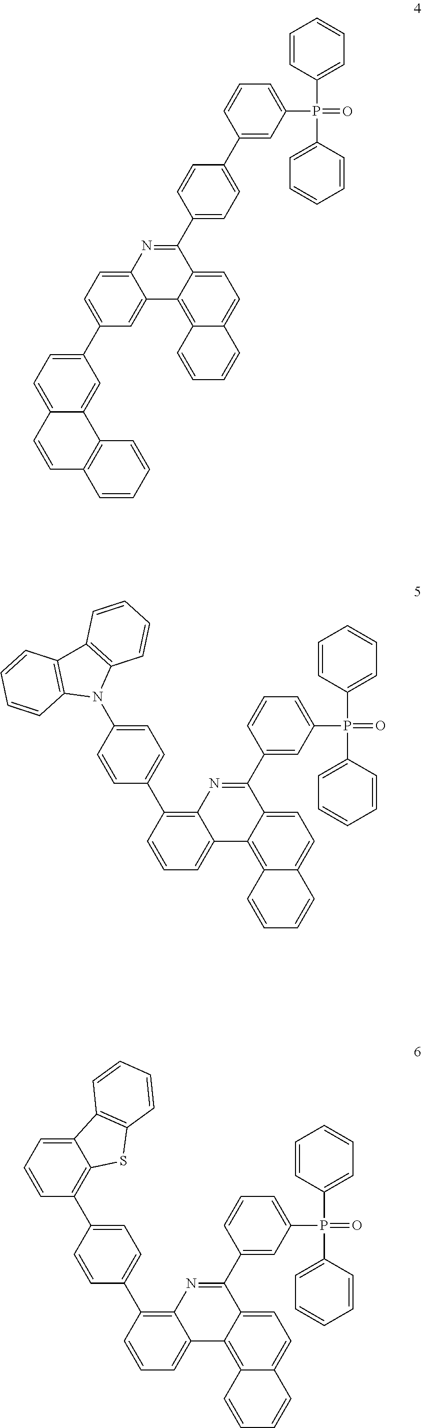 [SMILES: O=P(c1ccccc1)(c1ccccc1)c1cccc(-c2ccc(-c3nc4ccc(-c5ccc6ccc7ccccc7c6c5)cc4c4c3ccc3ccccc34)cc2)c1 ; O=P(c1ccccc1)(c1ccccc1)c1cccc(-c2nc3c(-c4ccc(-n5c6ccccc6c6ccccc65)cc4)cccc3c3c2ccc2ccccc23)c1 ; O=P(c1ccccc1)(c1ccccc1)c1cccc(-c2nc3c(-c4ccc(-c5cccc6c5sc5ccccc56)cc4)cccc3c3c2ccc2ccccc23)c1]

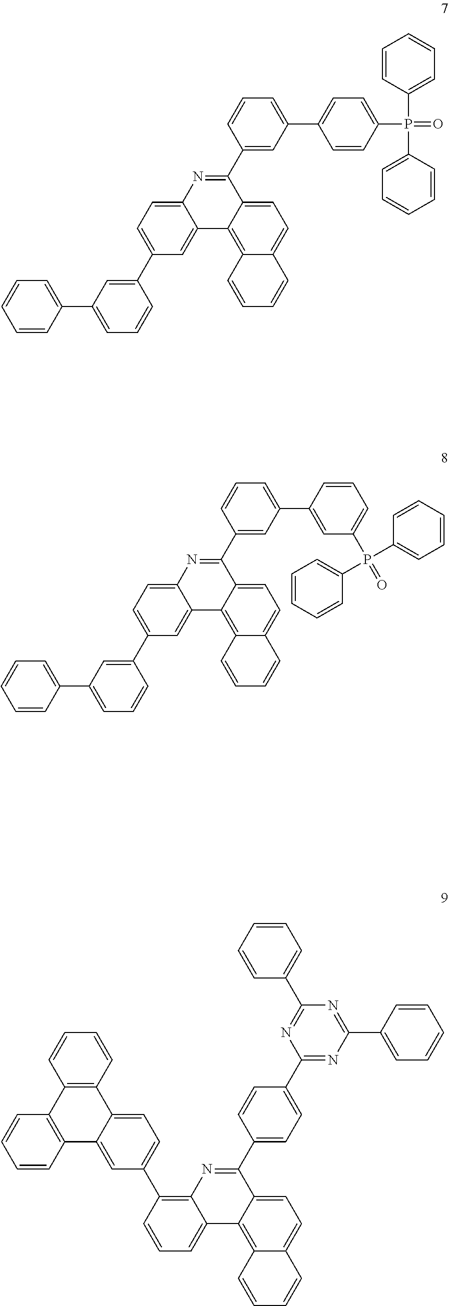 [SMILES: O=P(c1ccccc1)(c1ccccc1)c1ccc(-c2cccc(-c3nc4ccc(-c5cccc(-c6ccccc6)c5)cc4c4c3ccc3ccccc34)c2)cc1 ; O=P(c1ccccc1)(c1ccccc1)c1cccc(-c2cccc(-c3nc4ccc(-c5cccc(-c6ccccc6)c5)cc4c4c3ccc3ccccc34)c2)c1 ; c1ccc(-c2nc(-c3ccccc3)nc(-c3ccc(-c4nc5c(-c6ccc7c8ccccc8c8ccccc8c7c6)cccc5c5c4ccc4ccccc45)cc3)n2)cc1]

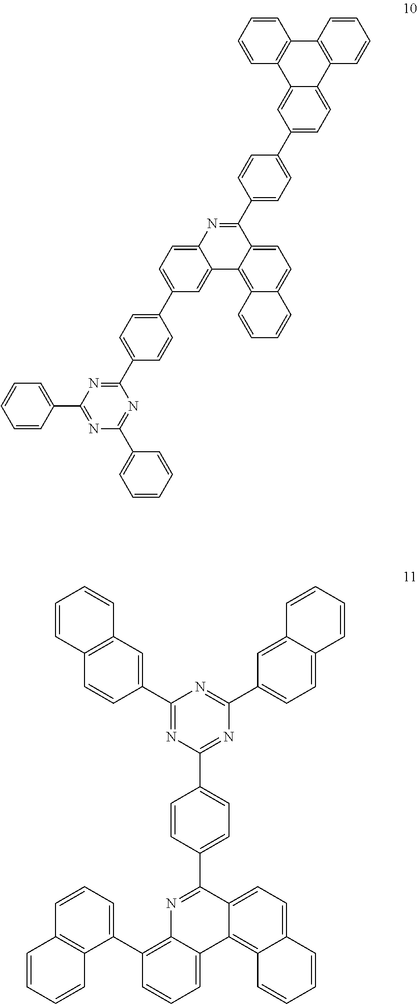 [SMILES: c1ccc(-c2nc(-c3ccccc3)nc(-c3ccc(-c4ccc5nc(-c6ccc(-c7ccc8c9ccccc9c9ccccc9c8c7)cc6)c6ccc7ccccc7c6c5c4)cc3)n2)cc1 ; c1ccc2cc(-c3nc(-c4ccc(-c5nc6c(-c7cccc8ccccc78)cccc6c6c5ccc5ccccc56)cc4)nc(-c4ccc5ccccc5c4)n3)ccc2c1]

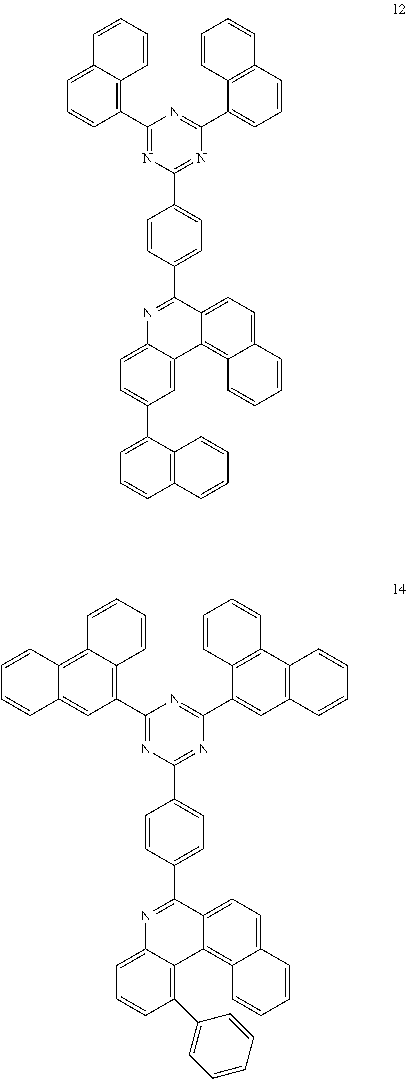 [SMILES: c1ccc2c(-c3ccc4nc(-c5ccc(-c6nc(-c7cccc8ccccc78)nc(-c7cccc8ccccc78)n6)cc5)c5ccc6ccccc6c5c4c3)cccc2c1 ; c1ccc(-c2cccc3nc(-c4ccc(-c5nc(-c6cc7ccccc7c7ccccc67)nc(-c6cc7ccccc7c7ccccc67)n5)cc4)c4ccc5ccccc5c4c23)cc1]

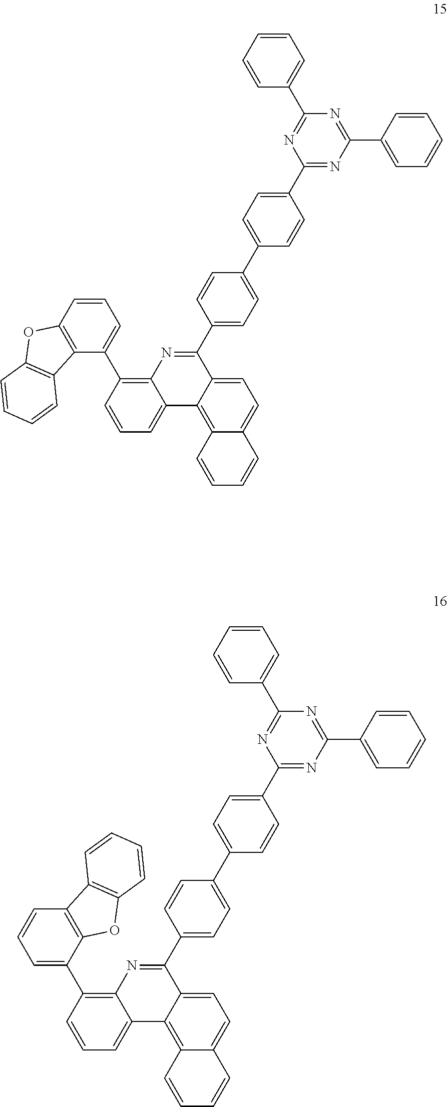 [SMILES: c1ccc(-c2nc(-c3ccccc3)nc(-c3ccc(-c4ccc(-c5nc6c(-c7cccc8oc9ccccc9c78)cccc6c6c5ccc5ccccc56)cc4)cc3)n2)cc1 ; c1ccc(-c2nc(-c3ccccc3)nc(-c3ccc(-c4ccc(-c5nc6c(-c7cccc8c7oc7ccccc78)cccc6c6c5ccc5ccccc56)cc4)cc3)n2)cc1]

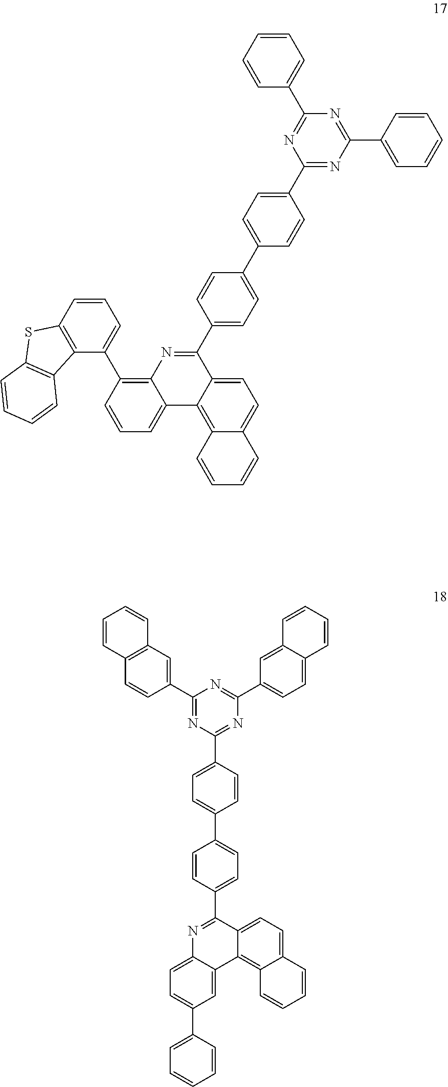 [SMILES: c1ccc(-c2nc(-c3ccccc3)nc(-c3ccc(-c4ccc(-c5nc6c(-c7cccc8sc9ccccc9c78)cccc6c6c5ccc5ccccc56)cc4)cc3)n2)cc1 ; c1ccc(-c2ccc3nc(-c4ccc(-c5ccc(-c6nc(-c7ccc8ccccc8c7)nc(-c7ccc8ccccc8c7)n6)cc5)cc4)c4ccc5ccccc5c4c3c2)cc1]

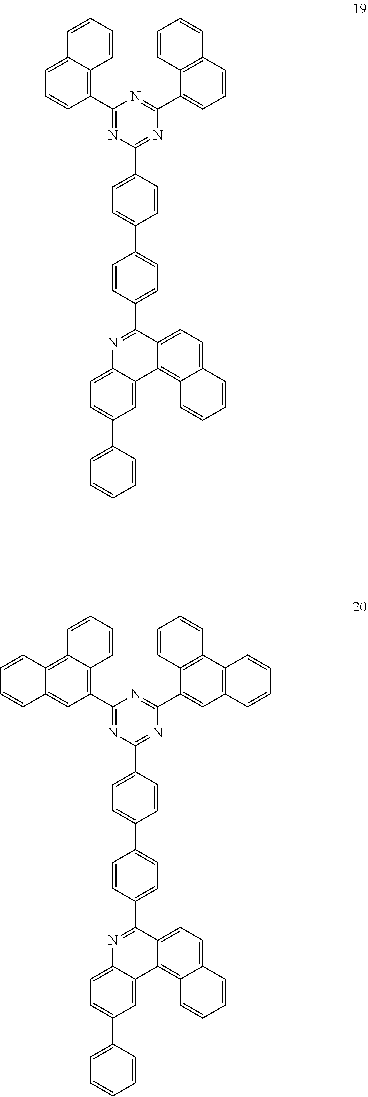 [SMILES: c1ccc(-c2ccc3nc(-c4ccc(-c5ccc(-c6nc(-c7cccc8ccccc78)nc(-c7cccc8ccccc78)n6)cc5)cc4)c4ccc5ccccc5c4c3c2)cc1 ; c1ccc(-c2ccc3nc(-c4ccc(-c5ccc(-c6nc(-c7cc8ccccc8c8ccccc78)nc(-c7cc8ccccc8c8ccccc78)n6)cc5)cc4)c4ccc5ccccc5c4c3c2)cc1]

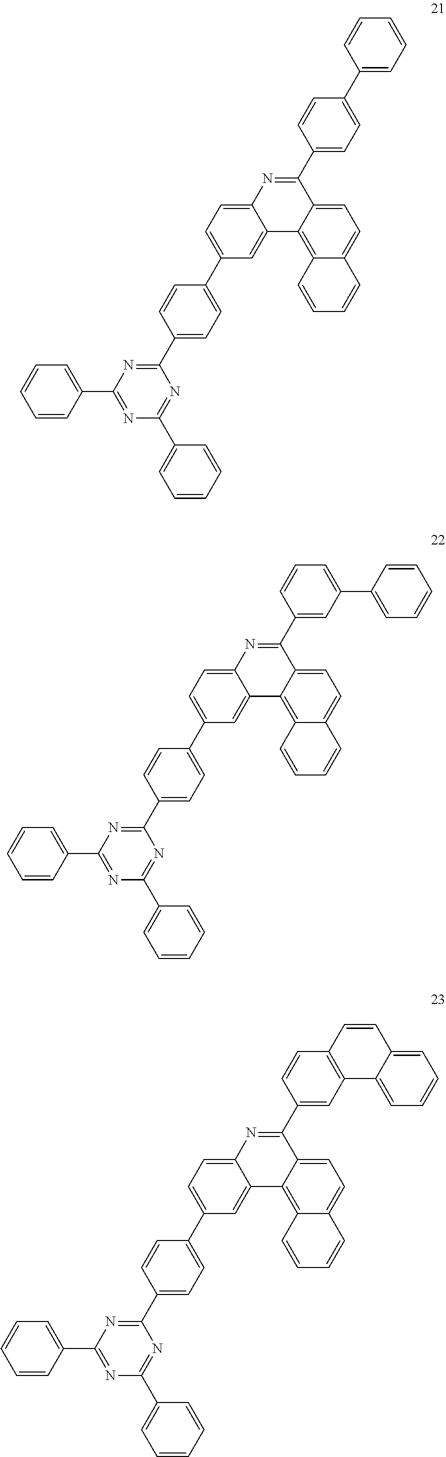 [SMILES: c1ccc(-c2ccc(-c3nc4ccc(-c5ccc(-c6nc(-c7ccccc7)nc(-c7ccccc7)n6)cc5)cc4c4c3ccc3ccccc34)cc2)cc1 ; c1ccc(-c2cccc(-c3nc4ccc(-c5ccc(-c6nc(-c7ccccc7)nc(-c7ccccc7)n6)cc5)cc4c4c3ccc3ccccc34)c2)cc1 ; c1ccc(-c2nc(-c3ccccc3)nc(-c3ccc(-c4ccc5nc(-c6ccc7ccc8ccccc8c7c6)c6ccc7ccccc7c6c5c4)cc3)n2)cc1]

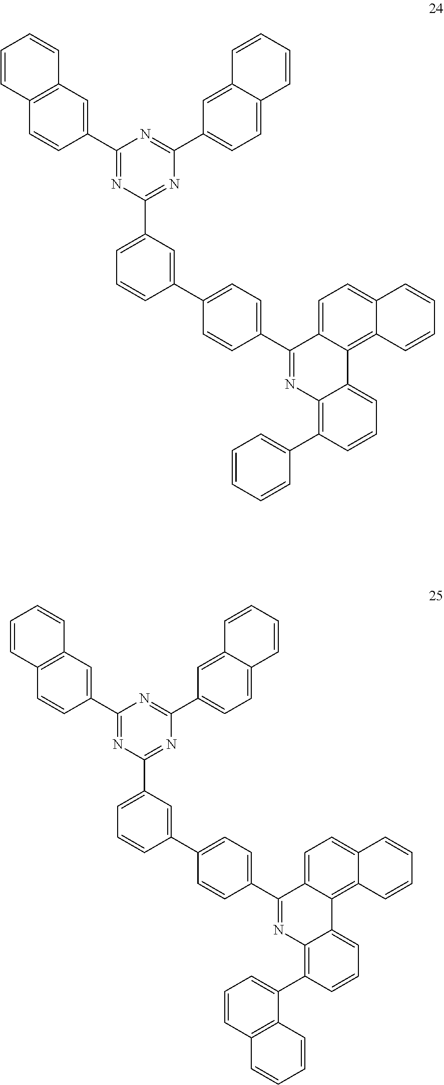 [SMILES: c1ccc(-c2cccc3c2nc(-c2ccc(-c4cccc(-c5nc(-c6ccc7ccccc7c6)nc(-c6ccc7ccccc7c6)n5)c4)cc2)c2ccc4ccccc4c23)cc1 ; c1cc(-c2ccc(-c3nc4c(-c5cccc6ccccc56)cccc4c4c3ccc3ccccc34)cc2)cc(-c2nc(-c3ccc4ccccc4c3)nc(-c3ccc4ccccc4c3)n2)c1]

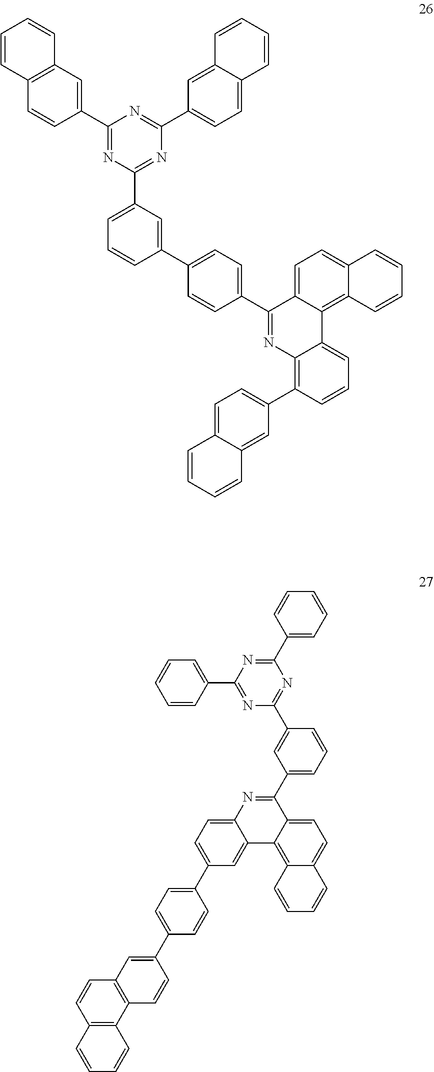 [SMILES: c1cc(-c2ccc(-c3nc4c(-c5ccc6ccccc6c5)cccc4c4c3ccc3ccccc34)cc2)cc(-c2nc(-c3ccc4ccccc4c3)nc(-c3ccc4ccccc4c3)n2)c1 ; c1ccc(-c2nc(-c3ccccc3)nc(-c3cccc(-c4nc5ccc(-c6ccc(-c7ccc8c(ccc9ccccc98)c7)cc6)cc5c5c4ccc4ccccc45)c3)n2)cc1]

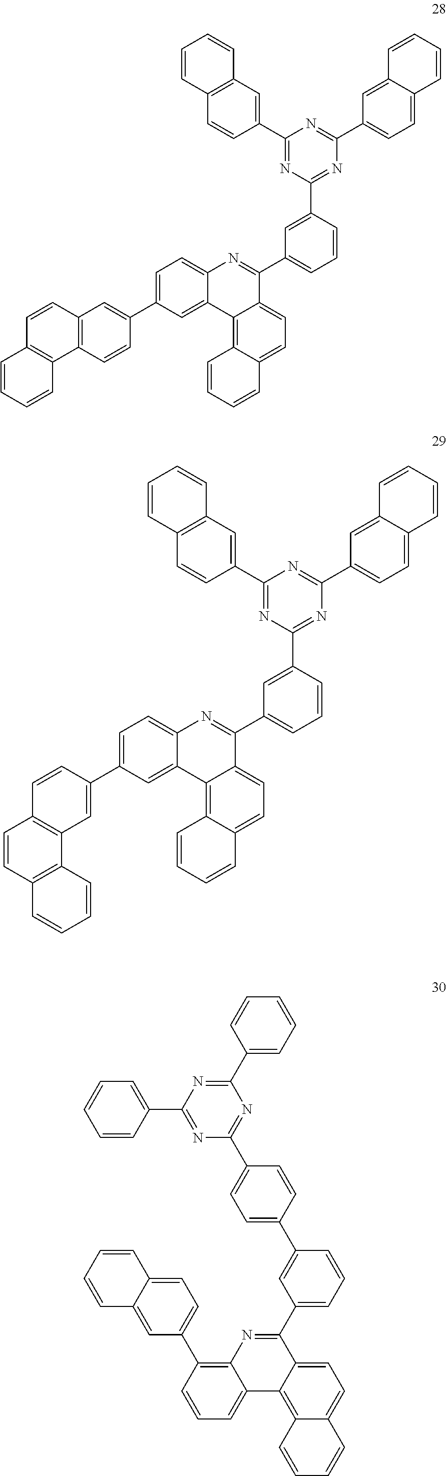 [SMILES: c1cc(-c2nc(-c3ccc4ccccc4c3)nc(-c3ccc4ccccc4c3)n2)cc(-c2nc3ccc(-c4ccc5c(ccc6ccccc65)c4)cc3c3c2ccc2ccccc23)c1 ; c1cc(-c2nc(-c3ccc4ccccc4c3)nc(-c3ccc4ccccc4c3)n2)cc(-c2nc3ccc(-c4ccc5ccc6ccccc6c5c4)cc3c3c2ccc2ccccc23)c1 ; c1ccc(-c2nc(-c3ccccc3)nc(-c3ccc(-c4cccc(-c5nc6c(-c7ccc8ccccc8c7)cccc6c6c5ccc5ccccc56)c4)cc3)n2)cc1]

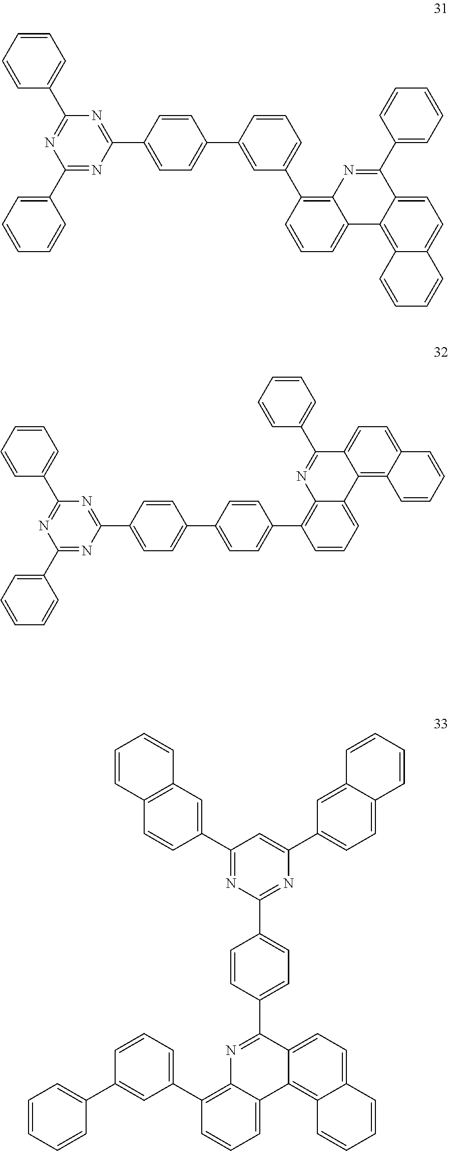 [SMILES: c1ccc(-c2nc(-c3ccccc3)nc(-c3ccc(-c4cccc(-c5cccc6c5nc(-c5ccccc5)c5ccc7ccccc7c56)c4)cc3)n2)cc1 ; c1ccc(-c2nc(-c3ccccc3)nc(-c3ccc(-c4ccc(-c5cccc6c5nc(-c5ccccc5)c5ccc7ccccc7c56)cc4)cc3)n2)cc1 ; c1ccc(-c2cccc(-c3cccc4c3nc(-c3ccc(-c5nc(-c6ccc7ccccc7c6)cc(-c6ccc7ccccc7c6)n5)cc3)c3ccc5ccccc5c34)c2)cc1]

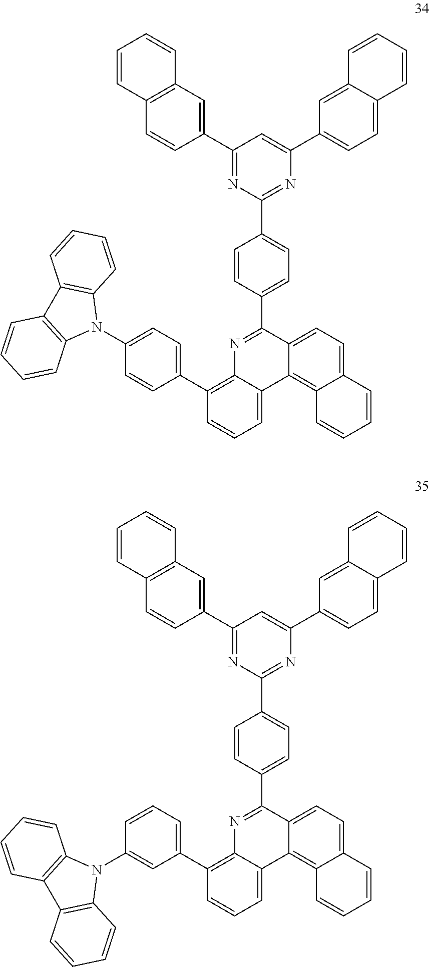 [SMILES: c1ccc2cc(-c3cc(-c4ccc5ccccc5c4)nc(-c4ccc(-c5nc6c(-c7ccc(-n8c9ccccc9c9ccccc98)cc7)cccc6c6c5ccc5ccccc56)cc4)n3)ccc2c1 ; c1cc(-c2cccc3c2nc(-c2ccc(-c4nc(-c5ccc6ccccc6c5)cc(-c5ccc6ccccc6c5)n4)cc2)c2ccc4ccccc4c23)cc(-n2c3ccccc3c3ccccc32)c1]

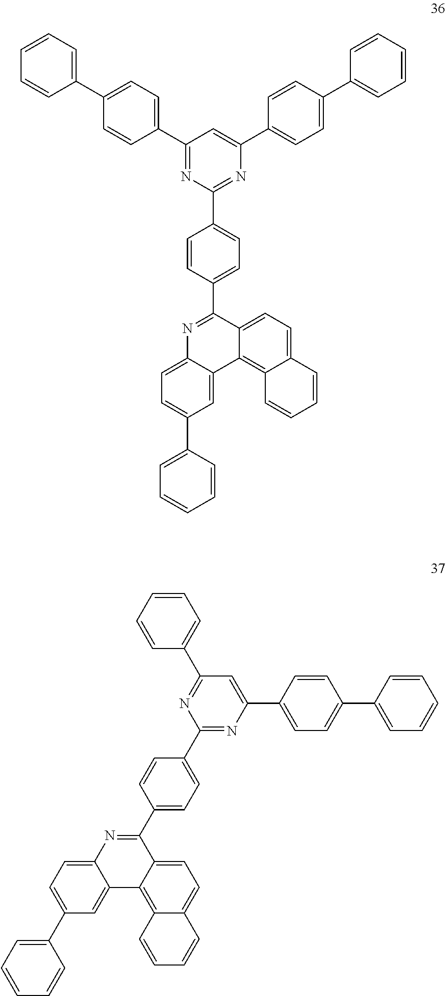 [SMILES: c1ccc(-c2ccc(-c3cc(-c4ccc(-c5ccccc5)cc4)nc(-c4ccc(-c5nc6ccc(-c7ccccc7)cc6c6c5ccc5ccccc56)cc4)n3)cc2)cc1 ; c1ccc(-c2ccc(-c3cc(-c4ccccc4)nc(-c4ccc(-c5nc6ccc(-c7ccccc7)cc6c6c5ccc5ccccc56)cc4)n3)cc2)cc1]

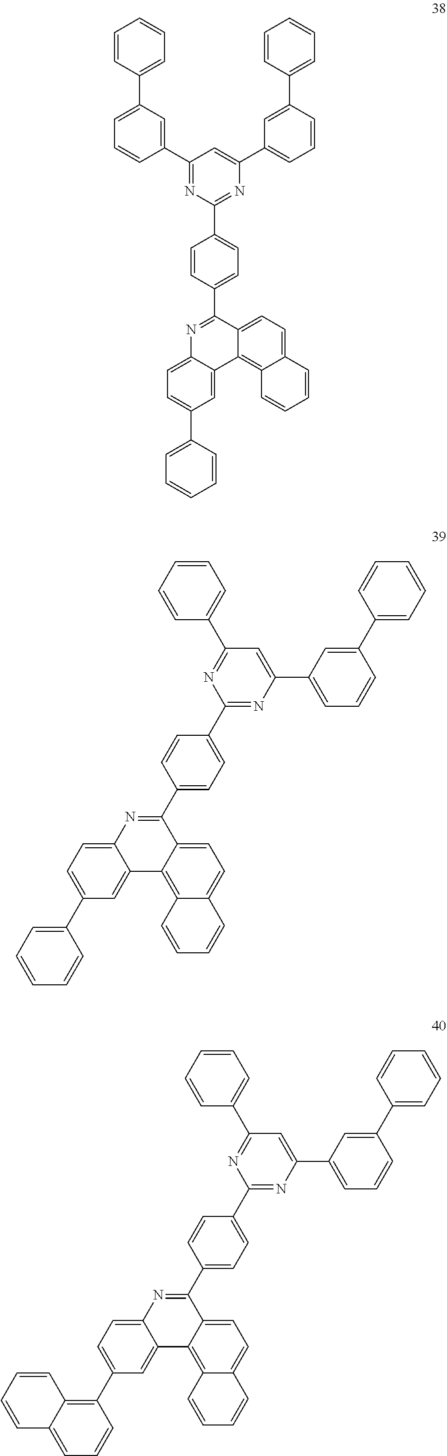 [SMILES: c1ccc(-c2cccc(-c3cc(-c4cccc(-c5ccccc5)c4)nc(-c4ccc(-c5nc6ccc(-c7ccccc7)cc6c6c5ccc5ccccc56)cc4)n3)c2)cc1 ; c1ccc(-c2cccc(-c3cc(-c4ccccc4)nc(-c4ccc(-c5nc6ccc(-c7ccccc7)cc6c6c5ccc5ccccc56)cc4)n3)c2)cc1 ; c1ccc(-c2cccc(-c3cc(-c4ccccc4)nc(-c4ccc(-c5nc6ccc(-c7cccc8ccccc78)cc6c6c5ccc5ccccc56)cc4)n3)c2)cc1]

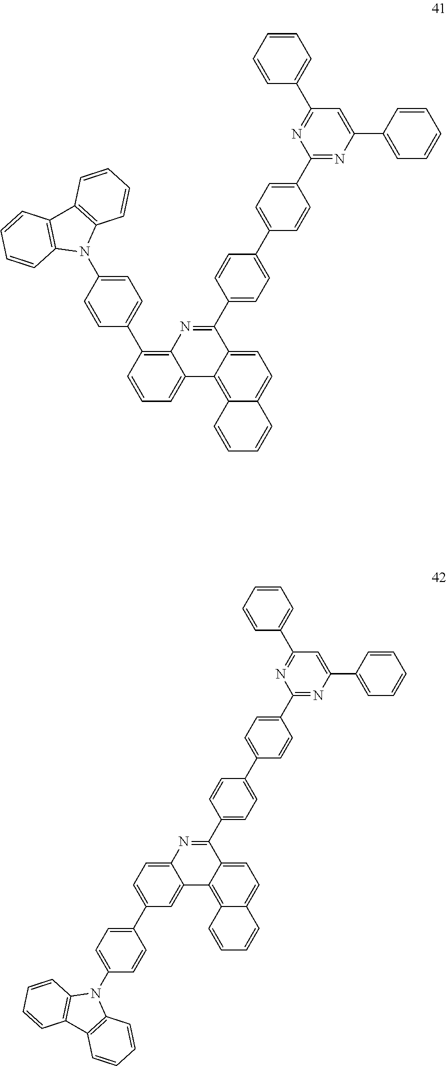 [SMILES: c1ccc(-c2cc(-c3ccccc3)nc(-c3ccc(-c4ccc(-c5nc6c(-c7ccc(-n8c9ccccc9c9ccccc98)cc7)cccc6c6c5ccc5ccccc56)cc4)cc3)n2)cc1 ; c1ccc(-c2cc(-c3ccccc3)nc(-c3ccc(-c4ccc(-c5nc6ccc(-c7ccc(-n8c9ccccc9c9ccccc98)cc7)cc6c6c5ccc5ccccc56)cc4)cc3)n2)cc1]

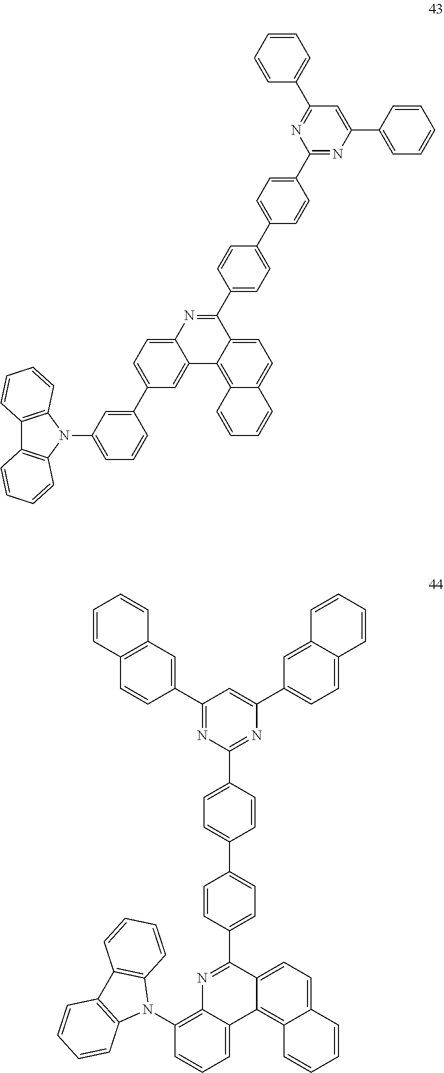 [SMILES: c1ccc(-c2cc(-c3ccccc3)nc(-c3ccc(-c4ccc(-c5nc6ccc(-c7cccc(-n8c9ccccc9c9ccccc98)c7)cc6c6c5ccc5ccccc56)cc4)cc3)n2)cc1 ; c1ccc2cc(-c3cc(-c4ccc5ccccc5c4)nc(-c4ccc(-c5ccc(-c6nc7c(-n8c9ccccc9c9ccccc98)cccc7c7c6ccc6ccccc67)cc5)cc4)n3)ccc2c1]

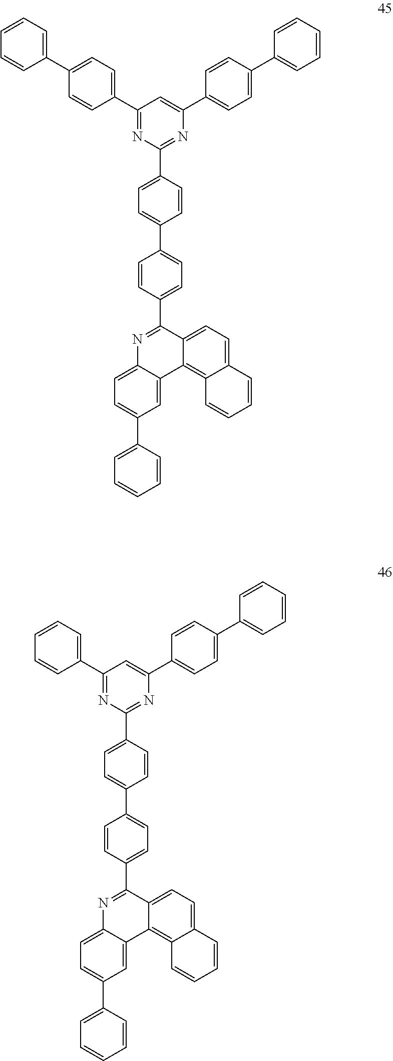 [SMILES: c1ccc(-c2ccc(-c3cc(-c4ccc(-c5ccccc5)cc4)nc(-c4ccc(-c5ccc(-c6nc7ccc(-c8ccccc8)cc7c7c6ccc6ccccc67)cc5)cc4)n3)cc2)cc1 ; c1ccc(-c2ccc(-c3cc(-c4ccccc4)nc(-c4ccc(-c5ccc(-c6nc7ccc(-c8ccccc8)cc7c7c6ccc6ccccc67)cc5)cc4)n3)cc2)cc1]

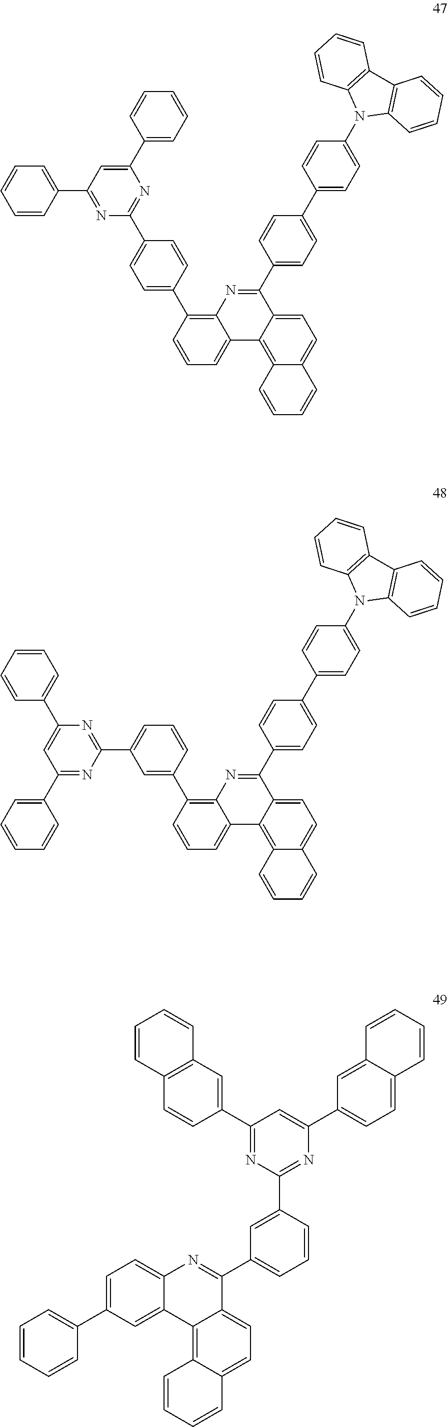 [SMILES: c1ccc(-c2cc(-c3ccccc3)nc(-c3ccc(-c4cccc5c4nc(-c4ccc(-c6ccc(-n7c8ccccc8c8ccccc87)cc6)cc4)c4ccc6ccccc6c45)cc3)n2)cc1 ; c1ccc(-c2cc(-c3ccccc3)nc(-c3cccc(-c4cccc5c4nc(-c4ccc(-c6ccc(-n7c8ccccc8c8ccccc87)cc6)cc4)c4ccc6ccccc6c45)c3)n2)cc1 ; c1ccc(-c2ccc3nc(-c4cccc(-c5nc(-c6ccc7ccccc7c6)cc(-c6ccc7ccccc7c6)n5)c4)c4ccc5ccccc5c4c3c2)cc1]

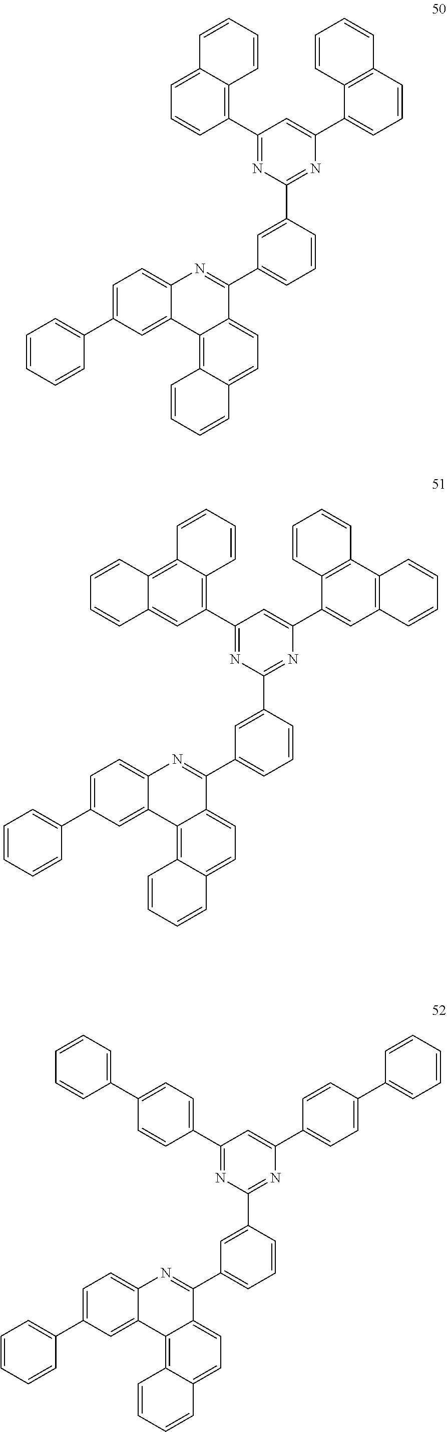 [SMILES: c1ccc(-c2ccc3nc(-c4cccc(-c5nc(-c6cccc7ccccc67)cc(-c6cccc7ccccc67)n5)c4)c4ccc5ccccc5c4c3c2)cc1 ; c1ccc(-c2ccc3nc(-c4cccc(-c5nc(-c6cc7ccccc7c7ccccc67)cc(-c6cc7ccccc7c7ccccc67)n5)c4)c4ccc5ccccc5c4c3c2)cc1 ; c1ccc(-c2ccc(-c3cc(-c4ccc(-c5ccccc5)cc4)nc(-c4cccc(-c5nc6ccc(-c7ccccc7)cc6c6c5ccc5ccccc56)c4)n3)cc2)cc1]

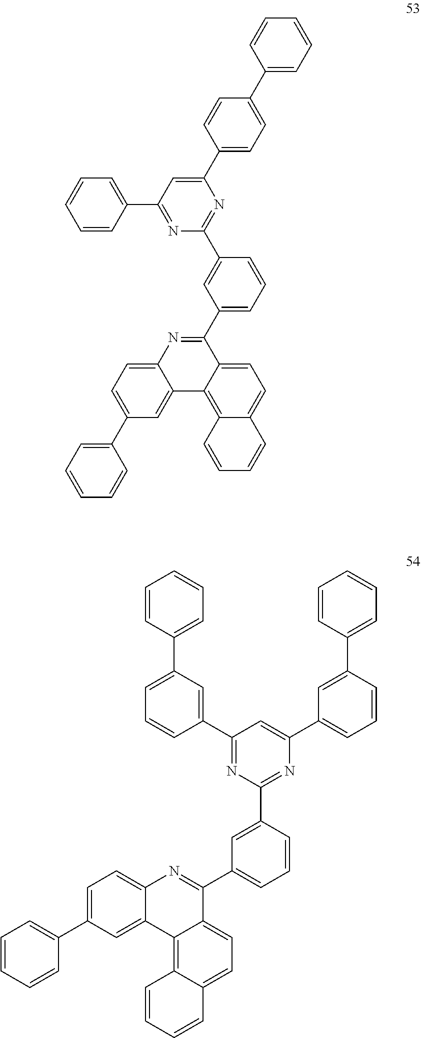 [SMILES: c1ccc(-c2ccc(-c3cc(-c4ccccc4)nc(-c4cccc(-c5nc6ccc(-c7ccccc7)cc6c6c5ccc5ccccc56)c4)n3)cc2)cc1 ; c1ccc(-c2cccc(-c3cc(-c4cccc(-c5ccccc5)c4)nc(-c4cccc(-c5nc6ccc(-c7ccccc7)cc6c6c5ccc5ccccc56)c4)n3)c2)cc1]

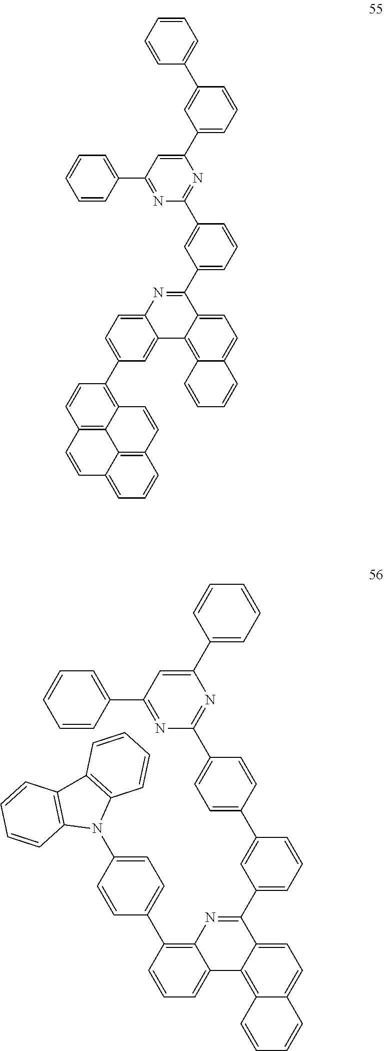 [SMILES: c1ccc(-c2cccc(-c3cc(-c4ccccc4)nc(-c4cccc(-c5nc6ccc(-c7ccc8ccc9cccc%10ccc7c8c9%10)cc6c6c5ccc5ccccc56)c4)n3)c2)cc1 ; c1ccc(-c2cc(-c3ccccc3)nc(-c3ccc(-c4cccc(-c5nc6c(-c7ccc(-n8c9ccccc9c9ccccc98)cc7)cccc6c6c5ccc5ccccc56)c4)cc3)n2)cc1]

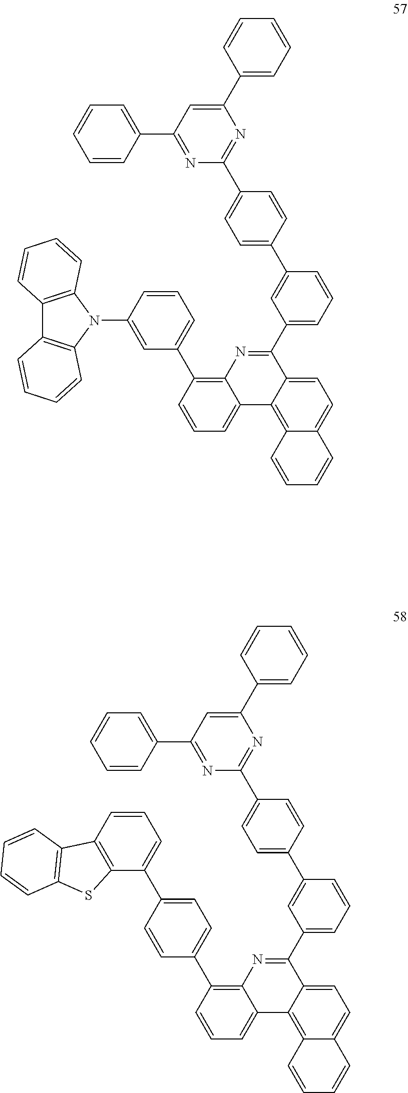 [SMILES: c1ccc(-c2cc(-c3ccccc3)nc(-c3ccc(-c4cccc(-c5nc6c(-c7cccc(-n8c9ccccc9c9ccccc98)c7)cccc6c6c5ccc5ccccc56)c4)cc3)n2)cc1 ; c1ccc(-c2cc(-c3ccccc3)nc(-c3ccc(-c4cccc(-c5nc6c(-c7ccc(-c8cccc9c8sc8ccccc89)cc7)cccc6c6c5ccc5ccccc56)c4)cc3)n2)cc1]

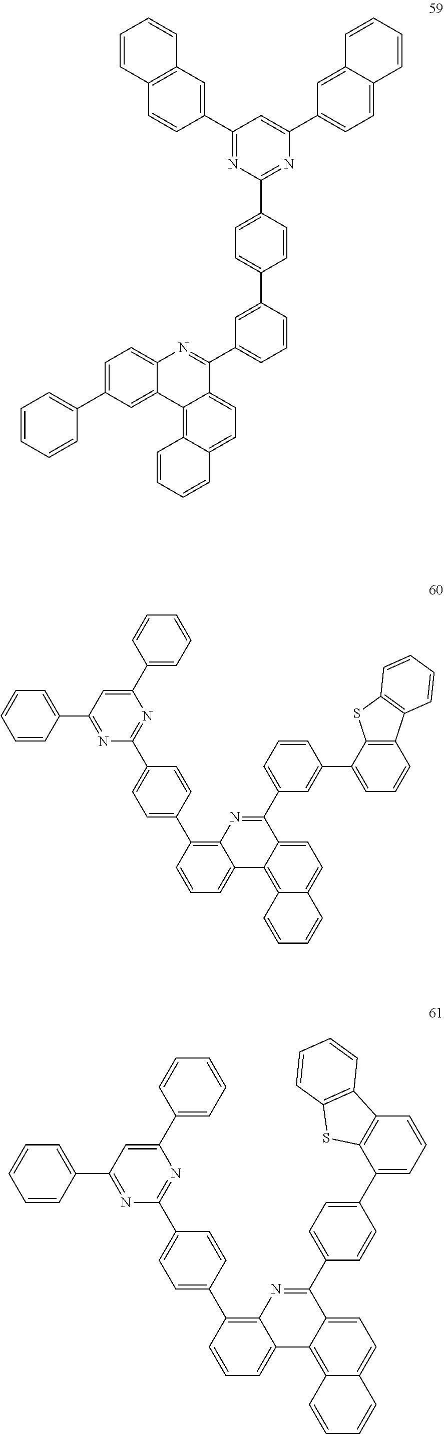 [SMILES: c1ccc(-c2ccc3nc(-c4cccc(-c5ccc(-c6nc(-c7ccc8ccccc8c7)cc(-c7ccc8ccccc8c7)n6)cc5)c4)c4ccc5ccccc5c4c3c2)cc1 ; c1ccc(-c2cc(-c3ccccc3)nc(-c3ccc(-c4cccc5c4nc(-c4cccc(-c6cccc7c6sc6ccccc67)c4)c4ccc6ccccc6c45)cc3)n2)cc1 ; c1ccc(-c2cc(-c3ccccc3)nc(-c3ccc(-c4cccc5c4nc(-c4ccc(-c6cccc7c6sc6ccccc67)cc4)c4ccc6ccccc6c45)cc3)n2)cc1]

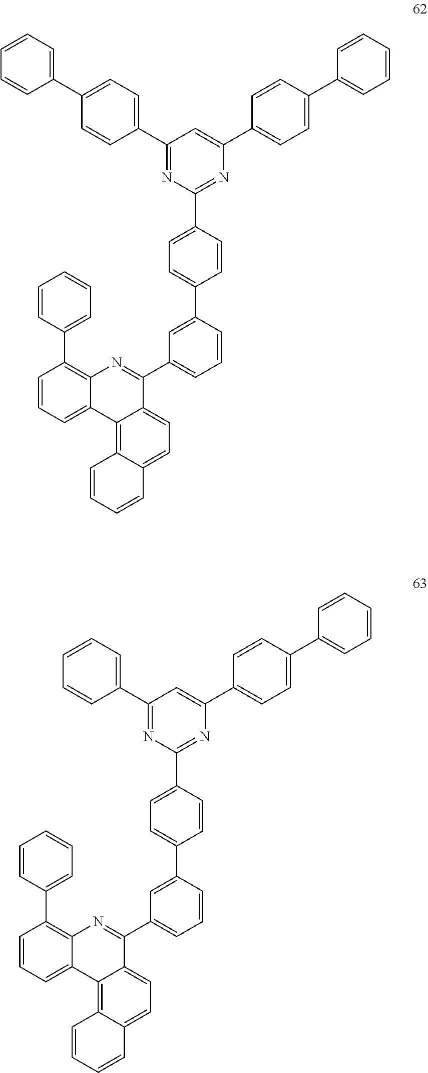 [SMILES: c1ccc(-c2ccc(-c3cc(-c4ccc(-c5ccccc5)cc4)nc(-c4ccc(-c5cccc(-c6nc7c(-c8ccccc8)cccc7c7c6ccc6ccccc67)c5)cc4)n3)cc2)cc1 ; c1ccc(-c2ccc(-c3cc(-c4ccccc4)nc(-c4ccc(-c5cccc(-c6nc7c(-c8ccccc8)cccc7c7c6ccc6ccccc67)c5)cc4)n3)cc2)cc1]

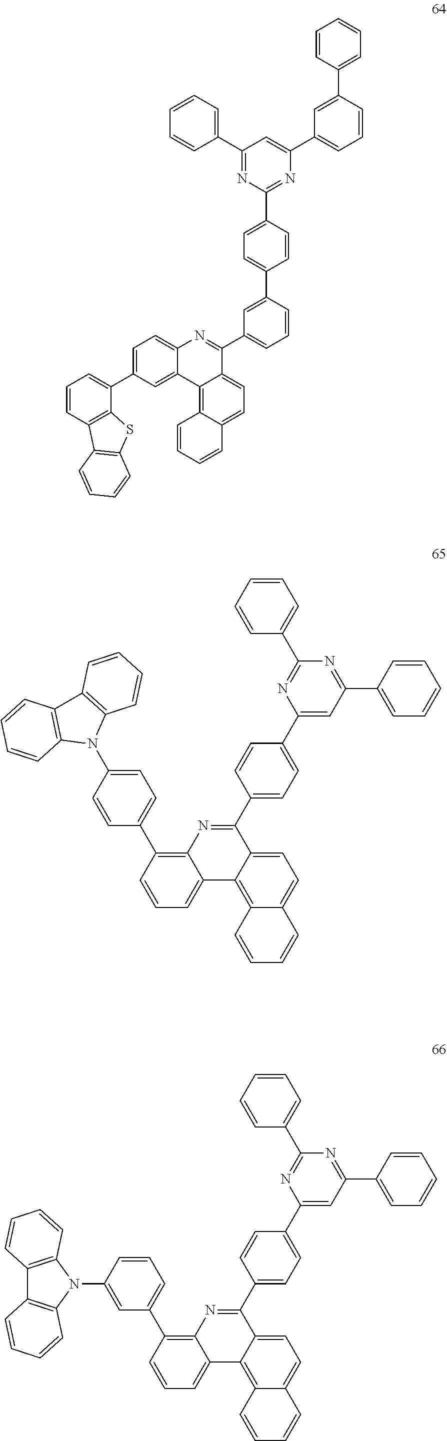 [SMILES: c1ccc(-c2cccc(-c3cc(-c4ccccc4)nc(-c4ccc(-c5cccc(-c6nc7ccc(-c8cccc9c8sc8ccccc89)cc7c7c6ccc6ccccc67)c5)cc4)n3)c2)cc1 ; c1ccc(-c2cc(-c3ccc(-c4nc5c(-c6ccc(-n7c8ccccc8c8ccccc87)cc6)cccc5c5c4ccc4ccccc45)cc3)nc(-c3ccccc3)n2)cc1 ; c1ccc(-c2cc(-c3ccc(-c4nc5c(-c6cccc(-n7c8ccccc8c8ccccc87)c6)cccc5c5c4ccc4ccccc45)cc3)nc(-c3ccccc3)n2)cc1]

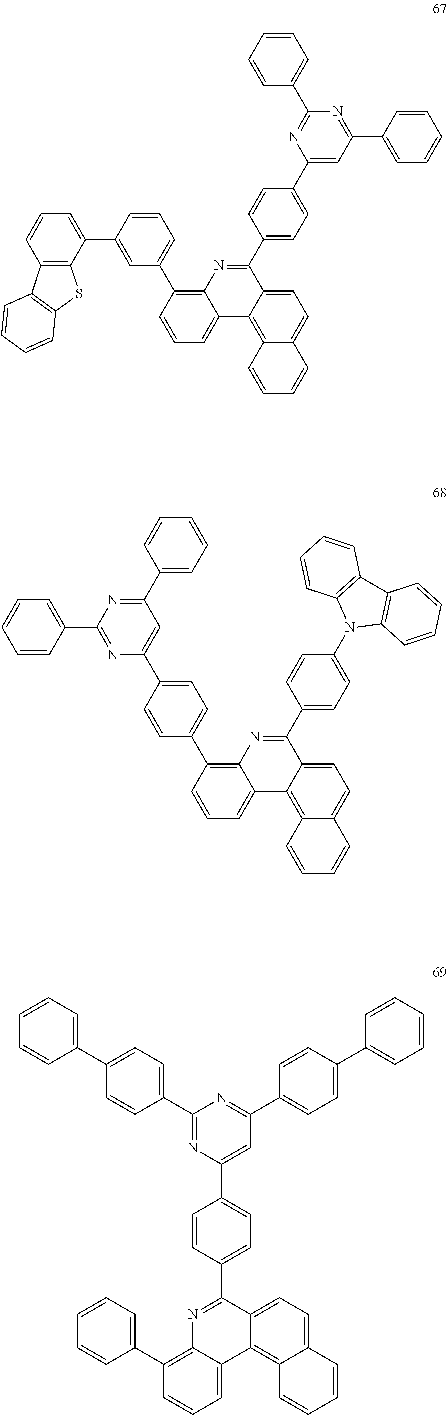 [SMILES: c1ccc(-c2cc(-c3ccc(-c4nc5c(-c6cccc(-c7cccc8c7sc7ccccc78)c6)cccc5c5c4ccc4ccccc45)cc3)nc(-c3ccccc3)n2)cc1 ; c1ccc(-c2cc(-c3ccc(-c4cccc5c4nc(-c4ccc(-n6c7ccccc7c7ccccc76)cc4)c4ccc6ccccc6c45)cc3)nc(-c3ccccc3)n2)cc1 ; c1ccc(-c2ccc(-c3cc(-c4ccc(-c5nc6c(-c7ccccc7)cccc6c6c5ccc5ccccc56)cc4)nc(-c4ccc(-c5ccccc5)cc4)n3)cc2)cc1]

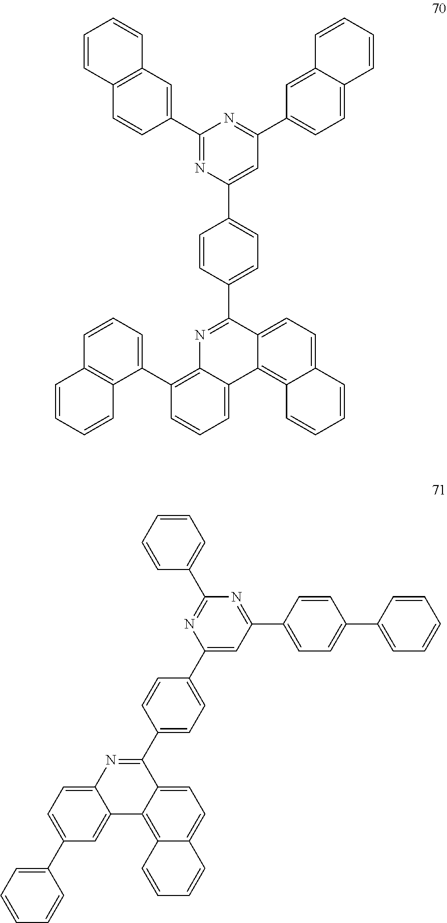 [SMILES: c1ccc2cc(-c3cc(-c4ccc(-c5nc6c(-c7cccc8ccccc78)cccc6c6c5ccc5ccccc56)cc4)nc(-c4ccc5ccccc5c4)n3)ccc2c1 ; c1ccc(-c2ccc(-c3cc(-c4ccc(-c5nc6ccc(-c7ccccc7)cc6c6c5ccc5ccccc56)cc4)nc(-c4ccccc4)n3)cc2)cc1]

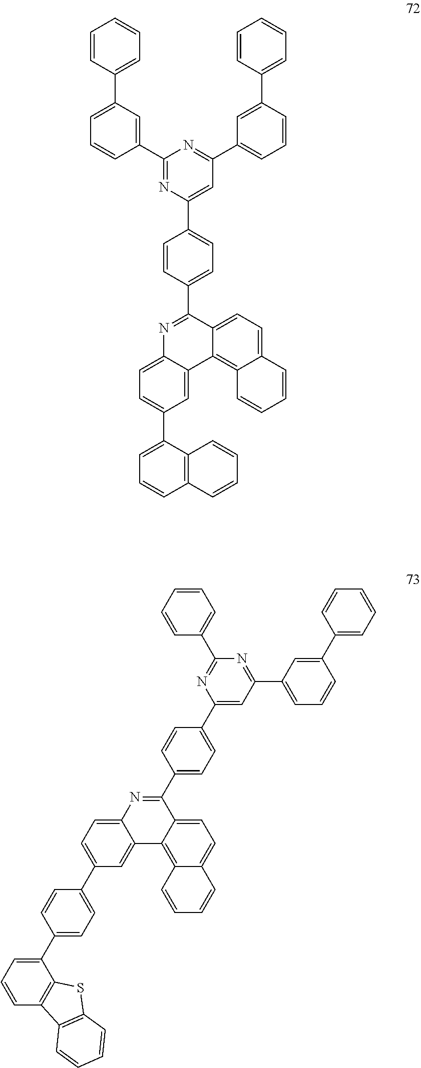 [SMILES: c1ccc(-c2cccc(-c3cc(-c4ccc(-c5nc6ccc(-c7cccc8ccccc78)cc6c6c5ccc5ccccc56)cc4)nc(-c4cccc(-c5ccccc5)c4)n3)c2)cc1 ; c1ccc(-c2cccc(-c3cc(-c4ccc(-c5nc6ccc(-c7ccc(-c8cccc9c8sc8ccccc89)cc7)cc6c6c5ccc5ccccc56)cc4)nc(-c4ccccc4)n3)c2)cc1]

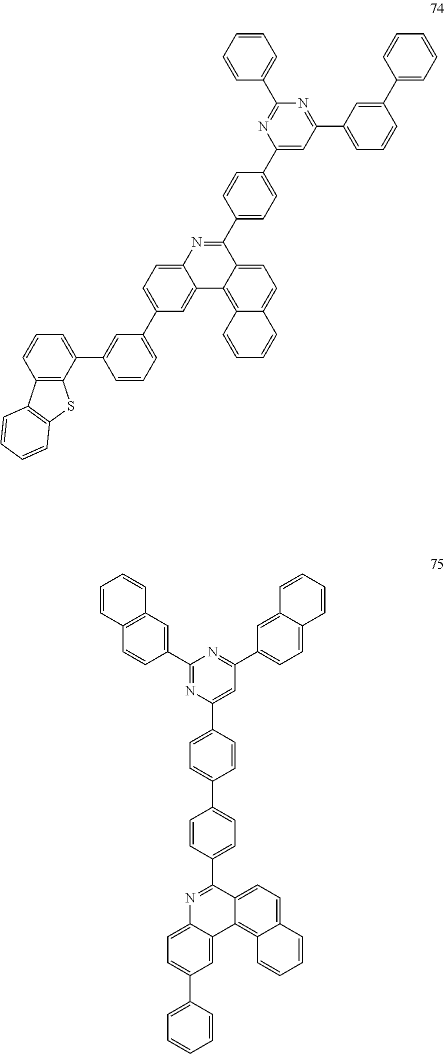 [SMILES: c1ccc(-c2cccc(-c3cc(-c4ccc(-c5nc6ccc(-c7cccc(-c8cccc9c8sc8ccccc89)c7)cc6c6c5ccc5ccccc56)cc4)nc(-c4ccccc4)n3)c2)cc1 ; c1ccc(-c2ccc3nc(-c4ccc(-c5ccc(-c6cc(-c7ccc8ccccc8c7)nc(-c7ccc8ccccc8c7)n6)cc5)cc4)c4ccc5ccccc5c4c3c2)cc1]

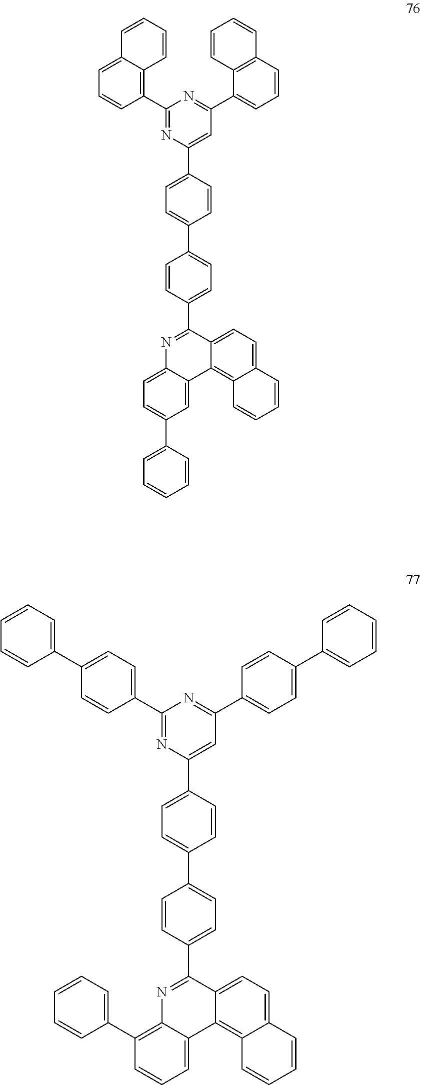 [SMILES: c1ccc(-c2ccc3nc(-c4ccc(-c5ccc(-c6cc(-c7cccc8ccccc78)nc(-c7cccc8ccccc78)n6)cc5)cc4)c4ccc5ccccc5c4c3c2)cc1 ; c1ccc(-c2ccc(-c3cc(-c4ccc(-c5ccc(-c6nc7c(-c8ccccc8)cccc7c7c6ccc6ccccc67)cc5)cc4)nc(-c4ccc(-c5ccccc5)cc4)n3)cc2)cc1]

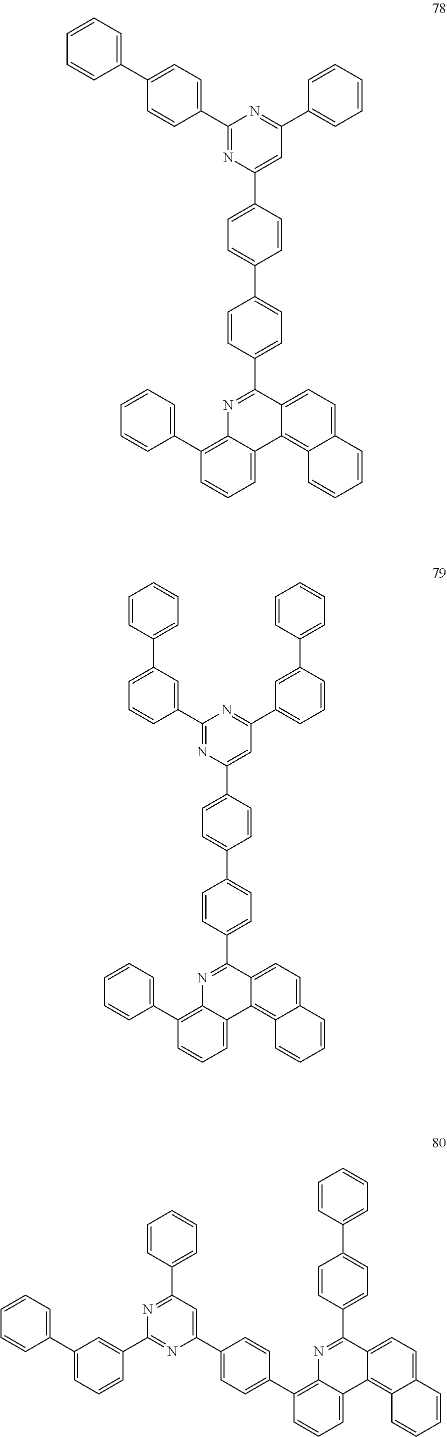 [SMILES: c1ccc(-c2ccc(-c3nc(-c4ccccc4)cc(-c4ccc(-c5ccc(-c6nc7c(-c8ccccc8)cccc7c7c6ccc6ccccc67)cc5)cc4)n3)cc2)cc1 ; c1ccc(-c2cccc(-c3cc(-c4ccc(-c5ccc(-c6nc7c(-c8ccccc8)cccc7c7c6ccc6ccccc67)cc5)cc4)nc(-c4cccc(-c5ccccc5)c4)n3)c2)cc1 ; c1ccc(-c2ccc(-c3nc4c(-c5ccc(-c6cc(-c7ccccc7)nc(-c7cccc(-c8ccccc8)c7)n6)cc5)cccc4c4c3ccc3ccccc34)cc2)cc1]

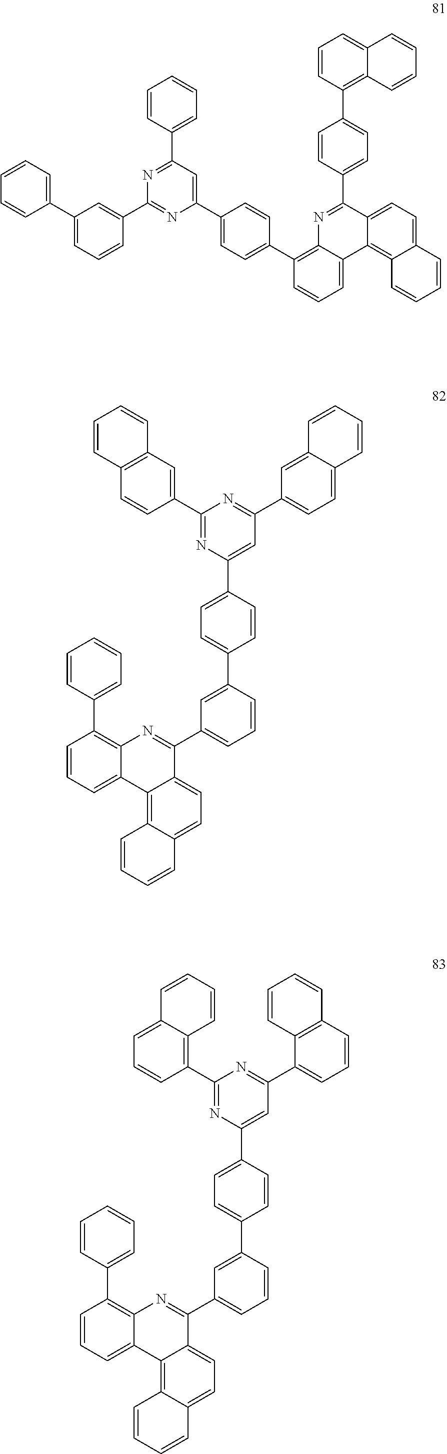 [SMILES: c1ccc(-c2cccc(-c3nc(-c4ccccc4)cc(-c4ccc(-c5cccc6c5nc(-c5ccc(-c7cccc8ccccc78)cc5)c5ccc7ccccc7c56)cc4)n3)c2)cc1 ; c1ccc(-c2cccc3c2nc(-c2cccc(-c4ccc(-c5cc(-c6ccc7ccccc7c6)nc(-c6ccc7ccccc7c6)n5)cc4)c2)c2ccc4ccccc4c23)cc1 ; c1ccc(-c2cccc3c2nc(-c2cccc(-c4ccc(-c5cc(-c6cccc7ccccc67)nc(-c6cccc7ccccc67)n5)cc4)c2)c2ccc4ccccc4c23)cc1]

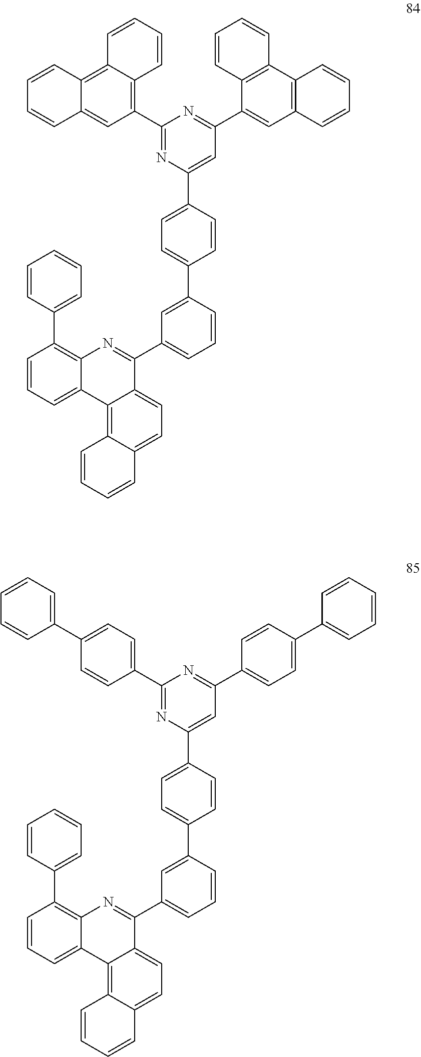 [SMILES: c1ccc(-c2cccc3c2nc(-c2cccc(-c4ccc(-c5cc(-c6cc7ccccc7c7ccccc67)nc(-c6cc7ccccc7c7ccccc67)n5)cc4)c2)c2ccc4ccccc4c23)cc1 ; c1ccc(-c2ccc(-c3cc(-c4ccc(-c5cccc(-c6nc7c(-c8ccccc8)cccc7c7c6ccc6ccccc67)c5)cc4)nc(-c4ccc(-c5ccccc5)cc4)n3)cc2)cc1]

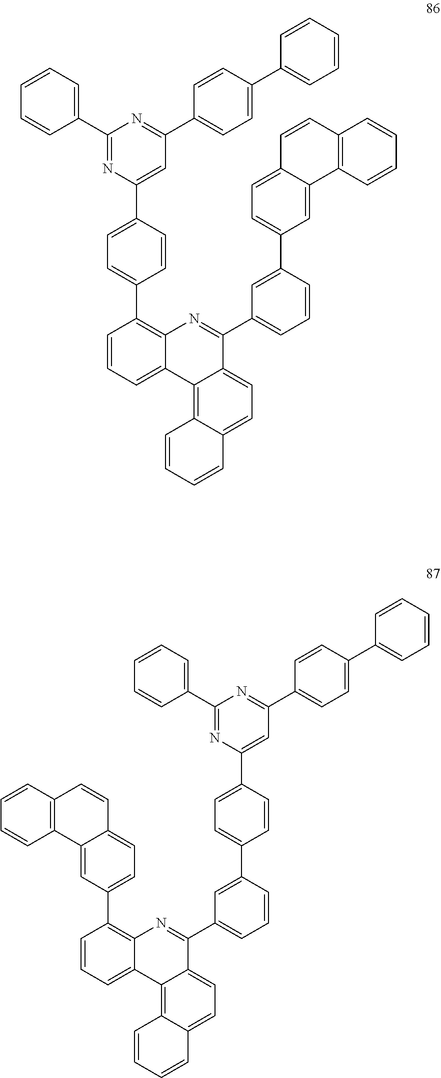 [SMILES: c1ccc(-c2ccc(-c3cc(-c4ccc(-c5cccc6c5nc(-c5cccc(-c7ccc8ccc9ccccc9c8c7)c5)c5ccc7ccccc7c56)cc4)nc(-c4ccccc4)n3)cc2)cc1 ; c1ccc(-c2ccc(-c3cc(-c4ccc(-c5cccc(-c6nc7c(-c8ccc9ccc%10ccccc%10c9c8)cccc7c7c6ccc6ccccc67)c5)cc4)nc(-c4ccccc4)n3)cc2)cc1]

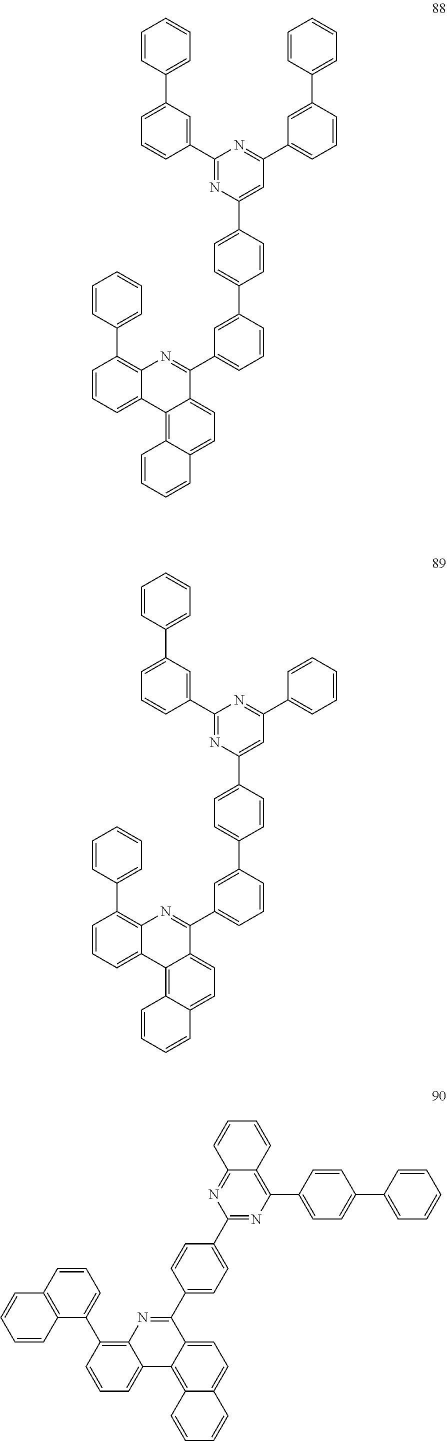 [SMILES: c1ccc(-c2cccc(-c3cc(-c4ccc(-c5cccc(-c6nc7c(-c8ccccc8)cccc7c7c6ccc6ccccc67)c5)cc4)nc(-c4cccc(-c5ccccc5)c4)n3)c2)cc1 ; c1ccc(-c2cccc(-c3nc(-c4ccccc4)cc(-c4ccc(-c5cccc(-c6nc7c(-c8ccccc8)cccc7c7c6ccc6ccccc67)c5)cc4)n3)c2)cc1 ; c1ccc(-c2ccc(-c3nc(-c4ccc(-c5nc6c(-c7cccc8ccccc78)cccc6c6c5ccc5ccccc56)cc4)nc4ccccc34)cc2)cc1]

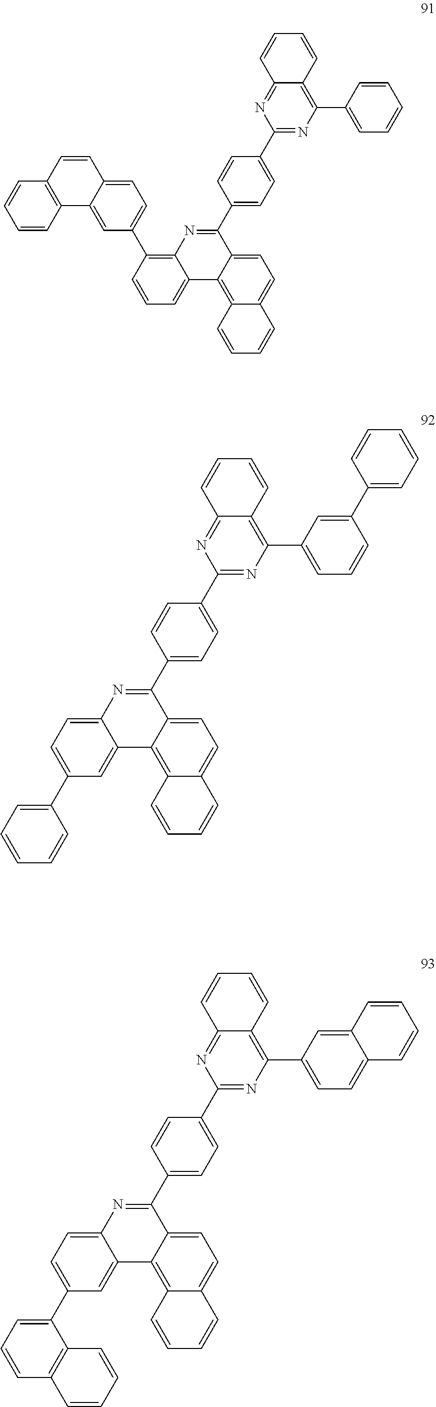 [SMILES: c1ccc(-c2nc(-c3ccc(-c4nc5c(-c6ccc7ccc8ccccc8c7c6)cccc5c5c4ccc4ccccc45)cc3)nc3ccccc23)cc1 ; c1ccc(-c2cccc(-c3nc(-c4ccc(-c5nc6ccc(-c7ccccc7)cc6c6c5ccc5ccccc56)cc4)nc4ccccc34)c2)cc1 ; c1ccc2cc(-c3nc(-c4ccc(-c5nc6ccc(-c7cccc8ccccc78)cc6c6c5ccc5ccccc56)cc4)nc4ccccc34)ccc2c1]

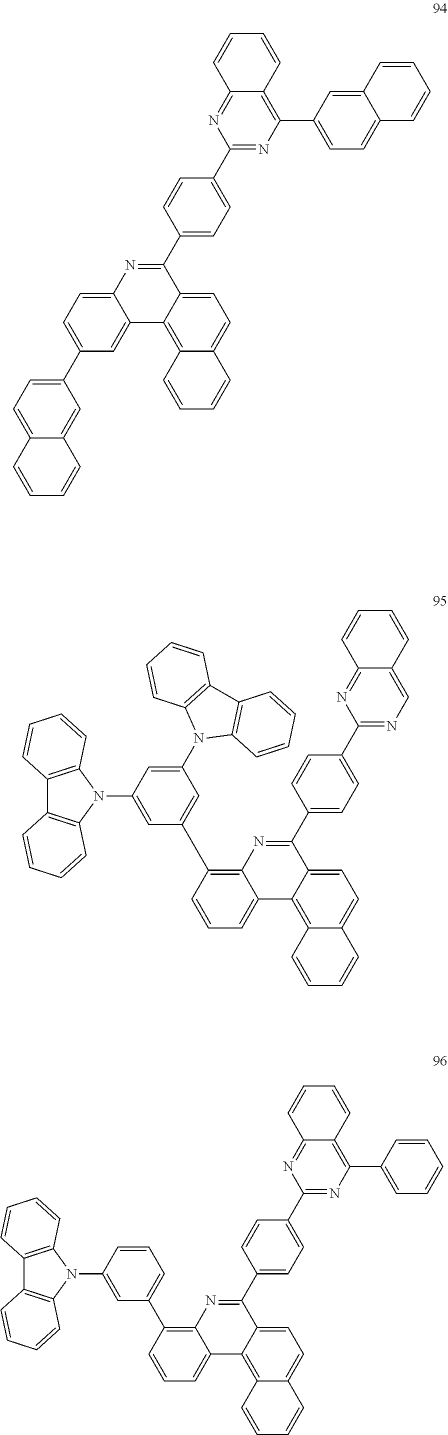 [SMILES: c1ccc2cc(-c3ccc4nc(-c5ccc(-c6nc(-c7ccc8ccccc8c7)c7ccccc7n6)cc5)c5ccc6ccccc6c5c4c3)ccc2c1 ; c1ccc2nc(-c3ccc(-c4nc5c(-c6cc(-n7c8ccccc8c8ccccc87)cc(-n7c8ccccc8c8ccccc87)c6)cccc5c5c4ccc4ccccc45)cc3)ncc2c1 ; c1ccc(-c2nc(-c3ccc(-c4nc5c(-c6cccc(-n7c8ccccc8c8ccccc87)c6)cccc5c5c4ccc4ccccc45)cc3)nc3ccccc23)cc1]

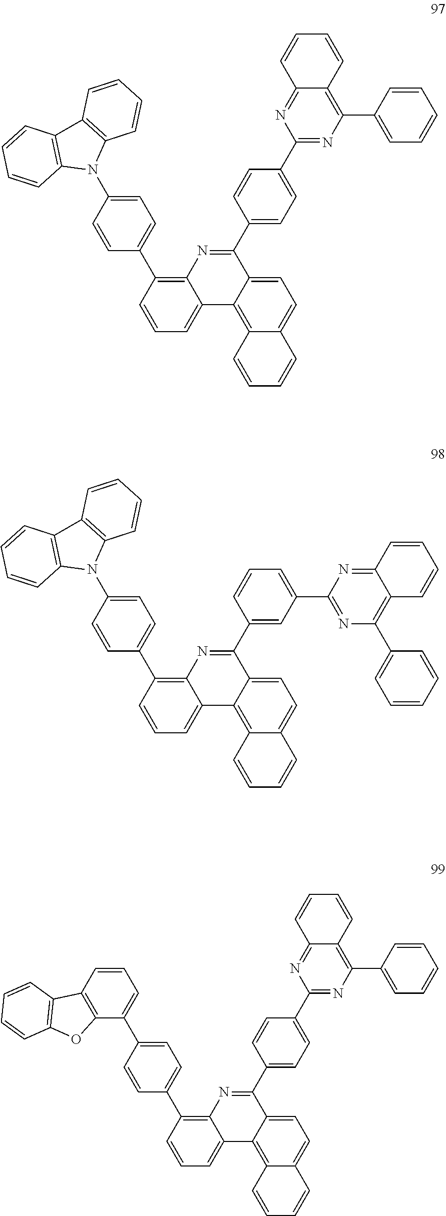 [SMILES: c1ccc(-c2nc(-c3ccc(-c4nc5c(-c6ccc(-n7c8ccccc8c8ccccc87)cc6)cccc5c5c4ccc4ccccc45)cc3)nc3ccccc23)cc1 ; c1ccc(-c2nc(-c3cccc(-c4nc5c(-c6ccc(-n7c8ccccc8c8ccccc87)cc6)cccc5c5c4ccc4ccccc45)c3)nc3ccccc23)cc1 ; c1ccc(-c2nc(-c3ccc(-c4nc5c(-c6ccc(-c7cccc8c7oc7ccccc78)cc6)cccc5c5c4ccc4ccccc45)cc3)nc3ccccc23)cc1]

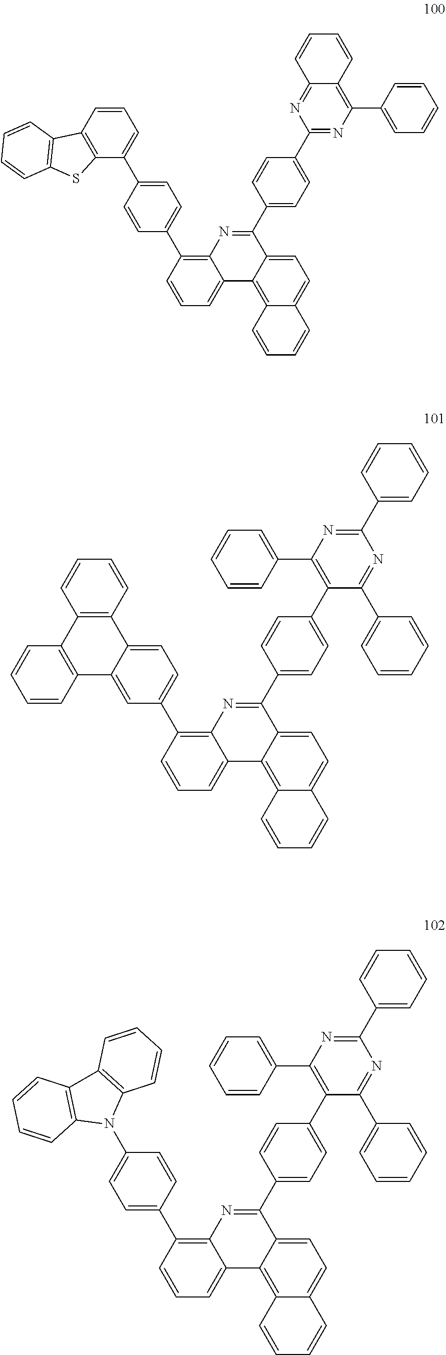 [SMILES: c1ccc(-c2nc(-c3ccc(-c4nc5c(-c6ccc(-c7cccc8c7sc7ccccc78)cc6)cccc5c5c4ccc4ccccc45)cc3)nc3ccccc23)cc1 ; c1ccc(-c2nc(-c3ccccc3)c(-c3ccc(-c4nc5c(-c6ccc7c8ccccc8c8ccccc8c7c6)cccc5c5c4ccc4ccccc45)cc3)c(-c3ccccc3)n2)cc1 ; c1ccc(-c2nc(-c3ccccc3)c(-c3ccc(-c4nc5c(-c6ccc(-n7c8ccccc8c8ccccc87)cc6)cccc5c5c4ccc4ccccc45)cc3)c(-c3ccccc3)n2)cc1]

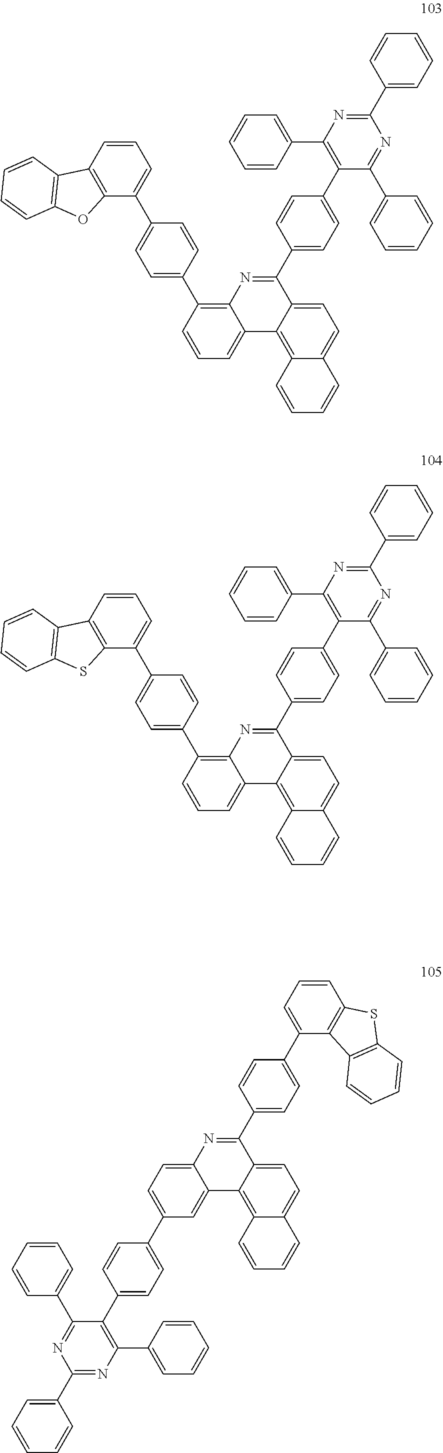 [SMILES: c1ccc(-c2nc(-c3ccccc3)c(-c3ccc(-c4nc5c(-c6ccc(-c7cccc8c7oc7ccccc78)cc6)cccc5c5c4ccc4ccccc45)cc3)c(-c3ccccc3)n2)cc1 ; c1ccc(-c2nc(-c3ccccc3)c(-c3ccc(-c4nc5c(-c6ccc(-c7cccc8c7sc7ccccc78)cc6)cccc5c5c4ccc4ccccc45)cc3)c(-c3ccccc3)n2)cc1 ; c1ccc(-c2nc(-c3ccccc3)c(-c3ccc(-c4ccc5nc(-c6ccc(-c7cccc8sc9ccccc9c78)cc6)c6ccc7ccccc7c6c5c4)cc3)c(-c3ccccc3)n2)cc1]

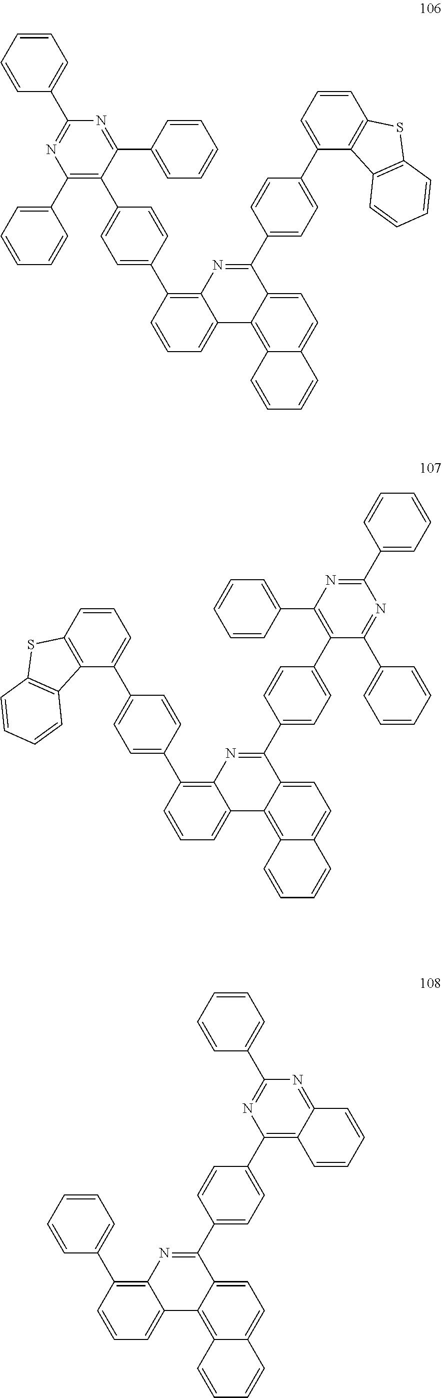 [SMILES: c1ccc(-c2nc(-c3ccccc3)c(-c3ccc(-c4cccc5c4nc(-c4ccc(-c6cccc7sc8ccccc8c67)cc4)c4ccc6ccccc6c45)cc3)c(-c3ccccc3)n2)cc1 ; c1ccc(-c2nc(-c3ccccc3)c(-c3ccc(-c4nc5c(-c6ccc(-c7cccc8sc9ccccc9c78)cc6)cccc5c5c4ccc4ccccc45)cc3)c(-c3ccccc3)n2)cc1 ; c1ccc(-c2nc(-c3ccc(-c4nc5c(-c6ccccc6)cccc5c5c4ccc4ccccc45)cc3)c3ccccc3n2)cc1]

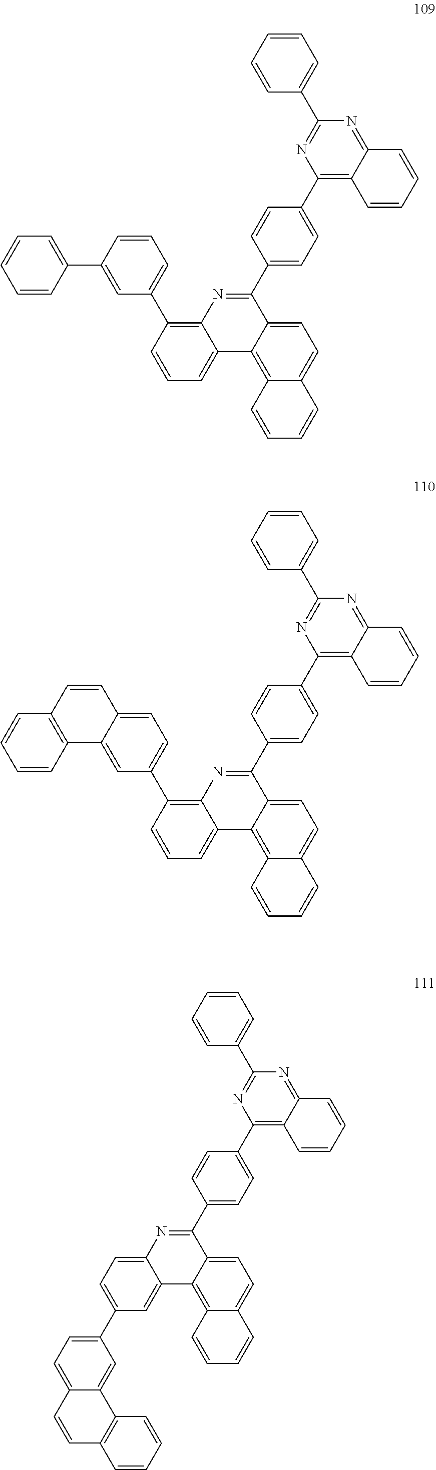 [SMILES: c1ccc(-c2cccc(-c3cccc4c3nc(-c3ccc(-c5nc(-c6ccccc6)nc6ccccc56)cc3)c3ccc5ccccc5c34)c2)cc1 ; c1ccc(-c2nc(-c3ccc(-c4nc5c(-c6ccc7ccc8ccccc8c7c6)cccc5c5c4ccc4ccccc45)cc3)c3ccccc3n2)cc1 ; c1ccc(-c2nc(-c3ccc(-c4nc5ccc(-c6ccc7ccc8ccccc8c7c6)cc5c5c4ccc4ccccc45)cc3)c3ccccc3n2)cc1]

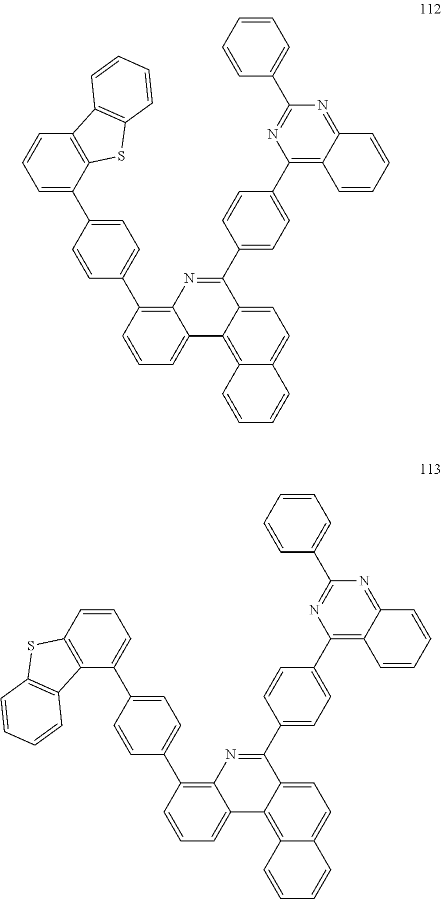 [SMILES: c1ccc(-c2nc(-c3ccc(-c4nc5c(-c6ccc(-c7cccc8c7sc7ccccc78)cc6)cccc5c5c4ccc4ccccc45)cc3)c3ccccc3n2)cc1 ; c1ccc(-c2nc(-c3ccc(-c4nc5c(-c6ccc(-c7cccc8sc9ccccc9c78)cc6)cccc5c5c4ccc4ccccc45)cc3)c3ccccc3n2)cc1]

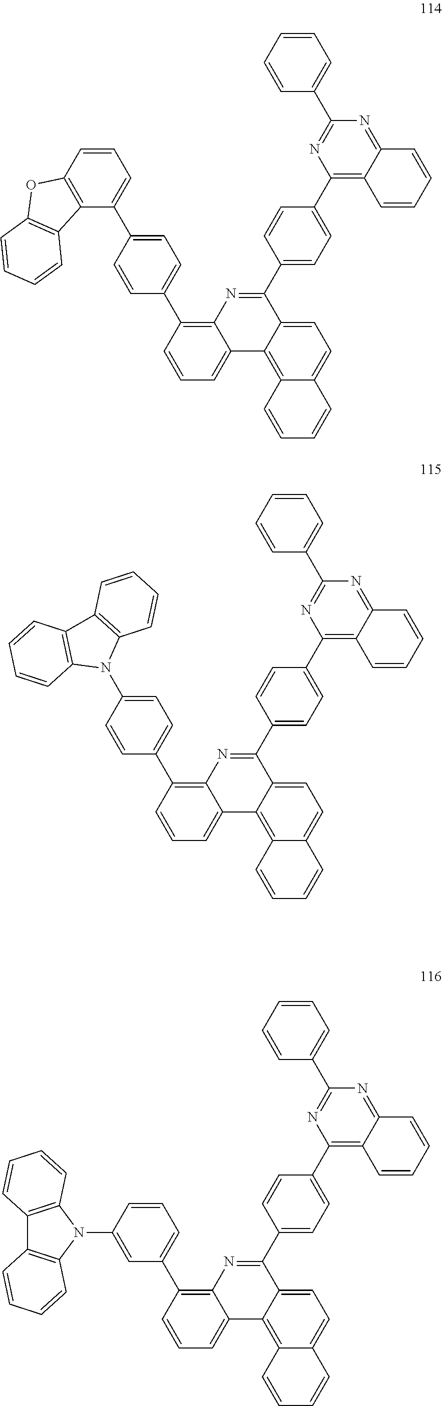 [SMILES: c1ccc(-c2nc(-c3ccc(-c4nc5c(-c6ccc(-c7cccc8oc9ccccc9c78)cc6)cccc5c5c4ccc4ccccc45)cc3)c3ccccc3n2)cc1 ; c1ccc(-c2nc(-c3ccc(-c4nc5c(-c6ccc(-n7c8ccccc8c8ccccc87)cc6)cccc5c5c4ccc4ccccc45)cc3)c3ccccc3n2)cc1 ; c1ccc(-c2nc(-c3ccc(-c4nc5c(-c6cccc(-n7c8ccccc8c8ccccc87)c6)cccc5c5c4ccc4ccccc45)cc3)c3ccccc3n2)cc1]

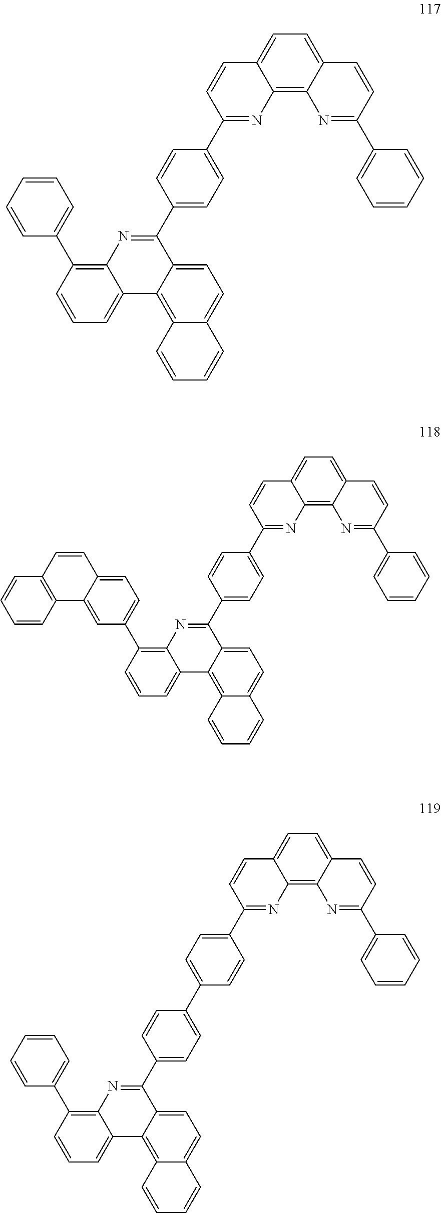 [SMILES: c1ccc(-c2ccc3ccc4ccc(-c5ccc(-c6nc7c(-c8ccccc8)cccc7c7c6ccc6ccccc67)cc5)nc4c3n2)cc1 ; c1ccc(-c2ccc3ccc4ccc(-c5ccc(-c6nc7c(-c8ccc9ccc%10ccccc%10c9c8)cccc7c7c6ccc6ccccc67)cc5)nc4c3n2)cc1 ; c1ccc(-c2ccc3ccc4ccc(-c5ccc(-c6ccc(-c7nc8c(-c9ccccc9)cccc8c8c7ccc7ccccc78)cc6)cc5)nc4c3n2)cc1]

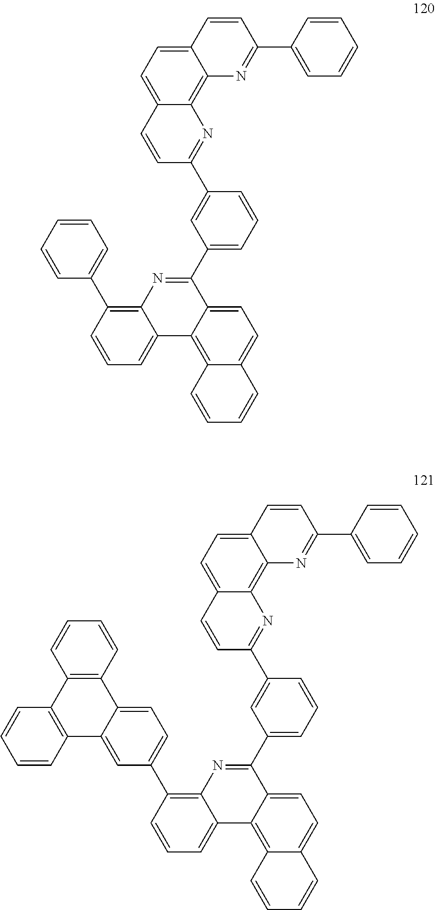 [SMILES: c1ccc(-c2ccc3ccc4ccc(-c5cccc(-c6nc7c(-c8ccccc8)cccc7c7c6ccc6ccccc67)c5)nc4c3n2)cc1 ; c1ccc(-c2ccc3ccc4ccc(-c5cccc(-c6nc7c(-c8ccc9c%10ccccc%10c%10ccccc%10c9c8)cccc7c7c6ccc6ccccc67)c5)nc4c3n2)cc1]

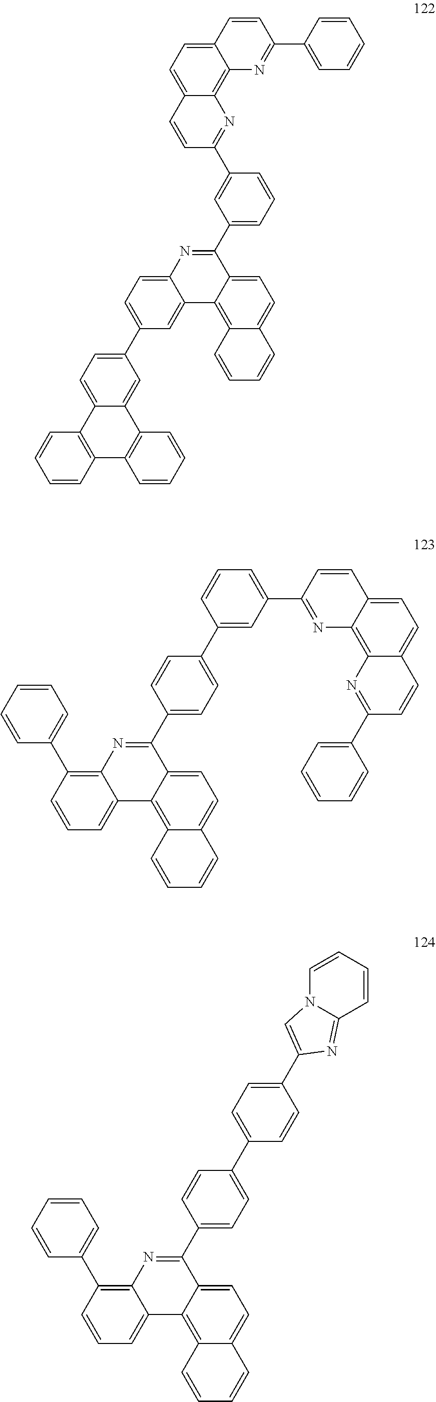 [SMILES: c1ccc(-c2ccc3ccc4ccc(-c5cccc(-c6nc7ccc(-c8ccc9c%10ccccc%10c%10ccccc%10c9c8)cc7c7c6ccc6ccccc67)c5)nc4c3n2)cc1 ; c1ccc(-c2ccc3ccc4ccc(-c5cccc(-c6ccc(-c7nc8c(-c9ccccc9)cccc8c8c7ccc7ccccc78)cc6)c5)nc4c3n2)cc1 ; c1ccc(-c2cccc3c2nc(-c2ccc(-c4ccc(-c5cn6ccccc6n5)cc4)cc2)c2ccc4ccccc4c23)cc1]

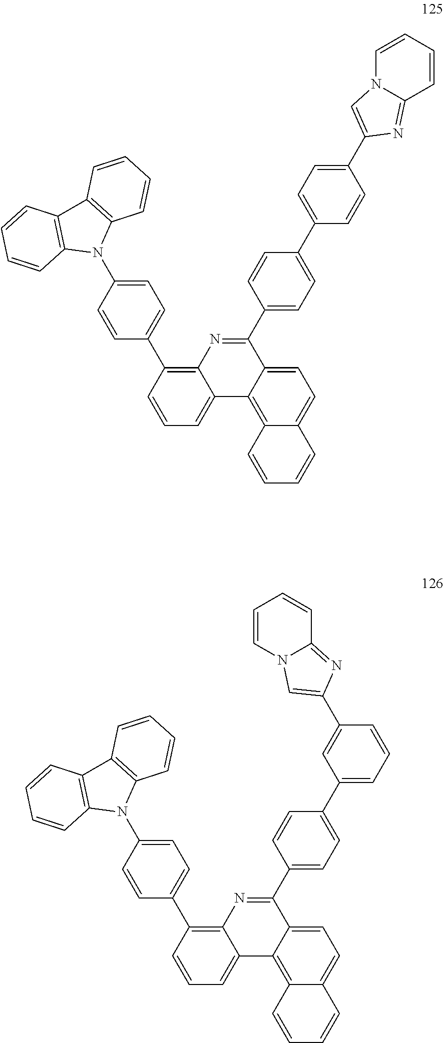 [SMILES: c1ccc2c(c1)ccc1c(-c3ccc(-c4ccc(-c5cn6ccccc6n5)cc4)cc3)nc3c(-c4ccc(-n5c6ccccc6c6ccccc65)cc4)cccc3c12 ; c1cc(-c2ccc(-c3nc4c(-c5ccc(-n6c7ccccc7c7ccccc76)cc5)cccc4c4c3ccc3ccccc34)cc2)cc(-c2cn3ccccc3n2)c1]

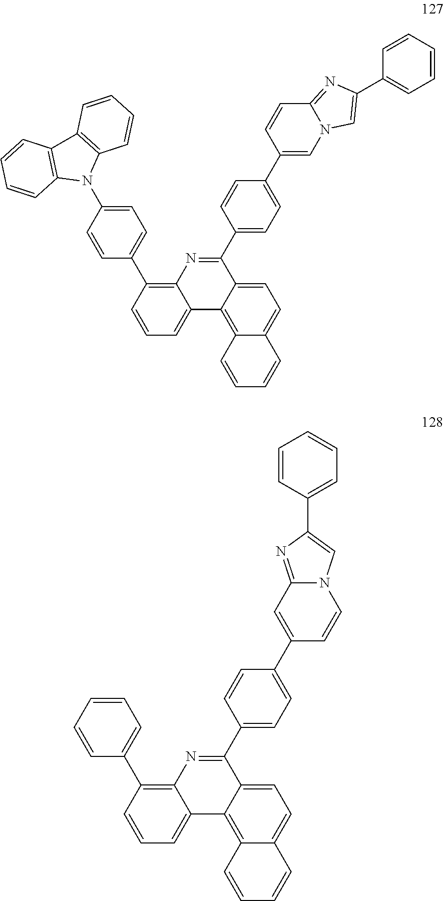 [SMILES: c1ccc(-c2cn3cc(-c4ccc(-c5nc6c(-c7ccc(-n8c9ccccc9c9ccccc98)cc7)cccc6c6c5ccc5ccccc56)cc4)ccc3n2)cc1 ; c1ccc(-c2cn3ccc(-c4ccc(-c5nc6c(-c7ccccc7)cccc6c6c5ccc5ccccc56)cc4)cc3n2)cc1]

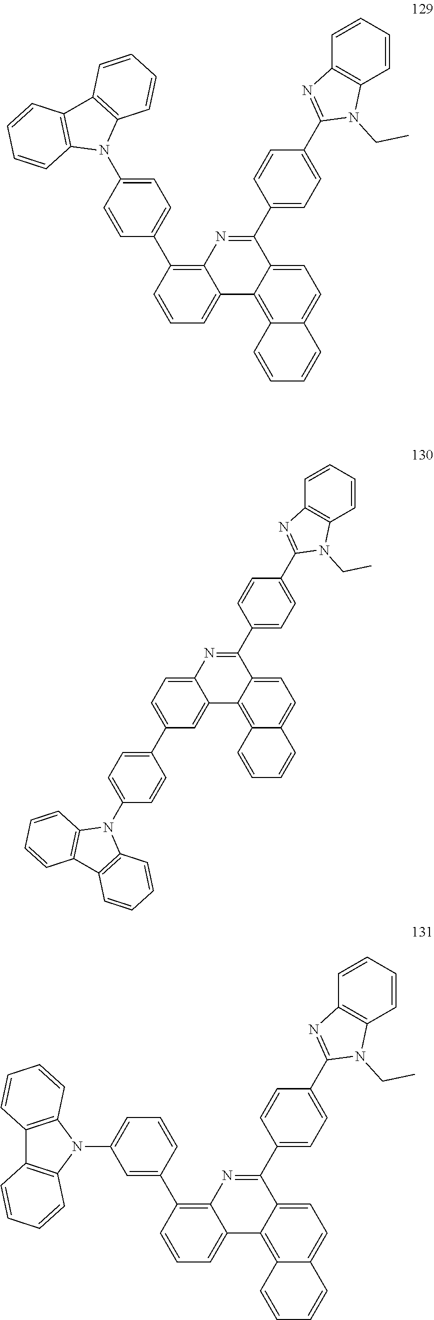 [SMILES: CCn1c(-c2ccc(-c3nc4c(-c5ccc(-n6c7ccccc7c7ccccc76)cc5)cccc4c4c3ccc3ccccc34)cc2)nc2ccccc21 ; CCn1c(-c2ccc(-c3nc4ccc(-c5ccc(-n6c7ccccc7c7ccccc76)cc5)cc4c4c3ccc3ccccc34)cc2)nc2ccccc21 ; CCn1c(-c2ccc(-c3nc4c(-c5cccc(-n6c7ccccc7c7ccccc76)c5)cccc4c4c3ccc3ccccc34)cc2)nc2ccccc21]

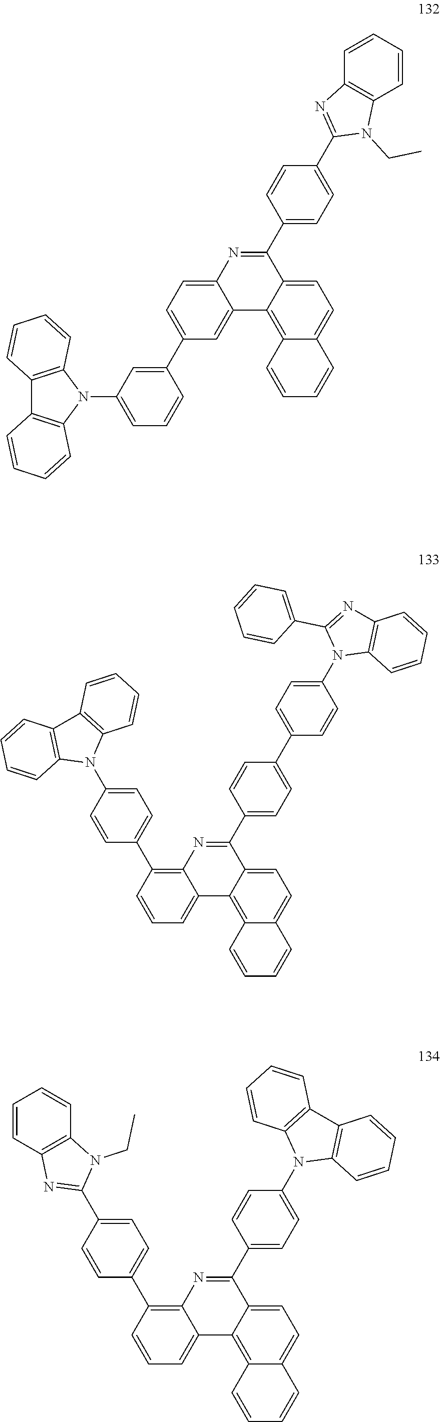 [SMILES: CCn1c(-c2ccc(-c3nc4ccc(-c5cccc(-n6c7ccccc7c7ccccc76)c5)cc4c4c3ccc3ccccc34)cc2)nc2ccccc21 ; c1ccc(-c2nc3ccccc3n2-c2ccc(-c3ccc(-c4nc5c(-c6ccc(-n7c8ccccc8c8ccccc87)cc6)cccc5c5c4ccc4ccccc45)cc3)cc2)cc1 ; CCn1c(-c2ccc(-c3cccc4c3nc(-c3ccc(-n5c6ccccc6c6ccccc65)cc3)c3ccc5ccccc5c34)cc2)nc2ccccc21]

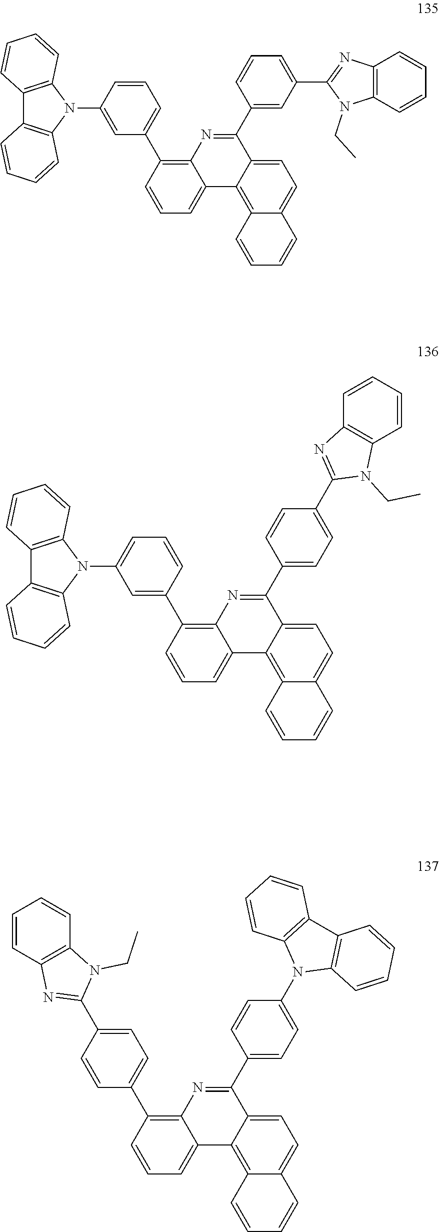 [SMILES: CCn1c(-c2cccc(-c3nc4c(-c5cccc(-n6c7ccccc7c7ccccc76)c5)cccc4c4c3ccc3ccccc34)c2)nc2ccccc21 ; CCn1c(-c2ccc(-c3nc4c(-c5cccc(-n6c7ccccc7c7ccccc76)c5)cccc4c4c3ccc3ccccc34)cc2)nc2ccccc21 ; CCn1c(-c2ccc(-c3cccc4c3nc(-c3ccc(-n5c6ccccc6c6ccccc65)cc3)c3ccc5ccccc5c34)cc2)nc2ccccc21]

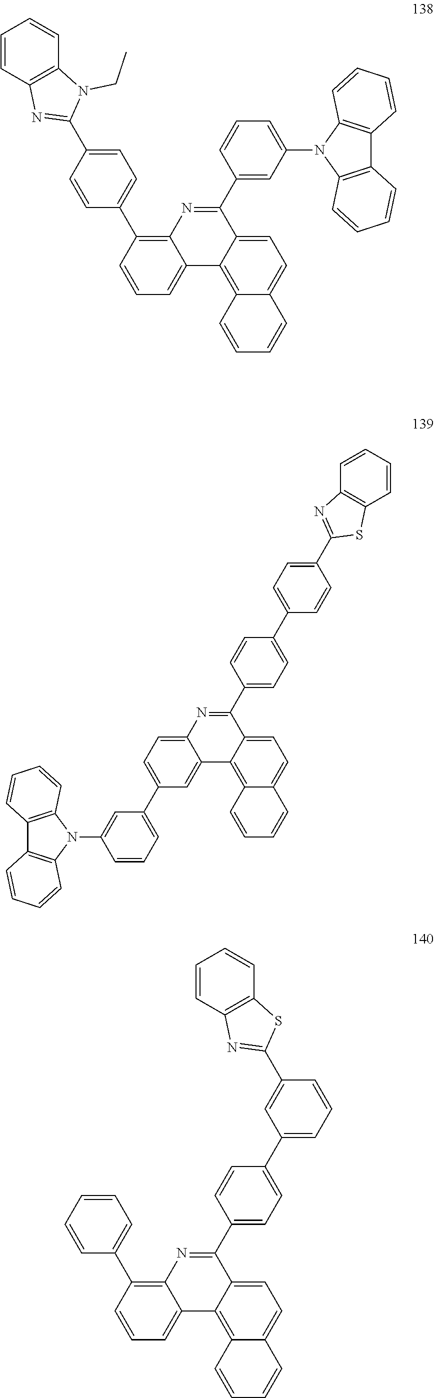 [SMILES: CCn1c(-c2ccc(-c3cccc4c3nc(-c3cccc(-n5c6ccccc6c6ccccc65)c3)c3ccc5ccccc5c34)cc2)nc2ccccc21 ; c1cc(-c2ccc3nc(-c4ccc(-c5ccc(-c6nc7ccccc7s6)cc5)cc4)c4ccc5ccccc5c4c3c2)cc(-n2c3ccccc3c3ccccc32)c1 ; c1ccc(-c2cccc3c2nc(-c2ccc(-c4cccc(-c5nc6ccccc6s5)c4)cc2)c2ccc4ccccc4c23)cc1]

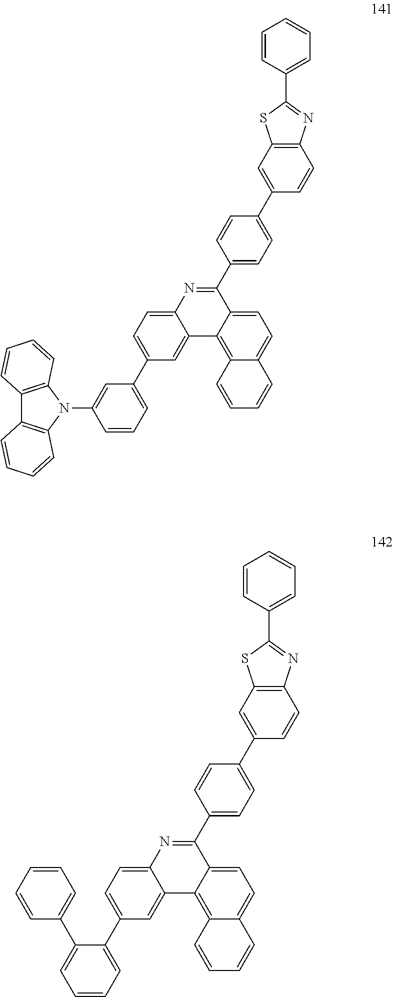 [SMILES: c1ccc(-c2nc3ccc(-c4ccc(-c5nc6ccc(-c7cccc(-n8c9ccccc9c9ccccc98)c7)cc6c6c5ccc5ccccc56)cc4)cc3s2)cc1 ; c1ccc(-c2nc3ccc(-c4ccc(-c5nc6ccc(-c7ccccc7-c7ccccc7)cc6c6c5ccc5ccccc56)cc4)cc3s2)cc1]

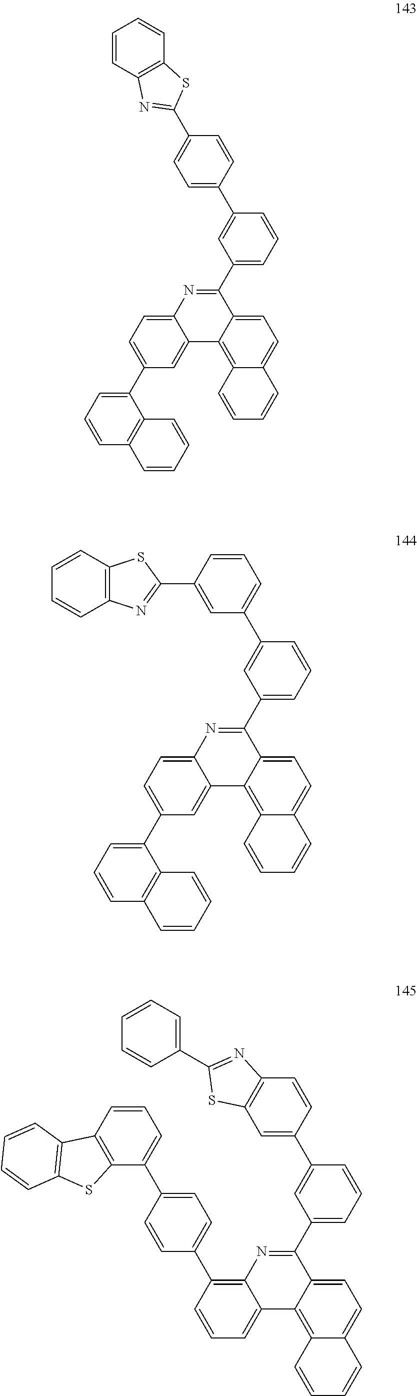 [SMILES: c1cc(-c2ccc(-c3nc4ccccc4s3)cc2)cc(-c2nc3ccc(-c4cccc5ccccc45)cc3c3c2ccc2ccccc23)c1 ; c1cc(-c2cccc(-c3nc4ccc(-c5cccc6ccccc56)cc4c4c3ccc3ccccc34)c2)cc(-c2nc3ccccc3s2)c1 ; c1ccc(-c2nc3ccc(-c4cccc(-c5nc6c(-c7ccc(-c8cccc9c8sc8ccccc89)cc7)cccc6c6c5ccc5ccccc56)c4)cc3s2)cc1]

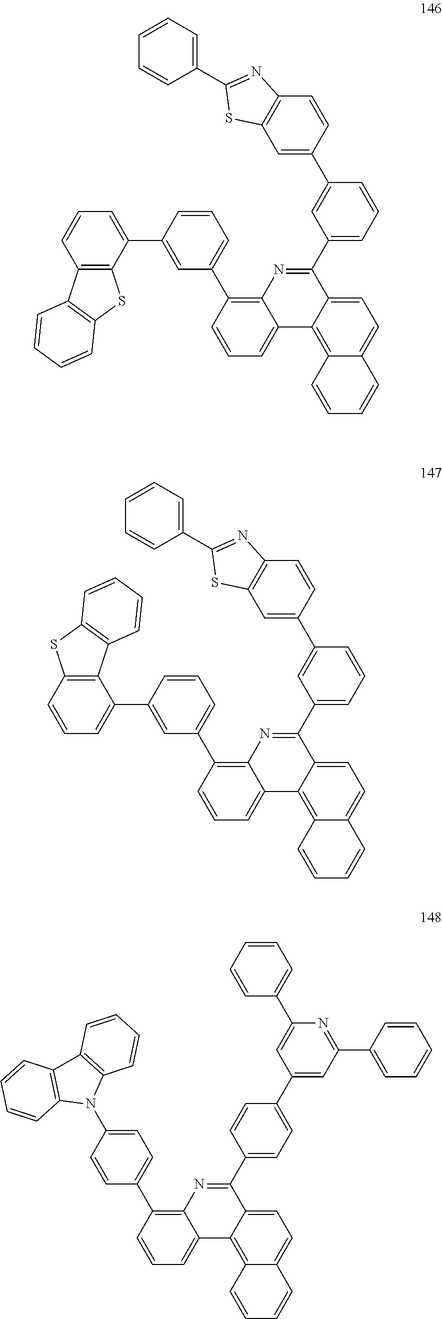 [SMILES: c1ccc(-c2nc3ccc(-c4cccc(-c5nc6c(-c7cccc(-c8cccc9c8sc8ccccc89)c7)cccc6c6c5ccc5ccccc56)c4)cc3s2)cc1 ; c1ccc(-c2nc3ccc(-c4cccc(-c5nc6c(-c7cccc(-c8cccc9sc%10ccccc%10c89)c7)cccc6c6c5ccc5ccccc56)c4)cc3s2)cc1 ; c1ccc(-c2cc(-c3ccc(-c4nc5c(-c6ccc(-n7c8ccccc8c8ccccc87)cc6)cccc5c5c4ccc4ccccc45)cc3)cc(-c3ccccc3)n2)cc1]

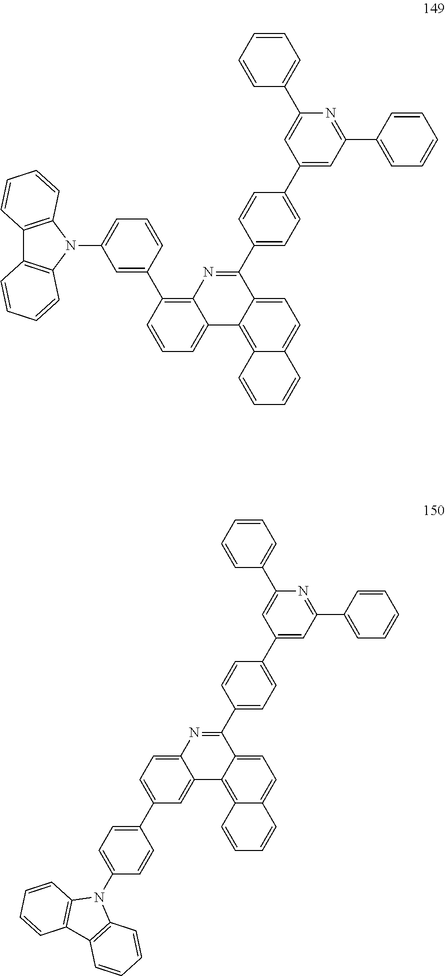 [SMILES: c1ccc(-c2cc(-c3ccc(-c4nc5c(-c6cccc(-n7c8ccccc8c8ccccc87)c6)cccc5c5c4ccc4ccccc45)cc3)cc(-c3ccccc3)n2)cc1 ; c1ccc(-c2cc(-c3ccc(-c4nc5ccc(-c6ccc(-n7c8ccccc8c8ccccc87)cc6)cc5c5c4ccc4ccccc45)cc3)cc(-c3ccccc3)n2)cc1]

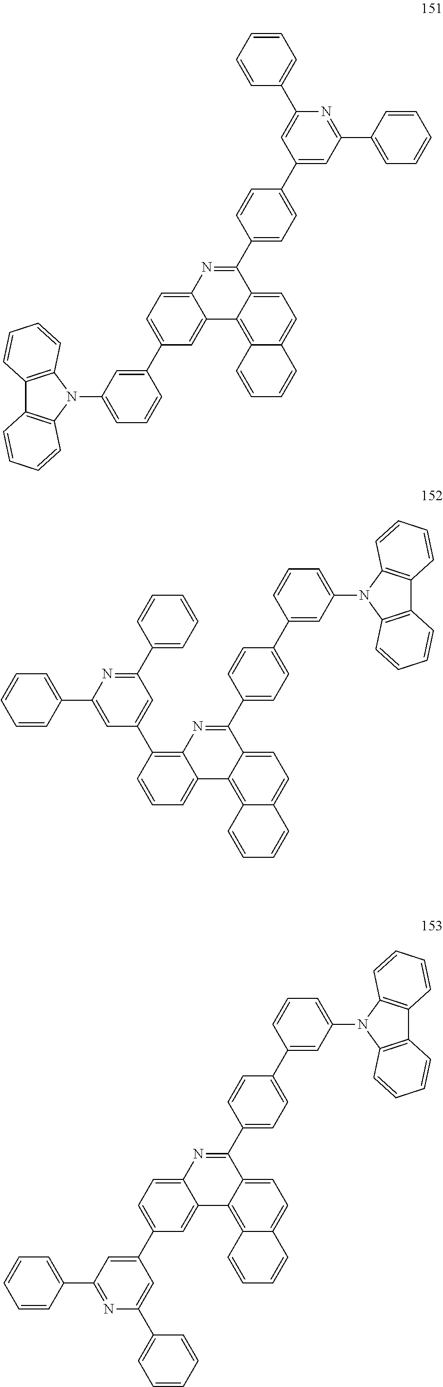 [SMILES: c1ccc(-c2cc(-c3ccc(-c4nc5ccc(-c6cccc(-n7c8ccccc8c8ccccc87)c6)cc5c5c4ccc4ccccc45)cc3)cc(-c3ccccc3)n2)cc1 ; c1ccc(-c2cc(-c3cccc4c3nc(-c3ccc(-c5cccc(-n6c7ccccc7c7ccccc76)c5)cc3)c3ccc5ccccc5c34)cc(-c3ccccc3)n2)cc1 ; c1ccc(-c2cc(-c3ccc4nc(-c5ccc(-c6cccc(-n7c8ccccc8c8ccccc87)c6)cc5)c5ccc6ccccc6c5c4c3)cc(-c3ccccc3)n2)cc1]

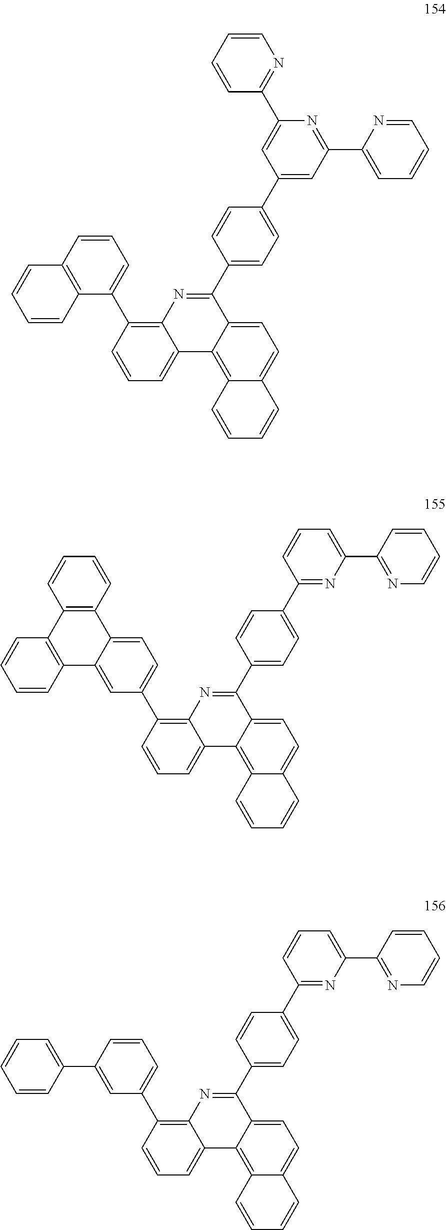 [SMILES: c1ccc(-c2cc(-c3ccc(-c4nc5c(-c6cccc7ccccc67)cccc5c5c4ccc4ccccc45)cc3)cc(-c3ccccn3)n2)nc1 ; c1ccc(-c2cccc(-c3ccc(-c4nc5c(-c6ccc7c8ccccc8c8ccccc8c7c6)cccc5c5c4ccc4ccccc45)cc3)n2)nc1 ; c1ccc(-c2cccc(-c3cccc4c3nc(-c3ccc(-c5cccc(-c6ccccn6)n5)cc3)c3ccc5ccccc5c34)c2)cc1]

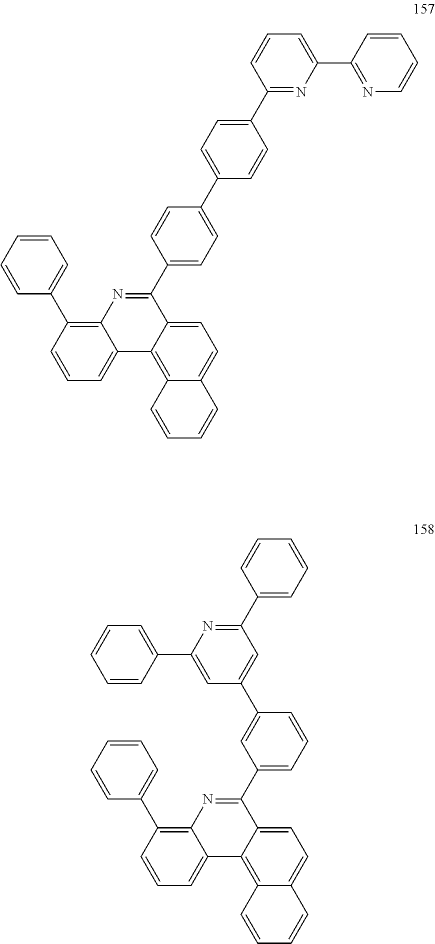 [SMILES: c1ccc(-c2cccc3c2nc(-c2ccc(-c4ccc(-c5cccc(-c6ccccn6)n5)cc4)cc2)c2ccc4ccccc4c23)cc1 ; c1ccc(-c2cc(-c3cccc(-c4nc5c(-c6ccccc6)cccc5c5c4ccc4ccccc45)c3)cc(-c3ccccc3)n2)cc1]

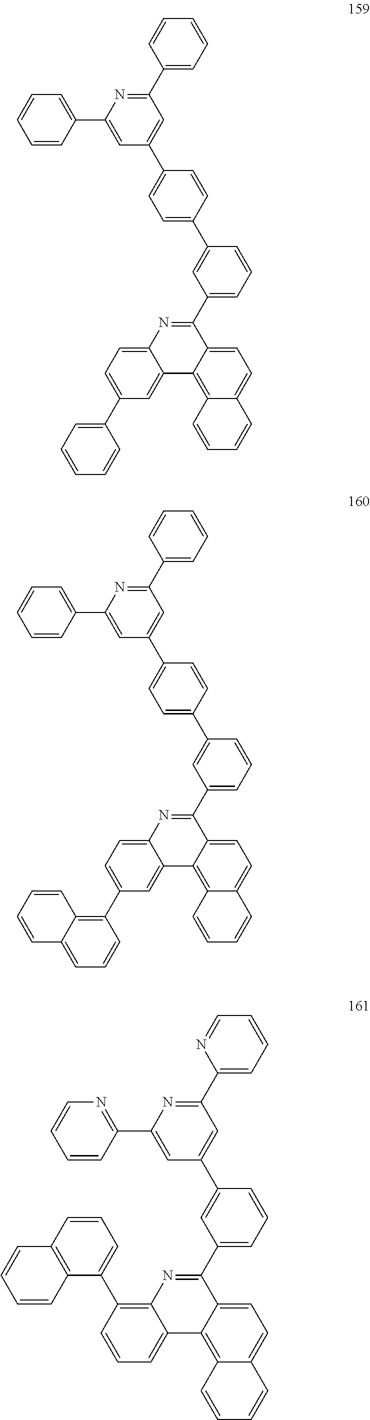 [SMILES: c1ccc(-c2ccc3nc(-c4cccc(-c5ccc(-c6cc(-c7ccccc7)nc(-c7ccccc7)c6)cc5)c4)c4ccc5ccccc5c4c3c2)cc1 ; c1ccc(-c2cc(-c3ccc(-c4cccc(-c5nc6ccc(-c7cccc8ccccc78)cc6c6c5ccc5ccccc56)c4)cc3)cc(-c3ccccc3)n2)cc1 ; c1ccc(-c2cc(-c3cccc(-c4nc5c(-c6cccc7ccccc67)cccc5c5c4ccc4ccccc45)c3)cc(-c3ccccn3)n2)nc1]

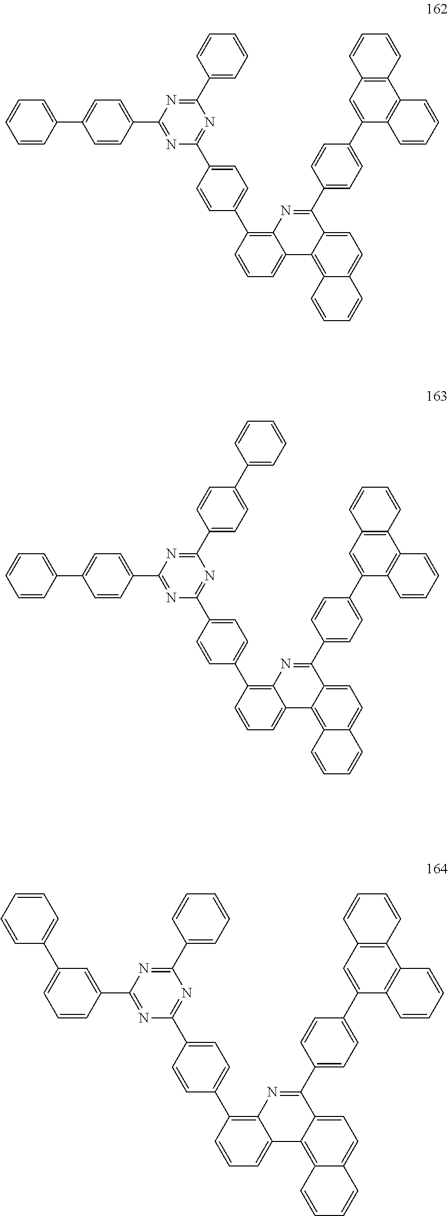 [SMILES: c1ccc(-c2ccc(-c3nc(-c4ccccc4)nc(-c4ccc(-c5cccc6c5nc(-c5ccc(-c7cc8ccccc8c8ccccc78)cc5)c5ccc7ccccc7c56)cc4)n3)cc2)cc1 ; c1ccc(-c2ccc(-c3nc(-c4ccc(-c5ccccc5)cc4)nc(-c4ccc(-c5cccc6c5nc(-c5ccc(-c7cc8ccccc8c8ccccc78)cc5)c5ccc7ccccc7c56)cc4)n3)cc2)cc1 ; c1ccc(-c2cccc(-c3nc(-c4ccccc4)nc(-c4ccc(-c5cccc6c5nc(-c5ccc(-c7cc8ccccc8c8ccccc78)cc5)c5ccc7ccccc7c56)cc4)n3)c2)cc1]

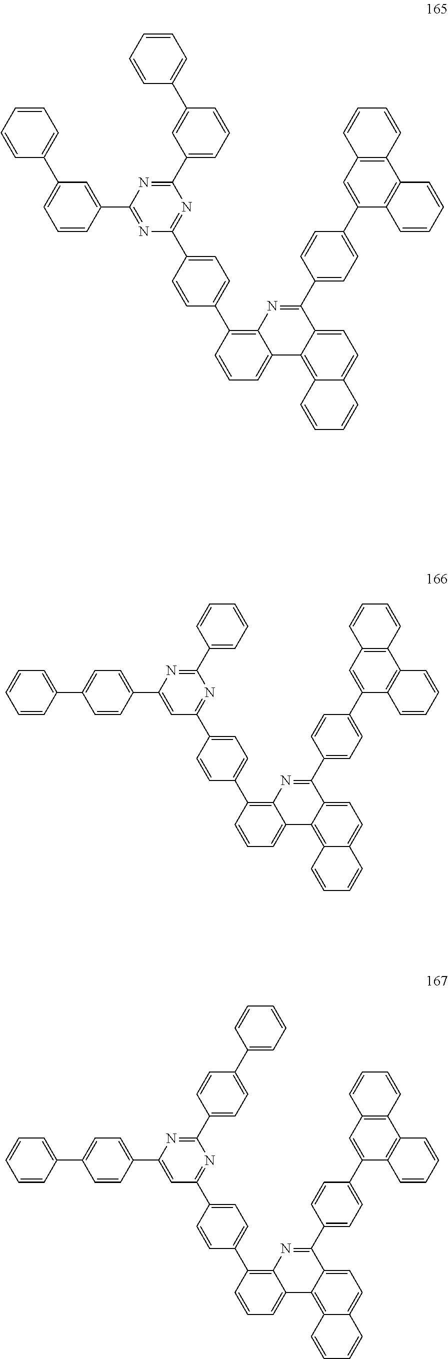 [SMILES: c1ccc(-c2cccc(-c3nc(-c4ccc(-c5cccc6c5nc(-c5ccc(-c7cc8ccccc8c8ccccc78)cc5)c5ccc7ccccc7c56)cc4)nc(-c4cccc(-c5ccccc5)c4)n3)c2)cc1 ; c1ccc(-c2ccc(-c3cc(-c4ccc(-c5cccc6c5nc(-c5ccc(-c7cc8ccccc8c8ccccc78)cc5)c5ccc7ccccc7c56)cc4)nc(-c4ccccc4)n3)cc2)cc1 ; c1ccc(-c2ccc(-c3cc(-c4ccc(-c5cccc6c5nc(-c5ccc(-c7cc8ccccc8c8ccccc78)cc5)c5ccc7ccccc7c56)cc4)nc(-c4ccc(-c5ccccc5)cc4)n3)cc2)cc1]

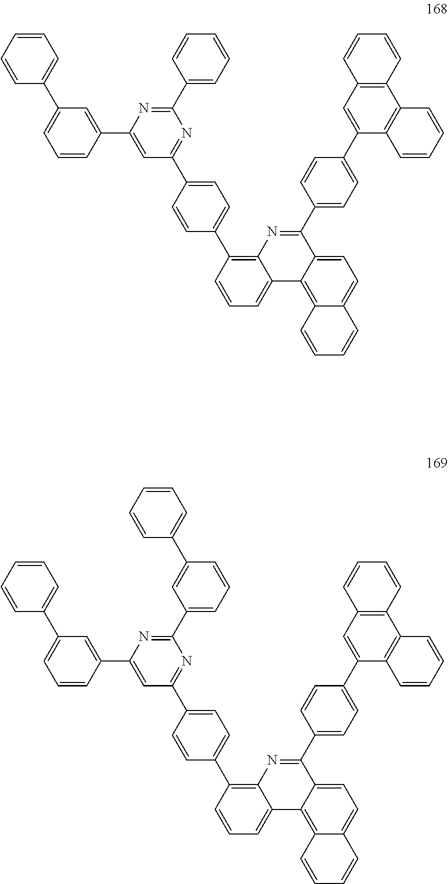 [SMILES: c1ccc(-c2cccc(-c3cc(-c4ccc(-c5cccc6c5nc(-c5ccc(-c7cc8ccccc8c8ccccc78)cc5)c5ccc7ccccc7c56)cc4)nc(-c4ccccc4)n3)c2)cc1 ; c1ccc(-c2cccc(-c3cc(-c4ccc(-c5cccc6c5nc(-c5ccc(-c7cc8ccccc8c8ccccc78)cc5)c5ccc7ccccc7c56)cc4)nc(-c4cccc(-c5ccccc5)c4)n3)c2)cc1]

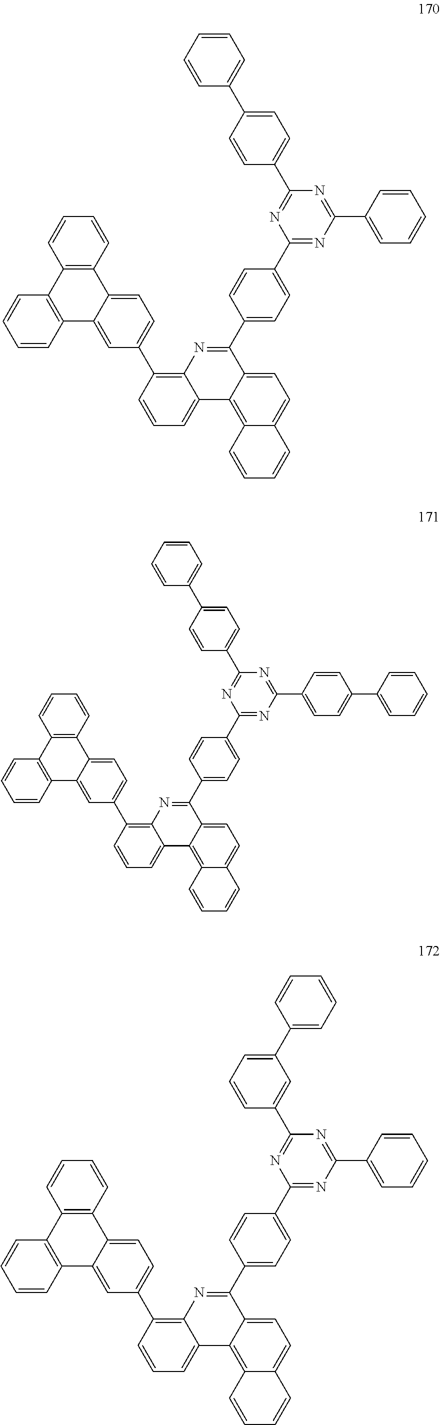 [SMILES: c1ccc(-c2ccc(-c3nc(-c4ccccc4)nc(-c4ccc(-c5nc6c(-c7ccc8c9ccccc9c9ccccc9c8c7)cccc6c6c5ccc5ccccc56)cc4)n3)cc2)cc1 ; c1ccc(-c2ccc(-c3nc(-c4ccc(-c5ccccc5)cc4)nc(-c4ccc(-c5nc6c(-c7ccc8c9ccccc9c9ccccc9c8c7)cccc6c6c5ccc5ccccc56)cc4)n3)cc2)cc1 ; c1ccc(-c2cccc(-c3nc(-c4ccccc4)nc(-c4ccc(-c5nc6c(-c7ccc8c9ccccc9c9ccccc9c8c7)cccc6c6c5ccc5ccccc56)cc4)n3)c2)cc1]

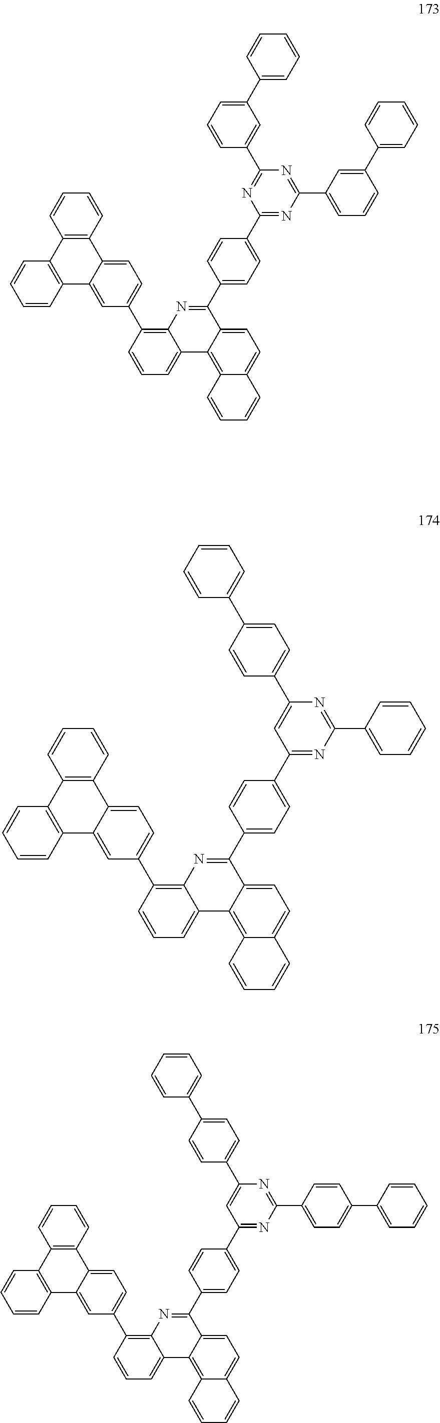 [SMILES: c1ccc(-c2cccc(-c3nc(-c4ccc(-c5nc6c(-c7ccc8c9ccccc9c9ccccc9c8c7)cccc6c6c5ccc5ccccc56)cc4)nc(-c4cccc(-c5ccccc5)c4)n3)c2)cc1 ; c1ccc(-c2ccc(-c3cc(-c4ccc(-c5nc6c(-c7ccc8c9ccccc9c9ccccc9c8c7)cccc6c6c5ccc5ccccc56)cc4)nc(-c4ccccc4)n3)cc2)cc1 ; c1ccc(-c2ccc(-c3cc(-c4ccc(-c5nc6c(-c7ccc8c9ccccc9c9ccccc9c8c7)cccc6c6c5ccc5ccccc56)cc4)nc(-c4ccc(-c5ccccc5)cc4)n3)cc2)cc1]

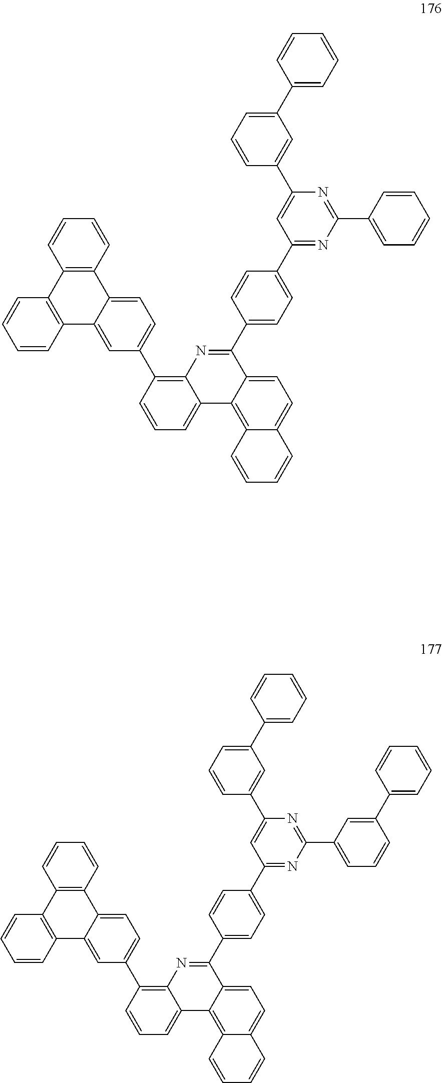 [SMILES: c1ccc(-c2cccc(-c3cc(-c4ccc(-c5nc6c(-c7ccc8c9ccccc9c9ccccc9c8c7)cccc6c6c5ccc5ccccc56)cc4)nc(-c4ccccc4)n3)c2)cc1 ; c1ccc(-c2cccc(-c3cc(-c4ccc(-c5nc6c(-c7ccc8c9ccccc9c9ccccc9c8c7)cccc6c6c5ccc5ccccc56)cc4)nc(-c4cccc(-c5ccccc5)c4)n3)c2)cc1]

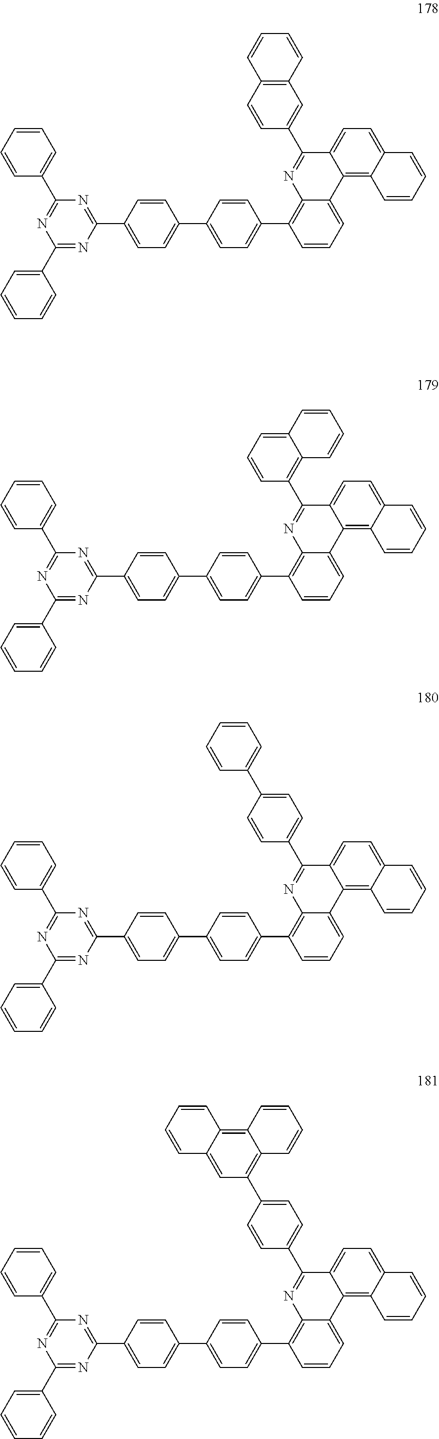 [SMILES: c1ccc(-c2nc(-c3ccccc3)nc(-c3ccc(-c4ccc(-c5cccc6c5nc(-c5ccc7ccccc7c5)c5ccc7ccccc7c56)cc4)cc3)n2)cc1 ; c1ccc(-c2nc(-c3ccccc3)nc(-c3ccc(-c4ccc(-c5cccc6c5nc(-c5cccc7ccccc57)c5ccc7ccccc7c56)cc4)cc3)n2)cc1 ; c1ccc(-c2ccc(-c3nc4c(-c5ccc(-c6ccc(-c7nc(-c8ccccc8)nc(-c8ccccc8)n7)cc6)cc5)cccc4c4c3ccc3ccccc34)cc2)cc1 ; c1ccc(-c2nc(-c3ccccc3)nc(-c3ccc(-c4ccc(-c5cccc6c5nc(-c5ccc(-c7cc8ccccc8c8ccccc78)cc5)c5ccc7ccccc7c56)cc4)cc3)n2)cc1]

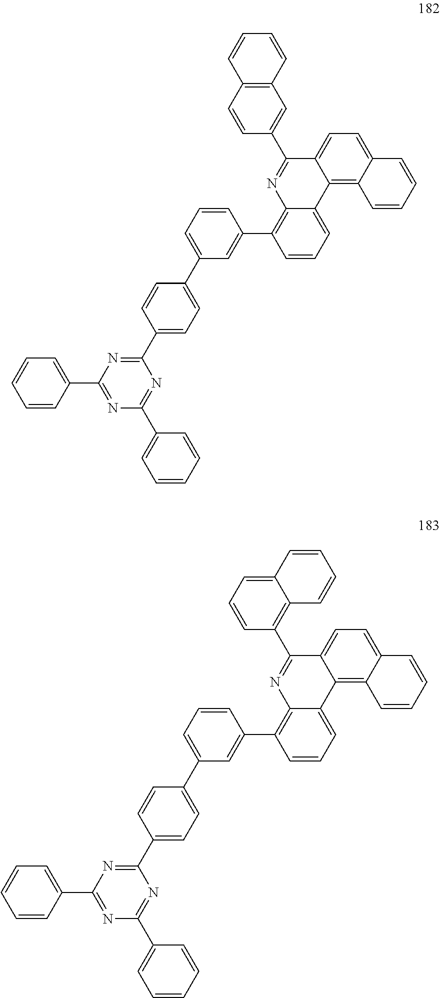 [SMILES: c1ccc(-c2nc(-c3ccccc3)nc(-c3ccc(-c4cccc(-c5cccc6c5nc(-c5ccc7ccccc7c5)c5ccc7ccccc7c56)c4)cc3)n2)cc1 ; c1ccc(-c2nc(-c3ccccc3)nc(-c3ccc(-c4cccc(-c5cccc6c5nc(-c5cccc7ccccc57)c5ccc7ccccc7c56)c4)cc3)n2)cc1]

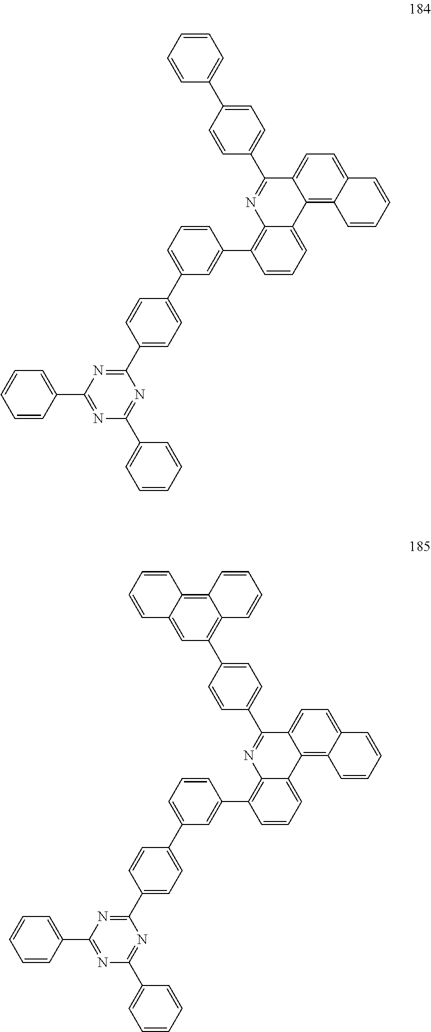 [SMILES: c1ccc(-c2ccc(-c3nc4c(-c5cccc(-c6ccc(-c7nc(-c8ccccc8)nc(-c8ccccc8)n7)cc6)c5)cccc4c4c3ccc3ccccc34)cc2)cc1 ; c1ccc(-c2nc(-c3ccccc3)nc(-c3ccc(-c4cccc(-c5cccc6c5nc(-c5ccc(-c7cc8ccccc8c8ccccc78)cc5)c5ccc7ccccc7c56)c4)cc3)n2)cc1]

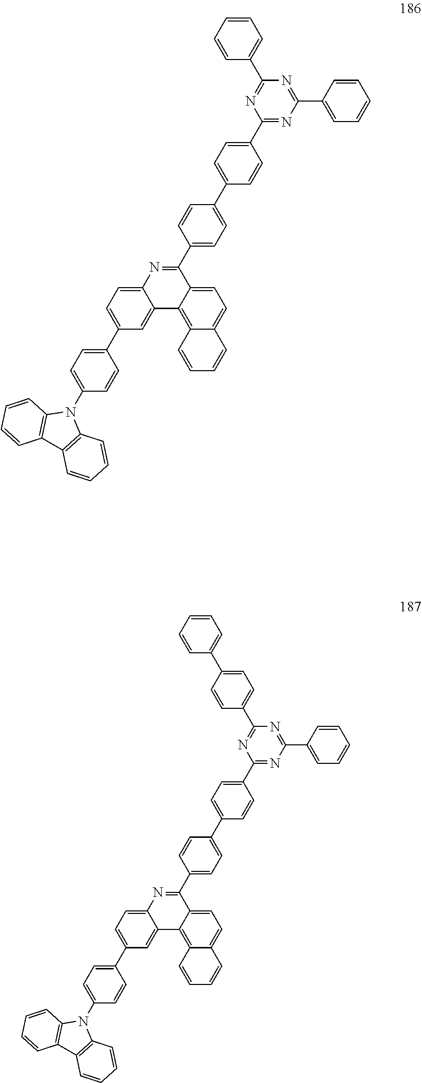 [SMILES: c1ccc(-c2nc(-c3ccccc3)nc(-c3ccc(-c4ccc(-c5nc6ccc(-c7ccc(-n8c9ccccc9c9ccccc98)cc7)cc6c6c5ccc5ccccc56)cc4)cc3)n2)cc1 ; c1ccc(-c2ccc(-c3nc(-c4ccccc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)cc2)cc1]

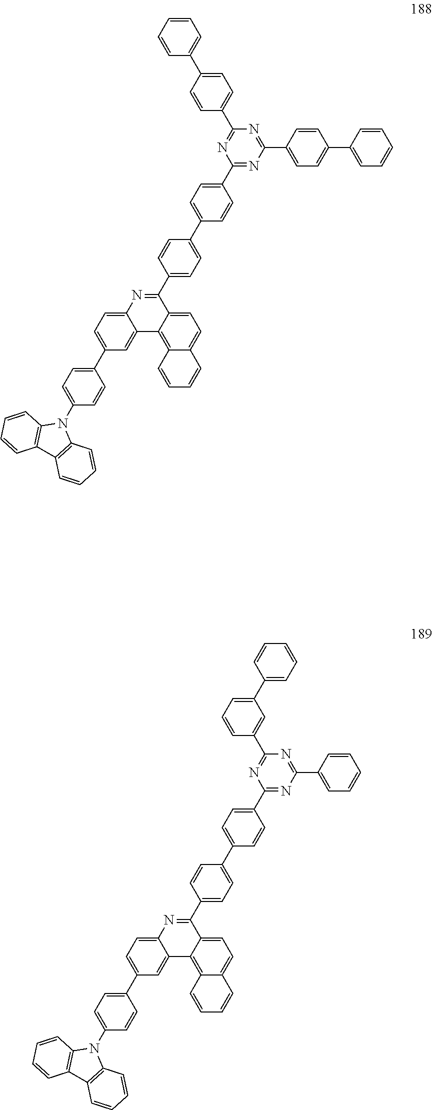 [SMILES: c1ccc(-c2ccc(-c3nc(-c4ccc(-c5ccccc5)cc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)cc2)cc1 ; c1ccc(-c2cccc(-c3nc(-c4ccccc4)nc(-c4ccc(-c5ccc(-c6nc7ccc(-c8ccc(-n9c%10ccccc%10c%10ccccc%109)cc8)cc7c7c6ccc6ccccc67)cc5)cc4)n3)c2)cc1]

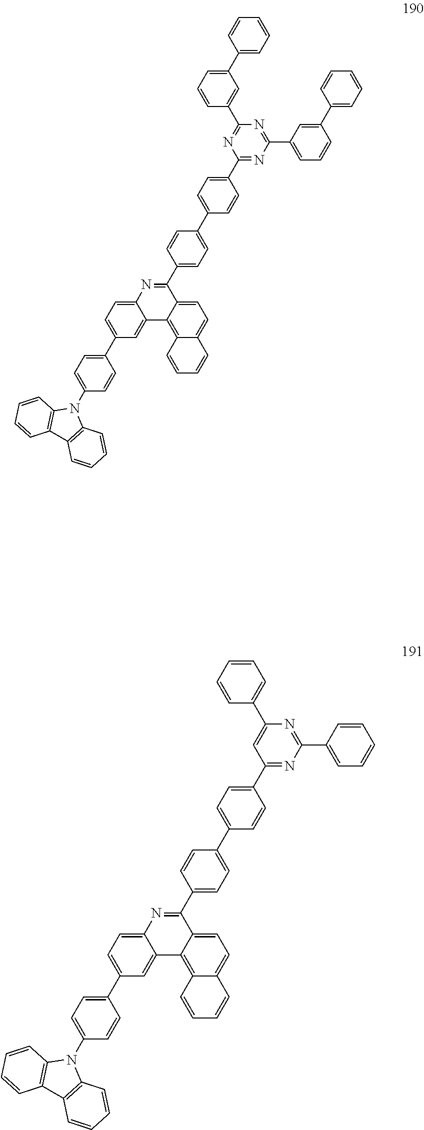 [SMILES: c1ccc(-c2cccc(-c3nc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4cccc(-c5ccccc5)c4)n3)c2)cc1 ; c1ccc(-c2cc(-c3ccc(-c4ccc(-c5nc6ccc(-c7ccc(-n8c9ccccc9c9ccccc98)cc7)cc6c6c5ccc5ccccc56)cc4)cc3)nc(-c3ccccc3)n2)cc1]

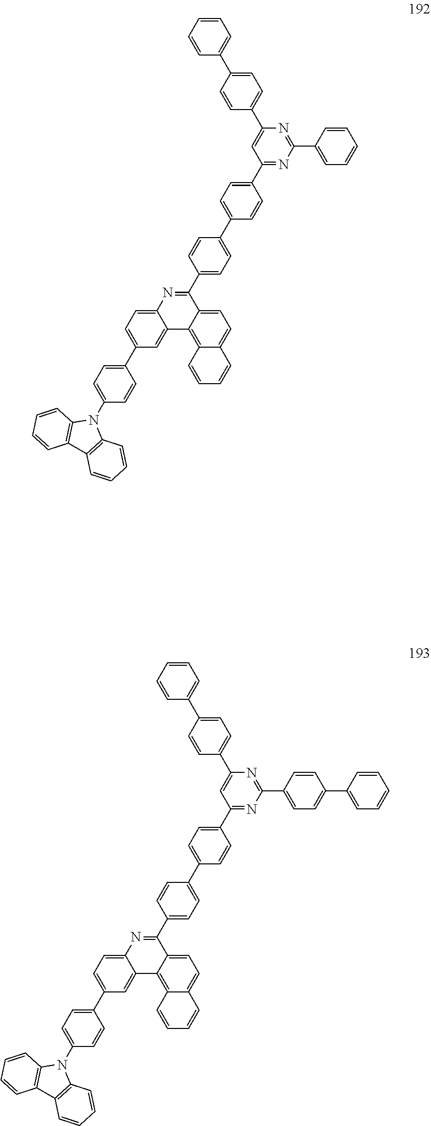 [SMILES: c1ccc(-c2ccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccccc4)n3)cc2)cc1 ; c1ccc(-c2ccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccc(-c5ccccc5)cc4)n3)cc2)cc1]

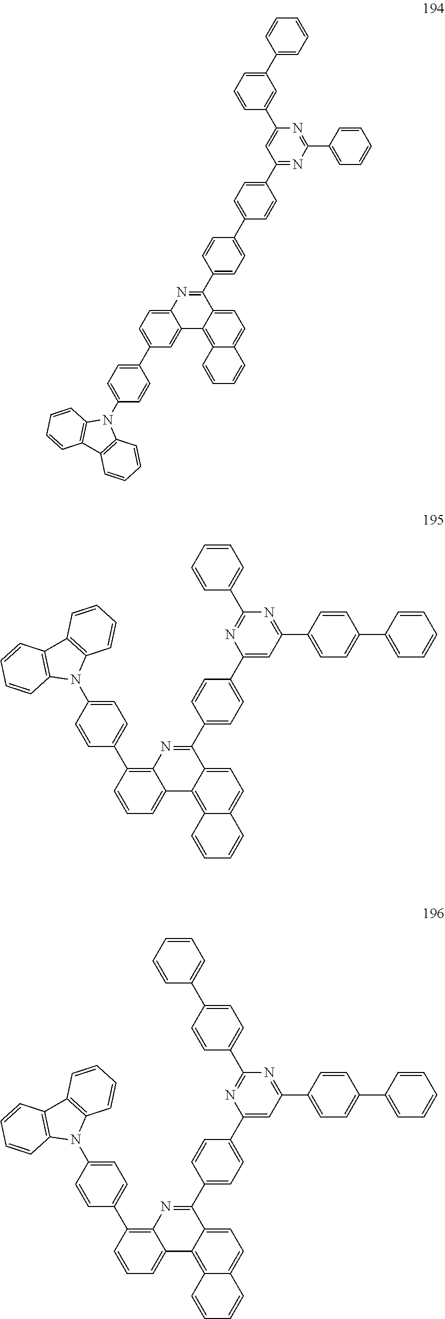 [SMILES: c1ccc(-c2cccc(-c3cc(-c4ccc(-c5ccc(-c6nc7ccc(-c8ccc(-n9c%10ccccc%10c%10ccccc%109)cc8)cc7c7c6ccc6ccccc67)cc5)cc4)nc(-c4ccccc4)n3)c2)cc1 ; c1ccc(-c2ccc(-c3cc(-c4ccc(-c5nc6c(-c7ccc(-n8c9ccccc9c9ccccc98)cc7)cccc6c6c5ccc5ccccc56)cc4)nc(-c4ccccc4)n3)cc2)cc1 ; c1ccc(-c2ccc(-c3cc(-c4ccc(-c5nc6c(-c7ccc(-n8c9ccccc9c9ccccc98)cc7)cccc6c6c5ccc5ccccc56)cc4)nc(-c4ccc(-c5ccccc5)cc4)n3)cc2)cc1]

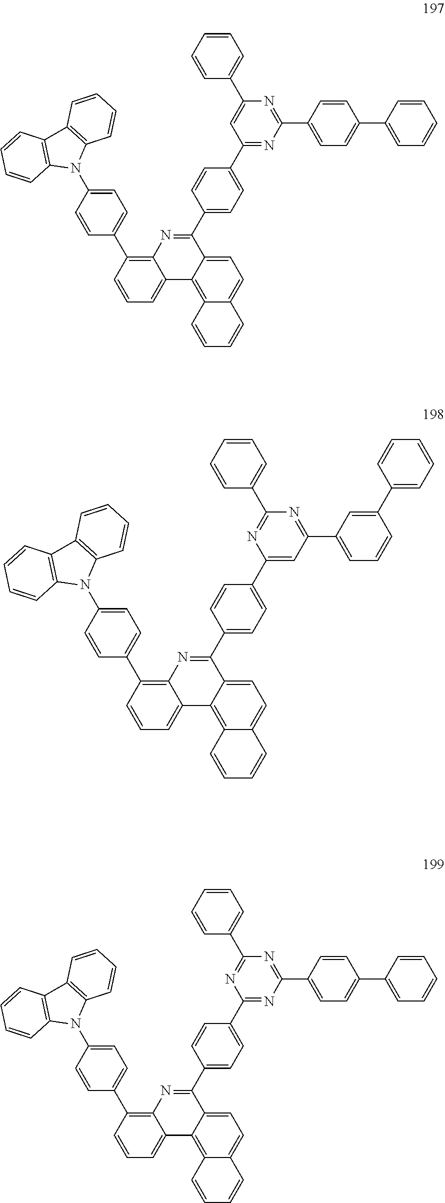 [SMILES: c1ccc(-c2ccc(-c3nc(-c4ccccc4)cc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2cccc(-c3cc(-c4ccc(-c5nc6c(-c7ccc(-n8c9ccccc9c9ccccc98)cc7)cccc6c6c5ccc5ccccc56)cc4)nc(-c4ccccc4)n3)c2)cc1 ; c1ccc(-c2ccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)cc2)cc1]

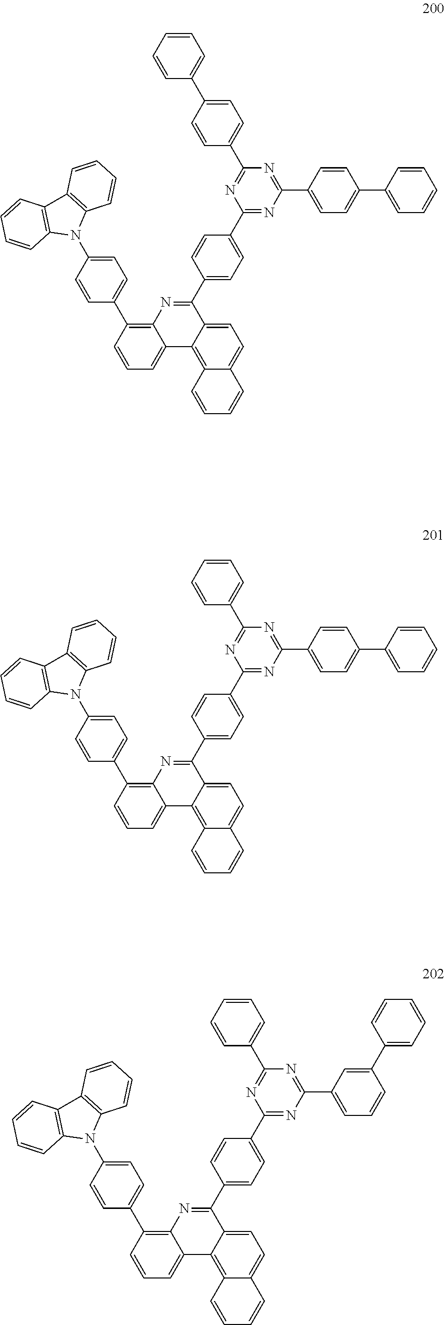 [SMILES: c1ccc(-c2ccc(-c3nc(-c4ccc(-c5ccccc5)cc4)nc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2ccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)cc2)cc1 ; c1ccc(-c2cccc(-c3nc(-c4ccccc4)nc(-c4ccc(-c5nc6c(-c7ccc(-n8c9ccccc9c9ccccc98)cc7)cccc6c6c5ccc5ccccc56)cc4)n3)c2)cc1]

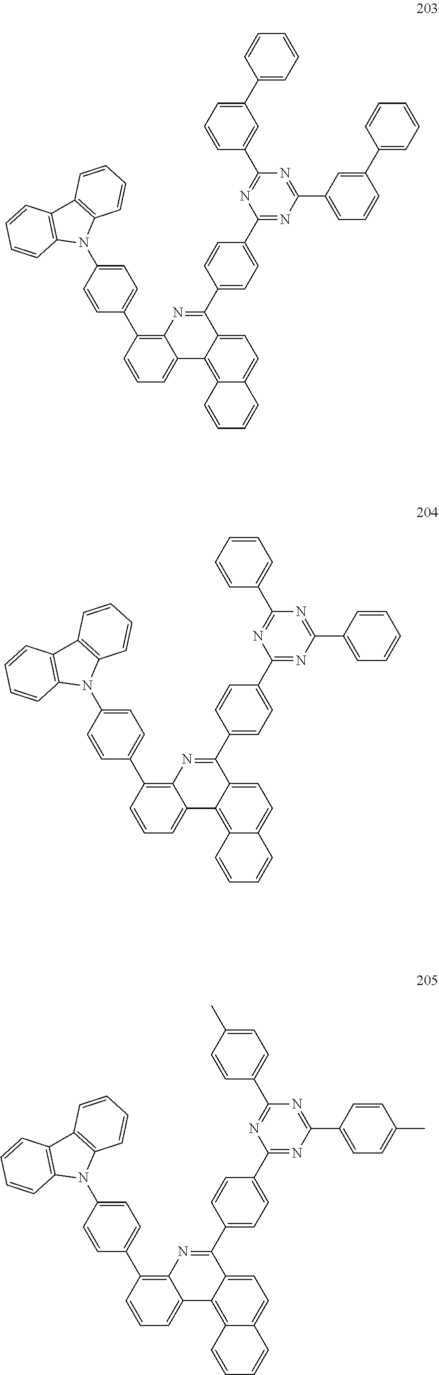 [SMILES: c1ccc(-c2cccc(-c3nc(-c4ccc(-c5nc6c(-c7ccc(-n8c9ccccc9c9ccccc98)cc7)cccc6c6c5ccc5ccccc56)cc4)nc(-c4cccc(-c5ccccc5)c4)n3)c2)cc1 ; c1ccc(-c2nc(-c3ccccc3)nc(-c3ccc(-c4nc5c(-c6ccc(-n7c8ccccc8c8ccccc87)cc6)cccc5c5c4ccc4ccccc45)cc3)n2)cc1 ; Cc1ccc(-c2nc(-c3ccc(C)cc3)nc(-c3ccc(-c4nc5c(-c6ccc(-n7c8ccccc8c8ccccc87)cc6)cccc5c5c4ccc4ccccc45)cc3)n2)cc1]

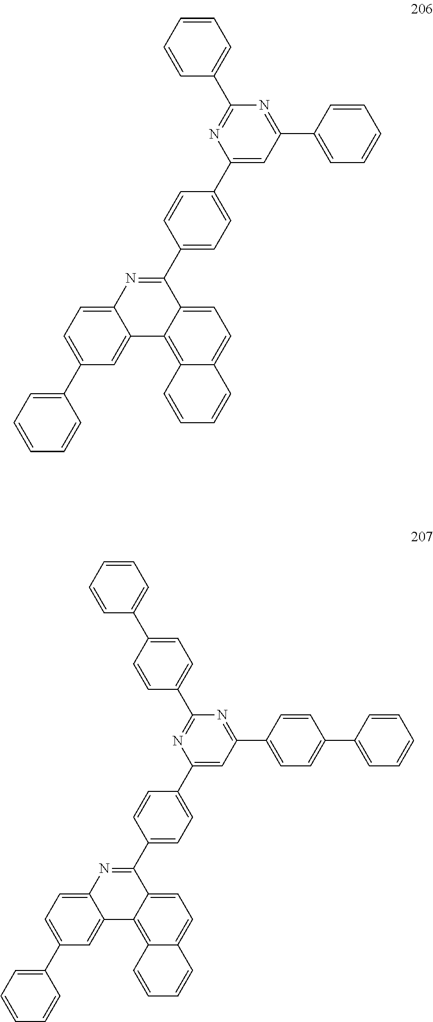 [SMILES: c1ccc(-c2ccc3nc(-c4ccc(-c5cc(-c6ccccc6)nc(-c6ccccc6)n5)cc4)c4ccc5ccccc5c4c3c2)cc1 ; c1ccc(-c2ccc(-c3cc(-c4ccc(-c5nc6ccc(-c7ccccc7)cc6c6c5ccc5ccccc56)cc4)nc(-c4ccc(-c5ccccc5)cc4)n3)cc2)cc1]

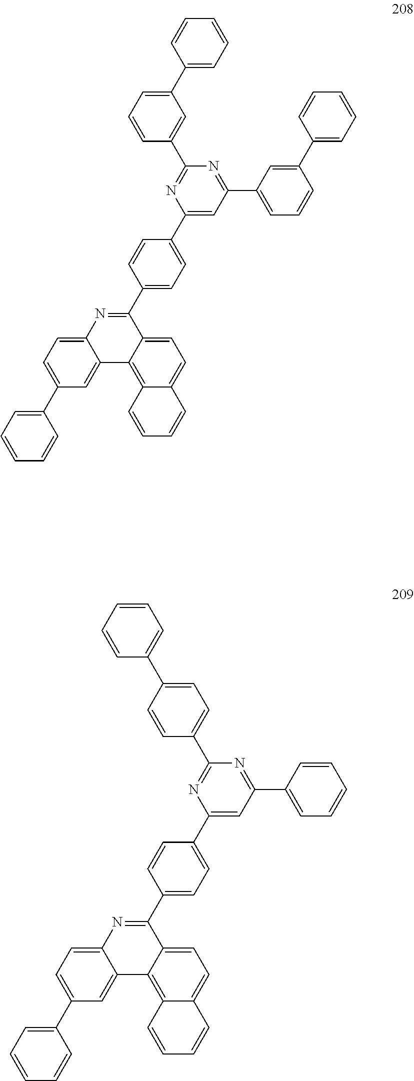 [SMILES: c1ccc(-c2cccc(-c3cc(-c4ccc(-c5nc6ccc(-c7ccccc7)cc6c6c5ccc5ccccc56)cc4)nc(-c4cccc(-c5ccccc5)c4)n3)c2)cc1 ; c1ccc(-c2ccc(-c3nc(-c4ccccc4)cc(-c4ccc(-c5nc6ccc(-c7ccccc7)cc6c6c5ccc5ccccc56)cc4)n3)cc2)cc1]

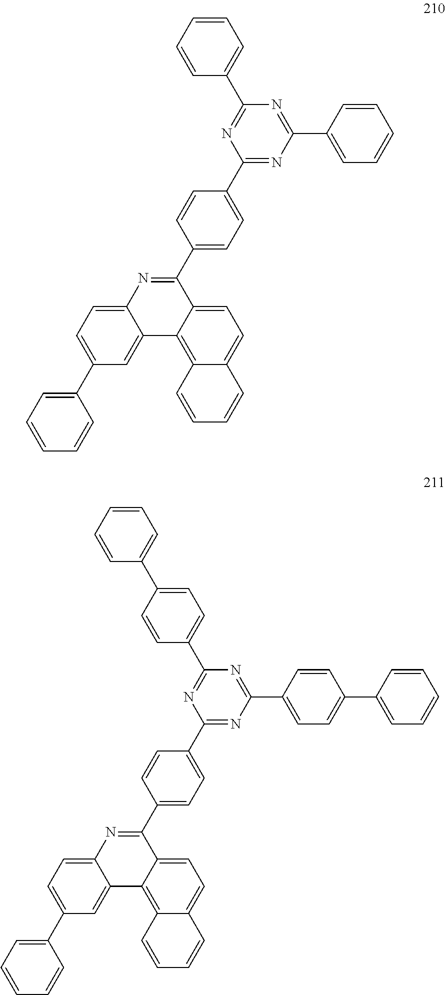 [SMILES: c1ccc(-c2ccc3nc(-c4ccc(-c5nc(-c6ccccc6)nc(-c6ccccc6)n5)cc4)c4ccc5ccccc5c4c3c2)cc1 ; c1ccc(-c2ccc(-c3nc(-c4ccc(-c5ccccc5)cc4)nc(-c4ccc(-c5nc6ccc(-c7ccccc7)cc6c6c5ccc5ccccc56)cc4)n3)cc2)cc1]

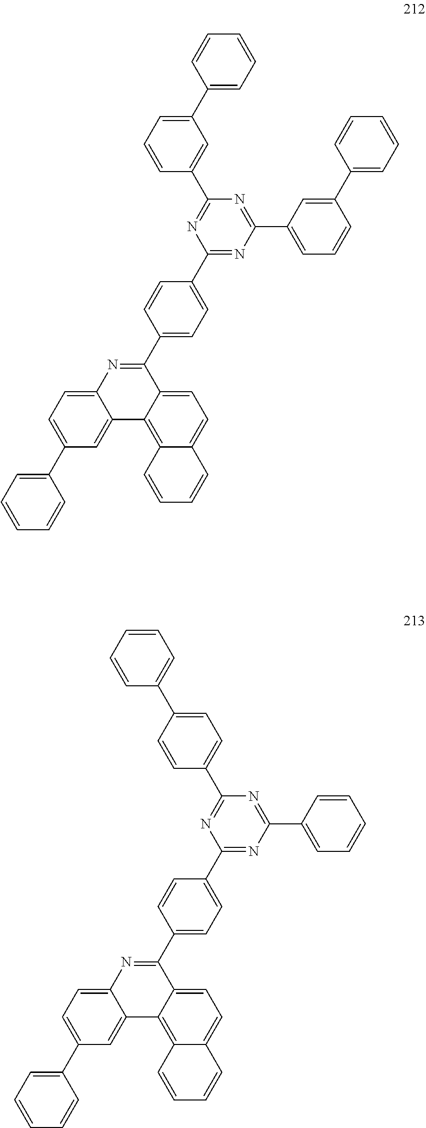 [SMILES: c1ccc(-c2cccc(-c3nc(-c4ccc(-c5nc6ccc(-c7ccccc7)cc6c6c5ccc5ccccc56)cc4)nc(-c4cccc(-c5ccccc5)c4)n3)c2)cc1 ; c1ccc(-c2ccc(-c3nc(-c4ccccc4)nc(-c4ccc(-c5nc6ccc(-c7ccccc7)cc6c6c5ccc5ccccc56)cc4)n3)cc2)cc1]

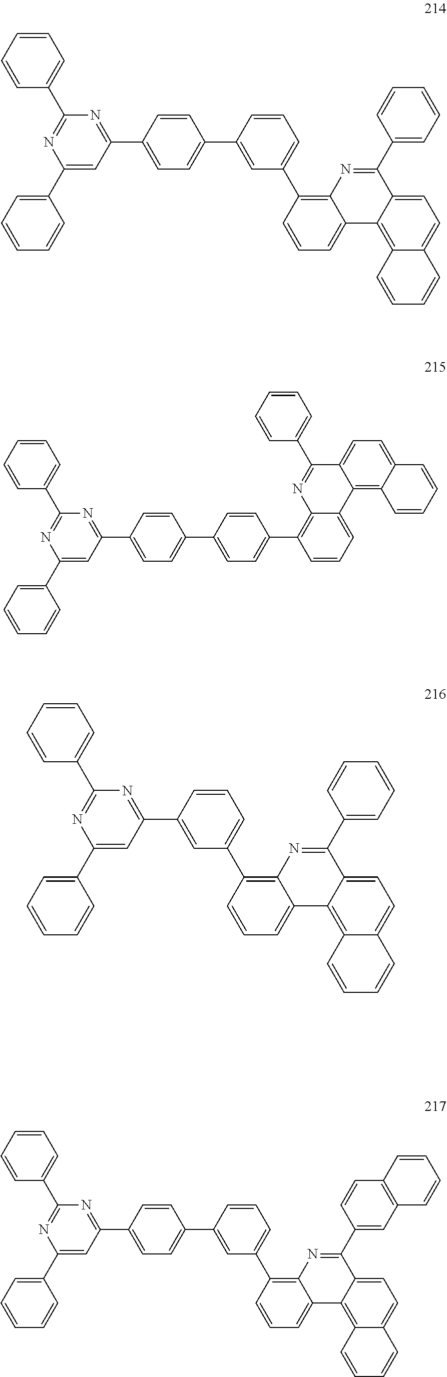 [SMILES: c1ccc(-c2cc(-c3ccc(-c4cccc(-c5cccc6c5nc(-c5ccccc5)c5ccc7ccccc7c56)c4)cc3)nc(-c3ccccc3)n2)cc1 ; c1ccc(-c2cc(-c3ccc(-c4ccc(-c5cccc6c5nc(-c5ccccc5)c5ccc7ccccc7c56)cc4)cc3)nc(-c3ccccc3)n2)cc1 ; c1ccc(-c2cc(-c3cccc(-c4cccc5c4nc(-c4ccccc4)c4ccc6ccccc6c45)c3)nc(-c3ccccc3)n2)cc1 ; c1ccc(-c2cc(-c3ccc(-c4cccc(-c5cccc6c5nc(-c5ccc7ccccc7c5)c5ccc7ccccc7c56)c4)cc3)nc(-c3ccccc3)n2)cc1]

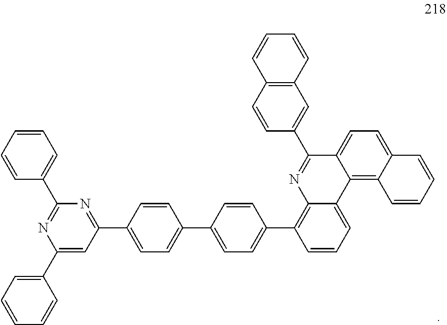 [SMILES: c1ccc(-c2cc(-c3ccc(-c4ccc(-c5cccc6c5nc(-c5ccc7ccccc7c5)c5ccc7ccccc7c56)cc4)cc3)nc(-c3ccccc3)n2)cc1]

8. An organic light emitting device comprising:
a first electrode;
a second electrode provided opposite to the first electrode; and
one of more organic material layers provided between the first electrode and the second electrode,
wherein one or more layers of the organic material layers comprise the heterocyclic compound of claim 1.

9. The organic light emitting device of claim 8, wherein the organic material layer comprises a light emitting layer, and the light emitting layer comprises the heterocyclic compound.

10. The organic light emitting device of claim 8, wherein the organic material layer comprises an electron injection layer or an electron transfer layer, and the electron injection layer or the electron transfer layer comprises the heterocyclic compound.

11. The organic light emitting device of claim 8, wherein the organic material layer comprises an electron blocking layer or a hole blocking layer, and the electron blocking layer or the hole blocking layer comprises the heterocyclic compound.

12. The organic light emitting device of claim 8, further comprising one, two or more layers selected from the group consisting of a light emitting layer, a hole injection layer, a hole transfer layer, an electron injection layer, an electron transfer layer, an electron blocking layer and a hole blocking layer.

13. The organic light emitting device of claim 8, comprising:
a first electrode;
a first stack provided on the first electrode and comprising a first light emitting layer;
a charge generation layer provided on the first stack;
a second stack provided on the charge generation layer and comprising a second light emitting layer; and
a second electrode provided on the second stack.

14. The organic light emitting device of claim 13, wherein the charge generation layer comprises the heterocyclic compound.

15. The organic light emitting device of claim 13, wherein the charge generation layer is an N-type charge generation layer, and the charge generation layer comprises the heterocyclic compound.
